(19) United States
(12) Patent Application Publication
(10) Pub. No.: US 2025/0011774 A1
(43) Pub. Date: Jan. 9, 2025

(54) ANTISENSE COMPOUNDS AND METHODS FOR TARGETING CUG REPEATS

(71) Applicant: ENTRADA THERAPEUTICS, INC., Boston, MA (US)

(72) Inventors: Xiulong SHEN, Waltham, MA (US); Ziqing QIAN, Wellesley, MA (US); Patrick DOUGHERTY, Independence, OH (US); Mahboubeh KHEIRABADI, Natick, MA (US); Xiang LI, Belmont, MA (US)

(21) Appl. No.: 18/390,717

(22) Filed: Dec. 20, 2023

Related U.S. Application Data
(63) Continuation of application No. PCT/US2022/034517, filed on Jun. 22, 2022.
(60) Provisional application No. 63/213,900, filed on Jun. 23, 2021.
(60) Provisional application No. 63/239,671, filed on Sep. 1, 2021.
(60) Provisional application No. 63/239,847, filed on Sep. 1, 2021.
(60) Provisional application No. 63/290,892, filed on Dec. 17, 2021.
(60) Provisional application No. 63/290,960, filed on Dec. 17, 2021.
(60) Provisional application No. 63/298,565, filed on Jan. 11, 2022.
(60) Provisional application No. 63/305,071, filed on Jan. 31, 2022.
(60) Provisional application No. 63/268,577, filed on Feb. 25, 2022.
(60) Provisional application No. 63/314,369, filed on Feb. 26, 2022.
(60) Provisional application No. 63/316,634, filed on Mar. 4, 2022.
(60) Provisional application No. 63/317,856, filed on Mar. 8, 2022.
(60) Provisional application No. 63/326,201, filed on Mar. 31, 2022.
(60) Provisional application No. 63/327,179, filed on Apr. 4, 2022.
(60) Provisional application No. 63/339,250, filed on May 6, 2022.

(51) Int. Cl. C12N 15/113; C07K 7/64

(52) U.S. Cl. CPC C12N 15/113; C07K 7/64; C12N 2310/11; C12N 2310/3233; C12N 2310/3513

(57) ABSTRACT

Compounds comprising a cyclic peptide, such as a cyclic cell penetrating peptide, and an antisense compound are provided. The antisense compound binds to a gene having an expanded CTG repeat or a gene transcript having an expanded CUG repeat. The compounds can be delivered to subjects to treat diseases associated with expanded CTG CUG repeats, such as myotonic dystrophy type 1 (DM1), spinocerebellar ataxia-8 (SCA8), and Huntington disease like-2 (HDL2).

### CROSS REFERENCE TO RELATED APPLICATIONS

This application claims the benefit of U.S. Provisional Application Ser. No. 63/213,900, filed on Jun. 23, 2021; 63/239,847, filed on Sep. 1, 2021; 63/290,892, filed on Dec. 17, 2021; 63/305,071, filed on Jan. 31, 2022; 63/314,369, filed on Feb. 26, 2022; 63/316,634, filed on Mar. 4, 2022; 63/317,856, filed on Mar. 8, 2022; 63/326,201, filed on Mar. 31, 2022; 63/327,179, filed on Apr. 4, 2022; 63/339,250, filed on May 6, 2022; 63/362,295, filed on Mar. 31, 2022; 63/239,671, filed on Sep. 1, 2021; 63/290,960, filed on Dec. 17, 2021; 63/298,565, filed on Jan. 11, 2022; and 63/268,577, filed on Feb. 25, 2022.

### SEQUENCE LISTING

The instant application contains a Sequence Listing which has been submitted electronically in XML format and is hereby incorporated by reference in its entirety. Said XML copy, created on Mar. 26, 2024, is named 0755_000040US01_ST26.xml and is 1,194,543 bytes in size.

### FIELD

The present disclosure relates to compounds, compositions, and methods for modulating the activity and/or levels of genes that include expanded nucleotide repeats, in particular expanded CTG·CUG repeats. The compounds and compositions containing the same may be used to treat diseases associated with genes that include an expanded nucleotide repeat, in particular expanded CTG·CUG repeats.

### INTRODUCTION

Several diseases are associated with genes have expanded nucleotide repeats, that is, a greater number of nucleotide repeats than is observed in a healthy phenotype. The expanded repeat may cause aggregation and/or nucleation of the expanded repeat containing transcript and/or cause nucleation of proteins that bind to expanded repeat containing transcript. The expanded repeats may result in some proteins, such as pre-mRNA processing proteins, being sequestered on the repeat, thus inhibiting the proteins from performing their normal functions, such as processing pre-mRNA transcripts of other genes that do not contain the expanded repeat.

There are several diseases associated with genes having expanded CTG·CUG trinucleotide repeats (CTG refers to the DNA repeat and CUG refers to the corresponding RNA repeat that occurs upon transcription). Diseases associated with genes having expanded CTG·CUG trinucleotide repeats include, but are not limited to, myotonic dystrophy type 1 (DM1), Spinocerebellar Ataxia-8 (SCA8), Huntington's disease like-2 (HDL2), and Fuchs' endothelial corneal dystrophy (FECD).

Myotonic dystrophy type 1 (DM1), the most common cause of muscular dystrophy in adults, affecting 1 in 8500 individuals worldwide, is associated with a gene that has an expanded trinucleotide repeat (Lee and Cooper. (2009) “Pathogenic mechanisms of myotonic dystrophy,” Biochem Soc Trans. 37(06): 10.1042/BST0371281). DM1 is a disorder that affects skeletal and smooth muscle, as well as the eye, heart, endocrine system, and central nervous system. DM1 is caused by abnormal expansion of a CTG-trinucleotide repeat in the non-coding region of the gene encoding Dystrophia Myotonica Protein Kinase (DMPK). The CTG expansion lies within a region corresponding to the 3′ untranslated region (3′-UTR) of the DMPK mRNA. Whereas the DMPK gene in healthy individuals contains between 5 and 40 CTG trinucleotide repeats, patients with DM1 have from 50 and up to several thousand CTG trinucleotide repeats. CTG-trinucleotide repeat expansion results in global deregulation of gene expression in affected individuals due to nucleation of some regulatory RNA-binding proteins in the CUG-expansion in the 3′ untranslated region (3′-UTR), rendering the RNA-binding proteins, such as muscleblind-like protein (MBNL1-3) unable to perform their normal cellular function. The nucleated RNA-binding proteins are not available to bind and affect translation of other mRNA transcripts. These CUG-expanded mRNA-protein aggregates form distinct nuclear foci. The activity of additional splicing factors, such as CUGBP Elav-like family member 1 (CELF1), is also disrupted, leading to the mis-splicing of a large number of downstream gene transcripts associated with symptoms of DM1. Disease severity increases and age of onset decreases with an increasing number of repeats (Pettersson et al. (2015) “Molecular mechanisms in DM1—a focus on foci.” Nucleic Acids Res. 43(4):2433-2441).

The CUG-trinucleotide repeats in the 3′ untranslated region of DMPK mRNA form imperfect stable hairpin structures that accumulate in the cell nucleus in small ribonuclear complexes or microscopically visible inclusions, and impair the function of proteins implicated in transcription, splicing or RNA export. Although DMPK genes with CUG repeats are transcribed into mRNA, the mutant transcripts are sequestered in the nucleus as aggregates (foci), which results in a decrease in cytoplasmic DMPK mRNA levels. These aggregations lead to the deregulation of the alternative splicing of many different transcripts due to sequestration of two RNA-binding proteins: MBNL1 (muscleblind-like 1) and CUGBP1 (CUG-binding protein 1), resulting in loss-of-function of MBNL1 and upregulation of CUGBP1 (Lee and Cooper. (2009) “Pathogenic mechanisms of myotonic dystrophy,” Biochem Soc Trans. 37(06): 10.1042/BST0371281). MBNL1 and CUGBP-ETR-3 like factor 1 (CELF1) are developmental regulators of splicing events during fetal to adult transition and modification of their activities in DM1 leads to expression of a fetal splicing pattern in adult tissues. The downstream impact of decreased MBNL1 and increased CELF1 levels includes disruption of alternative splicing, mRNA translation and mRNA decay in proteins such as cardiac troponin T (cTNT), insulin receptor (INSR), muscle-specific chloride ion channel (CLCN1) and sarcoplasmic/endoplasmic reticulum calcium ATPase 1 (ATP2A1) transcripts, in addition to MBNL1 (Konieczny et al. (2017) “Myotonic dystrophy: candidate small molecule therapeutics.” Drug Discov Today. 22(11):1740-174).

Possible therapeutic approaches to treat DM1, or other diseases associated with expanded CTG·CUG repeats, include the use of therapeutic oligonucleotide containing compounds.

However, a major problem associated with the use of oligonucleotide compounds in therapeutics is their limited ability to gain access to the intracellular compartment when administered systemically. Intracellular delivery of oligonucleotide compounds can be facilitated by use of carrier systems such as polymers, cationic liposomes or by chemical modification of the construct, for example by the covalent attachment of cholesterol molecules. However, intracellular delivery efficiency of oligonucleotide compounds remains low. Improved delivery systems are still required to increase the potency of these compounds.

There is an unmet need for effective compositions to deliver therapeutic oligonucleotide compounds to intracellular compartments to treat diseases that are caused by expanded CTG·CUG repeats, such as DM1.

### SUMMARY

Compounds, compositions, and methods for treating a disease associated with an expanded CTG·CUG repeat are described herein. In embodiments, this disclosure relates to compounds that include an antisense compound (AC) and a cyclic peptide, such as a cyclic cell penetrating peptide (cCPP). In embodiments, the AC binds to a gene or gene transcript comprising an expanded CUG repeat. In embodiments, the cyclic peptide facilitates intracellular localization of the AC. The compounds may comprise an endosomal escape vehicle (EEV). The EEV may comprise the cyclic peptide and an exocyclic peptide.

In embodiments, provided herein is a compound comprising: (a) at least one cyclic peptide and (b) an antisense compound (AC) that is complementary to a target nucleotide. In embodiments, the target nucleotide comprises at least one expanded CUG or CTG repeat. In embodiments, the target nucleotide is a gene that comprises at least one expanded CTG repeat. In embodiments, the target nucleotide is RNA that comprises at least one expanded CUG repeat. In embodiments, the RNA that comprises at least one expanded CUG repeat is a pre-mRNA sequence. In embodiments, the expanded CUG repeat corresponds to an expanded CTG repeat in a gene from which the pre-mRNA is transcribed. In embodiments, the antisense compound binds to the expanded CTG repeat or the expanded CUG repeat. In embodiments, the AC comprises 5-40 CAG repeats (SEQ ID NO: 398) (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 repeats). In embodiments, the AC comprises a sequence of a nucleotide listed in Table 2, Table 10, or Table 11. In embodiments, the AC comprises a sequence of a nucleotide listed in Table 2.

In embodiments, the AC comprises at least one modified nucleotide or nucleic acid selected from a phosphorothioate (PS) nucleotide, a phosphorodiamidate morpholino (PMO) nucleotide, a locked nucleic acid (LNA), a peptide nucleic acid (PNA), a nucleotide comprising a 2′-O-methyl (2′-OMe) modified backbone, a 2′O-methoxy-ethyl (2′-MOE) nucleotide, a 2′,4′ constrained ethyl (cEt) nucleotide, a 2′-deoxy-2′-fluoro-beta-D-arabinonucleic acid (2′F-ANA), and combinations thereof. In embodiments, the AC comprises a PMO nucleotide.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, the antisense compound (AC) is complementary to at least a portion of an expanded CUG repeat in a target mRNA sequence. In embodiments, the AC is a phosphorodiamidate morpholino (PMO) nucleotide.

In embodiments, at least two charged amino acids of the cyclic peptide are arginine. In embodiments, at least two aromatic, hydrophobic amino acids of the cyclic peptide are phenylalanine, naphthylalanine (3-Naphth-2-yl-alanine), or a combination thereof. In embodiments, at least two uncharged, non-aromatic amino acids of the cyclic peptide are citrulline, glycine, or a combination thereof. In embodiments, the compound is a cyclic peptide having 6 to 12 amino acids wherein two amino acids of the cyclic peptide are arginine, at least two amino acids are aromatic, hydrophobic amino acids selected from phenylalanine, naphthylalanine, and combinations thereof, and at least two amino acids are uncharged, non-aromatic amino acids selected from citrulline, glycine, and combinations thereof.

In embodiments, the compound comprises an endosomal escape vehicle comprising a cyclic peptide and an exocyclic peptide (EP). In embodiments, the EP is conjugated to a linker at an amino group. The linker may be a linker as described herein. In embodiments, the EP is conjugated to the cyclic peptide via the linker. In embodiments, the EP is conjugated to the AC via the linker. In embodiments, the EP is conjugated to the linker that conjugates the AC to the cyclic peptide.

In embodiments, the EP comprises from 2 to 10 amino acids. In embodiments, the EP comprises from 4 to 8 amino acid residues. In embodiments, the EP comprises 1 or 2 amino acids comprising a side chain comprising a guanidine group, or a protonated form thereof. In embodiments, the EP comprises 1, 2, 3, or 4 lysine residues. In embodiments, the amino group on the side chain of each lysine residue is substituted with a trifluoroacetyl (—COCF3) group, allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. In embodiments, EP comprises at least 2 amino acid residues with a hydrophobic side chain. In embodiments, the amino acid residue with a hydrophobic side chain is selected from valine, proline, alanine, leucine, isoleucine, and methionine. In embodiments, the exocyclic peptide comprises one of the following sequences: PKKKRKV (SEQ ID NO: 42); KR; RR; KKK; KGK; KBK; KBR; KRK; KRR; RKK; RRR; KKKK (SEQ ID NO: 7); KKRK (SEQ ID NO: 8); KRKK (SEQ ID NO: 9); KRRK (SEQ ID NO: 10); RKKR (SEQ ID NO: 11); RRRR (SEQ ID NO: 12); KGKK (SEQ ID NO: 13); KKGK (SEQ ID NO: 14); KKKKK (SEQ ID NO: 18); KKKRK (SEQ ID NO: 19); KBKBK; KKKRKV (SEQ ID NO: 33); PGKKRKV (SEQ ID NO: 43); PKGKRKV (SEQ ID NO: 44); PKKGRKV (SEQ ID NO: 45); PKKKGKV (SEQ ID NO: 46); PKKKRGV (SEQ ID NO: 47); or PKKKRKG (SEQ ID NO: 48). In embodiments, the exocyclic peptide consists of one of the following sequences: PKKKRKV (SEQ ID NO: 42); KR; RR; KKK; KGK; KBK; KBR; KRK; KRR; RKK; RRR; KKKK (SEQ ID NO: 7); KKRK (SEQ ID NO: 8); KRKK (SEQ ID NO: 9); KRRK (SEQ ID NO: 10); RKKR (SEQ ID NO: 11); RRRR (SEQ ID NO: 12); KGKK (SEQ ID NO: 13); KKGK (SEQ ID NO: 14); KKKKK (SEQ ID NO: 18); KKKRK (SEQ ID NO: 19); KBKBK (SEQ ID NO: 24); KKKRKV (SEQ ID NO: 33); PGKKRKV (SEQ ID NO: 43); PKGKRKV (SEQ ID NO: 44); PKKGRKV (SEQ ID NO: 45); PKKKGKV (SEQ ID NO: 46); PKKKRGV (SEQ ID NO: 47); or PKKKRKG (SEQ ID NO: 48). In embodiments, the exocyclic peptide has the structure: Ac-P-K-K-K-R-K-V-(SEQ ID NO: 370).

In embodiments, the cyclic peptide comprises 4 to 12 amino acids. In embodiments, the cyclic peptide comprises 6 to 12 amino acids. In embodiments, at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine, at least two aromatic hydrophobic amino acids of the cyclic peptide are phenylalanine, napthylalanine, or combinations thereof, and at least two uncharged, non-aromatic amino acids are citrulline, glycine, or combinations thereof.

In embodiments, the cyclic peptide has 4 to 12 amino acids, wherein at least two amino acids are arginine and at least two amino acids comprise a hydrophobic side chain, provided that the cyclic peptide is not a cyclic peptide having a sequence of SEQ ID NO: 89-117. In embodiments, the cyclic peptide is not a cyclic peptide having a sequence of SEQ ID NO: 89-117.

CPP sequences and SEQ ID NOs

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| rRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

where F is L-phenylalanine, f is D-phenylalanine, <D is L-3-(2-naphthyl)-alanine, #is D-3-(2-naphthyl)-alanine, R is L-arginine, r is D-arginine, Q is L-glutamine, q is D-glutamine, C is L-cysteine, U is L-selenocysteine, W is L-tryptophan, K is L-lysine, D is L-aspartic acid, and Q is L-norleucine.

In embodiments, the cyclic peptide has the following structure:

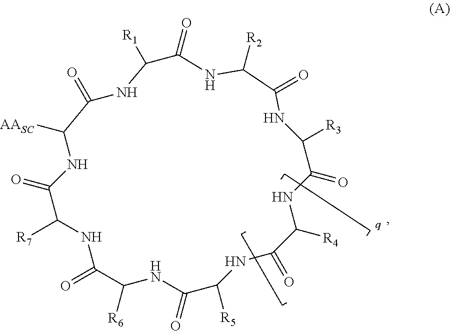

a protonated form thereof,

wherein:
R₁, R₂, and R₃ are each independently H or an aromatic or heteroaromatic side chain of an amino acid;
at least one of R₁, R₂, and R₃ is an aromatic or heteroaromatic side chain of an amino acid; R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, 0-homoarginine, 3-(1-piperidinyl)alanine;
AA_{SC} is an amino acid side chain to which the antisense compound is conjugated; and
q is 1, 2, 3 or 4.

In embodiments, at least one of R₄, R₅, R₆, R₇ are independently a uncharged, non-aromatic side chain of an amino acid. In embodiments, at least one of R₄, R₅, R₆, R₇ are independently H or a side chain of citrulline.

In embodiments, the cyclic peptide has the structure of Formula I:

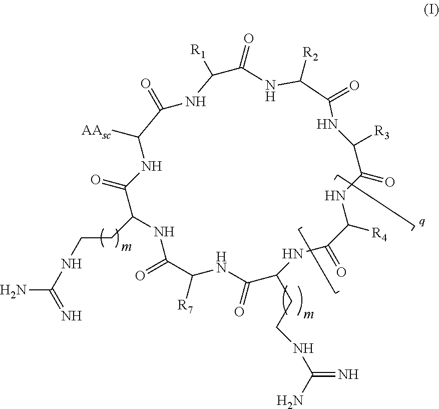

or a protonated form thereof,

wherein:
R₁, R₂, and R₃ are each independently H or an amino acid residue having a side chain comprising an aromatic group;
at least one of R₁, R₂, and R₃ is an aromatic or heteroaromatic side chain of an amino acid; R₄ and R₇ are independently H or an amino acid side chain; AA_{SC} is an amino acid side chain to which the antisense compound is conjugated;
q is 1, 2, 3 or 4; and
each m is independently an integer of 0, 1, 2, or 3.

In embodiments, the cyclic peptide of Formula (I) has one of the following structures:

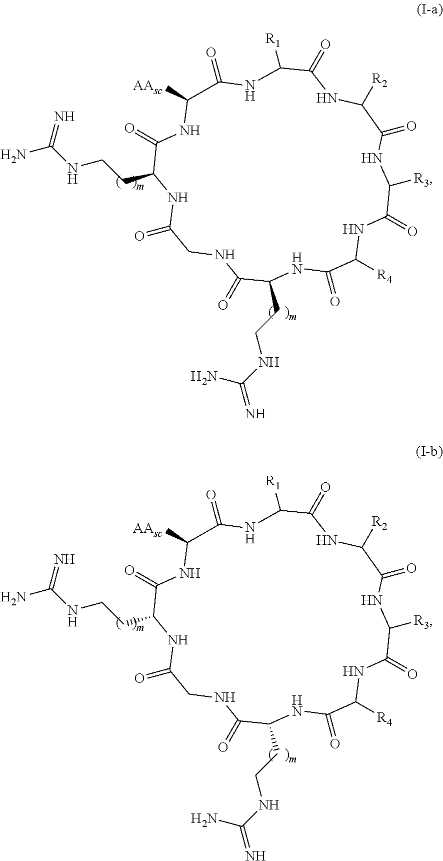

protonated form thereof.

In embodiments, the cyclic peptide of Formula (I) has one of the following structures (SEQ ID NOS: 646-651, respectively, in order of appearance):

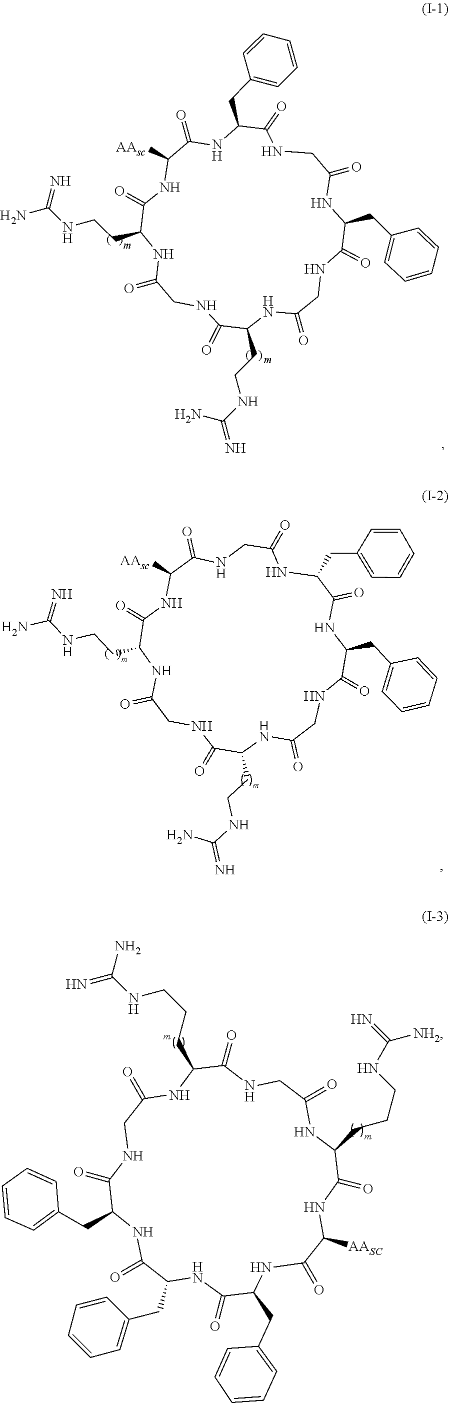

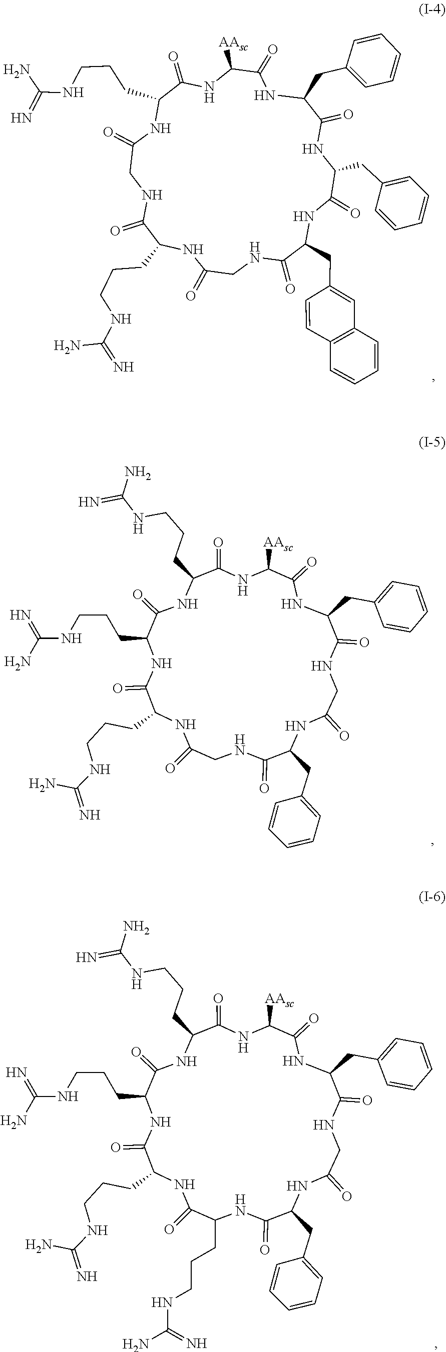

or a protonated form thereof.

In embodiments, the cyclic peptide of Formula (I) has one of the following structures (SEQ ID NOS: 646-647, respectively, in order of appearance):

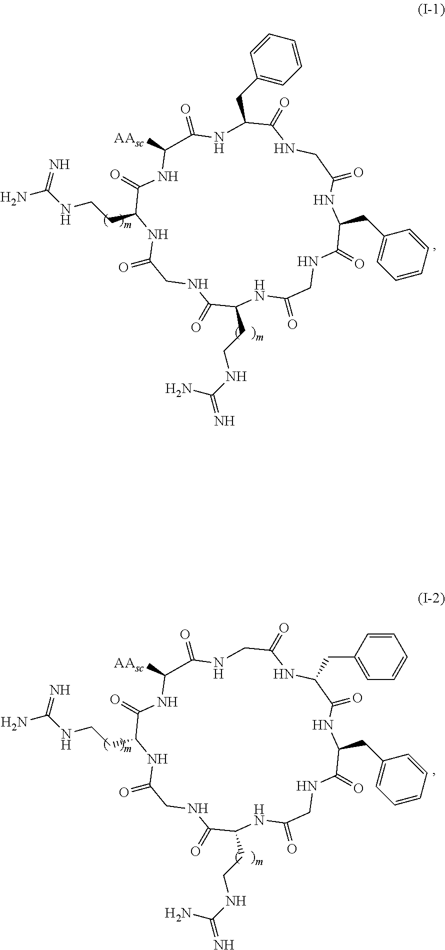

a protonated form thereof.

In embodiments, the cyclic peptide of Formula (I) has the following structure (SEQ ID NO: 646):

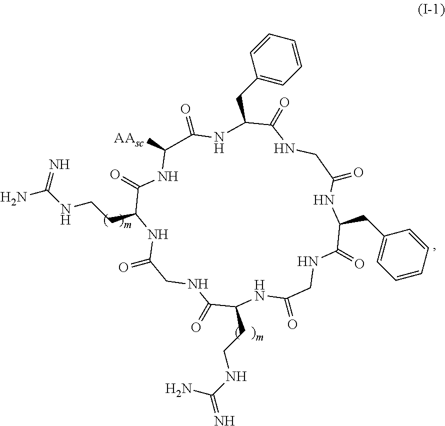

or a protonated form thereof.

In embodiments, the compound has a structure of Formula C:

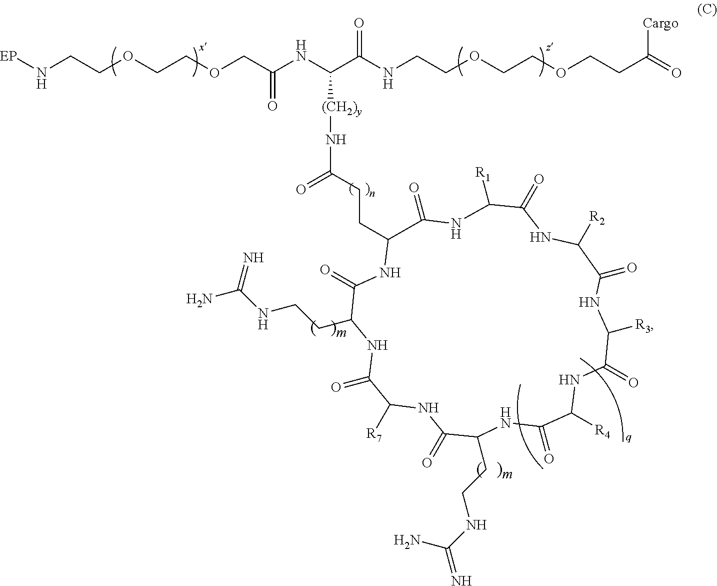

or a protonated form or salt thereof,

wherein:
R₁, R₂, and R₃ are each independently H or a side chain comprising an aryl or heteroaryl group, wherein at least one of R₁, R₂, and R₃ is a side chain comprising an aryl or heteroaryl group;
R₄ and R₇ are independently H or an amino acid side chain;
EP is the exocyclic peptide;
each m is independently an integer from 0-3;
n is an integer from 0-2;
x′ is an integer from 1-23;
y is an integer from 1-5;
q is an integer from 1-4;
z′ is an integer from 1-23, and Cargo is the antisense compound.

In embodiments, the compound has one or the following structures (SEQ ID NOS 652-655, respectively, in order of appearance):

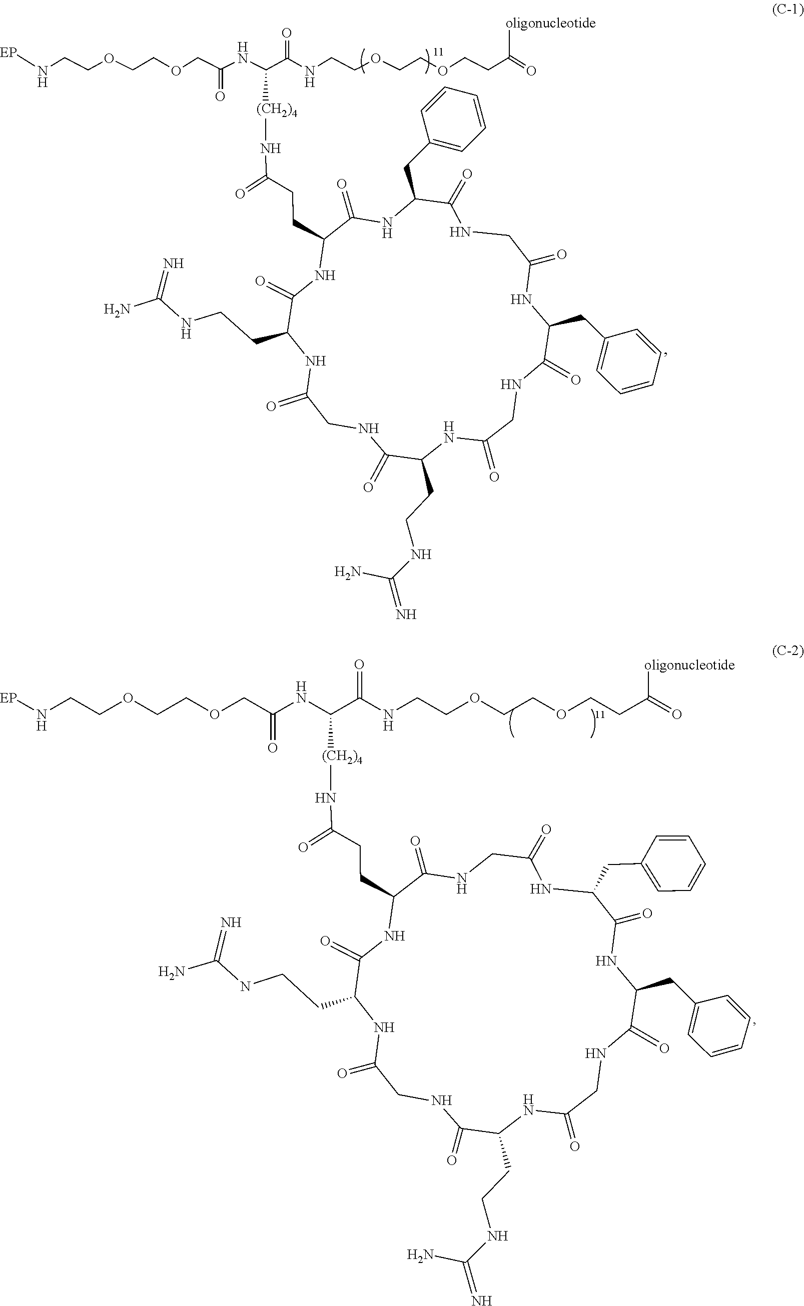

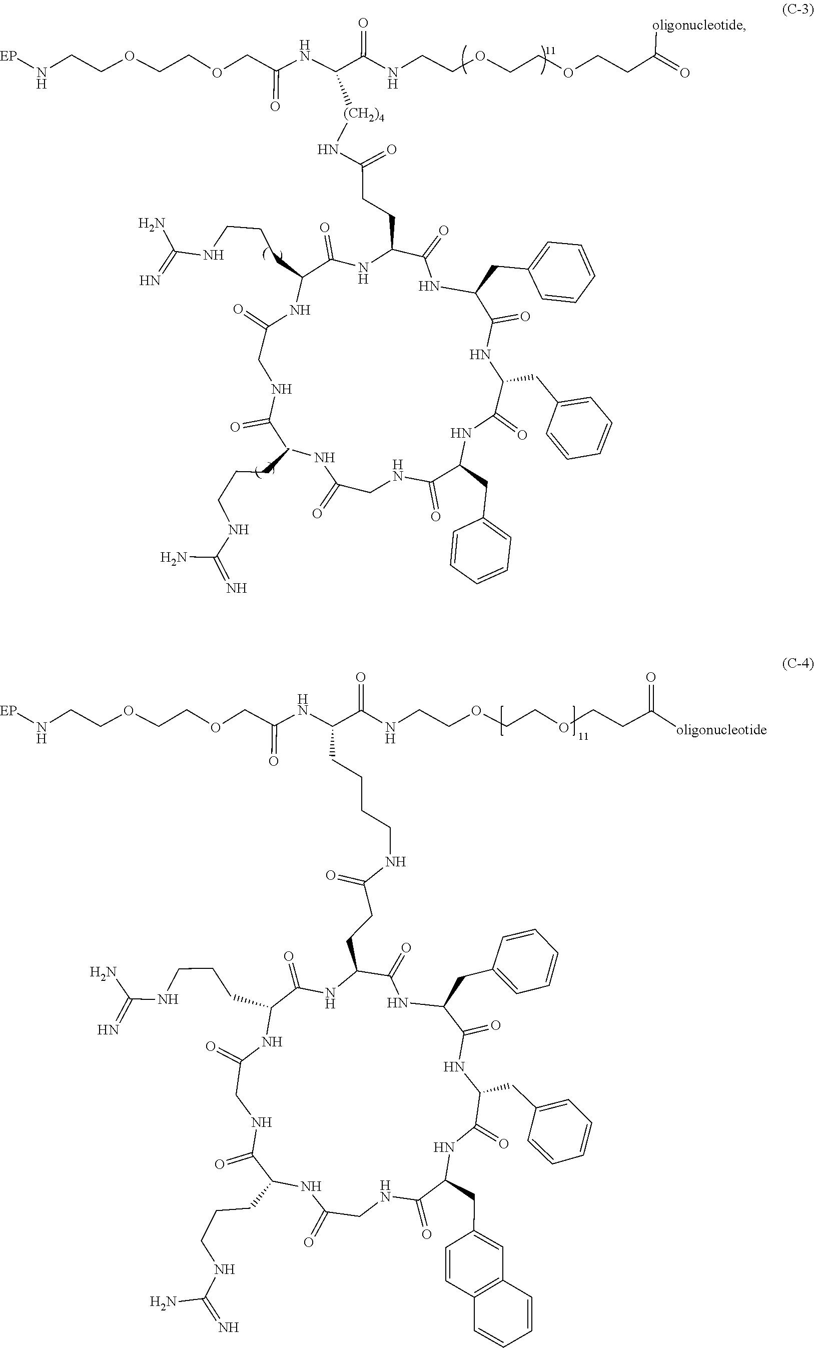

or a protonated form or salt thereof,

wherein EP is the exocyclic peptide, and

oligonucleotide is the antisense compound.

In embodiments, the oligonucleotide of the compound of Formula (C-1), (C-2), (C-3), or (C-4) comprises the following sequence: 5′-CAG CAG CAG CAG CAG CAG CAG-3′ (SEQ ID NO: 146).

In embodiments, the EP of the compound of Formula (C-1), (C-2), (C-3), or (C-4) comprises the following sequence: PKKKRKV (SEQ ID NO: 42).

### BRIEF DESCRIPTION OF THE DRAWINGS

FIG. 1 is a schematic showing multiple strategies for targeting CUG repeats in mRNA.

FIG. 2 shows modified nucleotides used in antisense oligonucleotides described herein. Structures 1-3 (1=Phosphorothioate; 2=(S_{C5}-Rₚ)-α,β-CAN; 3=PMO) are phosphate backbone modifications; 4 (2-thio-dT) is a base modification; 5-8 (5=2′-OMe-RNA; 6=2′O-MOE-RNA; 7=2′F-RNA; 8=2′F-ANA) are 2′ sugar modifications; 9-11 are constrained nucleotides; 12-14 (9=LNA; 10=(S)-cET; 11=tcDNA; 12=FHNA; 13=(S)5′-C-methyl; 14=UNA) are additional sugar modification; and 15-18 (15=E-VP; 16=Methyl phosphonate; 17=5′ phosphorothioate; 18═(S)-5′-C-methyl with phosphate) are 5′ phosphate stabilization modifications; 19 is a morpholino sugar. Reformatted from Khvorova, A., et al., Nat. Biotechnol. 2017 March; 35(3): 238-248.

FIGS. 3A-3D illustrate conjugation chemistries for connecting an AC to a cyclic cell penetrating peptide. FIG. 3A shows the amide bond formation between peptides with a carboxylic acid group or with TFP activated ester and primary amine residues at the 5′ end of an AC. FIG. 3B shows the conjugation of secondary amine or primary amine modified AC at 3′ and peptide-TFP ester through amide bond formation. FIG. 3C shows the conjugation of a peptide-azide to the 5′ cyclooctyne modified AC via copper-free azide-alkyne cycloaddition. FIG. 3D demonstrates another exemplary conjugation between a 3′ modified cyclooctyne ACs or 3′ modified azide ACs and CPP containing linker-azide or linker-alkyne/cyclooctyne moiety, via a copper-free azide-alkyne cycloaddition or cupper catalyzed azide-alkyne cycloaddition, respectively (click reaction).

FIG. 4 shows the conjugation chemistry for connecting an AC and CPP with an additional linker modality containing a polyethylene glycol (PEG) moiety.

FIGS. 5A-5D provide structures of the adenine (5A), cytosine (5B), guanine (5C), and thymine (5D) morpholino subunit monomers that may be used to synthesize phosphorodiamidate-linked morpholino oligomers (PMOs).

FIG. 6A-6F show RT-PCR analysis of alternative RNA splicing events (e.g., exon inclusion or exclusion) of MBNL1 (exon 5; FIG. 6A, 6B, 6E) and CLASP1 (exon 19; FIG. 6B, 6D, 6F) 24 hours (6A-6B) and 48 hours (6C-6D) after HeLa-48 cells were treated with 1 μM, 3 μM, or 10 μM of various PMOs or PMO-EEV compounds using the Endo-Porter transfection agent (6A-6C) or without the Endo-Porter agent (6E-6F). The parental HeLa cell line and the HeLa-480 cell line treated with (6A-6D) or without (6E-6F) the Endo-Porter agent were included as controls.

FIG. 7A-7B show RT-PCR analysis of alternative RNA splicing events (e.g., exon inclusion or exclusion) of MBNL1 (exon 5; FIG. 7A) and CLASP1 (exon 19; FIG. 7B) 48 hours after DM1 myoblasts were treated with 1 μM of various PMO or PMO-EEV compounds without the Endo-Porter transfection reagent. Two controls, DM-04 without endo-porter treated and DM-05 without endo-porter were included as controls.

FIG. 8A-8D show RT-PCR analysis of the alternative RNA splicing events (e.g., exon inclusion or exclusion) of Atp2al (exon 22; FIG. 8A), Nfix (exon 7; FIG. 8B), Clcn1 (exon 7a; FIG. 8C) and Mbn11 (exon 5; FIG. 8D) from gastrocnemius muscle tissues one week after HSA-LR (DM1-mouse model) mice were treated with a PMO, 20 mpk PMO-EEV 221-1106, or 40 mpk PMO-EEV 221-1106. FVB/NJ (wild type inbred mouse) and HSA-LR (without treatment) mice were included as control groups.

FIG. 9A-9D show RT-PCR analysis of the alternative RNA splicing events (e.g., exon inclusion or exclusion) of Atp2al (exon 22; FIG. 9A), Nfix (exon 7; FIG. 9B), Clcn1 (exon 7a;

FIG. 9C) and Mbn11 (exon 5; FIG. 9D) from quadricep muscle tissues one week after HSA-LR (DM1-mouse model) mice were treated with a PMO, 20 mpk PMO-EEV 221-1106, or 40 mpk PMO-EEV 221-1106. FVB/NJ (wild type inbred mouse) and HSA-LR (without treatment) mice were included as control groups.

FIG. 10A-10D show RT-PCR analysis of the alternative RNA splicing events (e.g., exon inclusion or exclusion) of Atp2al (exon 22; FIG. 10A), Nfix (exon 7; FIG. 10B), Clcn1 (exon 7a; FIG. 10C) and Mbn11 (exon 5; FIG. 10D) from tibialis anterior muscle tissues one week after HSA-LR (DM1-mouse model) mice were treated with a PMO, 20 mpk PMO-EEV 221-1106, or 40 mpk PMO-EEV 221-1106. FVB/NJ (wild type inbred mouse) and HSA-LR (without treatment) mice were included as control groups.

FIG. 11A-11F show RT-PCR analysis of alternative RNA splicing events (e.g., exon inclusion or exclusion) of MBNL1 (exon 5, FIG. 11A), SOS1 (exon 25, FIG. 11B), IR (exon 11, FIG. 11C), DMD (exon 78, FIG. 11D), BIN1 (exon 11, FIG. 11E) and LDB3 (exon 11, FIG. 11F) after DM1 patient derived muscle cells were treated with different concentrations (10 μm, 3 μm, 1 μm, 0.3 μm) of DMPK CUG-targeting EEV-PMOs (CUG^{exP} 197-777 and CUG^{exP} 221-1106). Muscle cells from two groups, healthy people (negative control) and DM1 patients (positive control), were tested for the alternative RNA splicing events as control. All data was collected from three individual experiments (n=3). T-test of treated versus untreated DM1 myotubes was conducted; * p<0.05; ** p<0.01; * * * p<0.001.

FIG. 12A-12F shows RT-PCR analysis of the alternative RNA splicing events (e.g., exon inclusion or exclusion) of MBL1 (exon 5, FIG. 12A), SOS1 (exon 25, FIG. 12B), INSR (exon 11, FIG. 12C), DMD (exon 78, FIG. 12D), BIN1 (exon 11, FIG. 12E) and LDB3 (exon 11, FIG. 12F) after patient derived DM1 myoblasts and myotubes were treated with 10 μm, 3 μm, or 1 μm of DMPK CUG-targeting EEV-PMO 197-777. Healthy patient cells and DM1 cells were used as controls. All data was collected from three individual experiments (n=3). T-test of treated versus untreated DM1 myotubes was conducted; * p<0.05; ** p<0.01; * * * p<0.001.

FIG. 13A-13B show the relative levels of mRNA after HSA-LR mice were treated with various concentrations of PMO-EEV 221-1120. FIG. 13A shows the relative mRNA level for the gastrocnemius, triceps, tibialis anterior, and diaphragm. FIG. 13B shows the relative mRNA levels in the diaphragm.

FIG. 14A-14D show the relative levels of mRNA in the quadricep (14A), gastrocnemius (14B), tricep (14C) and tibialis anterior (14D) tissues after HSA-LR mice were treated with various concentrations of PMO-EEV 221-1120.

FIG. 15A-15D show the mouse DM1 splicing index (mDSI) for various genes in quadricep (FIG. 15A), gastrocnemius (FIG. 15B), tricep (FIG. 15C), and tibialis anterior (FIG. 15D) tissues after HSA-LR mice were treated with various concentrations of PMO-EEV 221-1120.

FIGS. 16A-16C show the prevalence of RNA foci in the tibialis anterior muscle after HSA-LR mice were either untreated or treated with EEV-PMO 221-1120 (EEV-PMO-DM1-3; DM1-3). FIG. 16A-16B show images of tibialis anterior muscle tissue stained for RNA CUG foci (red) and nuclei (blue). FIG. 16C is a plot quantifying the percent of nuclei that have a CUG foci from data associated with the images in FIG. 16A-16B.

FIGS. 17A-17C and 17E-17J are plots showing a dose-dependent response for drug levels in the quadricep (17A), tricep (17B), heart (17C), gastrocnemius (17E), tibialis anterior (17F), diaphragm (17G), brain (1711), liver (17I), and kidney (17J) tissues after HSA-LR mice were treated with various concentrations of EEV-PMO-DM1-3. FIG. 17K shows drug exposure of various tissues at a 60 mpk dosage level.

FIG. 18 shows a dose dependent myotonia reduction in HSA-LR mice 7 days after treatment with EEV-PMO-DM1-3 at 15, 30, 60 and 90 mpk.

FIG. 19A-19D are plots show the results of a principal component analysis comparing gene expression in un-diseased mice (WT), DM1 mice (HSA-LR), and HSA-LR mice treated with PMO-EEV 221-1120. FIGS. 19A and 19C are plots showing three principal components and FIGS. 19B and 19D are plots showing two principal components.

FIG. 20A-20B show heatmaps of differentially expressed genes between un-diseased mice (WT), DM1 mice (HSA-LR), and HSA-LR mice treated with 60 mpk PMO-EEV 221-1120. FIG. 20A is a clustered heatmap showing 513 differentially expressed genes. FIG. 20B is a clustered heatmap showing 40 genes that are known to have CTG-CUG repeats.

FIG. 21 is a volcano plot showing the global transcriptional change across the untreated HSA-LR mice and mice treated with PMO-EEV 221-1120.

FIG. 22A-22E are plots show the result of a principal component analysis for the Scube2 (22A), Greb1 (22B), Ttc7 (22C), Txlnb(CUG)9 (22D), and Ndrg3 (22E) genes from undiseased mice, HSA-LR mice, and HSA-LR mice treated with PMO-EEV 221-1120.

FIG. 23A-23D show RNA sequencing (RNAseq) data for Atp2al (23A; exon 22 is boxed), Clcn1 (23B; exon 7a is boxed), Nfix (23C; exon 7 is boxed), and Mbn1 (23D; exon 5 is boxed) for un-diseased mice (WT-saline), HSA-LR mice (HSA-LR saline), and HSA-LR mice treated with PMO-EEV 221-1120. Two reads are shown for each treatment group.

FIG. 24 shows the percent spliced index (PSI) of individual exons for various genes of interest for undiseased mice (WT-saline), HSA-LR mice (HSA-LR saline), and HSA-LR mice treated with PMO-EEV 221-1120.

FIG. 25A-25D shows the drug levels in HSA-LR mice treated with 80 mpk (60 mpk oligo, 80 mpk whole drug) EEV-PMO-DM1-3 after 1 week to 4 weeks in the tibialis anterior (25A), gastrocnemius (25B), triceps (25C) and quadricep (25D) tissues.

FIG. 26A-26D show the drug levels in mice after HSA-LR mice were treated with a single 80 mpk dose of EEV-PMO-DM1-3. FIG. 26A-26B show the drug levels in the liver from 1 week to 12 weeks post treatment. FIG. 26C-26D show the drug levels in the kidney from 1 week to 12 weeks post treatment.

FIG. 27A-27C are plots showing the level of exon inclusion for MBNL1 (exon 5; 26A), SOS1 (exon 25; 26B), and NFIX (exon 7; 26C) after DM1 patient derived muscle cells were treated with 30 μM EEV-PMO-DM1-3.

FIG. 28A-28C shows that EEV-PMO-DM1-3 reduces CUG nuclear foci (green) in the nucleus (blue) in DM1 patient-derived muscle cells. FIG. 28A-28B are images of DM1 patient derived muscle cells that are either untreated or treated with EEV-PMO-DM1-3 or untreated. FIG. 28C is the quantification of the number of CUG foci per nucleus for data associated with the images in FIG. 28A.

FIG. 29A-29B show the raw data (29A) and the normalized data (29B) of a CELLTITER-GLO luminescent viability assay where RPTEC cells were treated with various concentrations of PMO-DM1 or EEV-PMO-DM1-3. Melittin was used as a positive control.

FIGS. 30A-30C show images depicting RNA CUG repeat foci in DM1 patient-derived cells (30A) and DM1 patient-derived cells treated with an EEV-PMO 221-1113 (30B). Cells were stained for nuclei (blue; Hoechst) and RNA CUG foci (green). FIG. 30C is a plot of the CUG RNA foci per nuclear area for data associated with the images of FIG. 30A-30B.

FIG. 31A-31B show the prevalence of RNA CUG7 foci in HeLa, untreated HeLa480 cells, and HeLa480 cells treated with EEV-PMO 221-1113. FIG. 31A shows images of cells stained for RNA CUG7 foci (green) and nuclei (blue). FIG. 31B is a plot quantifying the CUG7 foci per nuclear are for data associated with the images in FIG. 31A.

FIGS. 32A-32C are plots showing the percent inclusion of exon 5 in MBNL1 (32A), exon 25 in SOS1 (32B), and exon 7 in NFIX (32C) after DM1 patient-derived cells were treated with 30 μM of EEV-PMO 221-1113.

FIGS. 33A-33E show RT-PCR analysis of the alternative RNA splicing events (e.g., exon inclusion) of MBNL 1 (exon 5; 33A), SOS1 (exon 25; 33B), CLASP1 (exon 19, 33C), NFIX (exon 7, 33D), and INSR (exon 11, 33E) after DM1 patient derived muscle cells were treated with various concentrations of PMO-EEV 221-1113. T-test was used to determine significance; * p<0.05; ** p<0.01; *** p<0.001.

FIGS. 34A-34D show RT-PCR analysis of the alternative RNA splicing events (e.g., exon inclusion) Atp2al (exon 22, 34A), Nfix (exon 7, 34B), Clcn1 (exon 7a, 34C), and Mbn11 (exon 5, 34D) in the gastrocnemius tissue of mice treated with various concentrations of PMO 221 or EEV-PMO 221-1106.

FIGS. 35A-35C show RT-PCR analysis of the alternative RNA splicing events (e.g., exon inclusion or exclusion) of Mbn11 (exon 5, 35A), Nfix (exon 7, 35B), and Atp2al (exon 22, 35C) in the tibialis anterior tissue of HSA-LR mice treated with either PMO-EEV 0221-1121 (21-mer) or PMO-EEV 0325-1121 (24-mer).

FIGS. 36A-36C show RT-PCR analysis of the alternative RNA splicing events (e.g., exon inclusion) of Mbn11 (exon 5, 36A), Nfix (exon 7, 36B), and Atp2al (exon 22, 36C) in the gastrocnemius tissue of HSA-LR mice treated with either PMO-EEV 0221-1121 (21-mer) or PMO-EEV 0325-1121 (24-mer).

FIG. 37 shows PMO-0221a, the major metabolite of PMO-EEV 220-1120 detected in vivo. Figure discloses SEQ ID NOS: 660 and 659, respectively, in order of appearance.

FIGS. 38A-38B show the percent exon inclusion in the tibialis anterior (38A) and the gastrocnemius (38B) for MBNL1 (exon 5) after Hela480 cells were treated with various concentration of EEV-PMO 221-1120.

FIGS. 39A-39B show the percent exon inclusion in the tibialis anterior (39A) and the gastrocnemius (39B) for NFIX (exon 7) after Hela480 cells were treated with various concentration of EEV-PMO 221-1120.

FIGS. 40A-40B show the percent exon inclusion in the tibialis anterior (40A) and the gastrocnemius (40B) for Atp2al (exon 22) after Hela480 cells were treated with various concentration of EEV-PMO 221-1120.

FIG. 41A-41B show images (41A) depicting RNA CUG repeat foci in Hela480 cells after treatment with various concentrations of EEV-PMO 221-1120. FIG. 41B is a plot of the RNA foci per nuclear area for data associated with the images of FIG. 41A.

FIGS. 42A-42D show the relative r(CUG480) repeat mRNA levels, (42A), the relative DMPK mRNA levels (42B), percent exon 5 inclusion of MBNL1 (42C), and percent exon 25 inclusion in SOS1 (42D) in HeLa480 cells after treatment with various concentrations of EEV-PMO 221-1120.

FIG. 43 is a bar chart showing examples of genes expressed in muscle tissue that are known to have CTG-CUG repeats.

FIGS. 44A-44D show phenotypic myotonia reduction in HSA-LR mouse model treated with 20 mpk PMO-EEV 221-1106. FIGS. 44A and 44C show plots of relaxation. FIG. 44B shows an example raw force trace. FIG. 44D shows representative electromyography traces.

### DETAILED DESCRIPTION

### Compounds

In embodiments, compounds are provided that modulate the level and/or activity of a gene transcript having an expanded CUG trinucleotide repeat. In embodiments, the compounds of the present disclosure include at least one cyclic cell penetrating peptide (cCPP) and a therapeutic moiety™. The cCPP faciliates entry of the TM into the cell. In embodiments, the compound includes an ensomal escape vehicle (EEV) that comprises the cCPP and an exocyclic peptide (EP).

The cCPP or the EEV may permit the TM to enter the cytosol or a cellular compartment to interact with the target transcript.

### Therapeutic Moieties

Generally, the TM is the effector moiety that elicits a response. In embodiments, the TM elicits a response by modulating the expression, activity, and/or level of a target transcript and/or a target protein. In embodiments, the target transcript includes an expanded CUG trinucleotide repeat. In embodiments, the TM modulates the levels or a target transcript and/or target protein within a cell. In embodiments, the TM decreases the level of the target transcript and/or target protein within a cell.

In embodiments, the TM modulates the activity of the target transcript by reducing the affinity between the target transcript and one or more proteins that bind to the target transcript. By reducing the affinity between the target transcript and the one or more proteins, the TM may effectively modulate the activity of the one or more proteins that would otherwise be associated with the target transcript. For example, if the one or more proteins are not bound to the target transcript, they are available to carry out their functions on other molecules. For example, if the one or more proteins are involved in pre-mRNA processing, reducing the affinity of the one or more proteins for a transcript comprising an expanded CUG repeat may allow the one or more proteins to process pre-mRNA transcripts that do not comprise expanded CUG repeats. As such, the TM may modulate the activity, expression, and/or levels of the downstream genes (genes that do not contain the expanded CTG repeat) that are regulated by the one or more proteins whose interaction with the target transcript is disrupted by the TM.

In embodiments, the TM comprises an oligonucleotide, a peptide, an antibody, and/or a small molecule. The class and identity of the TM depends on the mechanism being used to modulate the level and/or activity of the target transcript that includes an expanded CUG trinucleotide repeat.

### Antisense Compound

In various embodiments, the compounds disclosed herein comprise a cell penetrating peptide (CPP) conjugated to an antisense compound (AC).

The term “antisense compound” refers to an oligonucleotide sequence that is complementary, or at least partially complementary, to a target nucleotide sequence. An AC is an oligonucleotide that includes natural DNA bases, modified DNA bases, natural RNA bases, modified RNA bases, natural RNA sugars, modified RNA sugars, natural DNA sugars, modified DNA sugars, natural internucleoside linkages, modified internucleoside linkages, or any combination thereof. ACs include, but are not limited to, antisense oligonucleotides RNAi, microRNA, antagomirs, aptamers, ribozymes, immunostimulatory oligonucleotides, decoy oligonucleotides, supermir, miRNA mimics, miRNA inhibitors, U1 adapters, and combinations thereof.

In embodiments, the AC includes a nucleotide sequence that is at least partially complementary to a target transcript that has an expanded CUG trinucleotide repeat. In embodiments, the AC includes a nucleotide sequence that is at least partially complementary to an expanded CUG trinucleotide repeat in a target mRNA sequence. Several diseases are associated with expanded CUG trinucleotide repeats, for example, myotonic dystrophy type 1 (DM1), Fuchs' Endothelial Corneal Dystrophy (FECD), Spinocerebellar Ataxia-8 (SCA8), and Huntington's Disease-Like (HDL2). Table 1 provides examples of nucleotide repeat disorders, and characteristics of genes with expanded nucleotide repeats associated with such disorders. The following document describes exemplary oligonucleotides for treating tandem repeat diseases and is incorporated by reference herein in its entirety: Zain et al. Neurotherapeutics. 2019; 16(2): 248-262; Zarouchlioti et al. Am J Hum Genet. 2018; 102(4):528-539; Fautsch et al. Prog Retin Eye Res. 2021; 81:100883.

TABLE 1
Diseases associated with expanded CUG trinucleotide repeats

| | | Normal | Expanded | | | |
|---|---|---|---|---|---|---|
| Disease | | repeat | repeat | Gene | Repeat | Location |
| (abbreviation) | Gene | length | length | product | sequence | of Repeat |
| DM1 | DMPK | 5-35 | >50 | Dystrophia | CTG•CAG | 3′ UTR |
| | | | | myotonica | | |
| | | | | protein | | |
| | | | | kinase | | |
| FECD | TCF4 | <30 | >40 | Transcription | CTG•CAG | Intron 3 |
| | | | | factor 4 | | |
| SCA8 | ATXN8OS | 15-50 | >50 | Ataxin 8 and | CTG•CAG | 3′ UTR |
| | and/or | | | ataxin 8 | | |
| | ATXN8 | | | opposite | | |
| | | | | strand | | |
| HDL2 | JPH3 | 6-27 | >40 | Junctophilin | CTG•CAG | 3′ UTR |
| | | | | 3 | | |

In embodiments, the AC includes a nucleotide sequence that is at least partially complementary to a nucleotide sequence that is within a target mRNA transcript that includes an expanded CTG-CUG trinucleotide repeat. In embodiments, the AC includes a nucleotide sequence that is at least partially complementary to an expanded CTG-CUG trinucleotide repeat in a target mRNA transcript.

In embodiments, the AC includes a nucleotide sequence that is at least partially complementary to a nucleotide sequence that is within a DMPK1 target transcript that includes an expanded CTG-CUG trinucleotide repeat. In embodiments, the AC includes a nucleotide sequence that is at least partially complementary to a nucleotide sequence that is within a TCF4 target transcript that includes an expanded CTG-CUG trinucleotide repeat. In embodiments, the AC includes a nucleotide sequence that is at least partially complementary to a nucleotide sequence that is within a ATXN8OS/ATXN8 target transcript that includes an expanded CTG-CUG trinucleotide repeat. In embodiments, the AC includes a nucleotide sequence that is at least partially complementary to a nucleotide sequence that is within a JPH3 target transcript that includes an expanded CTG-CUG trinucleotide repeat.

In embodiments, the AC includes a nucleotide sequence that is at least partially complementary to a trinucleotide repeat in a 3′UTR of a target mRNA transcript. In embodiments, the AC includes a nucleotide sequence that is at least partially complementary to an expanded CTG-CUG trinucleotide repeat in a 3′UTR of a DMPK1 target transcript. In embodiments, the AC includes a nucleotide sequence that is at least partially complementary to an expanded CTG-CUG trinucleotide repeat in a 3′UTR of a ATXN8OS/ATXN8 target transcript. In embodiments, the AC includes a nucleotide sequence that is at least partially complementary to an expanded CTG-CUG trinucleotide repeat in a 3′UTR of a JPH3 target transcript.

In embodiments, the AC includes a nucleotide sequence that is at least partially complementary to trinucleotide repeats, such as CTG-CUG repeats. In embodiments, the target nucleotide sequence comprises at least one expanded trinucleotide repeat (e.g., CTG-CUG repeats). In embodiments the target nucleotide sequence comprises at least 40, at least 45, at least 50, at least 60, at least 70, at least 80, at least 90, at least 100, at least 150, at least 200, at least 300, at least 400, at least 500, at least 600, at least 700, at least 800, at least 900, at least 1000, or at least 2000 CTG-CUG trinucleotide repeats. In embodiments, the expanded trinucleotide repeat is in the 3′UTR of the target nucleotide sequence.

In embodiments, the AC includes a nucleotide sequence that is at least partially complementary to, and may hybridize with, at least a portion of the contiguous expanded trinucleotide repeats present in the target transcript. In embodiments, the AC includes a nucleotide sequence that is at least partially complementary to, and may hybridize with, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, and up to 50,up to 100, up to 150, up to 200, up to 300, up to 400, up to 500, up to 600, up to 700, up to 800, up to 900, up to 1000, or up to 2000 trinucleotide repeats in a target transcript. In embodiments, the AC includes a nucleotide sequence that is at least partially complementary to, and may hybridize with, 2, 3, 4, 5, 6, 7, 8, 9, 10, 11, 12, 13, 14, 15, 16, 17, 18, 19 or 20 trinucleotide repeats in a target transcript. In embodiments, the AC includes a nucleotide sequence that is at least partially complementary to, and may hybridize with, 5 to 10 trinucleotide repeats in a target transcript. In embodiments, the AC includes a nucleotide sequence that is at least partially complementary to, and may hybridize with, 5 to 9 trinucleotide repeats in a target transcript. In embodiments, the AC includes a nucleotide sequence that is at least partially complementary to, and may hybridize with, 5 to 8 trinucleotide repeats in a target transcript. In embodiments, the AC includes a nucleotide sequence that is at least partially complementary to, and may hybridize with, 5 to 7 trinucleotide repeats in a target transcript. In embodiments, the AC includes a nucleotide sequence that is at least partially complementary to, and may hybridize with, 5 to 6 trinucleotide repeats in a target transcript. In embodiments, the AC includes a nucleotide sequence that is at least partially complementary to, and may hybridize with, 5 trinucleotide repeats in a target transcript. In embodiments, the AC includes a nucleotide sequence that is at least partially complementary to, and may hybridize with, 6 trinucleotide repeats in a target transcript. In embodiments, the AC includes a nucleotide sequence that is at least partially complementary to, and may hybridize with, 7 trinucleotide repeats in a target transcript. In embodiments, the AC includes a nucleotide sequence that is at least partially complementary to, and may hybridize with, 8 trinucleotide repeats in a target transcript. In embodiments, the AC includes a nucleotide sequence that is complementary at least partially to, and may hybridize with, 9 trinucleotide repeats in a target transcript. In embodiments, the AC includes a nucleotide sequence that

In embodiments, the AC may include a nucleotide sequence that is at least partially complementary to, and my hybridize with, at least a portion of the contiguous expanded trinucleotide repeats present at any location in a target transcript. In embodiments, the AC includes a nucleotide sequence that is at least partially complementary to, and may hybridize with, at least a portion of the contiguous expanded trinucleotide repeats present in the 3′ UTR of a target transcript. In embodiments, the AC includes a nucleotide sequence that is at least partially complementary to, and may hybridize with, at least a portion of the contiguous expanded trinucleotide repeats present in the 3′ UTR of DMPK1, SCA8, and/or HDL2 target transcript. In embodiments, the AC includes a nucleotide sequence that is at least partially complementary to, and may hybridize with, at least a portion of the contiguous expanded trinucleotide repeats present an intron of a target transcript. In embodiments, the AC includes a nucleotide sequence that is at least partially complementary to, and may hybridize with, at least a portion of the contiguous expanded trinucleotide repeats present in the intron 3 of a TCF4 target transcript. In embodiments, the AC includes a nucleotide sequence that is at least partially complementary to, and may hybridize with, at least a portion of the contiguous expanded trinucleotide repeats present in the CTG18.1 locus of the TCF4 transcript. In embodiments, the AC includes a nucleotide sequence that is at least partially complementary to, and may hybridize with, at least a portion of the contiguous expanded trinucleotide repeats present an exon of a target transcript.

In embodiments, the AC is 5 or more, 10 or more, 15 or more, 20 or more, 25 or more, 30 or more, 35 or more, 40 or more, or 45 or more nucleic acids in length. In embodiments, the AC is 50 or less, 45 or less, 40 or less, 35 or less, 30 or less, 25 or less, 20 or less, 15 or less, or 10 or less nucleic acids in length. In embodiments, the AC is 5 to 50, 5 to 45, 5 to 40, 5 to 35, 5 to 30, 5 to 25, 5 to 20, 5 to 15, or 5 to 10 nucleic acids in length. In embodiments, the AC is 10 to 50, 10 to 45, 10 to 40, 10 to 35, 10 to 30, 10 to 25, 10 to 20, or 10 to 15 nucleic acids in length. In embodiments, the AC is 15 to 50, 15 to 45, 15 to 40, 15 to 35, 15 to 30, 15 to 25, or 15 to 20 nucleic acids in length. In embodiments, the AC is 20 to 50, 20 to 45, 20 to 40, 20 to 35, 20 to 30, or 20 to 25 nucleic acids in length. In embodiments, the AC is 25 to 50, 25 to 45, 25 to 40, 25 to 35, or 25 to 30 nucleic acids in length. In embodiments, the AC is 30 to 50, 30 to 45, 30 to 40, or 30 to 35 nucleic acids in length. In embodiments, the AC is 35 to 50, 35 to 45, or 35 to 40 nucleic acids in length. In embodiments, the AC is 40 to 50 or 40 to 45 nucleic acids in length. In embodiments, the AC is 45 to 50 nucleic acids in length. In embodiments, the AC is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleic acids in length.

In embodiments, the AC has 100% complementarity to a target nucleotide sequence. In embodiments, the AC does not have 100% complementarity to a target nucleotide sequence. As used herein, the term “percent complementarity” refers to the number of nucleobases (e.g., natural nucleobase or modified nucleobase) of an AC that have nucleobase complementarity with a corresponding nucleobase of an oligomeric compound or nucleic acid (e.g., a target nucleotide sequence) divided by the total length (number of nucleobases) of the AC. One skilled in the art recognizes that the inclusion of mismatches is possible without eliminating the activity of the antisense compound.

In embodiments, the AC includes 20% or less, 15% or less, 10% or less, 5% or less, or zero mismatches to the target nucleotide sequence. In some embodiments, the AC includes 5% or more, 10% or more, or 15% or more mismatched. In embodiments, the AC includes zero to 5%, zero to 10%, zero to 15%, or zero to 20% mismatches to the target nucleotide sequence. In embodiments, the AC includes 5% to 10%, 5% to 15%, or 5% to 20% mismatches to the target nucleotide sequence. In embodiments, the AC includes 10% to 15% or 10% to 20% mismatches to the target nucleotide sequence. In embodiments, the AC includes 10% to 20% mismatches to the target nucleotide sequence.

In embodiments, the AC has 80% or greater, 85% or greater, 90% or greater, 95% or greater, 96% or greater, 97% or greater, 98% or greater, or 99% or greater complementarity to a target nucleotide sequence. In embodiments, the AC has 100% or less, 99% or less, 98% or less, 97% or less 96% or less 95% or less, 90% or less, 85% or less complementarity to a target nucleotide sequence. In embodiments, the AC has 80% to 100%, 80% to 99%, 80% to 98%, 80% to 97% 80% to 96%, 80% to 95%, 80% to 90% or 80% to 85% complementarity to a target nucleotide sequence. In embodiments, the AC has 85% to 100%, 85% to 99%, 85% to 98%, 85% to 97% 85% to 96%, 85% to 95%, or 85% to 90% complementarity to a target nucleotide sequence.

In embodiments, the AC has 90% to 100%, 90% to 99%, 90% to 98%, 90% to 97%, 90% to 96%, or 90% to 95% complementarity to a target nucleotide sequence. In embodiments, the AC has 95% to 100%, 95% to 99%, 95% to 98%, 95% to 97%, or 95% to 96% complementarity to a target nucleotide sequence. In embodiments, the AC has 96% to 100%, 96% to 99%, 96% to 98%, or 96% to 97% complementarity to a target nucleotide sequence. In embodiments, the AC has 97% to 100%, 97% to 99%, or 97% to 98% complementarity to a target nucleotide sequence. In embodiments, the AC has 98% to 100% or 98% to 99% complementarity to a target nucleotide sequence. In embodiments, the AC has 99% to 100% complementarity to a target nucleotide sequence.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between an AC and a target nucleotide sequence, such as by determining the thermal melting temperature (Tm). Tm or ATm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

In embodiments, the AC includes a nucleotide sequence that in itself is a trinucleotide repeat, that is, a CAG trinucleotide repeat. The reverse complement of a 5′-CAG-3′ has 100% complementarity and may hybridize with a 5′-CUG-3′ trinucleotide repeat. In embodiments, the AC includes one to 50 CAG repeats (SEQ ID NO: 399). In embodiments, the CAG repeats are contiguous. In embodiments, the CAG repeats are not contiguous. In embodiments, the AC includes a nucleotide sequence that includes incomplete CAG repeats on either the 5′ or 3′ end.

For example, in embodiments, the AC includes a sequence such as AG(CAG)ₙ(SEQ ID NO: 400), G(CAG)ₙ(SEQ ID NO: 401), (CAG)nAG (SEQ ID NO: 402), or (CAG)nA (SEQ ID NO: 403) where n is an integer from 1 to 50. In embodiments, the AC includes a nucleotide sequence that includes one or more, 2 or more, 3 or more, 4 or more, 5 or more, 6 or more, 7 or more, 8 or more, 9 or more, 10 or more, 20 or more, 30 or more, or 50 or more CAG repeats. In embodiments, the AC includes a nucleotide sequence that includes 50 or less, 40 or less, 30 or less, 20 or less, 10 or less, 9 or less, 8 or less, 7 or less, 6 or less, 5 or less, 4 or less, 3 or less, or 2 or less CAG repeats.

In embodiments, the AC includes a nucleotide sequence that includes 2 to 50 (SEQ ID NO: 404), 2 to 20 (SEQ ID NO: 405), 2 to 10 (SEQ ID NO: 406), 4 to 10 (SEQ ID NO: 407), 5 to 10 (SEQ ID NO: 408), 6 to 10 (SEQ ID NO: 409), 6 to 9 (SEQ ID NO: 410), 6 to 8 (SEQ ID NO: 411), or 6 to 7 CAG repeats (SEQ ID NO: 412). In embodiments, the AC includes any one of the nucleotide sequences in Table 2 (SEQ ID NO: 151-291).

TABLE 2
CAG repeat AC nucleotide sequences

| AC sequence (5′ to 3′) | SEQ ID NO: |
|---|---|
| CAG | NA |
| CAG-CAG | NA |
| CAG-CAG-CAG | NA |
| CAG-CAG-CAG-CAG | 151 |
| CAG-CAG-CAG-CAG-CAG | 152 |
| CAG-CAG-CAG-CAG-CAG-CAG | 153 |
| CAG-CAG-CAG-CAG-CAG-CAG-CAG | 154 |
| CAG-CAG-CAG-CAG-CAG-CAG-CAG-CAG | 155 |
| CAG-CAG-CAG-CAG-CAG-CAG-CAG-CAG-CAG | 156 |
| CAG-CAG-CAG-CAG-CAG-CAG-CAG-CAG-CAG-CAG | 157 |
| CAG-CAG-CAG-CAG-CAG-CAG-CAG-CAG-CAG-CAG-CAG | 158 |
| CAG-CAG-CAG-CAG-CAG-CAG-CAG-CAG-CAG-CAG-CAG-CAG | 159 |
| CAG-CAG-CAG-CAG-CAG-CAG-CAG-CAG-CAG-CAG-CAG-CAG-CAG | 160 |
| CAG-CAG-CAG-CAG-CAG-CAG-CAG-CAG-CAG-CAG-CAG-CAG- | 161 |
| CAG-CAG | |
| CAG-CAG-CAG-CAG-CAG-CAG-CAG-CAG-CAG-CAG-CAG-CAG- | 162 |
| CAG-CAG-CAG | |
| CAG-CAG-CAG-CAG-CAG-CAG-CAG-CAG-CAG-CAG-CAG-CAG- | 163 |
| CAG-CAG-CAG-CAG | |
| CAG-CAG-CAG-CAG-CAG-CAG-CAG-CAG-CAG-CAG-CAG-CAG- | 164 |
| CAG-CAG-CAG-CAG-CAG | |
| CAG-CAG-CAG-CAG-CAG-CAG-CAG-CAG-CAG-CAG-CAG-CAG- | 165 |
| CAG-CAG-CAG-CAG-CAG-CAG | |
| CAG-CAG-CAG-CAG-CAG-CAG-CAG-CAG-CAG-CAG-CAG-CAG- | 166 |
| CAG-CAG-CAG-CAG-CAG-CAG-CAG | |
| CAG-CAG-CAG-CAG-CAG-CAG-CAG-CAG-CAG-CAG-CAG-CAG- | 167 |
| CAG-CAG-CAG-CAG-CAG-CAG-CAG-CAG | |
| CAG-CAG-CAG-CAG-CAG-CAG-CAG-CAG-CAG-CAG-CAG-CAG- | 168 |
| CAG-CAG-CAG-CAG-CAG-CAG-CAG-CAG-CAG | |
| CAG-CAG-CAG-CAG-CAG-CAG-CAG-CAG-CAG-CAG-CAG-CAG- | 169 |
| CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 170 |
| CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 171 |
| CAG-CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 172 |
| CAG-CAG-CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 173 |
| CAG-CAG-CAG-CAG-CAG-CAG-CAG-CAG-CAG-CAG-CAG-CAG- | |
| CAG-CAG | |
| CAG-CAG-CAG-CAG-CAG-CAG-CAG-CAG-CAG-CAG-CAG-CAG- | 174 |
| CAG-CAG-CAG-CAG-CAG-CAG-CAG-CAG-CAG-CAG-CAG-CAG- | |
| CAG-CAG-CAG | |
| CAG-CAG-CAG-CAG-CAG-CAG-CAG-CAG-CAG-CAG-CAG-CAG- | 175 |
| CAG-CAG-CAG-CAG-CAG-CAG-CAG-CAG-CAG-CAG-CAG-CAG- | |
| CAG-CAG-CAG-CAG | |
| CAG-CAG-CAG-CAG-CAG-CAG-CAG-CAG-CAG-CAG-CAG-CAG- | 176 |
| CAG-CAG-CAG-CAG-CAG-CAG-CAG-CAG-CAG-CAG-CAG-CAG- | |
| CAG-CAG-CAG-CAG-CAG | |
| CAG-CAG-CAG-CAG-CAG-CAG-CAG-CAG-CAG-CAG-CAG-CAG- | 177 |
| CAG-CAG-CAG-CAG-CAG-CAG-CAG-CAG-CAG-CAG-CAG-CAG- | |
| CAG-CAG-CAG-CAG-CAG-CAG | |
| CAG-CAG-CAG-CAG-CAG-CAG-CAG-CAG-CAG-CAG-CAG-CAG- | 178 |
| CAG-CAG-CAG-CAG-CAG-CAG-CAG-CAG-CAG-CAG-CAG-CAG- | |
| CAG-CAG-CAG-CAG-CAG-CAG-CAG | |
| CAG-CAG-CAG-CAG-CAG-CAG-CAG-CAG-CAG-CAG-CAG-CAG- | 179 |
| CAG-CAG-CAG-CAG-CAG-CAG-CAG-CAG-CAG-CAG-CAG-CAG- | |
| CAG-CAG-CAG-CAG-CAG-CAG-CAG-CAG | |
| CAG-CAG-CAG-CAG-CAG-CAG-CAG-CAG-CAG-CAG-CAG-CAG- | 180 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG | |
| CAG-CAG-CAG-CAG-CAG-CAG-CAG-CAG-CAG-CAG-CAG-CAG- | 181 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 182 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 183 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 184 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 185 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG | |
| CAG-CAG-CAG-CAG-CAG-CAG-CAG-CAG-CAG-CAG-CAG-CAG- | 186 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG | |
| CAG-CAG-CAG-CAG-CAG-CAG-CAG-CAG-CAG-CAG-CAG-CAG- | 187 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG | |
| CAG-CAG-CAG-CAG-CAG-CAG-CAG-CAG-CAG-CAG-CAG-CAG- | 188 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG | |
| CAG-CAG-CAG-CAG-CAG-CAG-CAG-CAG-CAG-CAG-CAG-CAG- | 189 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG | |
| CAG-CAG-CAG-CAG-CAG-CAG-CAG-CAG-CAG-CAG-CAG-CAG- | 190 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG | |
| CAG-CAG-CAG-CAG-CAG-CAG-CAG-CAG-CAG-CAG-CAG-CAG- | 191 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG | |
| CAG-CAG-CAG-CAG-CAG-CAG-CAG-CAG-CAG-CAG-CAG-CAG- | 192 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG | |
| CAG-CAG-CAG-CAG-CAG-CAG-CAG-CAG-CAG-CAG-CAG-CAG- | 193 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 194 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 195 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 196 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG-CAG | |
| CAG-CAG-CAG-CAG-CAG-CAG-CAG-CAG-CAG-CAG-CAG-CAG- | 197 |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG-CAG-CAG-CAG-CAG-CAG-CAG-CAG-CAG-CAG-CAG- | |
| CAG-CAG | |
| AGC | NA |
| AGCAGC | NA |
| AGCAGCAGC | NA |
| AGCAGCAGCAGC | 198 |
| AGCAGCAGCAGCAGC | 199 |
| AGCAGCAGCAGCAGCAGC | 200 |
| AGCAGCAGCAGCAGCAGCAGC | 201 |
| AGCAGCAGCAGCAGCAGCAGCAGC | 202 |
| AGCAGCAGCAGCAGCAGCAGCAGCAGC | 203 |
| AGCAGCAGCAGCAGCAGCAGCAGCAGCAGC | 204 |
| AGCAGCAGCAGCAGCAGCAGCAGCAGCAGCAGC | 205 |
| AGCAGCAGCAGCAGCAGCAGCAGCAGCAGCAGCAGC | 206 |
| AGCAGCAGCAGCAGCAGCAGCAGCAGCAGCAGCAGCAGC | 207 |
| AGCAGCAGCAGCAGCAGCAGCAGCAGCAGCAGCAGCAGCAGC | 208 |
| AGCAGCAGCAGCAGCAGCAGCAGCAGCAGCAGCAGCAGCAGCAGC | 209 |
| AGCAGCAGCAGCAGCAGCAGCAGCAGCAGCAGCAGCAGCAGCAGC | 210 |
| AGC | |
| AGCAGCAGCAGCAGCAGCAGCAGCAGCAGCAGCAGCAGCAGCAGC | 211 |
| AGCAGC | |
| AGCAGCAGCAGCAGCAGCAGCAGCAGCAGCAGCAGCAGCAGCAGC | 212 |
| AGCAGCAGC | |
| AGCAGCAGCAGCAGCAGCAGCAGCAGCAGCAGCAGCAGCAGCAGC | 213 |
| AGCAGCAGCAGC | |
| AGCAGCAGCAGCAGCAGCAGCAGCAGCAGCAGCAGCAGCAGCAGC | 214 |
| AGCAGCAGCAGCAGC | |
| AGCAGCAGCAGCAGCAGCAGCAGCAGCAGCAGCAGCAGCAGCAGC | 215 |
| AGCAGCAGCAGCAGCAGC | |
| AGCAGCAGCAGCAGCAGCAGCAGCAGCAGCAGCAGCAGCAGCAGC | 216 |
| AGCAGCAGCAGCAGCAGCAGC | |
| AGCAGCAGCAGCAGCAGCAGCAGCAGCAGCAGCAGCAGCAGCAGC | 217 |
| AGCAGCAGCAGCAGCAGCAGCAGC | |
| AGCAGCAGCAGCAGCAGCAGCAGCAGCAGCAGCAGCAGCAGCAGC | 218 |
| AGCAGCAGCAGCAGCAGCAGCAGCAGC | |
| AGCAGCAGCAGCAGCAGCAGCAGCAGCAGCAGCAGCAGCAGCAGC | 219 |
| AGCAGCAGCAGCAGCAGCAGCAGCAGCAGC | |
| AGCAGCAGCAGCAGCAGCAGCAGCAGCAGCAGCAGCAGCAGCAGC | 220 |
| AGCAGCAGCAGCAGCAGCAGCAGCAGCAGCAGC | |
| AGCAGCAGCAGCAGCAGCAGCAGCAGCAGCAGCAGCAGCAGCAGC | 221 |
| AGCAGCAGCAGCAGCAGCAGCAGCAGCAGCAGCAGC | |
| AGCAGCAGCAGCAGCAGCAGCAGCAGCAGCAGCAGCAGCAGCAGC | 222 |
| AGCAGCAGCAGCAGCAGCAGCAGCAGCAGCAGCAGCAGC | |
| AGCAGCAGCAGCAGCAGCAGCAGCAGCAGCAGCAGCAGCAGCAGC | 223 |
| AGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | 224 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | 225 |
| AGCAGCAGCAGCAGCAGCAGCAGCAGCAGCAGCAGCAGCAGCAGC | |
| AGC | |
| AGCAGCAGCAGCAGCAGCAGCAGCAGCAGCAGCAGCAGCAGCAGC | 226 |
| AGCAGCAGCAGCAGCAGCAGCAGCAGCAGCAGCAGCAGCAGCAGC | |
| AGCAGC | |
| AGCAGCAGCAGCAGCAGCAGCAGCAGCAGCAGCAGCAGCAGCAGC | 227 |
| AGCAGCAGCAGCAGCAGCAGCAGCAGCAGCAGCAGCAGCAGCAGC | |
| AGCAGCAGC | |
| AGCAGCAGCAGCAGCAGCAGCAGCAGCAGCAGCAGCAGCAGCAGC | 228 |
| AGCAGCAGCAGCAGCAGCAGCAGCAGCAGCAGCAGCAGCAGCAGC | |
| AGCAGCAGCAGC | |
| AGCAGCAGCAGCAGCAGCAGCAGCAGCAGCAGCAGCAGCAGCAGC | 229 |
| AGCAGCAGCAGCAGCAGCAGCAGCAGCAGCAGCAGCAGCAGCAGC | |
| AGCAGCAGCAGCAGC | |
| AGCAGCAGCAGCAGCAGCAGCAGCAGCAGCAGCAGCAGCAGCAGC | 230 |
| AGCAGCAGCAGCAGCAGCAGCAGCAGCAGCAGCAGCAGCAGCAGC | |
| AGCAGCAGCAGCAGCAGC | |
| AGCAGCAGCAGCAGCAGCAGCAGCAGCAGCAGCAGCAGCAGCAGC | 231 |
| AGCAGCAGCAGCAGCAGCAGCAGCAGCAGCAGCAGCAGCAGCAGC | |
| AGCAGCAGCAGCAGCAGCAGC | |
| AGCAGCAGCAGCAGCAGCAGCAGCAGCAGCAGCAGCAGCAGCAGC | 232 |
| AGCAGCAGCAGCAGCAGCAGCAGCAGCAGCAGCAGCAGCAGCAGC | |
| AGCAGCAGCAGCAGCAGCAGCAGC | |
| AGCAGCAGCAGCAGCAGCAGCAGCAGCAGCAGCAGCAGCAGCAGC | 233 |
| AGCAGCAGCAGCAGCAGCAGCAGCAGCAGCAGCAGCAGCAGCAGC | |
| AGCAGCAGCAGCAGCAGCAGCAGCAGC | |
| AGCAGCAGCAGCAGCAGCAGCAGCAGCAGCAGCAGCAGCAGCAGC | 234 |
| AGCAGCAGCAGCAGCAGCAGCAGCAGCAGCAGCAGCAGCAGCAGC | |
| AGCAGCAGCAGCAGCAGCAGCAGCAGCAGC | |
| AGCAGCAGCAGCAGCAGCAGCAGCAGCAGCAGCAGCAGCAGCAGC | 235 |
| AGCAGCAGCAGCAGCAGCAGCAGCAGCAGCAGCAGCAGCAGCAGC | |
| AGCAGCAGCAGCAGCAGCAGCAGCAGCAGCAGC | |
| AGCAGCAGCAGCAGCAGCAGCAGCAGCAGCAGCAGCAGCAGCAGC | 236 |
| AGCAGCAGCAGCAGCAGCAGCAGCAGCAGCAGCAGCAGCAGCAGC | |
| AGCAGCAGCAGCAGCAGCAGCAGCAGCAGCAGCAGC | |
| AGCAGCAGCAGCAGCAGCAGCAGCAGCAGCAGCAGCAGCAGCAGC | 237 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGC | |
| AGCAGCAGCAGCAGCAGCAGCAGCAGCAGCAGCAGCAGCAGCAGC | 238 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | 239 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | 240 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | |
| AGC | |
| AGCAGCAGCAGCAGCAGCAGCAGCAGCAGCAGCAGCAGCAGCAGC | 241 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | |
| AGCAGC | |
| AGCAGCAGCAGCAGCAGCAGCAGCAGCAGCAGCAGCAGCAGCAGC | 242 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | |
| AGCAGCAGC | |
| AGCAGCAGCAGCAGCAGCAGCAGCAGCAGCAGCAGCAGCAGCAGC | 243 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | |
| AGCAGCAGCAGC | |
| AGCAGCAGCAGCAGCAGCAGCAGCAGCAGCAGCAGCAGCAGCAGC | 244 |
| AGCAGCAGCAGCAGCAGCAGCAGCAGCAGCAGCAGCAGCAGCAGC | |
| AGCAGCAGCAGCAGCAGCAGCAGCAGCAGCAGCAGCAGCAGCAGC | |
| AGCAGCAGCAGCAGC | |
| GCA | NA |
| GCAGCA | NA |
| GCAGCAGCA | NA |
| GCAGCAGCAGCA | 245 |
| GCAGCAGCAGCAGCA | 246 |
| GCAGCAGCAGCAGCAGCA | 247 |
| GCAGCAGCAGCAGCAGCAGCA | 248 |
| GCAGCAGCAGCAGCAGCAGCAGCA | 249 |
| GCAGCAGCAGCAGCAGCAGCAGCAGCA | 250 |
| GCAGCAGCAGCAGCAGCAGCAGCAGCAGCA | 251 |
| GCAGCAGCAGCAGCAGCAGCAGCAGCAGCAGCA | 252 |
| GCAGCAGCAGCAGCAGCAGCAGCAGCAGCAGCAGCA | 253 |
| GCAGCAGCAGCAGCAGCAGCAGCAGCAGCAGCAGCAGCA | 254 |
| GCAGCAGCAGCAGCAGCAGCAGCAGCAGCAGCAGCAGCAGCA | 255 |
| GCAGCAGCAGCAGCAGCAGCAGCAGCAGCAGCAGCAGCAGCAGCA | 256 |
| GCAGCAGCAGCAGCAGCAGCAGCAGCAGCAGCAGCAGCAGCAGCA | 257 |
| GCA | |
| GCAGCAGCAGCAGCAGCAGCAGCAGCAGCAGCAGCAGCAGCAGCA | 258 |
| GCAGCA | |
| GCAGCAGCAGCAGCAGCAGCAGCAGCAGCAGCAGCAGCAGCAGCA | 259 |
| GCAGCAGCA | |
| GCAGCAGCAGCAGCAGCAGCAGCAGCAGCAGCAGCAGCAGCAGCA | 260 |
| GCAGCAGCAGCA | |
| GCAGCAGCAGCAGCAGCAGCAGCAGCAGCAGCAGCAGCAGCAGCA | 261 |
| GCAGCAGCAGCAGCA | |
| GCAGCAGCAGCAGCAGCAGCAGCAGCAGCAGCAGCAGCAGCAGCA | 262 |
| GCAGCAGCAGCAGCAGCA | |
| GCAGCAGCAGCAGCAGCAGCAGCAGCAGCAGCAGCAGCAGCAGCA | 263 |
| GCAGCAGCAGCAGCAGCAGCA | |
| GCAGCAGCAGCAGCAGCAGCAGCAGCAGCAGCAGCAGCAGCAGCA | 264 |
| GCAGCAGCAGCAGCAGCAGCAGCA | |
| GCAGCAGCAGCAGCAGCAGCAGCAGCAGCAGCAGCAGCAGCAGCA | 265 |
| GCAGCAGCAGCAGCAGCAGCAGCAGCA | |
| GCAGCAGCAGCAGCAGCAGCAGCAGCAGCAGCAGCAGCAGCAGCA | 266 |
| GCAGCAGCAGCAGCAGCAGCAGCAGCAGCA | |
| GCAGCAGCAGCAGCAGCAGCAGCAGCAGCAGCAGCAGCAGCAGCA | 267 |
| GCAGCAGCAGCAGCAGCAGCAGCAGCAGCAGCA | |
| GCAGCAGCAGCAGCAGCAGCAGCAGCAGCAGCAGCAGCAGCAGCA | 268 |
| GCAGCAGCAGCAGCAGCAGCAGCAGCAGCAGCAGCA | |
| GCAGCAGCAGCAGCAGCAGCAGCAGCAGCAGCAGCAGCAGCAGCA | 269 |
| GCAGCAGCAGCAGCAGCAGCAGCAGCAGCAGCAGCAGCA | |
| GCAGCAGCAGCAGCAGCAGCAGCAGCAGCAGCAGCAGCAGCAGCA | 270 |
| GCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | 271 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | 272 |
| GCAGCAGCAGCAGCAGCAGCAGCAGCAGCAGCAGCAGCAGCAGCA | |
| GCA | |
| GCAGCAGCAGCAGCAGCAGCAGCAGCAGCAGCAGCAGCAGCAGCA | 273 |
| GCAGCAGCAGCAGCAGCAGCAGCAGCAGCAGCAGCAGCAGCAGCA | |
| GCAGCA | |
| GCAGCAGCAGCAGCAGCAGCAGCAGCAGCAGCAGCAGCAGCAGCA | 274 |
| GCAGCAGCAGCAGCAGCAGCAGCAGCAGCAGCAGCAGCAGCAGCA | |
| GCAGCAGCA | |
| GCAGCAGCAGCAGCAGCAGCAGCAGCAGCAGCAGCAGCAGCAGCA | 275 |
| GCAGCAGCAGCAGCAGCAGCAGCAGCAGCAGCAGCAGCAGCAGCA | |
| GCAGCAGCAGCA | |
| GCAGCAGCAGCAGCAGCAGCAGCAGCAGCAGCAGCAGCAGCAGCA | 276 |
| GCAGCAGCAGCAGCAGCAGCAGCAGCAGCAGCAGCAGCAGCAGCA | |
| GCAGCAGCAGCAGCA | |
| GCAGCAGCAGCAGCAGCAGCAGCAGCAGCAGCAGCAGCAGCAGCA | 277 |
| GCAGCAGCAGCAGCAGCAGCAGCAGCAGCAGCAGCAGCAGCAGCA | |
| GCAGCAGCAGCAGCAGCA | |
| GCAGCAGCAGCAGCAGCAGCAGCAGCAGCAGCAGCAGCAGCAGCA | 278 |
| GCAGCAGCAGCAGCAGCAGCAGCAGCAGCAGCAGCAGCAGCAGCA | |
| GCAGCAGCAGCAGCAGCAGCA | |
| GCAGCAGCAGCAGCAGCAGCAGCAGCAGCAGCAGCAGCAGCAGCA | 279 |
| GCAGCAGCAGCAGCAGCAGCAGCAGCAGCAGCAGCAGCAGCAGCA | |
| GCAGCAGCAGCAGCAGCAGCAGCA | |
| GCAGCAGCAGCAGCAGCAGCAGCAGCAGCAGCAGCAGCAGCAGCA | 280 |
| GCAGCAGCAGCAGCAGCAGCAGCAGCAGCAGCAGCAGCAGCAGCA | |
| GCAGCAGCAGCAGCAGCAGCAGCAGCA | |
| GCAGCAGCAGCAGCAGCAGCAGCAGCAGCAGCAGCAGCAGCAGCA | 281 |
| GCAGCAGCAGCAGCAGCAGCAGCAGCAGCAGCAGCAGCAGCAGCA | |
| GCAGCAGCAGCAGCAGCAGCAGCAGCAGCA | |
| GCAGCAGCAGCAGCAGCAGCAGCAGCAGCAGCAGCAGCAGCAGCA | 282 |
| GCAGCAGCAGCAGCAGCAGCAGCAGCAGCAGCAGCAGCAGCAGCA | |
| GCAGCAGCAGCAGCAGCAGCAGCAGCAGCAGCA | |
| GCAGCAGCAGCAGCAGCAGCAGCAGCAGCAGCAGCAGCAGCAGCA | 283 |
| GCAGCAGCAGCAGCAGCAGCAGCAGCAGCAGCAGCAGCAGCAGCA | |
| GCAGCAGCAGCAGCAGCAGCAGCAGCAGCAGCAGCA | |
| GCAGCAGCAGCAGCAGCAGCAGCAGCAGCAGCAGCAGCAGCAGCA | 284 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCA | |
| GCAGCAGCAGCAGCAGCAGCAGCAGCAGCAGCAGCAGCAGCAGCA | 285 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | 286 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | 287 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | |
| GCA | |
| GCAGCAGCAGCAGCAGCAGCAGCAGCAGCAGCAGCAGCAGCAGCA | 288 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | |
| GCAGCA | |
| GCAGCAGCAGCAGCAGCAGCAGCAGCAGCAGCAGCAGCAGCAGCA | 289 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | |
| GCAGCAGCA | |
| GCAGCAGCAGCAGCAGCAGCAGCAGCAGCAGCAGCAGCAGCAGCA | 290 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | |
| GCAGCAGCAGCA | |
| GCAGCAGCAGCAGCAGCAGCAGCAGCAGCAGCAGCAGCAGCAGCA | 291 |
| GCAGCAGCAGCAGCAGCAGCAGCAGCAGCAGCAGCAGCAGCAGCA | |
| GCAGCAGCAGCAGCAGCAGCAGCAGCAGCAGCAGCAGCAGCAGCA | |
| GCAGCAGCAGCAGCA | |

In embodiments, an AC that has a nucleotide that includes CAG repeats may include additional nucleotide sequences on the 5′ end, 3′ end, or both, of the CAG repeat. In embodiments, the additional nucleotide sequences may have 80% to 100% or 95% to 100% complementarity to portions of the target transcript to which they hybridize. The additional nucleotide sequences may be added to a CAG repeat nucleotide sequence in order to increase the selectivity of the AC for hybridizing to a specific target transcript.

In embodiments, the AC includes a nucleotide sequence that includes 1 to 50 CAG repeats (SEQ ID NO: 399) and that is a gapmer. Gapmers are oligonucleotides that are a DNA/RNA hybrid and that induce RNase decay mechanism. For example, gapmers may have a central DNA or DNA mimic segment that is flanked by an RNA or RNA mimic segment on both the 5′ and 3′ ends of the DNA or DNA mimetic segment. In embodiments, the AC includes a gapmer that includes a nucleotide sequence that hybridizes with a target nucleic acid sequence of the target transcript that is separate from the expanded CUG repeat of the target transcript.

In embodiments, an AC of the disclosure is a gapmer oligonucleotide as disclosed in U.S. Pat. No. 9,550,988, the disclosure of which is incorporated by reference herein.

In embodiments, an AC of the disclosure comprises the sequence and/or structure of any one of the ACs targeting DMPK disclosed in U.S. Patent Publication No. 2017/0260524, the disclosure of which is incorporated by reference herein.

In embodiments, an AC of the disclosure comprises the sequence and/or structure of any one of the ACs or oligonucleotides disclosed in U.S. Patent Publications US20030235845A1, US20060099616A1, US 2013/0072671 A1, US 2014/0275212 A1, US 2009/0312532 A1, US20100125099A1, US 2010/0125099 A1, US 2009/0269755 A1, US 2011/0294753 A1, US 2012/0022134 A1, US 2011/0263682 A1, US 2014/0128592 A1, US 2015/0073037 A1, and US20120059042A1, the contents of each of which are incorporated herein in their entirety for all purposes.

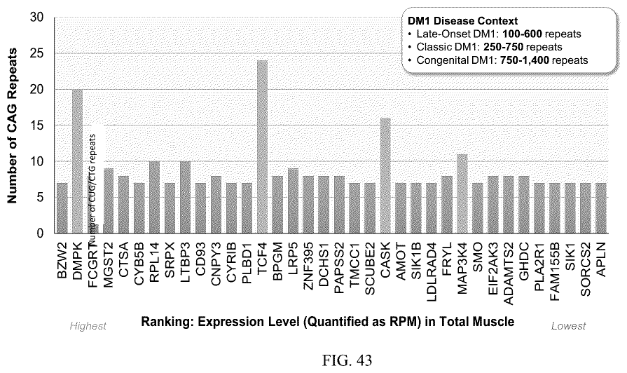

When using an AC to target and/or hybridize to an expanded CTG-CUG repeat, care must be taken to avoid off-target effects where the AC unintentionally binds to off-target transcripts that include CTG-CUG repeats (e.g., a transcript that includes CTG-CUG repats that are not an expanded CTG-CUG repeat). An in-silico analysis of the human genome reveals that in total, 63 human genes have CTG-CUG repeats (Uhlen, et. al., Science 2015.347(6220):1260419)). The 63 genes can be ranked by the expression of mRNA plus the amount of protein expressed in total muscle (cardiac, skeletal, and smooth muscle). The expression level can be quantified using RPM (reads per million) mRNA expression is FPKM (Fragments per kilo base of transcript per million mapped fragments) and protein expression is pTPM (transcripts per million protein coding genes), using greater than 10 RPM as the cutoff for non-insignificant expression. FIG. 43 shows the results of such an in-silico analysis. Thirty-six genes show an expression level of >10 RPM. Of the 36 genes, only three genes (besides DMPK) had >10 CTG-CUG repeats. Genes with 10 CTG-CUG repeats represent the lowest risk for off-target binding and toxicity. The number of CTG-CUG repeats (11-24) in these 3 genes (TCF4, CASK, MAP3k4) is nonetheless significantly lower than that seen in classic and congenital DM1 patients. For example, late-onset DM1 patients have 100-600 CTG-CUG repeats on DMPK, classical DM1 patients have 250-750 CTG-CUG repeats on DMPK, and congenital DM1 patients have 750-1,400 CTG-CUG repeats on DMPK. The same in-silico analysis may be performed for the liver and kidney. CASK is the only significant gene with >10 CTG-CUG repeats in the kidney. No genes with >10 CTG-CUG repeats were significant in the liver.

The ACs described herein may contain one or more asymmetric centers and thus give rise to enantiomers, diastereomers, and other stereoisomeric configurations that may be defined, in terms of absolute stereochemistry, as (R) or (S); a or P; or as (D) or (L). Included in the antisense compounds provided herein are all such possible isomers, as well as their racemic and optically pure forms.

The efficacy of the ACs may be assessed by evaluating the antisense activity effected by their administration. As used herein, the term “antisense activity” refers to any detectable and/or measurable activity attributable to the hybridization of an antisense compound to its target nucleotide sequence. Such detection and/or measuring may be direct or indirect. In embodiments, antisense activity is assessed by detecting and or measuring the amount of the protein expressed from the transcript of interest. In embodiments, antisense activity is assessed by detecting and/or measuring the amount of the transcript of interest. In embodiments, antisense activity is assessed by detecting and/or measuring the amount of alternatively spliced RNA and/or the amount of protein isoforms translated from the target transcript.

AC mechanisms of modulation

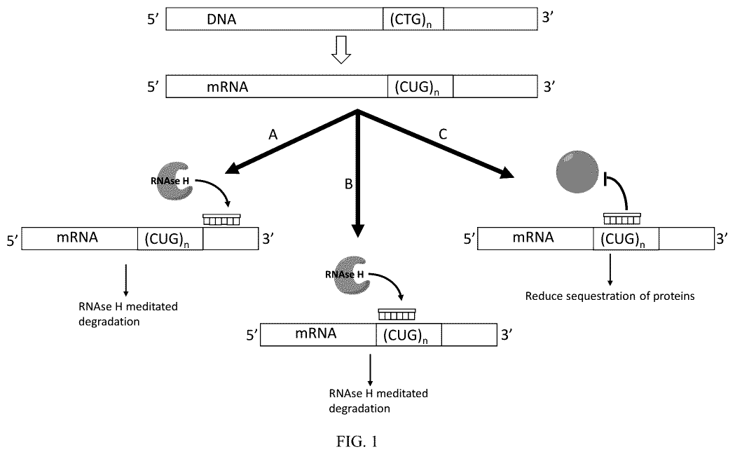

In embodiments, the AC may modulate the activity and/or level of the target transcript within the cell. FIG. 1 shows exemplary mechanisms of how an AC can modulate level and/or activity of a target transcript.

In embodiments, the AC may modulate the level of the target transcript within a cell. For example, in embodiments where the AC is a gamper, binding of the AC to the target transcript induces the degradation of the target transcript via RNase H pathways (FIG. 1, arrows A and B).

In embodiments, the gapmer hybridizes to a portion of the target transcript that is distinct from the expanded CUG trinucleotide repeat and thereby induces degradation of the target transcript via RNase H pathways (FIG. 1, Arrow A). In embodiments, the gapmer hybridizes to at least a portion of the expanded CUG repeat within the target transcript and thereby induces degradation of the target transcript via RNase H pathways (FIG. 1, Arrow B).

In embodiments, the AC may modulate the activity of the target transcript. Modulation of activity may include increasing or decreasing the ability of the target transcript to bind with a binding partner. In embodiments, the AC may modulate the activity of the target transcript by decreasing the ability of the target transcript to bind with one or more proteins that may associate with the transcript, particularly proteins that associate with at least a portion of the expanded CUG repeat of a target transcript (FIG. 1, arrow C). In embodiments, decreasing the ability of the target transcript to bind with one or more proteins includes decreasing the affinity of the target transcript for the one or more proteins. In embodiments, decreasing the ability of the target transcript to bind with one or more proteins includes partial or full steric blocking of the target transcript from binding the one or more proteins. For example, the AC may occupy at least a portion of the binding site that may be occupied by the one or more proteins if not sterically blocked. In embodiments, the binding site of the one or more proteins to the target transcript includes at least a portion of the expanded trinucleotide repeat of the target transcript. As such, in embodiments, the AC may occupy at least a portion of the expanded trinucleotide repeat (e.g., expanded CUG repeat) that may be occupied by the one or more proteins if not sterically blocked. Partial steric blocking of the target transcript may result in decreased affinity between the target transcript and the one or more proteins. For example, in embodiments, the AC binds to at least a portion of the expanded CUG repeat of the target transcript thereby sterically blocking and/or decreasing the affinity of target transcript for a protein that may bind to the expanded CUG repeat (FIG. 1, arrow C). The following review article describes additional applications for steric blocking antisense oligonucleotides and is incorporated by reference herein in its entirety: Roberts et al. Nature Reviews Drug Discovery (2020) 19: 673-694.

The CUG repeats of an expanded CUG repeat may form a double stranded hairpin structure. In the disease state, proteins bind to the double stranded hairpin structure and become sequestered and unable to perform other functions. In embodiments, the AC binds to at least a portion of the double stranded hairpin structure thereby sterically blocking and/or decreasing the affinity of the double stranded hairpin structure for a protein binding partner. In embodiments, the AC binds to at least a portion of a single stranded expanded CUG repeat thereby inhibiting the formation of the double stranded hairpin structure, and thus, inhibiting one or more protein from binding to the double stranded hairpin structure. In embodiments, hybridization of the AC to the double hairpin structure sterically blocks and/or decreases the affinity for one or more proteins for binding to the double hairpin structure. In embodiments, hybridization of the AC to at least a portion of a single stranded region of the expanded trinucleotide repeat, inhibits the formation of a double stranded hairpin structure. Decreasing the ability of the target transcript to bind with the one or more proteins, may allow the one or more proteins to carry out other functions such as, for example, regulating splicing of downstream transcripts (transcripts that do not contain an expanded CUG repeat). As such, in embodiments, decreasing the ability of the target transcript to bind with the one or more proteins, may increase the level of the one or more proteins within the cell that are available to provide other functions or function on other transcripts. In embodiments, decreasing the ability of the target transcript to bind with the one or more proteins, may increase the cytosolic level of the one or more proteins within the cell that are available to provide other functions or function on other transcripts.

Therefore, in embodiments, binding of the AC to the target transcript may result in the modulation of the level and/or activity of the one or more proteins that interact with the target transcript.

In embodiments, hybridization of the AC to at least a portion of the expanded CUG repeat, decreases the affinity and or sterically blocks the binding of MNBL1 to the target transcript. MNBL1 is a splicing factor that regulates the splicing of downstream gene transcripts. In a DM1 disease phenotype, MNBL1 binds to the expanded CUG repeat of a target transcript. While bound to target transcript, MNBL1 is sequestered in the nucleus and is not able to regulate the splicing of downstream gene transcripts (transcripts that do not contain an expanded CUG repeat). In embodiments, hybridization of the AC to at least a portion of the expanded CUG repeat in target transcript, sterically blocks and/or decreases the affinity of MNBL1 for the target transcript, thereby allowing it to regulate splicing of downstream gene transcripts. In embodiments, hybridization of the AC to at least a portion of the expanded CUG repeat in the target transcript, sterically blocks and/or decreases the affinity of MNBL1 for the target transcript, thereby increasing the amount of free (e.g., not bound to a transcript having a CUG repeat) MNBL1. In embodiments, hybridization of the AC to at least a portion of the expanded CUG repeat in the target transcript, sterically blocks and/or decreases the affinity of MNBL1 for the target transcript, thereby decreasing the amount of MBNL1 bound to and sequestered by the target transcript.

In embodiments where the target transcript is DMPK, hybridization of the AC to at least a portion of the expanded CUG repeat, decreases the affinity and or sterically blocks the binding of MNBL1 to the target transcript. MNBL1 is a splicing factor that regulates the splicing of downstream gene transcripts. In a DM1 disease phenotype, MNBL1 binds to the expanded CUG repeat of DMPK1. While bound to DMPK1, MNBL1 is sequestered in the nucleus and is not able to regulate the splicing of downstream gene transcripts (transcripts that do not contain an expanded CUG repeat). In embodiments, hybridization of the AC to at least a portion of the expanded CUG repeat in DMPK, sterically blocks and/or decreases the affinity of MNBL1 for the DMPK transcript, thereby allowing it to regulate splicing of downstream gene transcripts. In embodiments, hybridization of the AC to at least a portion of the expanded CUG repeat in DMPK, sterically blocks and/or decreases the affinity of MNBL1 for the DMPK transcript, thereby increasing the amount of free (e.g., not bound to a transcript having a CUG repeat) MNBL1. In embodiments, hybridization of the AC to at least a portion of the expanded CUG repeat in DMPK, sterically blocks and/or decreases the affinity of MNBL1 for the DMPK transcript, thereby decreasing the amount of MBNL1 bound to and sequestered by the DMPK1 transcript.

In embodiments where the target transcript is DMPK, hybridization of the AC to at least a portion of the expanded CUG repeat, results in the decrease of CUGBP1 levels. In the DM1 disease state, the level of free MBNL 1 (able to function) decreases while the level of free (able to function) of CUGBP1 increases. An increase in CUGBP1 levels is associated with the disease state. As such, in embodiments, hybridization of the AC to at least a portion of the expanded CUG repeat results in increased levels of free (able to function) MBNL1 and/or a decrease in free (able to function) CUGBP1 levels. Decreasing the ability of the target transcript to bind with the one or more proteins, may reduce, or inhibit the formation of CUG repeat foci. Transcripts that include expanded nucleotide repeats (e.g., expanded CUG repeats) may be transcribed and then sequestered in the nucleus.

Within the nucleus, the sequestered transcripts may form aggregates. Proteins that bind to the transcript may then being to nucleate on sequestered transcript and/or sequester transcript aggregate thereby forming expanded nucleotide repeat (e.g., CUG repeat) foci. The CUG repeat foci may be visible using microscopy. In embodiments, decreasing the ability of the target transcript to bind with the one or more proteins, may reduce, or inhibit the formation of aggregates that include the target transcript. In embodiments, decreasing the ability of the target transcript to bind with the one or more proteins, may reduce, or inhibit the nucleation of the one or more proteins on the target transcript, on a double stranded hairpin region of a transcript, or on an aggregate of target transcripts. In embodiments where the target transcript is DMPK, decreasing the ability of the DMPK1 target transcript to bind with MNBL1 may reduce, or inhibit the nucleation MNBL1 on a DMPK1 target transcript or DMPK1 target transcript aggregate. In embodiments, hybridization of the AC to the target transcript may result in inhibition or reduction in formation of CUG repeat nuclear foci. In embodiments, hybridization of the AC to the target transcript may result in inhibition or reduction in formation of CUG repeat nuclear foci formed from a DMPK, TCF4, JPH3, and/or ATXN8OS/ATXN8 target transcript.

In embodiments, hybridization of the AC to the target transcript may result in the modulation of the level, expression, and/or activity of one or more downstream genes. For example, hybridization of the AC to the target transcript may be used to induce target transcript degradation or sterically block or decreases the affinity of the target transcript for one or more proteins, thereby allowing the one or more proteins that were sequestered by the target transcript to regulate the expression, level, and/or activity of downstream genes. For example, in embodiments, the one or more proteins may include a protein that is involved in regulating the splicing of one or more downstream transcripts (transcripts that do not contain an expanded CUG repeat). In embodiments, the splicing of downstream transcripts is altered when the protein involved in splicing is bound and sequestered on the target transcript. For example, alteration of splicing may include the exclusion of one or more exons or the inclusion of one or more introns in a transcript thereby leading to the expression of various protein isoforms. In embodiments, the alteration of splicing may result in the inclusion of an exon and/or intron that includes a premature stop codon thereby resulting in a truncated isoform that may have no or deleterious activity.

Alteration of downstream gene transcript splicing may lead to a change in level, folding, and/or activity of the downstream gene product which may be associated with a disease phenotype. When not bound to the target transcript comprising the expanded CUG repeat, the protein involved in splicing is free to regulate splicing which may results in a correction (or rescue) of the splicing of the downstream gene transcript, thereby at least partially restoring the protein level, folding, and/or activity of the downstream gene product associated with a healthy phenotype.

In embodiments, AC hybridization to the target transcript may result in the modulation of the splicing of downstream gene transcripts that are regulated by proteins that are sequestered by the target transcript in a disease state associated with expanded nucleotide repeats (e.g., expanded trinucleotide repeats). In disease associated with expanded trinucleotide repeats, downstream gene transcripts are often mis-processed, for example, mis-spliced. The mis-splicing leads of the downstream gene transcripts may lead to gene products that are destroyed before translation or translated into proteins that have aberrant structure and/or function. For example, sequestration of proteins that regulate the processing of downstream gene transcripts may lead to the inclusion of exons and/or introns with premature stop codons, the inclusion of introns, the exclusion of exons, and/or the inclusion of alternative exons, which may lead to a transcript and or gene product that is destroyed prior to translation or that is translated into an gene product with aberrant function.

The change in levels of the downstream gene transcript and/or gene product and/or the aberrant structure and/or function of the downstream gene products are associated with expanded trinucleotide disease phenotypes. In embodiments, AC hybridization to the target transcript may result in the modulation of exon inclusion, exon exclusion, intron inclusion, and/or intron exclusion in downstream transcripts whose splicing is regulated by proteins that are sequestered to the target transcript during a disease state. As such, hybridization of the AC to the target transcript may result in the upregulation of downstream protein isomers and/or transcripts associated with a healthy phenotype. Similarly, hybridization of the AC to the target transcript may result in the downregulation (e.g., suppression) of downstream transcripts and/or protein isomers associated with a disease phenotype.

In embodiments where the target transcript is DMPK, AC hybridization to the target transcript may result in the modulation of the splicing of downstream gene transcripts that are regulated by proteins that are sequestered by the DMPK target transcript during a disease state. In DM1, several downstream gene transcripts are mis-spliced leading. The mis-spliced genes are associated with the disease phenotype. As such, modulation of gene splicing may include correcting (e.g., rescuing) the splicing of genes to result in gene products of downstream genes that are associated with a healthy phenotype. In embodiments where the target transcript is DMPK, AC hybridization to the target transcript may result in the modulation of the splicing of downstream gene transcripts that are regulated by MNBL1, a splicing regulator that is sequestered by the DMPK target transcript during a disease state. In embodiments where the target transcript is DMPK, AC hybridization to the target transcript may result in the modulation of the splicing of downstream gene transcripts that are regulated by CUGBP1, a protein whose activity is affected by expanded CUG repeats. In embodiments where the target transcript if DMPK, AC hybridization to the target transcript may result in the correct processing (e.g., splicing) of downstream genes that are regulated by MNBL1 and/or CUGBP1. In embodiments where the target transcript is DMPK, AC hybridization to the target transcript may result in the modulation of splicing of downstream genes including, but not limited to, 4833439L19Rik, Abcc9, Atp2a1, Arhgef10, Arhgap28, Armcx6, Angell, Best3, Bin1, Brd2, Cacnals, Cacna2dl, Cpd, Cpeb3, Ccpgl, Clasp1, ClC-1, Clcn1, Clk4, Cpeb2, Camk2g, Capzb, Copz2, Coch, cTNT, Ctu2, Cyp2sl, Dctn4, Dnmll, Eya4, Efna3, Efna2, Fbxo31, Fbxo21, Frem2, Fgd4, Fucal, Fnl, Gogla4, Gpr3711, Greb1, Hegl, Insr, Impdh2, IR, Itgav, Jag2, Klcl, Kcan6, Kifl3a, Ldb3, Lrrfip2, Mapt, Macfl, Map3k4, Mapkapl, Mbn11, Mllt3, Mbn12, Mef2c, Mpdz, Mrpll, Mxra7, Mybpc1, Myo9a, Ncapd3, Ngfr, Ndrg3, Ndufv3, Neb, Nfix, Numal, Opal, Pacsin2, Pcolce, Pdlim3, Pla2gl5, Phactr4, Phkal, Phtf2, Ppplrl2b, Ppp3cc, Ppplcc, Ramp2, Rapgefl, Rurn, Ryrl, Sorcs2, Spsb4, Scube2, Sema6c, Sfc8a3, Slain2, Sorbsl, Spag9, Tmem28, Tacc1, Tacc2, Ttc7, Tnik, Tnfrsf22, Tnfrsf25, Trappc9, Trim55, Ttn, Txnl4a, Txlnb, Ube2d3, Vsp39, or any combination thereof.

Mis-splicing of many of the above-mentioned downstream gene transcripts results in specific DM1 disease phenotypes. For example, MNBL1 is a splicing factor with loss of function in DM1 due to exon 5 inclusion. MNBL1 is sequestered by DMPK CUG expansion and forms RNA nuclear foci. Additionally, SOS1 promotes Ras activation to positively regulate RAS/MAPK signaling pathway. In DM1, exon 25 of SOS1 is excluded leading to inhibition of muscle hypertrophy pathways. In DM1, IR/INSR has exon 11 exclusion, which results in higher levels of low-signaling non-muscle isoform and decreased metabolic response to insulin in DM1 (insulin resistance). Similarly, exon 78 exclusion of DMD is observed in DM. Exon 78 exclusion results in out-of-frame transcript at C-terminal domain. This mutated protein is expressed in DM1 patients and associated with a mechanism responsible for muscle wasting in patients. BIN1 is required for proper muscle T-tubule formation (EC coupling). Exon 11 exclusion produces inactive isoform and is found in DM1 patients. LDB3 interacts with α-actinin at the Z-disc in striated muscle and maintains muscle structure. Exon 11 inclusion of LDB3 detected in DM1 results in reduction of affinity for Protein kinase C (PKC). Consequently, PKC becomes hyperactive in DM1. In embodiments modulation of one or more downstream genes results in the correction, or rescue, of transcript splicing that is associated with a healthy phenotype. As such, in embodiments, hybridization of the AC to the DMPK target transcript, results in the rescue of mis-splicing of downstream genes/transcripts, thereby, reducing the level of downstream genes/transcripts associated with a disease phenotype. As such, in embodiments, hybridization of the AC to the DMPK target transcript, results in the rescue of mis-splicing of downstream genes/transcripts, thereby, increasing the level of downstream genes/transcripts associated with a healthy phenotype.

In embodiments, the AC inhibits expression of the target transcript. In embodiments, the AC inhibits expression of the target transcript by blocking the pre-mRNA processing machinery and/or translation machinery from accessing and/or completing translation and/or pre-mRNA processing. In embodiments, the AC inhibits expression of the target transcript by inducing degradation of the target transcript, for example, through RNase H pathways.

### AC Structure

The AC includes an oligonucleotide and/or an oligonucleoside. Oligonucleotides and/or oligonucleotides are nucleotides or nucleosides linked through internucleoside linkages.

Nucleosides include a pentose sugar (e.g., ribose or deoxyribose) and a nitrogenous base covalently attached to sugar. The naturally occurring (or traditional basses) bases found in DNA and/or RNA are adenine (A), guanine (G), thymine (T), cytosine (C), and uracil (U). The naturally occurring sugars (or traditional sugars) found in DNA and/or RNA deoxyribose (DNA) and ribose (RNA). The naturally occurring nucleoside linkage (or traditional internucleoside linkage) is a phosphodiester bond. In embodiments, the ACs of the present disclosure may have all natural sugars, bases, and internucleoside linkages.

Chemically modified nucleosides are routinely used for incorporation into antisense compounds to enhance one or more properties, such as nuclease resistance, pharmacokinetics, or affinity for a target RNA. In embodiments, the ACs of the present disclosure may have one or more modified nucleosides. In embodiments, the ACs of the present disclosure may have one or more modified sugars. In embodiments, the ACs of the present disclosure may have one or more modified bases. In embodiments, the ACs of the present disclosure may have one or more modified internucleoside linkages.

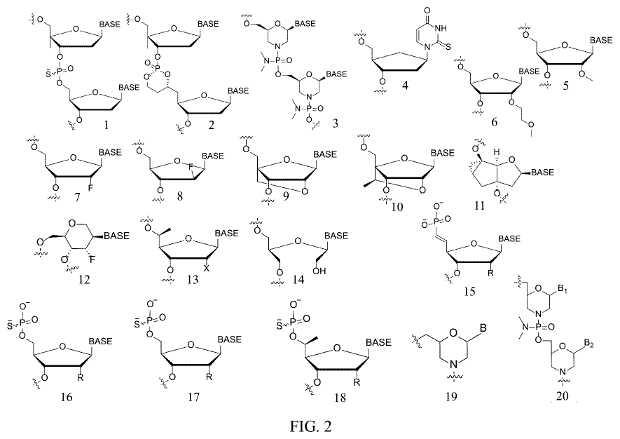

In general, a nucleobase is any group that contains one or more atom or groups of atoms capable of hydrogen bonding to a base of another nucleic acid. In addition to “unmodified” or “natural” nucleobases (A, G, T, C, and U) many modified nucleobases or nucleobase mimetics are known to those skilled in the art are amenable with the compounds described herein Generally a modified nucleobase refers to a nucleobase that is fairly similar in structure to the parent nucleobase, such as for example a 7-deaza purine, a 5-methyl cytosine, 2-thio-dT (FIG. 2) or a G-clamp. Generally, a nucleobase mimetic is a nucleobase that includes a structure that is more complicated than a modified nucleobase, such as for example a tricyclic phenoxazine nucleobase mimetic. Methods for preparation of the above noted modified nucleobases are well known to those skilled in the art.

In embodiments, the AC may include one or more nucleosides having a modified sugar moiety. In embodiments, the furanosyl sugar of a natural nucleoside may have a 2′ modification, modifications to make a constrained nucleoside, and others (see FIG. 2). For example, in embodiments, the furanosyl sugar ring of a natural nucleoside can be modified in a number of ways including, but not limited to, addition of a substituent group, bridging of two non-geminal ring atoms to form a bicyclic nucleic acid (BNA) or a locked nucleic acid; exchanging the oxygen of the furanosyl ring with C or N; and/or substitution of an atom or group such (see FIG. 2).

Modified sugars are well known and can be used to increase or decrease the affinity of the AC for its target nucleotide sequence. Modified sugars may also be used increase AC resistance to nucleases. Sugars can also be replaced with sugar mimetic groups among others. In embodiments, one or more sugars of the nucleosides of the AC is replaced with a methylenemorpholine ring as shown as 19 in FIG. 2.

In embodiments, the AC includes one or more nucleosides that include a bicyclic modified sugar (BNA; sometimes called bridged nucleic acids). Examples of BNAs include, but are not limited to LNA (4′—(CH₂)—O-2′ bridge), 2′-thio-LNA (4′—(CH₂)—S-2′ bridge), 2′-amino-LNA (4′—(CH₂)—NR-2′ bridge), ENA (4′—(CH₂)₂—O-2′ bridge), 4′—(CH₂)₃—2′ bridged BNA, 4′—(CH₂CH(CH₃))-2′ bridged BNA” cEt (4′—(CH(CH₃)—O-2′ bridge), and cMOE BNAs (4′—(CH(CH₂OCH₃)—O-2′ bridge). BNA's have been prepared and disclosed in the patent literature as well as in scientific literature (Srivastava, et al. J. Am. Chem. Soc. (2007), ACS Advanced online publication, 10.1021/ja071106y; Albaek et al. J. Org. Chem. (2006), 71, 7731-7740; Fluiter, et al. Chembiochem (2005), 6, 1104-1109; Singh et al., Chem. Commun. (1998), 4, 455-456; Koshkin et al., Tetrahedron (1998), 54, 3607-3630; Wahlestedt et al., Proc. Natl. Acad. Sci. U.S.A. (2000), 97, 5633-5638; Kumar et al., Bioorg. Med. Chem. Lett. (1998), 8, 2219-2222; WO 94/14226; WO 2005/021570; Singh et al., J. Org. Chem. (1998), 63, 10035-10039, WO 2007/090071; U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807).

In embodiments, the AC includes one or more nucleosides that include a locked nucleic acid (LNA). In LNAs the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs (2001), 2, 558-561; Braasch et al., Chem. Biol. (2001), 8 1-7; and Orum et al., Curr. Opinion Mol. Ther. (2001), 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun. (1998), 4, 455-456; ENA™; Morita et al., Bioorganic Medicinal Chemistry (2003), 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A. (2000), 97, 5633-5638).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research (2003), 21, 6365-6372).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of LNA analogs containing oligodeoxyribonucleotide duplexes as substrates for nucleic acid polymerases has also been described (Wengel et al., WO 99/14226). Furthermore, synthesis of 2′-amino-LNA, a conformationally restricted high-affinity oligonucleotide analog has been described (Singh et al., J. Org. Chem. (1998), 63, 10035-10039). In addition, 2′-amino- and 2′-methylamino-LNA's have been prepared and the thermal stability of their duplexes with complementary RNA and DNA strands has been previously reported.

Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and 6,600,032; and WO 2005/121371.

### Internucleoside Linkages

Described herein are internucleoside linking groups that link the nucleosides or otherwise modified nucleoside monomer units together thereby forming an oligonucleotide and/or an oligonucleotide containing AC. The ACs may include naturally occurring internucleoside linkages, unnatural internucleoside linkages, or both.

In naturally occurring DNA and RNA, the internucleoside linking group is a phosphodiester that covalently links adjacent nucleosides to one another to form a linear polymeric compound. In naturally occurring DNA and RNA, phosphodiester is linked to the 2′, 3′ or 5′ hydroxyl moiety of the sugar. Within oligonucleotides, the phosphate groups are commonly referred to as forming the internucleoside backbone of the oligonucleotide. In naturally occurring DNA and RNA, the linkage or backbone of RNA and DNA, is a 3′ to 5′ phosphodiester linkage. In embodiments, the internucleoside linking groups of the ACs are phosphodiesters. In embodiments, the internucleoside linking groups of the ACs are 3′ to 5′ phosphodiester linkages.

The two main classes of unnatural internucleoside linking groups are defined by the presence or absence of a phosphorus atom. Representative phosphorus containing internucleoside linkages include, but are not limited to, phosphotriesters, methylphosphonates, phosphoramidate, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H2—O—); and N,N′-dimethylhydrazine (—CH₂—N(CH₃)—N(CH₃)—). ACs having phosphorus internucleoside linking groups are referred to as oligonucleotides. Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared as racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, two or more nucleosides having modified sugars and/or modified nucleobases may be joined using a phosphoramidate. In embodiments, two or more nucleosides having a methylenemorpholine ring may be connected through a phosphoramidate internucleoside linkage.

Antisense compounds that include nucleobases with a methylenemorpholine ring that are linked through phosphoramidate internucleoside linkage may be referred to as phosphoramidate morpholino oligomers (PMOs).

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the AC or the CPP (CPP discussed elsewhere herein).

In embodiments, conjugate groups include lipid moieties such as a cholesterol moiety (Letsinger et al., Proc. Natl. Acad. Sci. USA (1989), 86, 6553); cholic acid (Manoharan et al., Bioorg. Med. Chem. Lett. (1994), 4, 1053); a thioether, e.g., hexyl-S-tritylthiol (Manoharan et al., Ann. N.Y. Acad. Sci. (1992), 660, 306; Manoharan et al., Bioorg. Med. Chem. Let. (1993), 3, 2765); a thiocholesterol (Oberhauser et al., Nucl. Acids Res. (1992), 20, 533); an aliphatic chain, e.g., dodecandiol or undecyl residues (Saison-Behmoaras et al., EMBO J. (1991), 10, 111; Kabanov et al., FEBS Lett. (1990), 259, 327; Svinarchuk et al., Biochimie (1993), 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett. (1995), 36, 3651; Shea et al., Nucl.

Acids Res. (1990), 18, 3777); a polyamine or a polyethylene glycol chain (Manoharan et al., Nucleosides & Nucleotides (1995), 14, 969); adamantane acetic acid (Manoharan et al., Tetrahedron Lett. (1995), 36, 3651); a palmityl moiety (Mishra et al., Biochim. Biophys. Acta. (1995), 1264, 229); or an octadecylamine or hexylamino-carbonyl-oxycholesterol moiety (Crooke et al., J. Pharmacol. Exp. Ther. (1996),277,923).

### Types of Antisense Compounds

Various types of AC may be used for example, including an antisense oligonucleotide, siRNA, microRNA, antagomir, aptamer, ribozyme, supermir, miRNA mimic, miRNA inhibitor, or combinations thereof.

### Antisense Oligonucleotides

In various embodiments, the antisense compound (AC) is an antisense oligonucleotide (ASO) that is complementary to a target nucleotide sequence. The term “antisense oligonucleotide (ASO)” or simply “antisense” is meant to include oligonucleotides that are complementary to a target nucleotide sequence. The term also encompasses ASOs that may not be fully complementary to the desired target nucleotide sequence. ASOs include single strands of DNA and/or RNA that are complementary to a chosen target nucleotide sequence or a target gene. ASOs may include one or more modified DNA and/or RNA bases, modified sugars, and/or unnatural internucleoside linkages. In embodiments, the ASOs may include one or more phosphoramidate internucleoside linkages. In embodiments, the ASO is phosphoramidate morpholino oligomers (PMOs). ASOs may have any characteristic, be any length, bind to any target nucleotide sequence and/or sequence element, and effect any mechanism as described relative to an AC.

Antisense oligonucleotides have been demonstrated to be effective as targeted inhibitors of protein synthesis, and, consequently, can be used to specifically inhibit protein synthesis by a targeted gene. The efficacy of ASO for inhibiting protein synthesis is well established. To date, these compounds have shown promise in several in vitro and in vivo models, including models of inflammatory disease, cancer, and HIV (Agrawal, Trends in Biotech. (1996), 14:376-387). Antisense can also affect cellular activity by hybridizing specifically with chromosomal DNA.

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et ai, Nucleic Acids Res. 1997, 25(17):3389-402).

### RNA Interference

In embodiments, the AC includes a molecule that mediates RNA interference (RNAi). As used herein, the phrase “mediates RNAi” refers to the ability to silence, in a sequence specific manner, a target transcript. While not wishing to be bound by theory, it is believed that silencing uses the RNAi machinery or process and a guide RNA, e.g., an siRNA compound of from about 21 to about 23 nucleotides. In embodiments, the AC targets the target transcript for degradation.

As such, in embodiments, RNAi molecule may be used to disrupt the expression of a gene or polynucleotide of interest. In embodiments, RNAi molecule is used to induce degradation of the target transcript, such as a pre-mRNA or a mature mRNA.

In embodiments, the AC includes a small interfering RNA (siRNA) that elicits an RNAi response.

Small interfering RNAs (siRNAs) are nucleic acid duplexes normally from about 16 to about 30 nucleotides long that can associate with a cytoplasmic multi-protein complex known as RNAi-induced silencing complex (RISC). RISC loaded with siRNA mediates the degradation of homologous transcripts, therefore siRNA can be designed to knock down protein expression with high specificity. Unlike other antisense technologies, siRNA function through a natural mechanism evolved to control gene expression through non-coding RNA. A variety of RNAi reagents, including siRNAs targeting clinically relevant targets, are currently under pharmaceutical development, as described, e.g., in de Fougerolles, A. et al., Nature Reviews (2007) 6:443-453.

While the first described RNAi molecules were RNA:RNA hybrids that include both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., Molecular Biotechnology (2003), 24:111-119). In embodiments, RNAi molecules are used that include any of these different types of double-stranded molecules. In addition, it is understood that RNAi molecules may be used and introduced to cells in a variety of forms. Accordingly, as used herein, RNAi molecules encompasses any and all molecules capable of mediating an RNAi in cells, including, but not limited to, double-stranded oligonucleotides that include two separate strands, i.e. a sense strand and an antisense strand, e.g., small interfering RNA (siRNA); double-stranded oligonucleotide that includes two separate strands that are linked together by non-nucleotidyl linker; oligonucleotides that include a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is at least about 14, at least about 15, at least about 20, at least about 25, at least about 30, at least about 35, at least about 40, or up to about 50 nucleotides in length. In certain embodiments, the single strand siRNA is less than about 200, about 100, or about 60 nucleotides in length.

Hairpin siRNA compounds may have a duplex region equal to or at least about 17, about 18, about 19, about 20, about 21, about 22, about 23, about 24, or about 25 nucleotide pairs. The duplex region may be equal to or less than about 200, about 100, or about 50 nucleotide pairs in length. In certain embodiments, ranges for the duplex region are from about 15 to about 30, from about 17 to about 23, from about 19 to about 23, and from about 19 to about 21 nucleotides pairs in length. The hairpin may have a single strand overhang or terminal unpaired region. In certain embodiments, the overhangs are from about 2 to about 3 nucleotides in length. In embodiments, the overhang is at the same side of the hairpin and in embodiments on the antisense side of the hairpin.

A “double stranded siRNA compound” as used herein, is an siRNA compound which includes more than one, and in some cases two, strands in which interchain hybridization can form a region of duplex structure.

The antisense strand of a double stranded siRNA compound may be equal to or at least about 14, about 15, about 16 about 17, about 18, about 19, about 20, about 25, about 30, about 40, or about 60 nucleotides in length. It may be equal to or less than about 200, about 100, or about 50 nucleotides in length. Ranges may be from about 17 to about 25, from about 19 to about 23, and from about 19 to about 21 nucleotides in length. As used herein, term “antisense strand” means the strand of an siRNA compound that is sufficiently complementary to a target molecule, e.g., the target nucleotide sequence of a target transcript.

The sense strand of a double stranded siRNA compound may be equal to or at least about 14, about 15, about 16, about 17, about 18, about 19, about 20, about 25, about 30, about 40, or about 60 nucleotides in length. It may be equal to or less than about 200, about 100, or about 50, nucleotides in length. Ranges may be from about 17 to about 25, from about 19 to about 23, and from about 19 to about 21 nucleotides in length.

The double strand portion of a double stranded siRNA compound may be equal to or at least about 14, about 15, about 16, about 17, about 18, about 19, about 20, about 21, about 22, about 23, about 24, about 25, about 30, about 40, or about 60 nucleotide pairs in length. It may be equal to or less than about 200, about 100, or about 50, nucleotides pairs in length. Ranges may be from about 15 to about 30, from about 17 to about 23, from about 19 to about 23, and from about 19 to about 21 nucleotides pairs in length.

In embodiments, the siRNA compound is sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1 to 3 nucleotides. The overhangs can be the result of one strand being longer than the other, or the result of two strands of the same length being staggered. Some embodiments will have at least one 3′ overhang. In embodiments, both ends of an siRNA molecule will have a 3′ overhang. In embodiments, the overhang is 2 nucleotides.

In embodiments, the length for the duplexed region is from about 15 to about 30, or about 18, about 19, about 20, about 21, about 22, or about 23 nucleotides in length, e.g., in the ssiRNA (siRNA with sticky overhangs) compound range discussed above. ssiRNA compounds can resemble in length and structure the natural Dicer processed products from long dsiRNAs.

Embodiments in which the two strands of the ssiRNA compound are linked, e.g., covalently linked are also included. In embodiments, hairpin, or other single strand structures which provide a double stranded region, and a 3′ over hangs are included.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene.

In embodiments, an siRNA compound is “sufficiently complementary” to a target transcript, such that the siRNA compound silences production of protein encoded by the target mRNA. In embodiments, the siRNA compound is “sufficiently complementary” to at least a portion of a target transcript, such that the siRNA compound silences production of the gene product encoded by the target transcript. In another embodiment, the siRNA compound is “exactly complementary” to a target nucleotide sequence (e.g., a portion of a target transcript) such that the target nucleotide sequence and the siRNA compound anneal, for example to form a hybrid made exclusively of Watson-Crick base pairs in the region of exact complementarity. A “sufficiently complementary” to a target nucleotide sequence can include an internal region (e.g., of at least about 10 nucleotides) that is exactly complementary to a target nucleotide sequence. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models, as well as in clinical studies MicroRNAs

In embodiments, the AC includes a microRNA molecule. MicroRNAs (miRNAs) are a highly conserved class of small RNA molecules that are transcribed from DNA in the genomes of plants and animals but are not translated into protein. Processed miRNAs are single stranded 17-25 nucleotide RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

### Antagomirs

In embodiments, the AC is an antagomir. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes that include the antagomir and endogenous miRNA, thereby preventing miRNA-induced gene silencing. An example of antagomir-mediated miRNA silencing is the silencing of miR-122, described in Krutzfeldt et al., Nature (2005), 438: 685-689, which is expressly incorporated by reference herein in its entirety. Antagomir RNAs may be synthesized using standard solid phase oligonucleotide synthesis protocols (U.S. patent application Ser. Nos. 11/502,158 and 11/657,341; the disclosure of each of which are incorporated herein by reference).

An antagomir can include ligand-conjugated monomer subunits and monomers for oligonucleotide synthesis. Monomers are described in U.S. application Ser. No. 10/916,185. An antagomir can have a ZXY structure, such as is described in PCT Application No. PCT/US2004/07070. An antagomir can be complexed with an amphipathic moiety. Amphipathic moieties for use with oligonucleotide agents are described in PCT Application No. PCT/US2004/07070.

### Aptamers

In embodiments, the AC includes an aptamer. Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules (Eaton, Curr. Opin. Chem. Biol. (1997), 1: 10-16; Famulok, Curr. Opin.

Struct. Biol. (1999), 9:324-9; and Hermann and Patel, Science (2000), 287:820-5). Aptamers may be RNA or DNA based and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, the term “aptamer” also includes “secondary aptamers” containing a consensus sequence derived from comparing two or more known aptamers to a given target. In embodiments, the aptamer is an “intracellular aptamer”, or “intramer”, which specifically recognize intracellular targets (Famulok et al., Chem Biol. (2001),8(10):931-939; Yoon and Rossi, Adv. Drug Deliv. Rev. (2018), 134:22-35; each incorporated by reference herein).

### Ribozymes

In embodiments, the AC is a ribozyme. Ribozymes are RNA molecules complexes having specific catalytic domains that possess endonuclease activity (Kim and Cech, Proc. Natl. Acad.

Sci. USA (1987),84(24):8788-92; Forster and Symons, Cell (1987) 24, 49(2):211-20). For example, a large number of ribozymes accelerate phosphoester transfer reactions with a high degree of specificity, often cleaving only one of several phosphoesters in an oligonucleotide substrate (Cech et al., Cell (1981), 27(3 Pt 2):487-96; Michel and Westhof, J. Mol. Biol. (1990), 5, 216(3):585-610; Reinhold-Hurek and Shub, Nature (1992), 14, 357(6374): 173-6). This specificity has been attributed to the requirement that the substrate bind via specific base-pairing interactions to the internal guide sequence (IGS) of the ribozyme prior to chemical reaction.

At least six basic varieties of naturally occurring enzymatic RNAs are known presently. Each can catalyze the hydrolysis of RNA phosphodiester bonds in trans (and thus can cleave other RNA molecules) under physiological conditions, In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis 6 virus, group I intron or RNaseP RNA (in association with an RNA guide sequence) or Neurospora VS RNA motif, for example. Specific examples of hammerhead motifs are described by Rossi et al. Nucleic Acids Res. (1992), 20(17):4559-65. Examples of hairpin motifs are described by Hampel et al. (Eur. Pat. Appl. Publ. No. EP 0360257), Hampel and Tritz, Biochemistry (1989), 28(12):4929-33; Hampel et al, Nucleic Acids Res. (1990),18(2):299-304 and U.S. Pat. No. 5,631,359. An example of the hepatitis virus motif is described by Perrotta and Been, Biochemistry (1992), 31(47): 11843-52; an example of the RNaseP motif is described by Guerrier-Takada et al., Cell (1983), 35(3 Pt 2):849-57; Neurospora VS RNA ribozyme motif is described by Collins (Saville and Collins, Cell (1990), 61(4):685-96; Saville and Collins, Proc. Natl. Acad. Sci. USA (1991),88(19):8826-30; Collins and Olive, Biochemistry (1993),32(1 1):2795-9); and an example of the Group I intron is described in U.S. Pat. No. 4,987,071. In embodiments, enzymatic nucleic acid molecules have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein. In embodiments, the ribozyme is targeted to a target nucleotide sequence of a target transcript.

Ribozyme activity can be increased by altering the length of the ribozyme binding arms or chemically synthesizing ribozymes with modifications that prevent their degradation by serum ribonucleases (see e.g. , Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U. S. Patent 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

### Supermir

In embodiments, the AC is a supermir. A supermir refers to a single stranded, double stranded, or partially double stranded oligomer or polymer of RNA, polymer of DNA, or both, or modifications thereof, which has a nucleotide sequence that is substantially identical to an miRNA and that is antisense with respect to its target, This term includes oligonucleotides composed of naturally-occurring nucleobases, sugars and covalent internucleoside (backbone) linkages and which contain at least one non-naturally-occurring portion which functions similarly. Such modified or substituted oligonucleotides have desirable properties such as, for example, enhanced cellular uptake, enhanced affinity for nucleic acid target and increased stability in the presence of nucleases. In embodiments, the supermir does not include a sense strand, and in another embodiment, the supermir does not self-hybridize to a significant extent. A supermir can have secondary structure, but it is substantially single-stranded under physiological conditions. A supermir that is substantially single-stranded is single-stranded to the extent that less than about 50% (e.g., less than about 40%, about 30%, about 20%, about 10%, or about 5%) of the supermir is duplexed with itself. The supermir can include a hairpin segment, e.g., sequence, for example, at the 3′ end can self-hybridize and form a duplex region, e.g., a duplex region of at least about 1, about 2, about 3, or about 4 or less than about 8, about 7, about 6, or about 5 nucleotides, or about 5 nucleotides. The duplexed region can be connected by a linker, e.g., a nucleotide linker, e.g., about 3, about 4, about 5, or about 6 dTs, e.g., modified dTs. In another embodiment the supermir is duplexed with a shorter oligo, e.g., of about 5, about 6, about 7, about 8, about 9, or about 10 nucleotides in length, e.g., at one or both of the 3′ and 5′ end or at one end and in the non-terminal or middle of the supermir.

miRNA Mimics

In embodiments, the AC is a miRNA mimic. miRNA mimics represent a class of molecules that can be used to imitate the gene silencing ability of one or more miRNAs. Thus, the term “microRNA mimic” refers to synthetic non-coding RNAs (i.e., the miRNA is not obtained by purification from a source of the endogenous miRNA) that are capable of entering the RNAi pathway and regulating gene expression. miRNA mimics can be designed as mature molecules (e.g., single stranded) or mimic precursors (e.g., pri- or pre-miRNAs). miRNA mimics can include nucleic acid (modified or modified nucleic acids) including oligonucleotides that include, without limitation, RNA, modified RNA, DNA, modified DNA, locked nucleic acids, or 2′-0,4′-C-ethylene-bridged nucleic acids (ENA), or any combination of the above (including DNA-RNA hybrids). In addition, miRNA mimics can include conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can include 2′ modifications (including 2′-0 methyl modifications and 2′ F modifications) on one or both strands of the molecule and internucleoside modifications (e.g., phosphorothioate modifications) that enhance nucleic acid stability and/or specificity. In addition, miRNA mimics can include overhangs. The overhangs can include from about 1 to about 6 nucleotides on either the 3′ or 5′ end of either strand and can be modified to enhance stability or functionality. In embodiments, a miRNA mimic includes a duplex region of from about 16 to about 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can include 2′ F modification of all of the Cs and Us, phosphorylation of the 5′ end of the oligonucleotide, and stabilized internucleoside linkages associated with a 2 nucleotide 3′ overhang. miRNA inhibitor

In embodiments, the AC is a miRNA inhibitor. The terms “antimir” “microRNA inhibitor”, “miR inhibitor”, or “miRNA inhibitor” are synonymous and refer to oligonucleotides or modified oligonucleotides that interfere with the ability of specific miRNAs. In general, the inhibitors are nucleic acid or modified nucleic acids in nature including oligonucleotides that include RNA, modified RNA, DNA, modified DNA, locked nucleic acids (LNAs), or any combination of the above.

Modifications include 2′ modifications (including 2′-0 alkyl modifications and 2′ F modifications) and internucleoside modifications (e.g., phosphorothioate modifications) that can affect delivery, stability, specificity, intracellular compartmentalization, or potency. In addition, miRNA inhibitors can include conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors include contain one or more sequences or portions of sequences that are complementary or partially complementary with the mature strand (or strands) of the miRNA to be targeted. In addition, the miRNA inhibitor may also include additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In embodiments, one or both of the additional sequences are arbitrary sequences capable of forming hairpins. Thus, in embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

Linking groups or bifunctional linking moieties such as those known in the art are amenable to the compounds provided herein. Linking groups are useful for attachment of chemical functional groups, conjugate groups, reporter groups and other groups to selective sites in a parent compound such as for example an AC. In general, a bifunctional linking moiety includes a hydrocarbyl moiety having two functional groups. One of the functional groups is selected to bind to a parent molecule or compound of interest and the other is selected to bind essentially any selected group such as chemical functional group or a conjugate group. Any of the linkers described here may be used. In embodiments, the linker includes a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of substituent groups includes hydroxyl, amino, alkoxy, carboxy, benzyl, phenyl, nitro, thiol, thioalkoxy, halogen, alkyl, aryl, alkenyl and alkynyl.

In embodiments, AC includes nucleotide modification designed to not support RNase H activity. Nucleotide modifications of antisense compounds that do not support RNase H activity are known and include, but are not limited to, 2′-O-methoxy ethyl/phosphorothioate (MOE) modifications. Advantageously, AC with MOE modifications have increased affinity for target RNA and increase nuclease stability.

### Immunostimulatory Oligonucleotides

In embodiments, the therapeutic moiety is an immunostimulatory oligonucleotide. Immunostimulatory oligonucleotides (ISS; single—or double-stranded) are capable of inducing an immune response when administered to a patient, which may be a mammal or other patient. ISS include, e.g., certain palindromes leading to hairpin secondary structures (see Yamamoto S., et al. (1992) J. Immunol. 148: 4072-4076), or CpG motifs, as well as other known ISS features (such as multi-G domains, see WO 96/11266).

The immune response may be an innate or an adaptive immune response. The immune system is divided into a more innate immune system, and acquired adaptive immune system of vertebrates, the latter of which is further divided into humoral cellular components. In particular embodiments, the immune response may be mucosal.

Immunostimulatory nucleic acids are considered to be non-sequence specific when it is not required that they specifically bind to and reduce the expression of a target polynucleotide in order to provoke an immune response. Thus, certain immunostimulatory nucleic acids may include a sequence corresponding to a region of a naturally occurring gene or mRNA, but they may still be considered non-sequence specific immunostimulatory nucleic acids.

In embodiments, the immunostimulatory nucleic acid or oligonucleotide includes at least one CpG dinucleotide. The oligonucleotide or CpG dinucleotide may be unmethylated or methylated. In another embodiment, the immunostimulatory nucleic acid includes at least one CpG dinucleotide having a methylated cytosine. In embodiments, the nucleic acid includes a single CpG dinucleotide, wherein the cytosine in said CpG dinucleotide is methylated. In a specific embodiment, the nucleic acid includes the sequence 5‘ TAACGTTGAGGG’CAT 3′ (SEQ ID NO: 369). In an alternative embodiment, the nucleic acid includes at least two CpG dinucleotides, wherein at least one cytosine in the CpG dinucleotides is methylated. In a further embodiment, each cytosine in the CpG dinucleotides present in the sequence is methylated. In another embodiment, the nucleic acid includes a plurality of CpG dinucleotides, wherein at least one of said CpG dinucleotides includes a methylated cytosine.

Additional specific nucleic acid sequences of oligonucleotides (ODNs) suitable for use in the compositions and methods are described in Raney et al, Journal of Pharmacology and Experimental Therapeutics, 298:1185-1192 (2001). In certain embodiments, ODNs used in the compositions and methods have a phosphodiester(“PO”) backbone or a phosphorothioate (“PS”) backbone, and/or at least one methylated cytosine residue in a CpG motif.

### Decoy Oligonucleotides

In embodiments, the therapeutic moiety is a decoy oligonucleotide. Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest, 2000, 106: 1071-1075, which is expressly incorporated by reference herein, in its entirety.

### U1 Adaptor

In some embodiments, the therapeutic moiety is a U1 adaptor. U1 adaptors inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49:77-95). Nucleotides 2-11 of′ the 5′end of U1 snRNA base pair bind with the 5′ss of the pre mRNA. In one embodiment, oligonucleotides are U1 adaptors. In one embodiment, the Ul adaptor can be administered in combination with at least one other iRNA agent.

### (CRISPR) Gene-Editing Machinery

In embodiments, the compounds disclosed herein include one or more CPP (or cCPP) conjugated to CRISPR gene-editing machinery. As used herein, “CRISPR gene-editing machinery” refers to protein, nucleic acids, or combinations thereof, which may be used to edit a genome. Non-limiting examples of gene-editing machinery include gRNAs, nucleases, nuclease inhibitors, and combinations and complexes thereof. The following patent documents describe CRISPR gene-editing machinery: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859. Each of the aforementioned patent documents is incorporated by reference herein in its entirety.

In embodiments, a linker conjugates the cCPP to the CRISPR gene-editing machinery. Any linker described in this disclosure or that is known to a person of skill in the art may be utilized.

gRNA

In embodiments, the compounds include the CPP (or cCPP) is conjugated to a gRNA. A gRNA targets a genomic loci in a prokaryotic or eukaryotic cell.

In embodiments, the gRNA is a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In embodiments, the spacer may be about 17-24 bases in length, such as about 20 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer sequence has between about 40% to about 80% GC content.

In embodiments, the spacer targets a site that immediately precedes a 5′ protospacer adjacent motif (PAM). The PAM sequence may be selected based on the desired nuclease. For example, the PAM sequence may be any one of the PAM sequences shown in Table 3 below, wherein N refers to any nucleic acid, R refers to A or G, Y refers to C or T, W refers to A or T, and V refers to A or C or G.

TABLE 3
Exemplary Nucleases and PAM sequences

| PAM sequence | | |
|---|---|---|
| (5′ to 3′) | Nuclease | Isolated from |
| NGG | SpCas9 | Streptococcus pyogenes |
| NGRRT or NGRRN | SaCas9 | Staphylococcus aureus |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

In embodiments, a spacer may target a sequence of a mammalian gene, such as a human gene. In embodiments, the spacer may target a mutant gene. In embodiments, the spacer may target a coding sequence. In embodiments, the spacer may target an exonic sequence. In embodiments, the spacer may target a polyadenylation site (PS). In embodiments, the spacer may target a sequence element of a PS. In embodiments, the spacer may target a polyadenylation signal (PAS), an intervening sequence (IS), a cleavage site (CS), a downstream element (DES), or a portion or combination thereof. In embodiments, a spacer may target a splicing element (SE) or a cis-splicing regulatory element (SRE).

The scaffold sequence is the sequence within the sgRNA that is responsible for nuclease (e.g., Cas9) binding. The scaffold sequence does not include the spacer/targeting sequence. In embodiments, the scaffold may be about 1 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, or about 120 to about 130 nucleotides in length. In embodiments, the scaffold may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60,about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In embodiments, the scaffold may be at least 10, at least 20, at least 30, at least 40, at least 50, at least 60, at least 70, at least 80, at least 90, at least 100, at least 110, at least 120, or at least 125 nucleotides in length.

In embodiments, the gRNA is a dual-molecule guide RNA, e.g, crRNA and tracrRNA. In embodiments, the gRNA may further include a poly(A) tail.

In embodiments, a compound that includes a CPP is conjugated to a nucleic acid that includes a gRNA. In embodiments, the nucleic acid includes about 1, about 2, about 3, about 4, about 5, about 6, about 7, about 8, about 9, about 10, about 11, about 12, about 13, about 14, about 15, about 16, about 17, about 18, about 19, or about 20 gRNAs. In embodiments, the gRNAs recognize the same target. In embodiments, the gRNAs recognize different targets. In embodiments, the nucleic acid that includes a gRNA includes a sequence encoding a promoter, wherein the promoter drives expression of the gRNA.

### Nuclease

In embodiments, the compounds include a cell penetrating peptide conjugated to a nuclease. In embodiments, the nuclease is a Type II, Type V-A, Type V-B, Type VC, Type V-U, Type VI-B nuclease. In embodiments, the nuclease is a transcription, activator-like effector nuclease (TALEN), a meganuclease, or a zinc-finger nuclease. In embodiments, the nuclease is a Cas9, Casl2a (Cpf1), Casl2b, Casl2c, Tnp-B like, Casl3a (C2c2), Casl3b, or Cas14 nuclease.

For example, in some embodiments, the nuclease is a Cas9 nuclease or a Cpf1 nuclease.

In embodiments, the nuclease is a modified form or variant of a Cas9, Casl2a (Cpf1), Casl2b, Casl2c, Tnp-B like, Casl3a (C2c2), Casl3b, or Cas14 nuclease. In embodiments, the nuclease is a modified form or variant of a TAL nuclease, a meganuclease, or a zinc-finger nuclease. A “modified” or “variant” nuclease is one that is, for example, truncated, fused to another protein (such as another nuclease), catalytically inactivated, etc. In embodiments, the nuclease may have at least about 80%, at least about 85%, at least about 90%, at least about 95%, at least about 98%, at least about 99%, or about 100% sequence identity to a naturally occurring Cas9, Casl2a (Cpf1), Casl2b, Casl2c, Tnp-B like, Casl3a (C2c2), Casl3b, Cas14 nuclease, or a TALEN, meganuclease, or zinc-finger nuclease. In embodiments, the nuclease is a Cas9 nuclease derived from S. pyogenes (SpCas9). In embodiments, a nuclease has at least about 90%, at least about 95%, at least about 96%, at least about 97%, at least about 98%, or at least about 99% sequence identity to a Cas9 nuclease derived from S. pyogenes (SpCas9). In embodiments, the nuclease is a Cas9 derived from S. aureus (SaCas9). In embodiments, the nuclease has at least about 90%, at least about 95%, at least about 96%, at least about 97%, at least about 98%, or at least about 99% sequence identity to a Cas9 derived from S. aureus (SaCas9). In embodiments, the Cpf1 is a Cpf1 enzyme from Acidaminococcus (species BV3L6, UniProt Accession No. U2UMQ6). In embodiments, the nuclease has at least about 90%, at least about 95%, at least about 96%, at least about 97%, at least about 98%, or at least about 99% sequence identity to a Cpf1 enzyme from Acidaminococcus (species BV3L6, UniProt Accession No. U2UMQ6).

In embodiments, the Cpf1 is a Cpf1 enzyme from Lachnospiraceae (species ND2006, UniProt Accession No. AOA182DWE3). In embodiments, the nuclease has at least about 90%, at least about 95%, at least about 96%, at least about 97%, at least about 98%, or at least about 99% sequence identity to a Cpf1 enzyme from Lachnospiraceae. In embodiments, a sequence encoding the nuclease is codon optimized for expression in mammalian cells. In embodiments, the sequence encoding the nuclease is codon optimized for expression in human cells or mouse cells.

In embodiments, a compound that includes a CPP is conjugated to a nuclease. In embodiments, the nuclease is a soluble protein.

In embodiments, a compound that includes a CPP is conjugated to a nucleic acid encoding a nuclease. In embodiments, the nucleic acid encoding a nuclease includes a sequence encoding a promoter, wherein the promoter drives expression of the nuclease. gRNA and Nuclease Combinations

In embodiments, the compounds include one or more CPP (or cCPP) conjugated to a gRNA and a nuclease. In embodiments, the one or more CPP (or cCPP) are conjugated to a nucleic acid encoding a gRNA and/or a nuclease. In embodiments, the nucleic acid encoding a nuclease and a gRNA includes a sequence encoding a promoter, wherein the promoter drives expression of the nuclease and the gRNA. In embodiments, the nucleic acid encoding a nuclease and a gRNA includes two promoters, wherein a first promoter controls expression of the nuclease and a second promoter controls expression of the gRNA. In embodiments, the nucleic acid encoding a gRNA and a nuclease encodes from about 1 to about 20 gRNAs, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. In embodiments, the gRNAs recognize different targets. In embodiments, the gRNAs recognize the same target.

In embodiments, the compounds include a cell penetrating peptide (or cCPP) conjugated to a ribonucleoprotein (RNP) that includes a gRNA and a nuclease.

In embodiments, a composition that includes: (a) a CPP conjugated to a gRNA and (b) a nuclease is delivered to a cell. In embodiments, a composition that includes: (a) a CPP conjugated to a nuclease and (b) an gRNA is delivered to a cell.

In embodiments, a composition that includes: (a) a first CPP conjugated to a gRNA and (b) a second CPP conjugated to a nuclease is delivered to a cell. In embodiments, the first CPP and second CPP are the same. In embodiments, the first CPP and second CPP are different.

Genetic Element ofInterest

In embodiments, the compounds disclosed herein include a cell penetrating peptide conjugated to a genetic element of interest. In embodiments, a genetic element of interest replaces a genomic DNA sequence cleaved by a nuclease. Non-limiting examples of genetic elements of interest include genes, a single nucleotide polymorphism, promoter, or terminators.

### Nuclease Inhibitors

In embodiments, the compounds disclosed herein include a cell penetrating peptide conjugated to an inhibitor of a nuclease (e.g., Cas9). A limitation of gene editing is potential off-target editing. The delivery of a nuclease inhibitor will limit off-target editing. In embodiments, the nuclease inhibitor is a polypeptide, polynucleotide, or small molecule. Exemplary nuclease inhibitors are described in U.S. Publication No. 2020/087354, International Publication No. 2018/085288, U.S. Publication No. 2018/0382741, International Publication No. 2019/089761, International Publication No. 2020/068304, International Publication No. 2020/041384, and International Publication No. 2019/076651, each of which is incorporated by reference herein in its entirety.

### Therapeutic Polypeptides

In embodiments, the therapeutic moiety includes a polypeptide. In embodiments, the therapeutic moiety includes a protein or a fragment thereof. In embodiments, the therapeutic moiety includes an RNA binding protein or an RNA binding fragment thereof. In embodiments, the therapeutic moiety includes an enzyme. In embodiments, the therapeutic moiety includes an RNA-cleaving enzyme or an active fragment thereof.

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the AC or the CPP.

Conjugate groups include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996,277,923).

Linking groups or bifunctional linking moieties such as those known in the art are amenable to the compounds provided herein. Linking groups are useful for attachment of chemical functional groups, conjugate groups, reporter groups and other groups to selective sites in a parent compound such as for example an AC. In general, a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. Any of the linkers described here may be used. In embodiments, the linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of substituent groups includes hydroxyl, amino, alkoxy, carboxy, benzyl, phenyl, nitro, thiol, thioalkoxy, halogen, alkyl, aryl, alkenyl and alkynyl.

In embodiments, the AC may be linked to a 10 arginine-serine dipeptide repeat. ACs linked to 10 arginine-serine dipeptide repeats for the artificial recruitment of splicing enhancer factors have been applied in vitro to induce inclusion of mutated BRCA1 and SMN2 exons that otherwise would be skipped. See Cartegni and Krainer 2003, incorporated by reference herein.

### Endosomal Escape Vehicles (EEVs)

An endosomal escape vehicle (EEV) can be used to transport a cargo across a cellular membrane, for example, to deliver the cargo to the cytosol or nucleus of a cell. Cargo can include a therapeutic moiety™. The EEV can comprise a cell penetrating peptide (CPP), for example, a cyclic cell penetrating peptide (cCPP). In embodiments, the EEV comprises a cCPP, which is conjugated to an exocyclic peptide (EP). The EP can be referred to interchangeably as a modulatory peptide (MP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 4 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF3), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF3) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO:1), KHKK (SEQ ID NO:2), KKHK (SEQ ID NO:3), KKKH (SEQ ID NO:4), KHKH (SEQ ID NO:5), HKHK (SEQ ID NO:6), KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), HBHBH, HBKBH, RRRRR (SEQ ID NO:17), KKKKK (SEQ ID NO:18), KKKRK (SEQ ID NO:19), RKKKK (SEQ ID NO:20), KRKKK (SEQ ID NO:21), KKRKK (SEQ ID NO:22), KKKKR (SEQ ID NO:23), KBKBK, RKKKKG (SEQ ID NO:25), KRKKKG (SEQ ID NO:26), KKRKKG (SEQ ID NO:27), KKKKRG (SEQ ID NO:28), RKKKKB (SEQ ID NO:29), KRKKKB (SEQ ID NO:30), KKRKKB (SEQ ID NO:31), KKKKRB (SEQ ID NO:32), KKKRKV (SEQ ID NO:33), RRRRRR (SEQ ID NO:34), HI*HIHH (SEQ ID NO:35), RHRHRH (SEQ ID NO:36), HRHRHR (SEQ ID NO:37), KRKRKR (SEQ ID NO:38), RKRKRK (SEQ ID NO:39), RBRBRB, KBKBKB, PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48), wherein B is beta-alanine. The amino acids in the EP can have D or L stereochemistry.

The EP can comprise KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), KKKKK (SEQ ID NO:18), KKKRK (SEQ ID NO:19), KBKBK, KKKRKV (SEQ ID NO:33), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48). The EP can comprise PKKKRKV (SEQ ID NO:42), RR, RRR, RHR, RBR, RBRBR, RBHBR, or HBRBH, wherein B is beta-alanine. The amino acids in the EP can have D or L stereochemistry.

The EP can consist of KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), KKKKK (SEQ ID NO:18), KKKRK (SEQ ID NO:19), KBKBK (SEQ ID NO:24), KKKRKV (SEQ ID NO:33), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48). The EP can consist of PKKKRKV (SEQ ID NO:42), RR, RRR, RHR, RBR, RBRBR, RBHBR, or HBRBH, wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO:42). The EP can consist of an NLS comprising the amino acid sequence PKKKRKV (SEQ ID NO:42). The EP can comprise an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NO:55), KAKKDEQILKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66). The EP can consist of an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NO:55), KAKKDEQTLKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66).

All exocyclic sequences can also contain an N-terminal acetyl group. Hence, for example, the EP can have the structure: Ac-PKKKRKV (SEQ ID NO:370).

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety™ such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

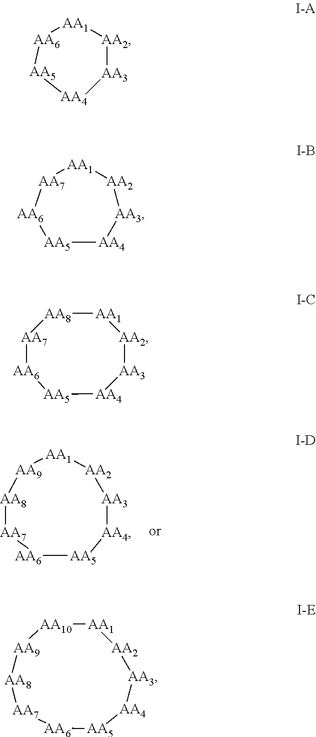

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 5 along with their abbreviations used herein.

TABLE 4
Amino Acid Abbreviations

| | Abbreviations* | Abbreviations* |
|---|---|---|
| Amino Acid | L-amino acid | D-amino acid |
| 2-[2-[2-aminoethoxy]ethoxy]- | AEEA, | NA |
| acetic acid | miniPEG, PEG2 | |
| Alanine | Ala (A) | ala (a) |
| Allo-isoleucine | Aile | Aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (⊖) |
| isoleucine | Ile (I) | ile (i) |
| leucine | Leu (L) | leu (l) |
| lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| norleucine | Nle (Ω) | nle |
| phenylalanine | Phe (F) | phe (f) |
| phenylglycine | Phg (Ψ) | phg |
| 4-(phosphonodifluoromethyl)- | F₂Pmp (Λ) | f₂pmp |
| phenylalanine | | |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: capital letters indicate the L-amino acid form, lower case letter indicate the D-amino acid form.

As used herein, “polyethylene glycol” and “PEG” are used interchangeably. “PEGm,” and “PEGₘ,” are, or are derived from, a molecule of the formula HO(CO)—(CH₂)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and m is 2. In embodiments, n is 1 and m is 4. In embodiments, n is 2 and m is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)ₙ—(OCH₂CH₂)ₘ—NH₂ where n is 1 and m is any integer from 1 to 23. For example, “miniPEG2” or “miniPEG2” is, or is derived from, (2-[2-[2-aminoethoxy]ethoxy]acetic acid), and “miniPEG4” or “miniPEG4” is, or is derived from, HO(CO)—(CH₂)ₙ—(OCH₂CH₂)ₘ—NH₂ where n is 1 and m is 4.

The cCPP can comprise 4 to 20 amino acids, wherein: (i) at least one amino acid has a side chain comprising a guanidine group, or a protonated form thereof; (ii) at least one amino acid has no side chain or a side chain comprising

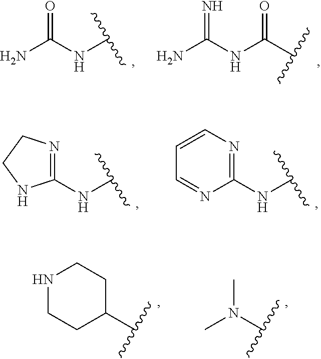

or a protonated form thereof; and (iii) at least two amino acids independently have a side chain comprising an aromatic or heteroaromatic group.

At least two amino acids can have no side chain or a side chain comprising

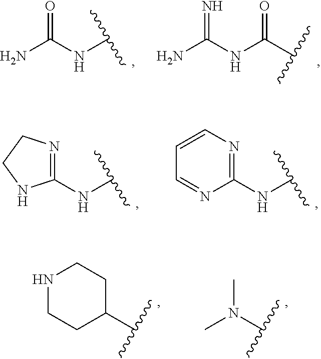

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or P-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

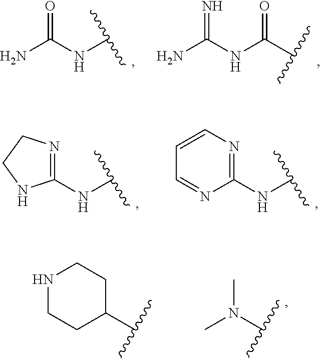

or a protonated form thereof.

The cCPP can comprise from 6 to 20 amino acid residues which form the cCPP, wherein: (i) at least two amino acid can independently be glycine, β-alanine, or 4-aminobutyric acid residues; (ii) at least one amino acid can have a side chain comprising an aryl or heteroaryl group; and (iii) at least one amino acid has a side chain comprising a guanidine group,

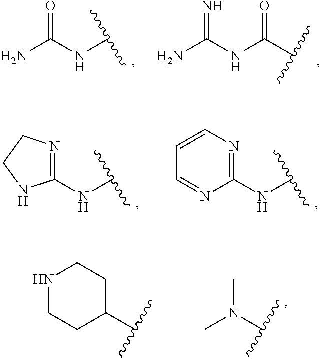

or a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

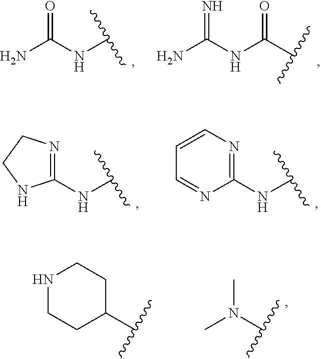

or a protonated form thereof

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, (3-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof.

The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4—or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group.

The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

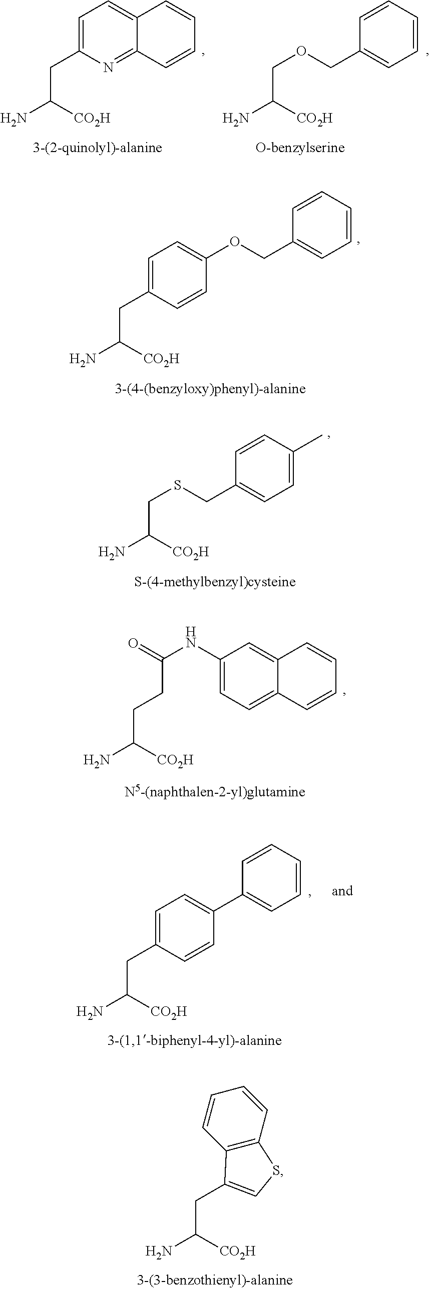

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents.

The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 5 lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 5
Amino Acid Hydrophobicity

| Amino | | Eisenberg | Engleman | Kyrie and | Hoop and | |
|---|---|---|---|---|---|---|
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å² at least about 220 Å², at least about 240 Å², at least about 250 Å², at least about 260 Å², at least about 270 Å², at least about 280 Å² at least about 290 Å², at least about 300 Å², at least about 310 Å² at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif, a cCPP having a Phe-Nal-Arg motif may exhibit improved cytosolic delivery efficiency compared to an otherwise identical cCPP having a Nal-Phe-Arg motif, and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Angstroms; Å²) of an amino acid side chain that is accessible to a solvent, SASA can be calculated using the ‘rolling ball’ algorithm developed by Shrake & Rupley (J Mol Biol. 79 (2): 351-71), which is herein incorporated by reference in its entirety for all purposes.

This algorithm uses a “sphere” of solvent of a particular radius to probe the surface of the molecule. A typical value of the sphere is 1.4 A, which approximates to the radius of a water molecule.

TABLE 6
Amino Acid SASA Values

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

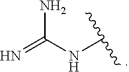 [SMILES: *NC(=N)N]

As used herein, a protonated form of guanidine refers to the structure:

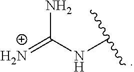 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

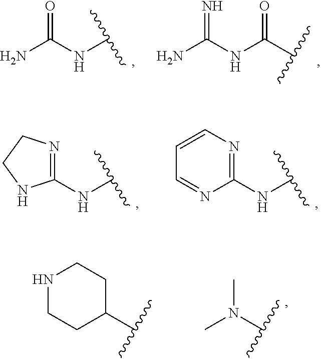

or a protonated form thereof.

The disclosure relates to a cCPP comprising from 4 to 20 amino acids residues, wherein: (i) at least one amino acid has a side chain comprising a guanidine group, or a protonated form thereof; (ii) at least one amino acid residue has no side chain or a side chain comprising

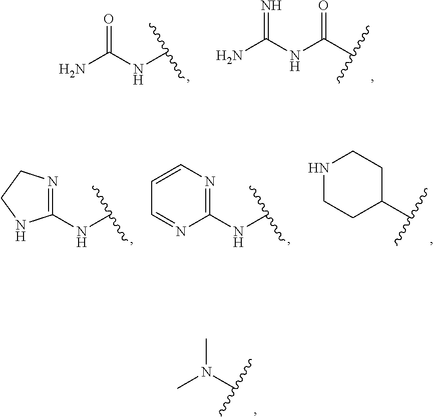

or a protonated form thereof; and (iii) at least two amino acids residues independently have a side chain comprising an aromatic or heteroaromatic group.

At least two amino acids residues can have no side chain or a side chain comprising

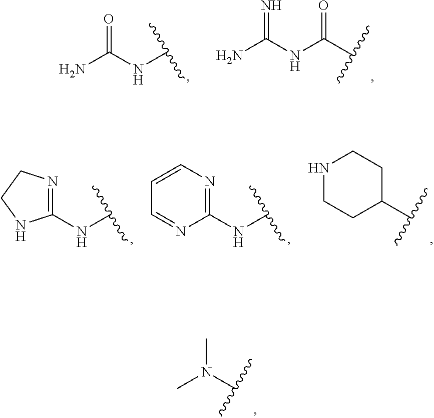

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

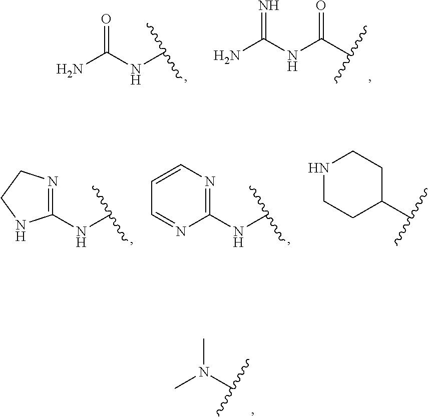

or a protonated form thereof.

The cCPP can comprise at least two amino acids each independently having one of the following moieties

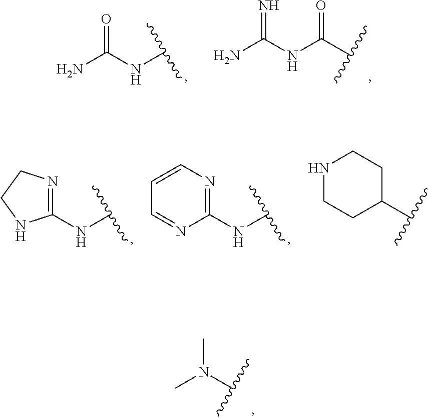

or a protonated form thereof. At least two amino acids can have a side chain comprising the same moiety selected from:

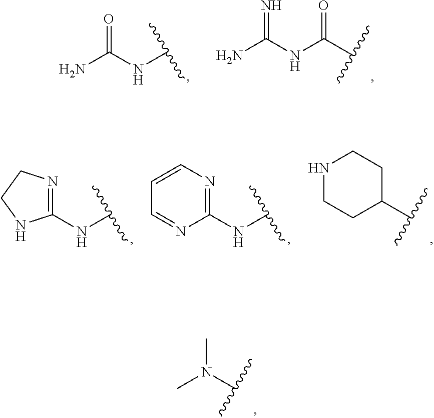

or a protonated form thereof. At least one amino acid can have a side chain comprising

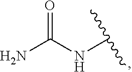 [SMILES: *NC(N)=O]

or a protonated form thereof. At least two amino acids can have a side chain comprising

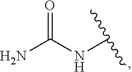 [SMILES: *NC(N)=O]

or a protonated form thereof. One, two, three, or four amino acids can have a side chain comprising

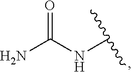 [SMILES: *NC(N)=O]

or a protonated form thereof. One amino acid can have a side chain comprising

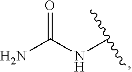 [SMILES: *NC(N)=O]

or a protonated form thereof. Two amino acids can have a side chain comprising

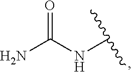 [SMILES: *NC(N)=O]

or a protonated form thereof.

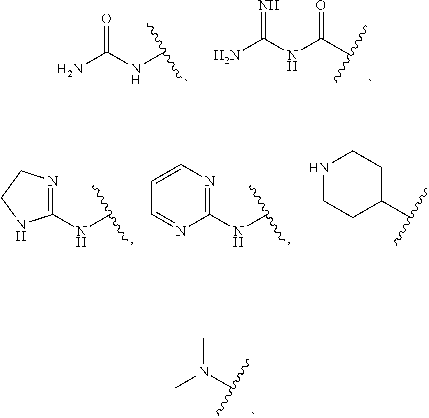

or a protonated form thereof, can be attached to the terminus of the amino acid side chain.

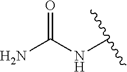 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

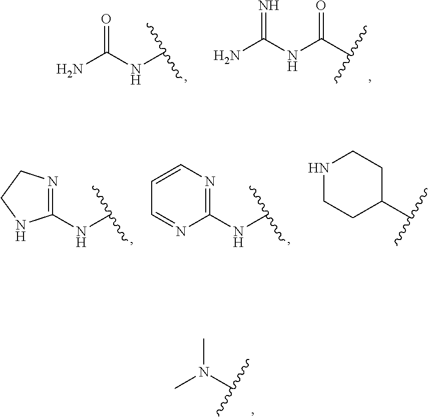

or a protonated form thereof

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamic acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

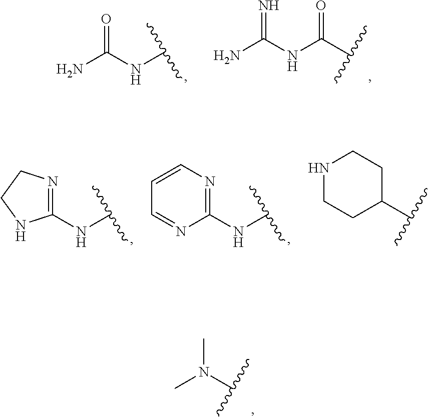

or a protonated form thereof, can be adjacent to an amino acid having a side chain comprising an aromatic or heteroaromatic group. An amino acid having a side chain comprising:

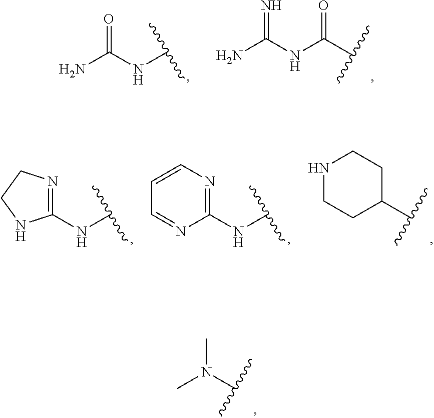

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

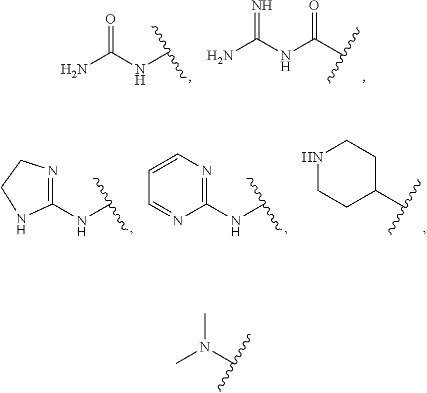

or protonated forms thereof, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

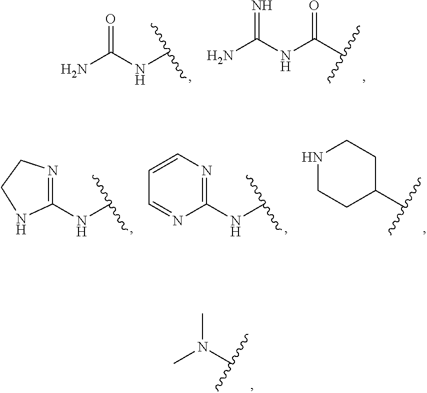

or a protonated form thereof. The cCPPs can comprise at least two contiguous amino acids having a side chain comprising an aromatic or heteroaromatic group and at least two non-adjacent amino acids having a side chain comprising

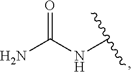 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

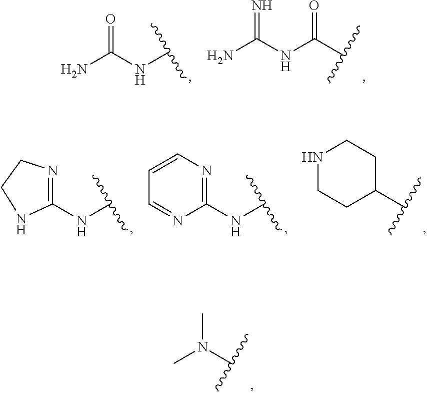

or a protonated form thereof, are alternating with at least two amino acids having a side chain comprising a guanidine group or protonated form thereof.

The cCPP can comprise the structure of Formula (A):

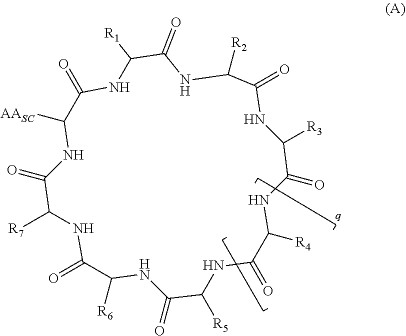

or a protonated form thereof,
wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, , 0-homoarginine, 3-(1-piperidinyl)alanine;
AA_{SC} is an amino acid side chain; and
q is 1, 2, 3 or 4.

In embodiments, at least one of R₄, R₅, R₆, R₇ are independently an uncharged, non-aromatic side chain of an amino acid. In embodiments, at least one of R₄, R₅, R₆, R₇ are independently H or a side chain of citrulline.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine or naphthylalanine. In embodiments, at least two uncharged, non-aromatic amino acids of the cyclic peptide are citrulline or glycine.

In embodiments, the cyclic peptide of Formula (A) is not selected from a cyclic peptide having a sequence of SEQ ID NO: 89-117.

In embodiments, the cyclic peptide of Formula (A) is selected from a cyclic peptide having a sequence of SEQ ID NO: 89-117.

CPP sequences and SEQ ID NOS

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| rRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Φ = L-naphthylalanine;
ϕ = D-naphthylalanine;
Ω = L-norleucine

The cCPP can comprise the structure of Formula I:

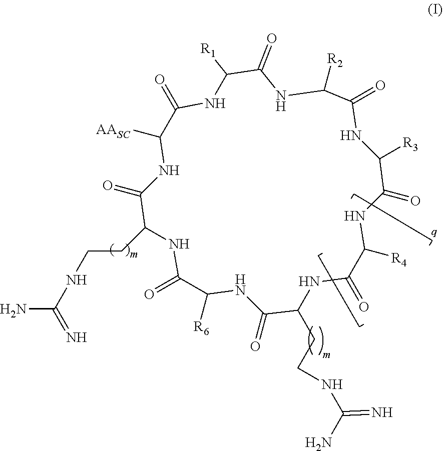

or a protonated form thereof,
wherein:
   R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₇ are independently H or an amino acid side chain;
AA_{SC} is an amino acid side chain;
q is 1, 2, 3 or 4; and
each m is independently an integer of 0, 1, 2, or 3.

R₁, R₂, and R₃ can each independently be H, -alkylene-aryl, or -alkylene-heteroaryl. R₁, R₂, and R-3 can each independently be H, -C₁₋₃alkylene-aryl, or -C₁₋₃alkylene-heteroaryl. R₁, R₂, and R₃ can each independently be H or -alkylene-aryl. R₁, R₂, and R₃ can each independently be H or -C₁₋₃alkylene-aryl. C1-3alkylene can be methylene. Aryl can be a 6- to 14-membered aryl.

Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, -C₁₋₃alkylene-Ph or -C₁₋₃alkylene-Naphthyl. R₁, R₂, and R₃ can each independently be H, —CH₂Ph, or —CH₂Naphthyl. R₁, R₂, and R₃ can each independently be H or -CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of P-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of P-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of 0-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl.

Heteroaryl can be pyridyl, quinolyl, and isoquinolyl. R₄ can be H, —C₁₋₃alkylene-Ph or -C₁₋₃alkylene-Naphthyl. R₄ can be H or the side chain of an amino acid in Table 4 or Table 6. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl.

Heteroaryl can be pyridyl, quinolyl, and isoquinolyl. R₅ can be H, —C₁₋₃alkylene-Ph or -C₁₋₃alkylene-Naphthyl. R₅ can be H or the side chain of an amino acid in Table 4 or Table 6. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃alkylene-heteroaryl. R₆ can be H or -alkylene-aryl. R₆ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl.

Heteroaryl can be pyridyl, quinolyl, and isoquinolyl. R₆ can be H, —C₁₋₃alkylene-Ph or -C₁₋₃alkylene-Naphthyl. R₆ can be H or the side chain of an amino acid in Table 4 or Table 6. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl.

Heteroaryl can be pyridyl, quinolyl, and isoquinolyl. R₇ can be H, —C₁₋₃alkylene-Ph or -C₁₋₃alkylene-Naphthyl. R₇ can be H or the side chain of an amino acid in Table 4 or Table 6. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are -CH₂Ph. One of R₁, R₂, R₃, and R₄ is —CH₂Ph.

Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R-3, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid.

At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, , 0-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid.

At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, , 0-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid.

At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine.

At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, , 0-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue.

The cCPP can further comprise a linker conjugated to the glutamine residue.
q can be 1, 2, or 3. q can 1 or 2. q can be 1. q can be 2. q can be 3. q can be 4.
m can be 1-3. m can be 1 or 2. m can be 0. m can be 1. m can be 2. m can be 3.

The cCPP of Formula (A) can comprise the structure of Formula (I)

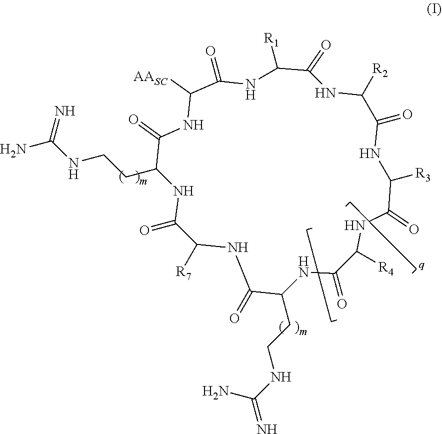

or protonated form thereof, wherein AA_{SC}, R₁, R₂, R₃, R₄, R₇, m, and q are as defined herein.

The cCPP of Formula (A) can comprise the structure of Formula (I-a) or Formula (I-b):

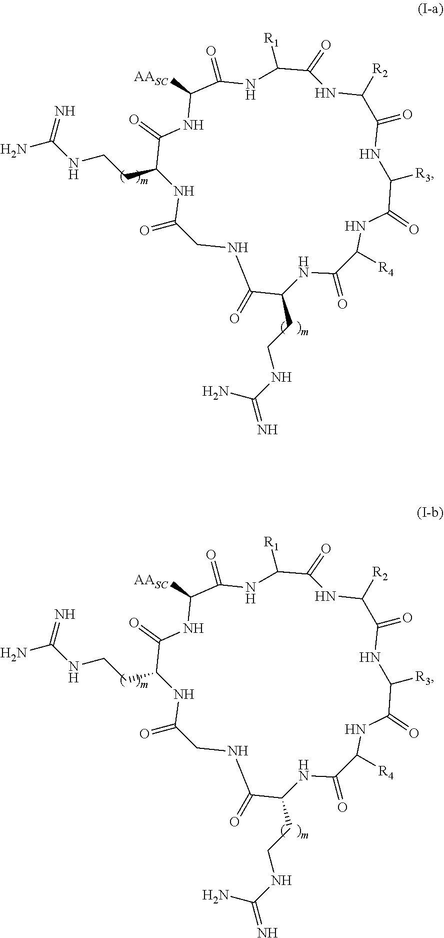

or protonated form thereof, wherein AA_{SC}, R₁, R₂, R₃, R₄, and m are as defined herein.

The cCPP of Formula (A) can comprise the structure of Formula (I-1), (I-2), (I-3), or (I-4) (SEQ ID NOS 646-649, respectively, in order of appearance):

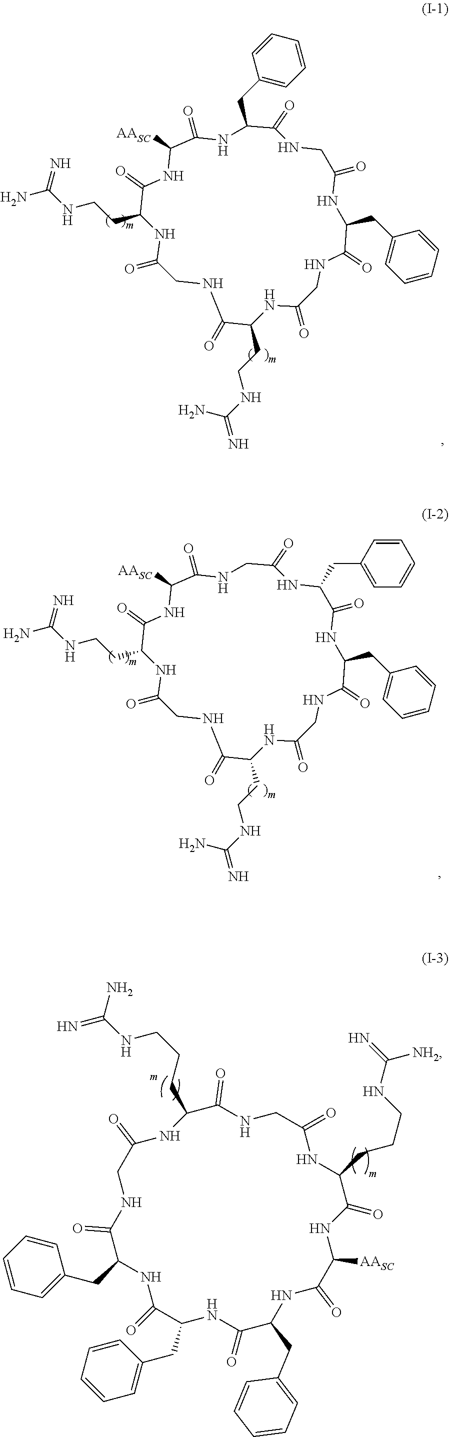

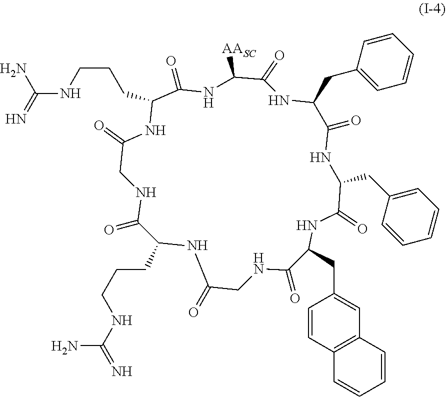

or protonated form thereof, wherein AA_{SC} and m are as defined herein.

The cCPP of Formula (A) can comprise the structure of Formula (I-5) or (I-6) (SEQ ID NOS 650-651, respectively, in order of appearance):

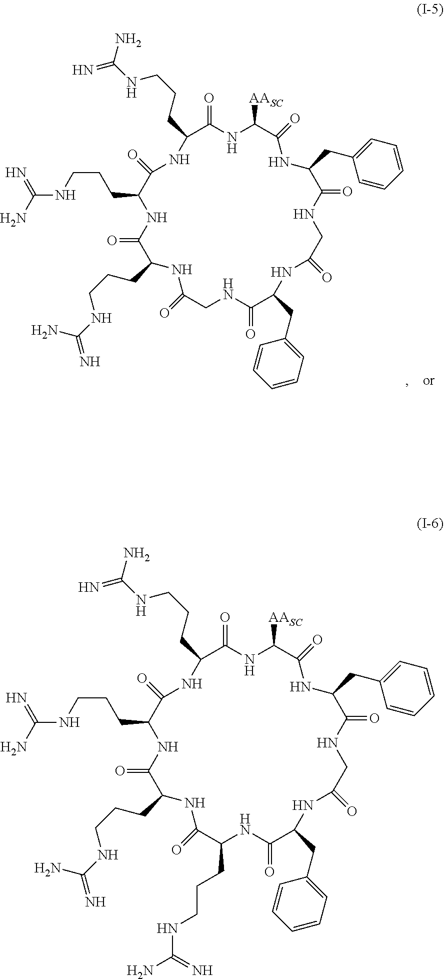

or protonated form thereof, wherein AA_{SC} is as defined herein.

The cCPP of Formula (A) can comprise the structure of Formula (I-1) (SEQ ID NO: 646):

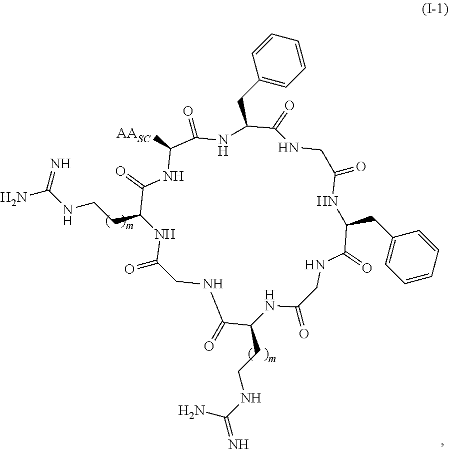

or a protonated form thereof, wherein AA_{SC} and m are as defined herein.

The cCPP of Formula (A) can comprise the structure of Formula (I-2) (SEQ ID NO: 647):

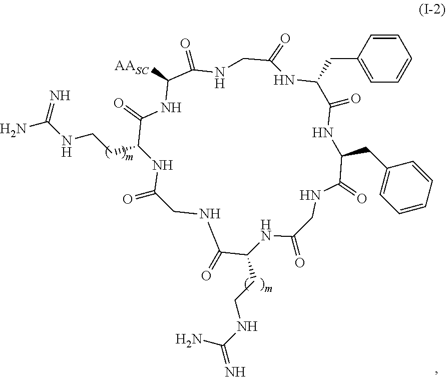

or a protonated form thereof, wherein AA_{SC} and m are as defined herein.

The cCPP of Formula (A) can comprise the structure of Formula (I-3) (SEQ ID NO: 648):

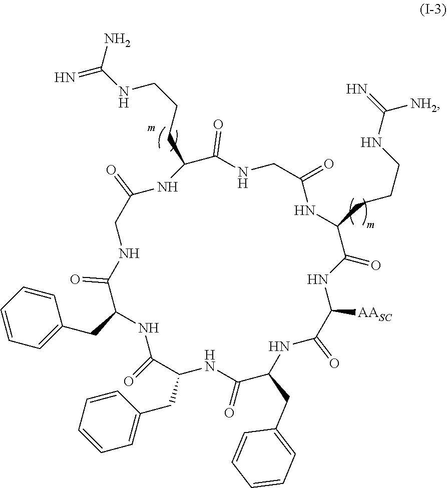

or a protonated form thereof, wherein AA_{SC} and m are as defined herein.

The cCPP of Formula (A) can comprise the structure of Formula (I-4) (SEQ ID NO: 649):

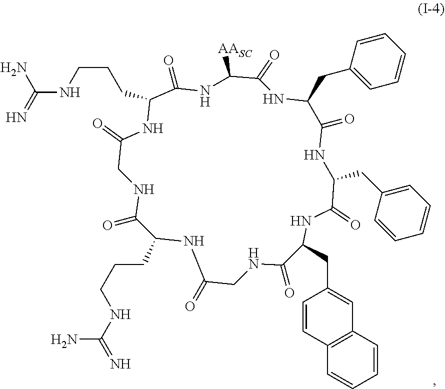

or a protonated form thereof, wherein AA_{SC} and m are as defined herein.

The cCPP of Formula (A) can comprise the structure of Formula (I-5) (SEQ ID NO: 650):

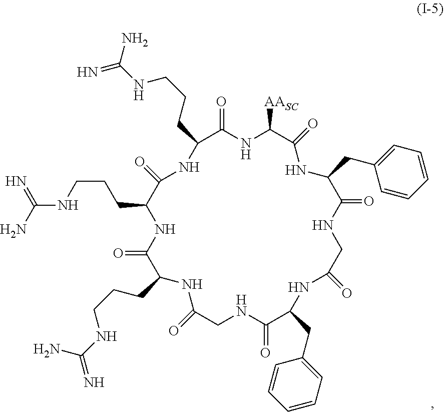

or a protonated form thereof, wherein AA_{SC} and m are as defined herein.

The cCPP of Formula (A) can comprise the structure of Formula (I-6) (SEQ ID NO: 651):

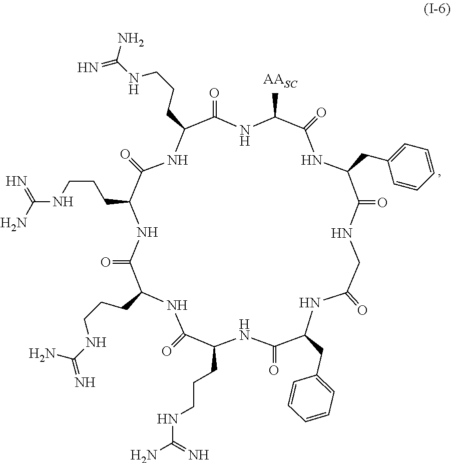 [SMILES: C[C@@H]1NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](Cc2ccccc2)NC(=O)CNC(=O)[C@H](Cc2ccccc2)NC1=O]

or a protonated form thereof, wherein AA_{SC} and m are as defined herein.

The cCPP can comprise one of the following sequences: FGFGRGR (SEQ ID NO:68); GfFGrGr (SEQ ID NO:69), FfΦGRGR (SEQ ID NO:70); FfFGRGR (SEQ ID NO:71); or FfΦGrGr (SEQ ID NO:72). The cCPP can have one of the following sequences: FGFΦ; GfFGrGrQ (SEQ ID NO:74), FfΦGRGRQ (SEQ ID NO:75); FfFGRGRQ (SEQ ID NO: 76); or FfΦGrGrQ (SEQ ID NO: 77).

The disclosure also relates to a cCPP having the structure of Formula (II):

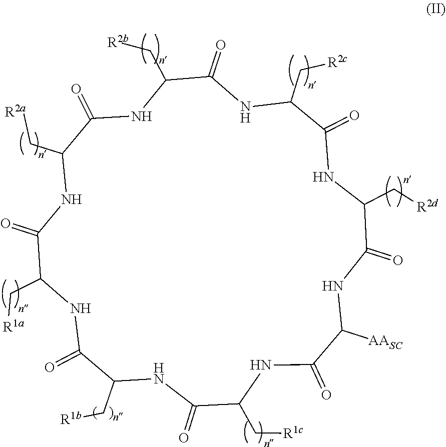

wherein:
AA_{SC} is an amino acid side chain;
R^{1a}, R^{1b}, and R^{1c} are each independently a 6- to 14-membered aryl or a 6- to 14-membered heteroaryl; R^{2a}, R^{2b}, R^{2c} and R^{2d} are independently an amino acid side chain; at least one of R^{2a}, R^{2b}, R^{2c} and R^{2d} is

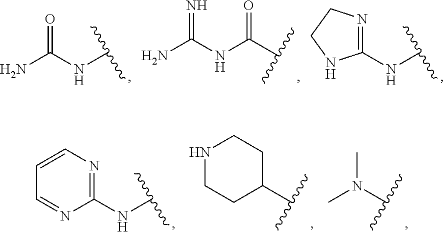

or a protonated form thereof,
at least one of R^{2a}, R^{2b}, R^{2c} and R^{2d} is guanidine or a protonated form thereof,
each n″ is independently an integer 0, 1, 2, 3, 4, or 5;
each n′ is independently an integer from 0, 1, 2, or 3; and
if n′ is 0 then R^{2a}, R^{2b}, R²b or R^{2d} is absent.

At least two of R^{2a}, R^{2b}, R^{2c} and R^{2d} can be

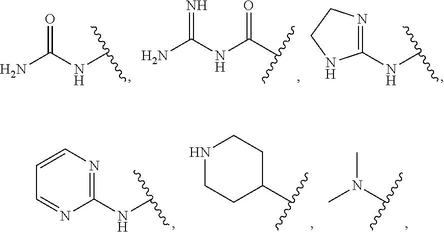

or a protonated form thereof. Two or three of R^{2a}, R^{2b}, R^{2c} and R^{2d} can be

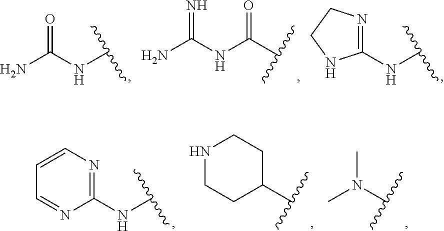

or a protonated form thereof one of R^{2a}, R^{2b}, R^{2c} and R^{2d} can be

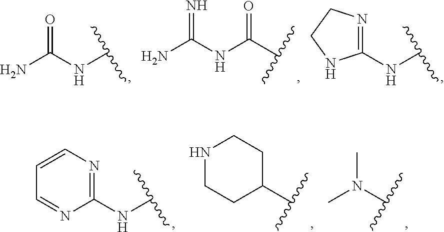

or a protonated form thereof. At least one of R^{2a}R^{2b}, R^{2c} and R^{2d} can be

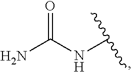 [SMILES: *NC(N)=O]

or a protonated form thereof, and the remaining of R^{2a}R^{2b}, R^{2c} and R^{2d} can be guanidine or a protonated form thereof. At least two of R^{2a}, R^{2b}, R^{2c} and

R^{2d} can be

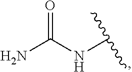 [SMILES: *NC(N)=O]

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

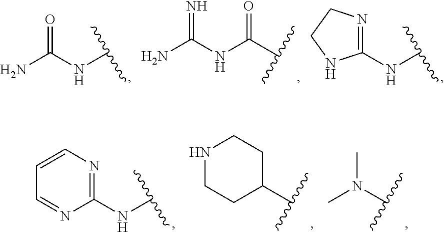

or a protonated form thereof. At least of R^{2a}, R^{2b}, R^{2c} and R^{2d} can be

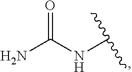 [SMILES: *NC(N)=O]

or a protonated form thereof, and the remaining of R^{2a}, R^{2b}, R^{2c} and R^{2d} can be guaninide or a protonated form thereof. At least two R^{2a}, R^{2b}, R^{2c} and R^{2d} groups can be

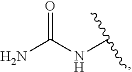 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

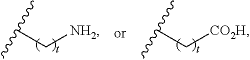

wherein t can be an integer from 0 to 5. AA_{SC} can be

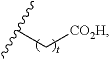

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1l}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1l}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

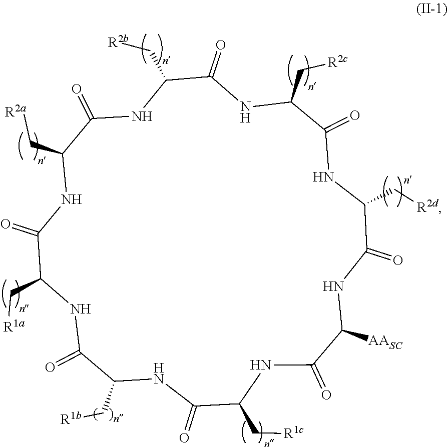

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

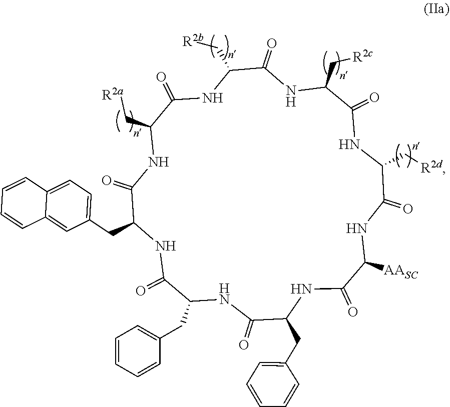

wherein Ria, R^{1b}, R^{1c}, R^{2a}, R^{2b}, R^{2c}, R^{2d}, AA_{SC} and n′ are as defined herein.

The cCPP of formula (II) can have the structure of Formula (IIb):

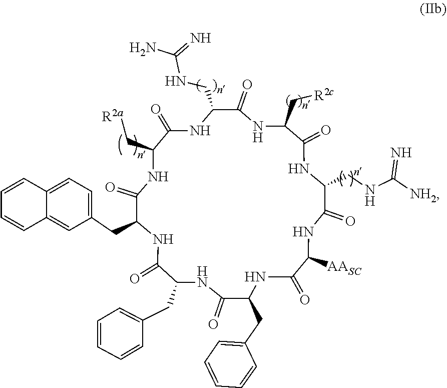

wherein R^{2a}, R^{2b}, AA_{SC}, and n′ are as defined herein.

The cCPP can have the structure of Formula (IIc) (SEQ ID NO: 656):

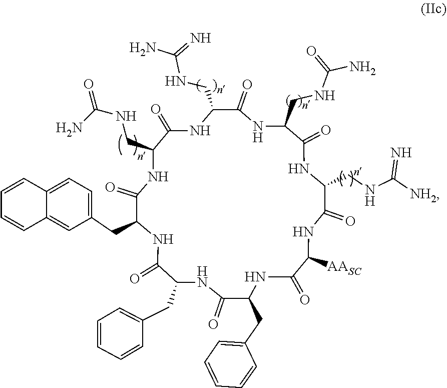

wherein:

AA_{SC} and n′ are as defined herein.

The cCPP of Formula (IIa) has one of the following structures:

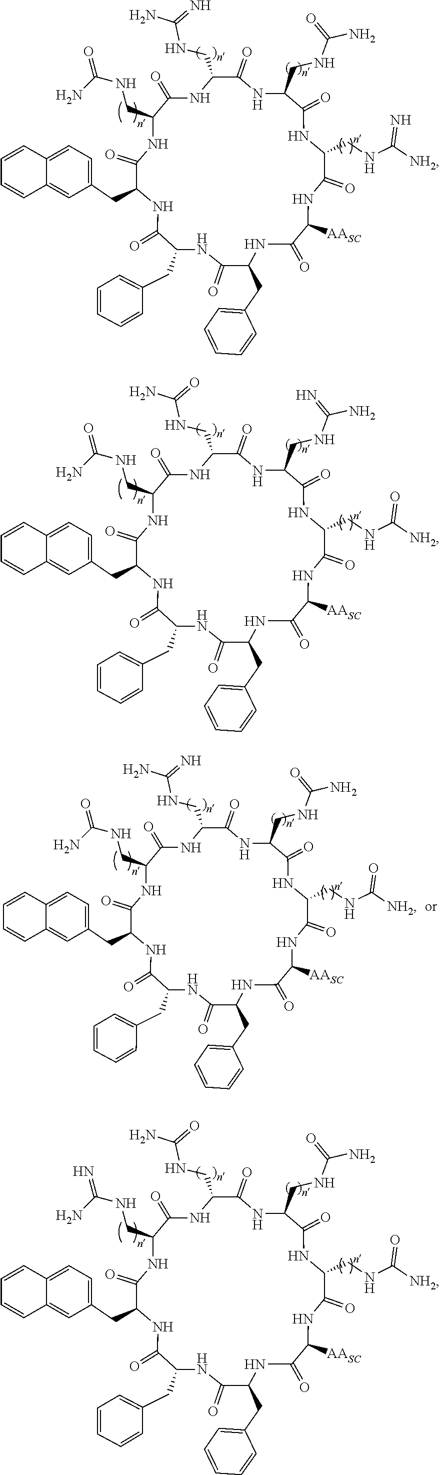

wherein AA_{SC} and n are as defined herein.

The cCPP of Formula (IIa) has one of the following structures (SEQ TD NOS 661-664, respectively, in order of appearance):

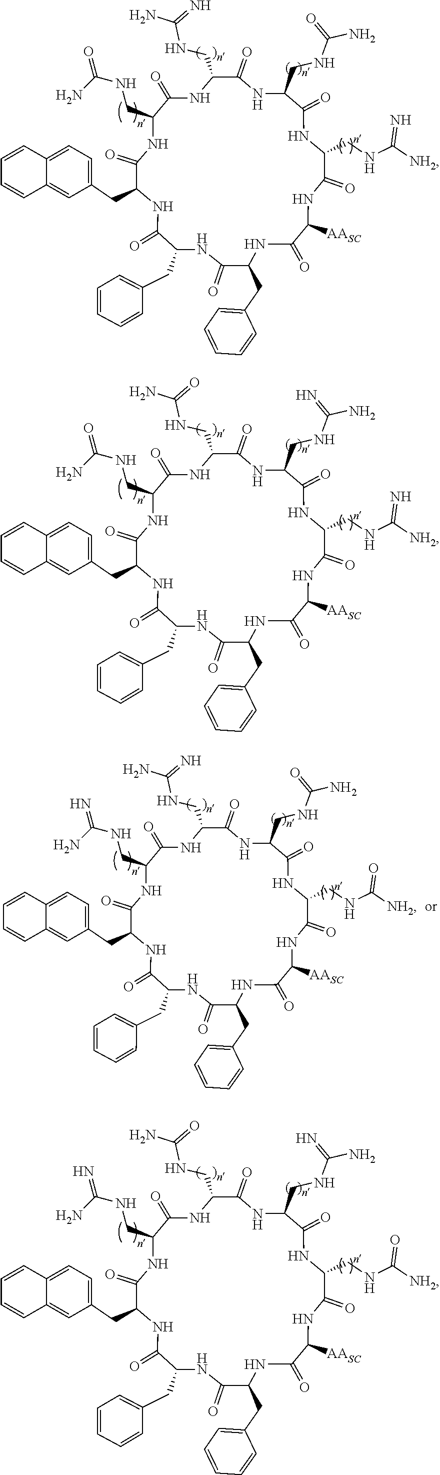

wherein AA_{SC} and n are defined herein

The cCPP of Formula (IIa) ha one of the following structures (SEQ ID NOS 665-668, respectively, in order of appearance):

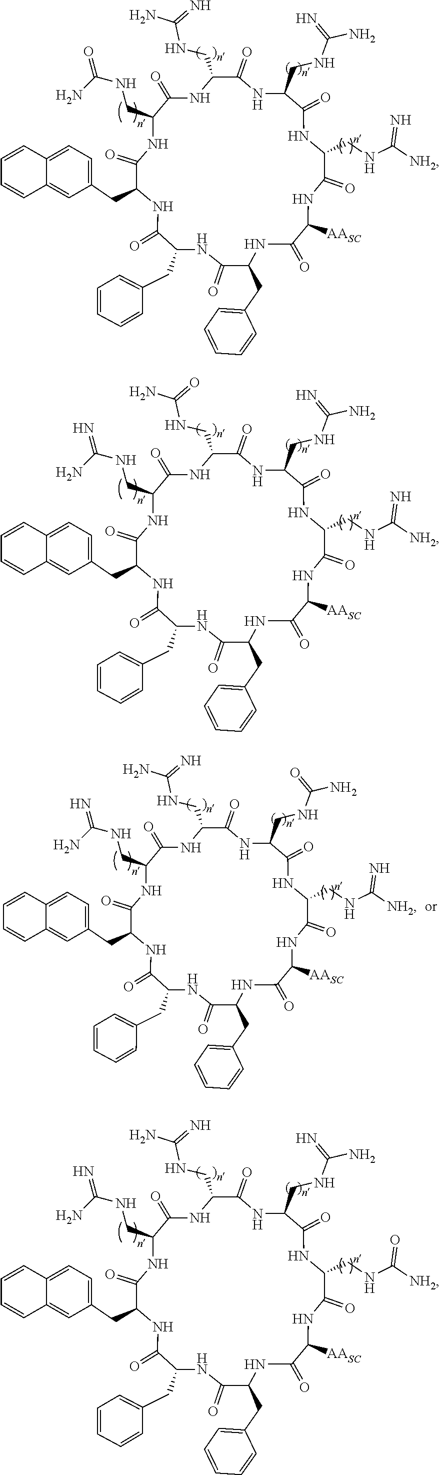

wherein AA_{SC} and n are as defined herein.

The cCPP of Formula (11) can have the structure (SEQ ID NO: 669):

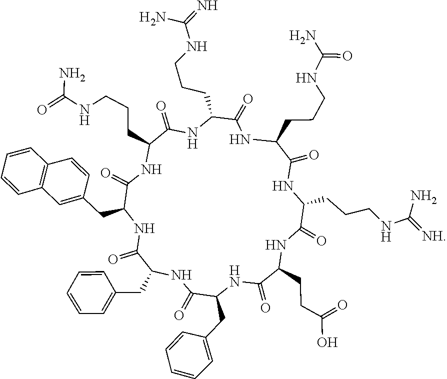 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure (SEQ ID NOS 670-671, respectively, in order of appearance):

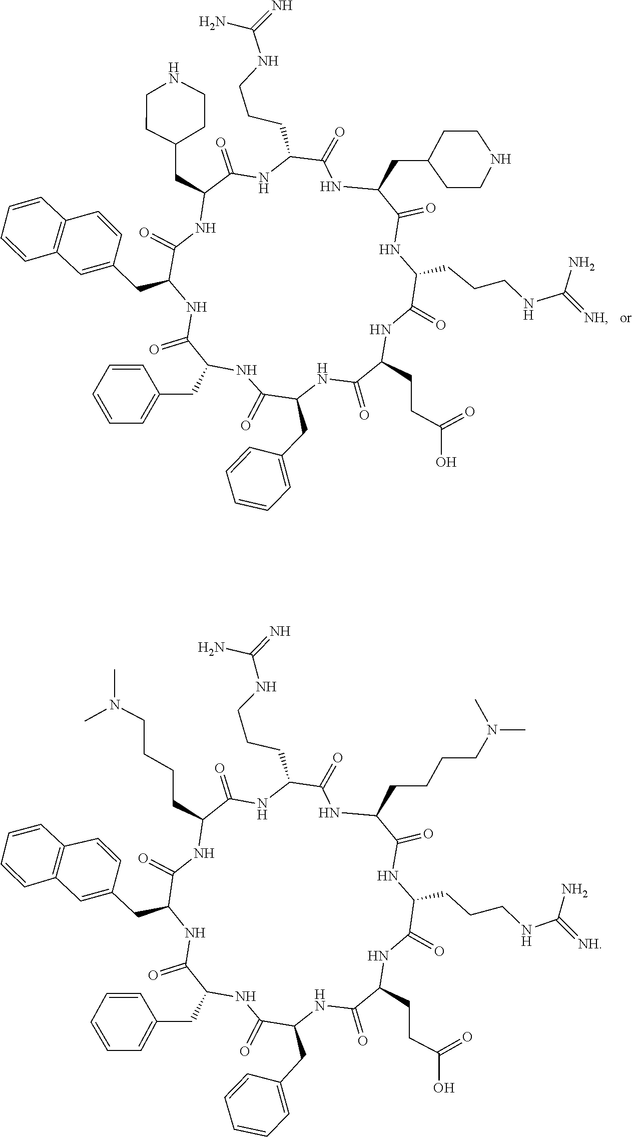 [SMILES: N=C(N)NCCC[C@H]1NC(=O)[C@H](CC2CCNCC2)NC(=O)[C@@H](CCCNC(=N)N)NC(=O)[C@H](CC2CCNCC2)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; CN(C)CCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)[C@H](CCCCN(C)C)NC(=O)[C@@H](CCCNC(=N)N)NC1=O]

The cCPP can have the structure of Formula (III):

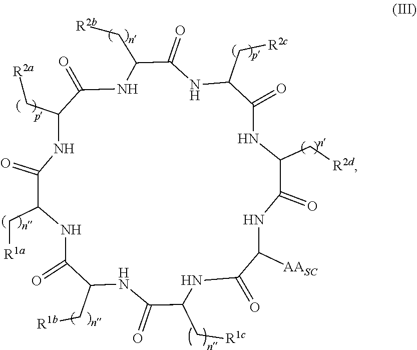

wherein:
AA_{SC} is an amino acid side chain;
R^{1a}, R^{1b}, and R^{1c} are each independently a 6- to 14-membered aryl or a 6- to 14-membered heteroaryl; R^{2a} and R²⁰ are each independently H,

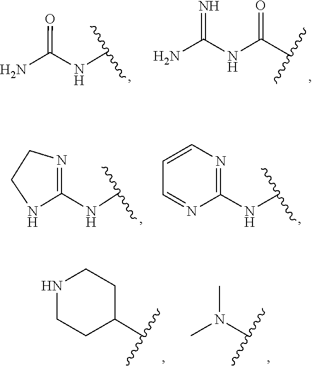

or a protonated form thereof;
R^{2b} and R^{2d} are each independently guanidine or a protonated form thereof,
each n″ is independently an integer from 1 to 3;
each n′ is independently an integer from 1 to 5; and
each p′ is independently an integer from 0 to 5.

The cCPP of Formula (III) can have the structure of Formula (III-1):

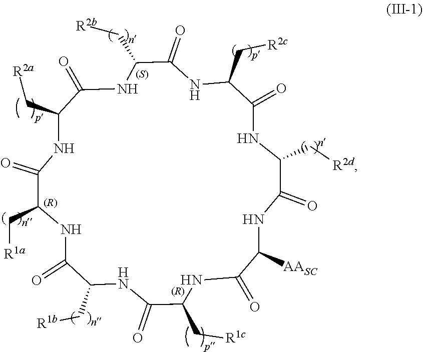

wherein:
AA_{SC}, Ria R^{1b}, R^{1c}, R^{2a}, R²⁰, R^{2b}, R^{2d} n′, n″, and p′ are as defined herein.

The cCPP of Formula (III) can have the structure of Formula (IIIa):

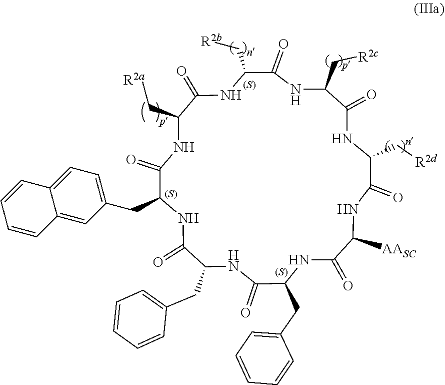

wherein:
AA_{SC}, R², R²⁰, , R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure (SEQ ID NO: 672):

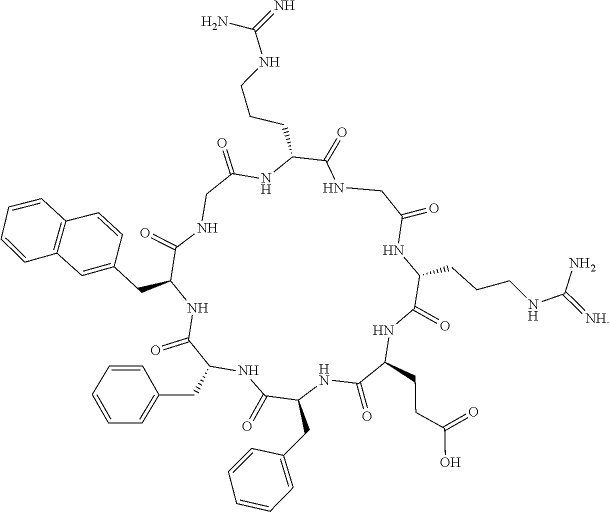 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

| CPP Sequence | SEQ ID NO: |
|---|---|
| (FfΦRrRrQ) | 78 |
| (FfΦCit-r-Cit-rQ) | 79 |
| (FfΦGrGrQ) | 80 |
| (FfFGRGRQ) | 81 |
| (FGFGRGRQ) | 82 |
| (GfFGrGrQ) | 83 |
| (FGFGRRRQ) | 84 |
| (FGFRRRRQ) | 85 |

The cCPP of Formula (A) can be selected from:

| CPP Sequence | SEQ ID NO: |
|---|---|
| FΦRRRRQ | 86 |
| fΦRrRrQ | 87 |
| FfΦRrRrQ | 78 |
| FfΦCit-r-Cit-rQ | 79 |
| FfΦGrGrQ | 80 |
| FfΦRGRGQ | 88 |
| FfFGRGRQ | 81 |
| FGFGRGRQ | 82 |
| GfFGrGrQ | 83 |
| FGFGRRRQ | 84 |
| FGFRRRRQ | 85 |

In embodiments, the cCPP is selected from:

CPP sequences and SEQ ID NOs

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| rRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Where Φ = L-naphthylalanine; ϕ = D-naphthylalanine; Ω = L-norleucine

In embodiments, the cCPP is not selected from:

CPP sequences and SEQ ID NOs

| | |
|---|---|
| FΦRRRQ | 89 |
| FΦRRRC | 90 |
| FΦRRRU | 91 |
| RRRΦFQ | 92 |
| RRRRΦF | 93 |
| FΦRRRR | 94 |
| FϕrRrRq | 95 |
| FϕrRrRQ | 96 |
| FΦRRRRQ | 97 |
| fΦRrRrQ | 98 |
| RRFRΦRQ | 99 |
| FRRRRΦQ | 100 |
| rRFRΦRQ | 101 |
| RRΦFRRQ | 102 |
| CRRRRFWQ | 103 |
| FfΦRrRrQ | 104 |
| FFΦRRRRQ | 105 |
| RFRFRΦRQ | 106 |
| URRRRFWQ | 107 |
| CRRRRFWQ | 108 |
| FΦRRRRQK | 109 |
| FΦRRRRQC | 110 |
| fΦRrRrRQ | 111 |
| FΦRRRRRQ | 112 |
| RRRRΦFDΩC | 113 |
| FΦRRR | 114 |
| FWRRR | 115 |
| RRRΦF | 116 |
| RRRWF | 117 |

Where Φ = L-naphthylalanine; ϕ = D-naphthylalanine; Ω = L-norleucine

The cCPP can comprise the structure of Formula (D)

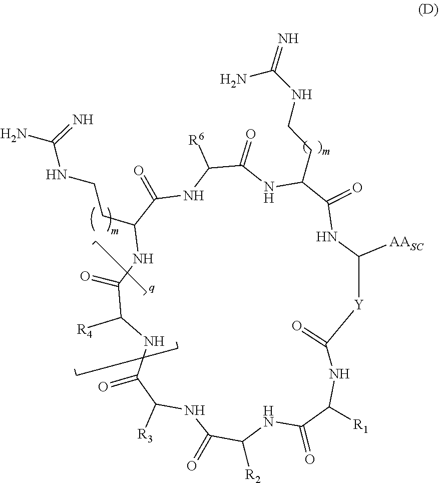

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain; Y is

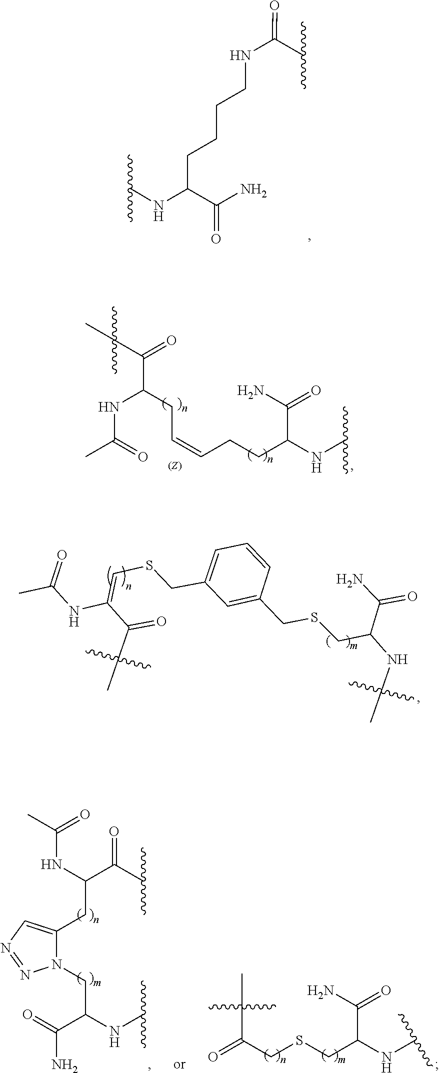

AA_{SC} is an amino acid side chain;
q is 1, 2, 3 or 4;
each m is independently an integer 0, 1, 2, or 3, and
each n is independently an integer 0, 1, 2, or 3.

The cCPP of Formula (D) can have the structure of Formula (D-I):

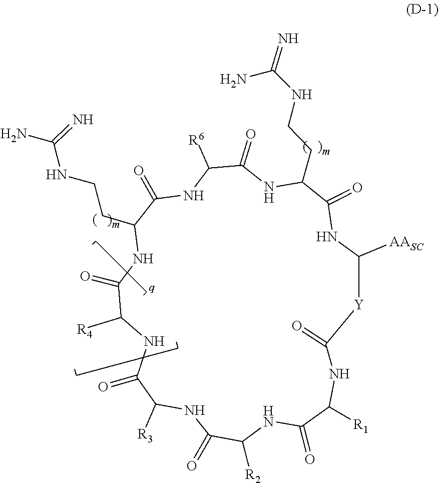

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and Y is

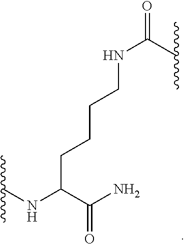 [SMILES: *NC(CCCCNC(*)=O)C(N)=O]

The cCPP of Formula (D) can have the structure of Formula (D-II):

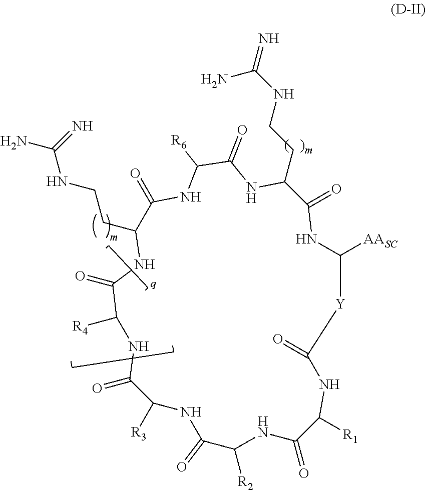

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3,
each m is independently an integer 0, 1, 2, or 3, and Y is

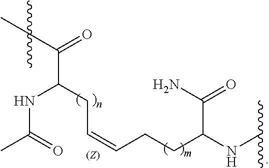

The cCPP of Formula (D) can have the structure of Formula (D-III):

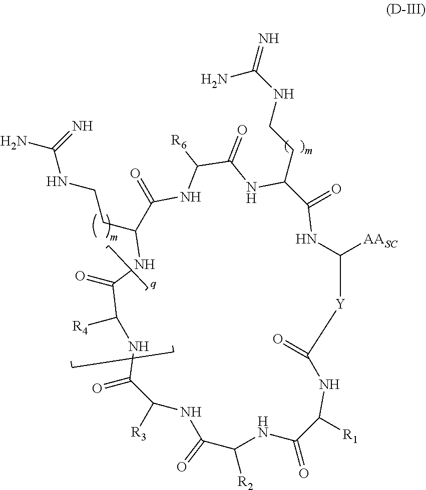

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3,
each n is independently an integer 0, 1, 2, or 3, and Y is

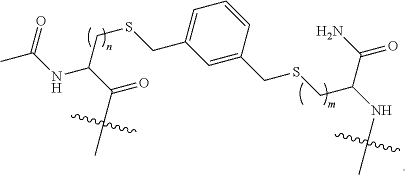

The cCPP of Formula (D) can have the structure of Formula (D-IV):

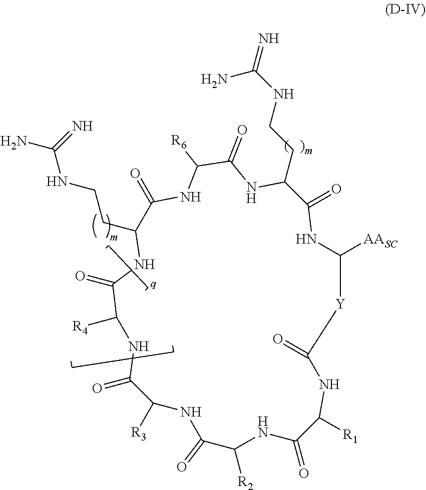

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and Y is

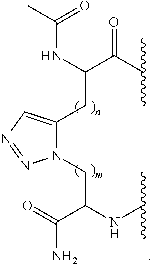

The cCPP of Formula (D) can have the structure of Formula (D-V):

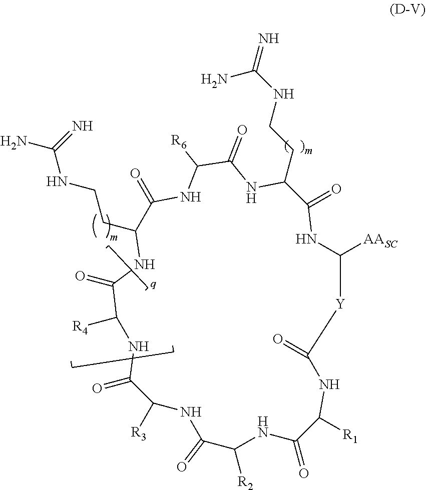

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and Y is

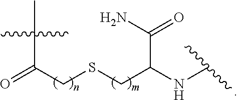

The AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more -(R¹-J-R²)z″-subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) -(R¹-J)z″- or —(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″-, wherein each of R¹ and R², at each instance, are independently alkylene, each J is independently C, NR³, -NR³C(O)—, S, and O, wherein R⁴is independently selected from H and alkyl, and z″ is an integer from 1 to 50; or combinations thereof.

The linker can comprise a —(OCH₂CH₂)_{z}′-(e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z″ can also be referred to as polyethylene glycol (PEG).

The linker can comprise one or more amino acids. The linker can comprise a peptide. The linker can comprise a —(OCH₂CH₂)_{z}′-, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a R alanine residue and lysine residue; (ii) -(J-R¹)_{z}″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be 0.

The linker can comprise (i) residues of β-alanine, glycine, lysine, 4-aminobutyric acid, 5-aminopentanoic acid, 6-aminohexanoic acid or combinations thereof, and (ii) -(R¹-J)_{z}″- or —(J-R¹)_{z}″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be 0. The linker can comprise glycine, beta-alanine, 4-aminobutyric acid, 5-aminopentanoic acid, 6-aminohexanoic acid, or a combination thereof.

The linker can be a trivalent linker. The linker can have the structure:

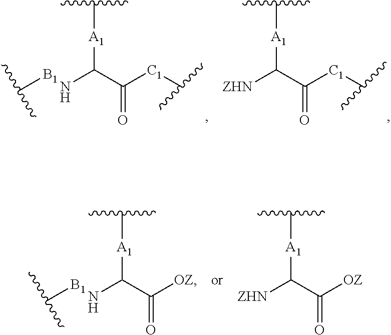

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)-, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)-, or a combination thereof.

The linker can have the structure:

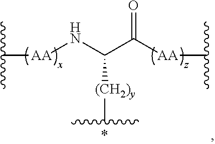

Wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

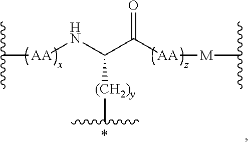

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

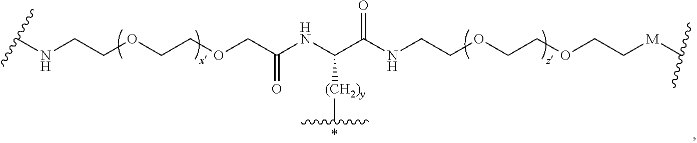

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

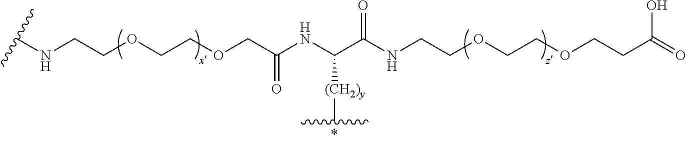

wherein: x′ is an integer from 1-23; y is an integer from 1-5; and z′ is an integer from 1-23; * is the point of attachment to the AA_{SC}, and AA_{SC} is a side chain of an amino acid residue of the cCPP.
x can be an integer from 1-10, e.g., 1, 2, 3, 4, 5, 6, 7, 8, 9, or 10, inclusive of all ranges and subranges therebetween.
x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.
y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.
z can be an integer from 1-10, e.g., 1, 2, 3, 4, 5, 6, 7, 8, 9, or 10, inclusive of all ranges and subranges therebetween.
z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

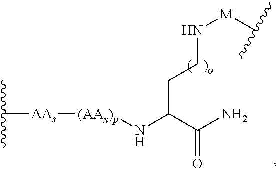

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

The linker can have a structure:

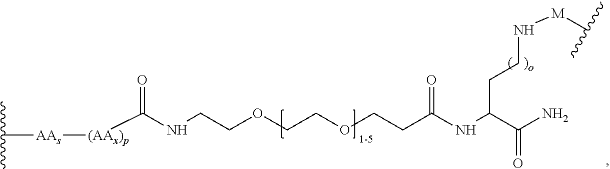

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

M can comprise an alkylene, alkenylene, alkynylene, carbocyclyl, or heterocyclyl, each of which is optionally substituted. M can be selected from:

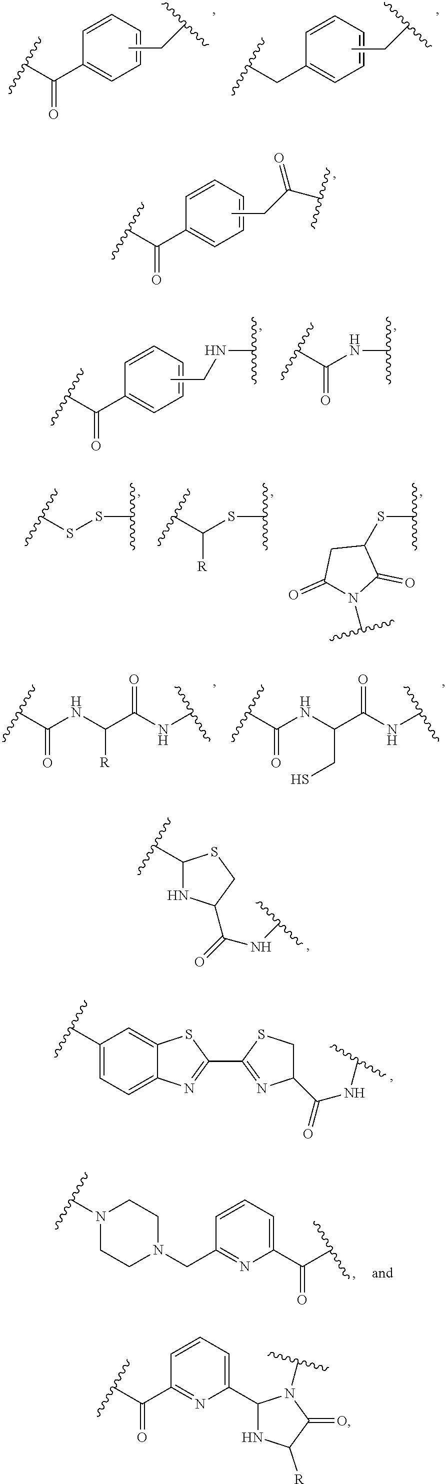

wherein R is alkyl, alkenyl, alkynyl, carbocyclyl, or heterocyclyl.

M can be selected from:

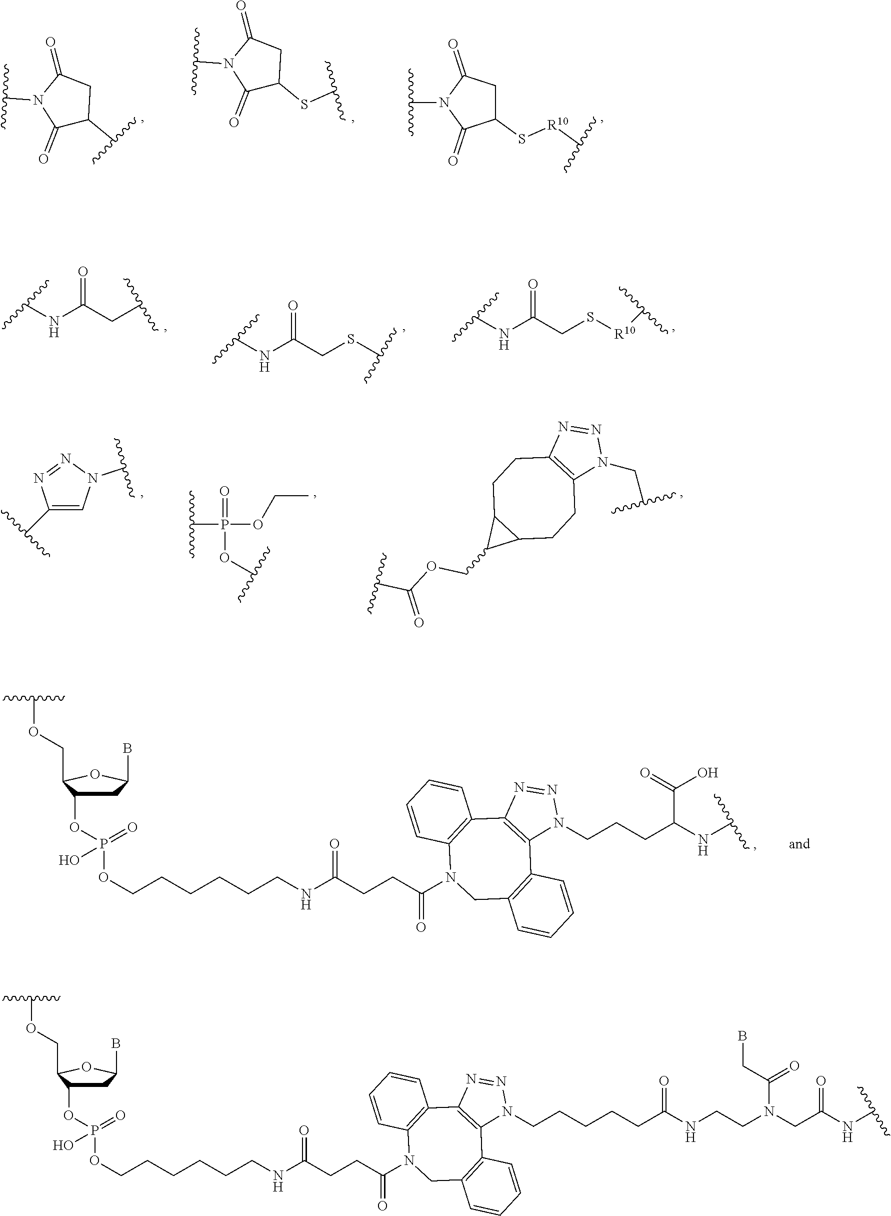

wherein: R¹⁰ is alkylene, cycloalkyl, or

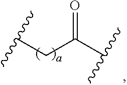

wherein a is 0 to 10.

M can be

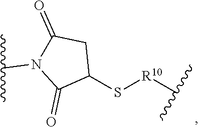

### R¹⁰ can be

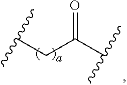

### and a is 0 to 10. M can be

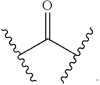 [SMILES: *C(*)=O]

M can be a heterobifunctional crosslinker, e.g., ,

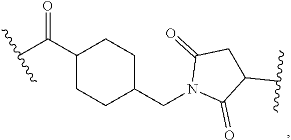 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.
can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.
p can be 0 to 5, e.g., 0, 1, 2, 3, 4, or 5. p can be 0. p can be 1. p can be 2. p can be 3. p can be 4. p can be 5.

The linker can have the structure:

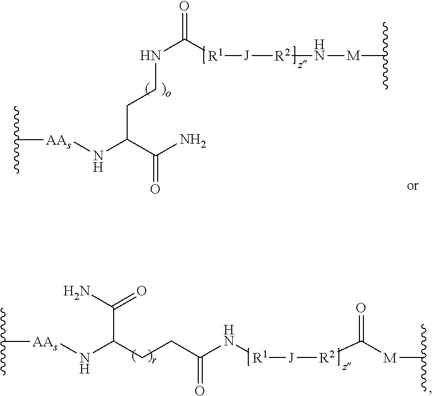

wherein M, AA₅, each -(R¹-J-R²)_{z}″-, o and z″ are defined herein; r can be 0 or 1.
r can be 0. r can be 1.

The linker can have the structure:

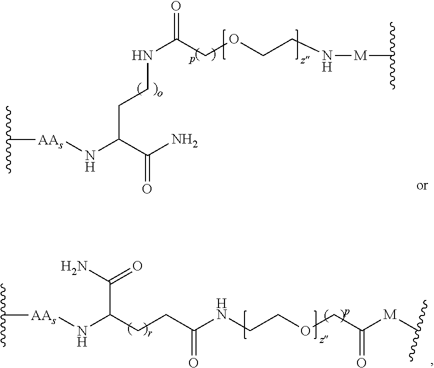

wherein each of M, AA₅, o, p, q, r and z″ can be as defined herein.
z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

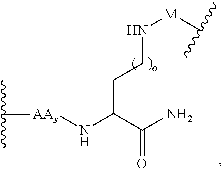

wherein:
M, AAₛ and o are as defined herein.

Other non-limiting examples of suitable linkers include:

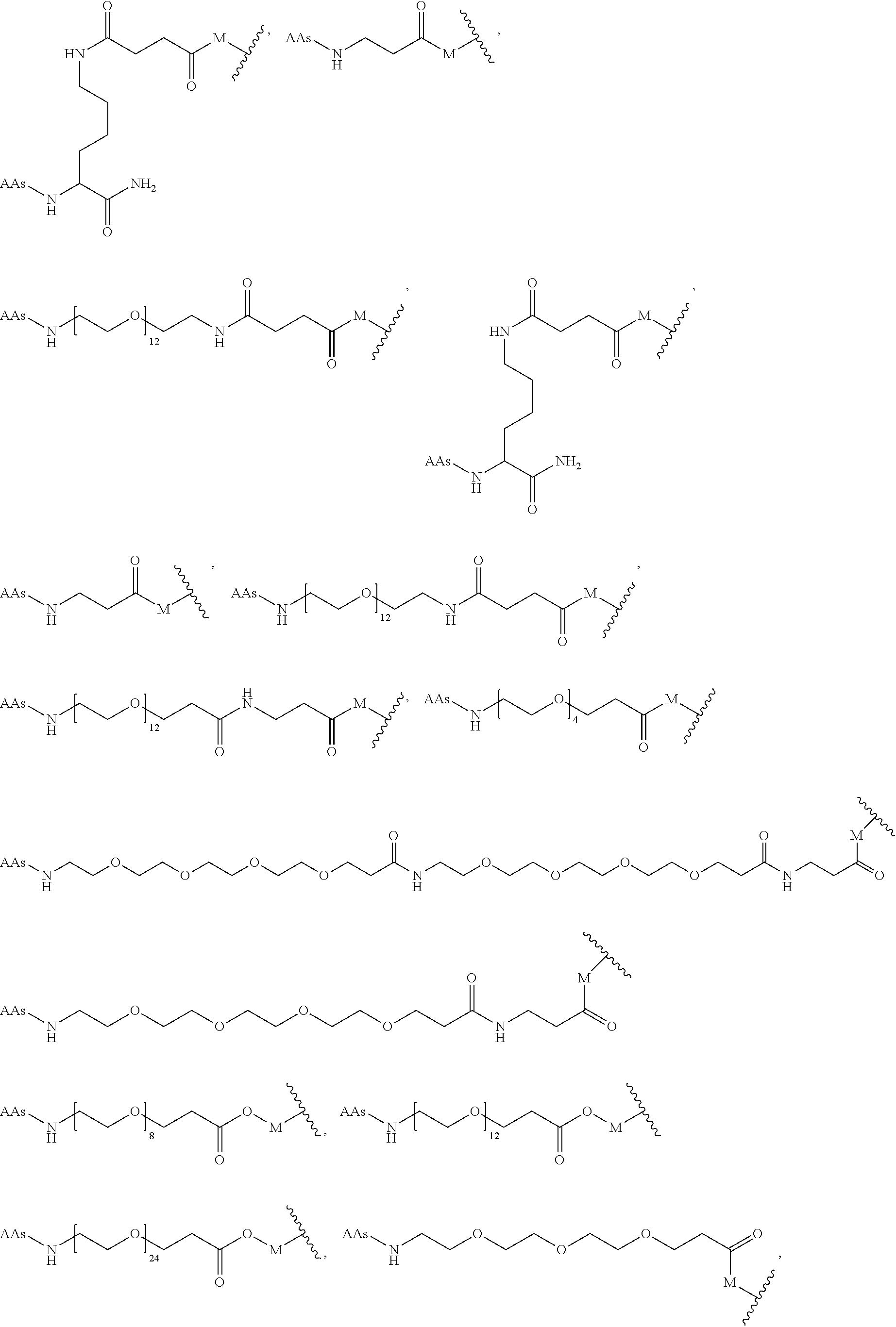

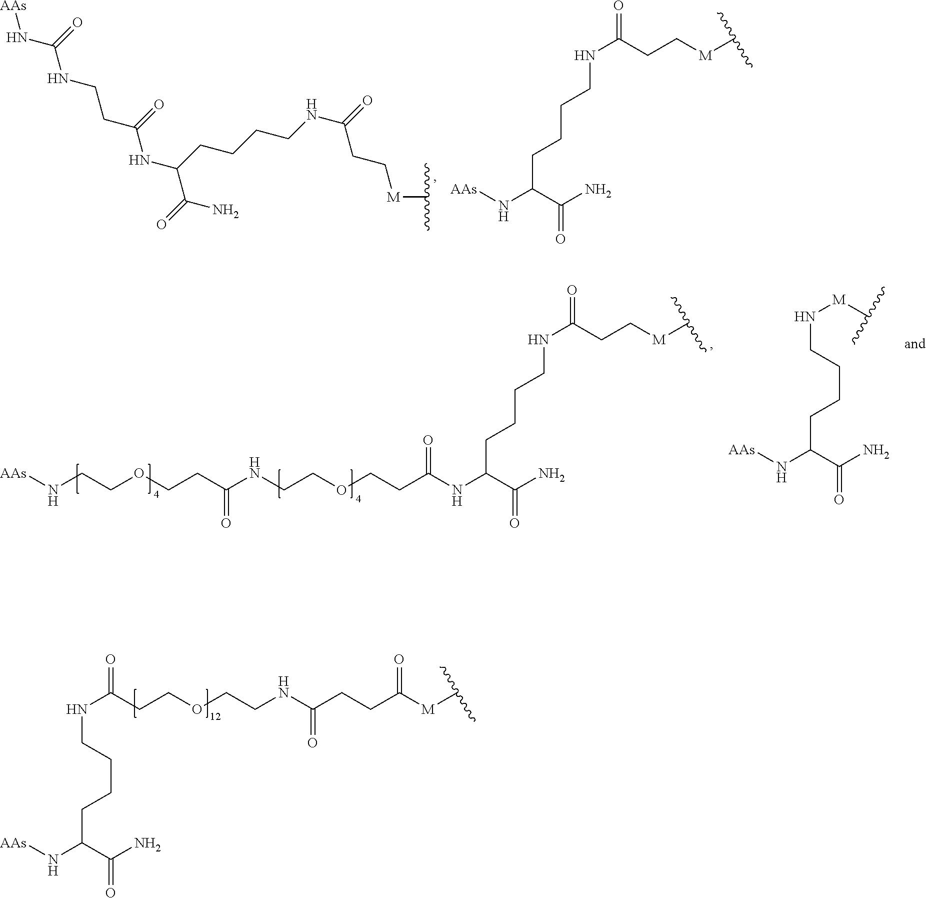

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

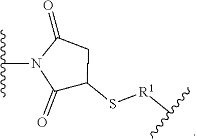

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

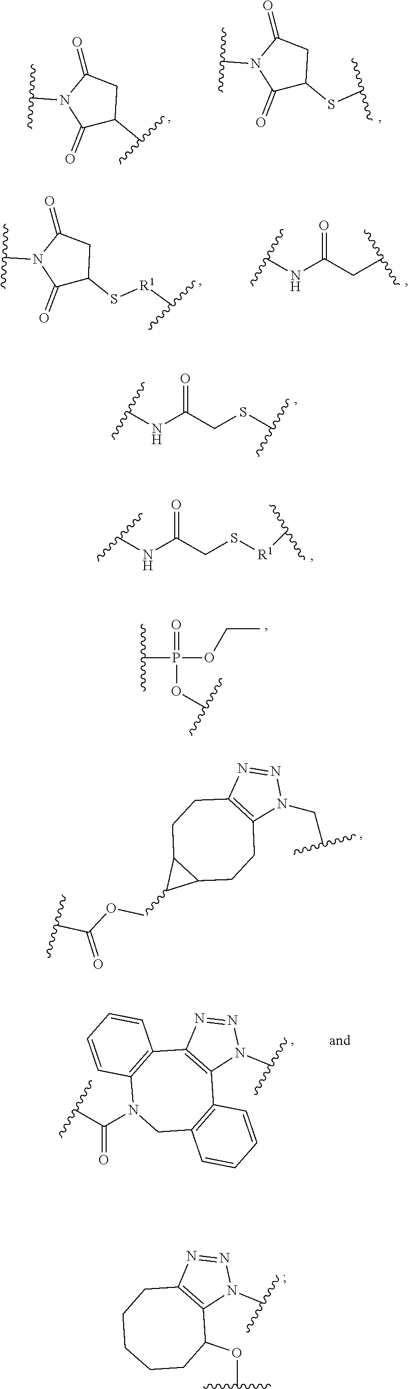

wherein: R¹ is alkylene, cycloalkyl, or

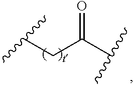

wherein t′ is 0 to 10 wherein each R is independently an alkyl, alkenyl, alkynyl, carbocyclyl, or heterocyclyl, wherein R¹ is

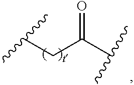

and t′ is 2.

The linker can have the structure:

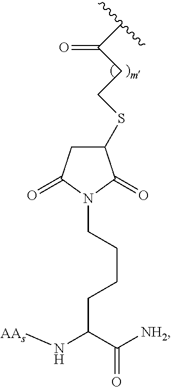

wherein AAₛ is as defined herein, and m′ is 0-10.

The linker can be of the formula:

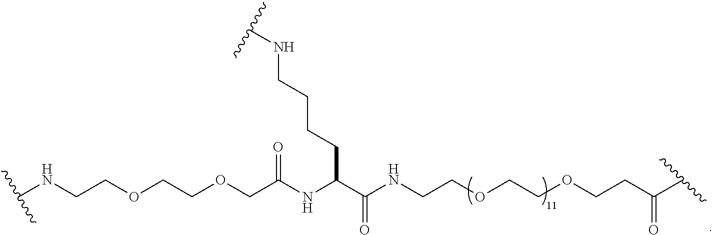

The linker can be of the formula:

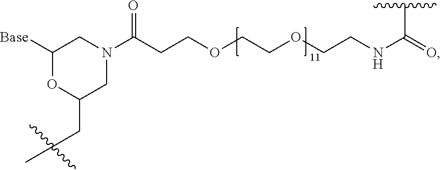

wherein “base” is a nucleobase at the 3′ end of a cargo phosphorodiamidate morpholino oligomer.

The linker can be of the formula:

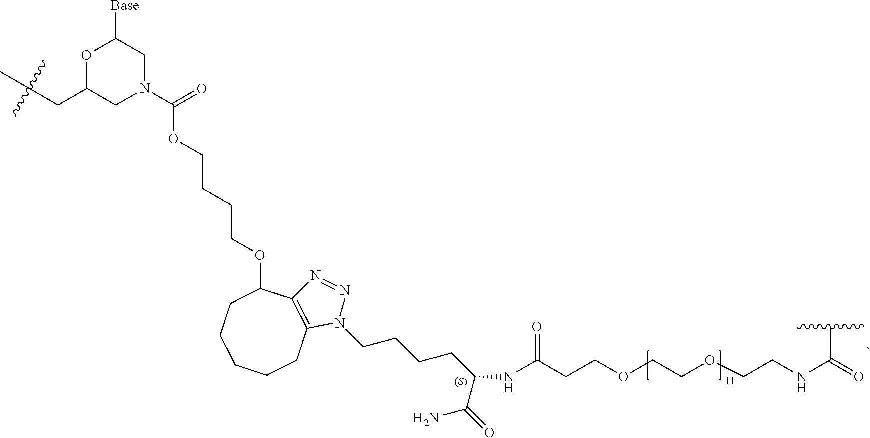

wherein “base” corresponds to a nucleobase at the 3′ end of a cargo phosphorodiamidate morpholino oligomer.

The linker can be of the formula:

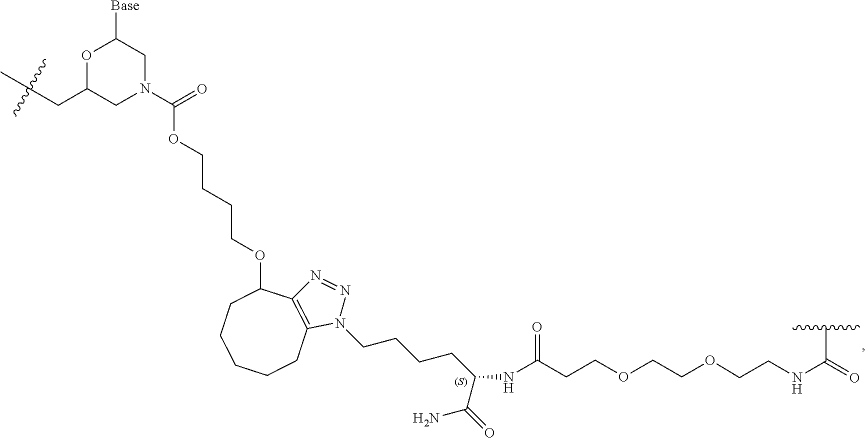

O0, wherein “base” is a nucleobase at the 3′ end of a cargo phosphorodiamidate morpholino oligomer.

The linker can be of the formula:

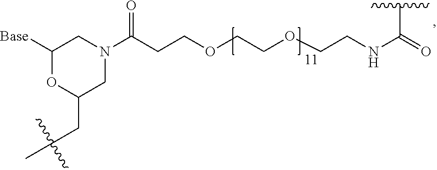

wherein “base” is a nucleobase at the 3′ end of a cargo phosphorodiamidate morpholino oligomer.

The linker can be of the formula:

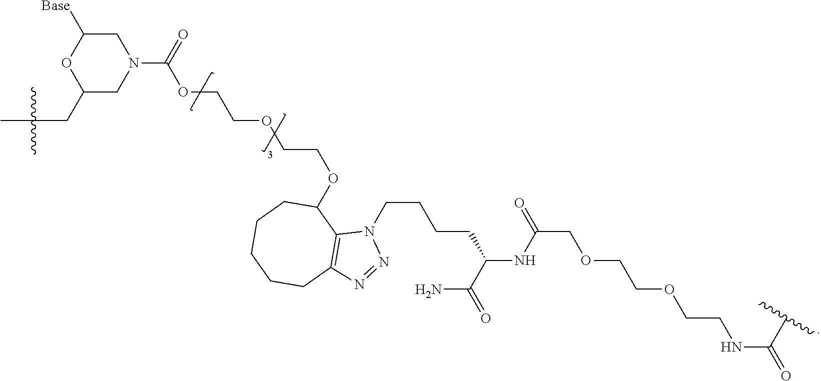

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-linker conjugates

The cCPP can be conjugated to a linker defined herein. The linker can be conjugated to an AA_{SC} of the cCPP as defined herein.

The linker can comprise a —(OCH₂CH₂)_{z}′-subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z}′ is also referred to as PEG. The cCPP-linker conjugate can have a structure selected from Table 7:

TABLE 7
cCPP-linker conjugates and SEQ ID NOs

| | |
|---|---|
| cyclo(FfΦ-4gp-r-4gp-rQ)-PEG₄-K-NH₂ | cyclo(SEQ ID NO: 118)- |
| | PEG₄-K-NH₂ |
| cyclo(FfΦ-Cit-r-Cit-rQ)-PEG₄-K-NH₂ | cyclo(SEQ ID NO: 119)- |
| | PEG₄-K-NH₂ |
| cyclo(FfΦ-Pia-r-Pia-rQ)-PEG₄-K-NH₂ | cyclo(SEQ ID NO: 120)- |
| | PEG₄-K-NH₂ |
| cyclo(FfΦ-Dml-r-Dml-rQ)-PEG₄-K-NH₂ | cyclo(SEQ ID NO: 121)- |
| | PEG₄-K-NH₂ |
| cyclo(FfΦ-Cit-r-Cit-rQ)-PEG₁₂-OH | cyclo(SEQ ID NO: 122)- |
| | PEG₁₂-OH |
| cyclo(fΦR-Cit-R-Cit-Q)-PEG₁₂-OH | cyclo(SEQ ID NO: 123)- |
| | PEG₁₂-OH |

The linker can comprise a —(OCH₂CH₂)_{z}′-subunit, wherein z′ is an integer from 1 to 23, and a peptide subunit. The peptide subunit can comprise from 2 to 10 amino acids. The cCPP-linker conjugate can have a structure selected from Table 8:

TABLE 8
cCPP-linker conjugate and SEQ ID NOs

| | |
|---|---|
| Ac-PKKKRKV-Lys(cyclo[FfΦ-R-r-Cit-rQ])- | SEQ ID NOS 415-416, respectively, in order |
| PEG₁₂-K(N₃)-NH₂ | of appearance; Ac-SEQ ID NO: 42- |
| | Lys(cyclo[SEQ ID NO: 127])-PEG₁₂-K(N₃)- |
| | NH₂ |
| Ac-PKKKRKV-Lys(cyclo[FfΦ-Cit-r-R-rQ])- | SEQ ID NOS 417-418, respectively, in order |
| PEG₁₂-K(N₃)-NH₂ | of appearance; Ac- SEQ ID NO: 42- |
| | Lys(cyclo[SEQ ID NO: 128])-PEG₁₂-K(N₃)- |
| | NH₂ |
| Ac-PKKKRKV-K(cyclo(FfΦR-cit-R-cit-Q))- | SEQ ID NOS 419-420, respectively, in order |
| PEG₁₂-K(N₃)-NH₂ | of appearance; Ac- SEQ ID NO: 42- |
| | K(cyclo(SEQ ID NO: 129))-PEG₁₂-K(N₃)- |
| | NH₂ |
| Ac-PKKKRKV-PEG2-Lys(cyclo[FfΦ-Cit-r-Cit- | SEQ ID NOS 421-422, respectively, in order |
| rQ])-B-k(N₃)-NH₂ | of appearance; Ac- SEQ ID NO: 42-PEG2- |
| | Lys(cyclo[SEQ ID NO: 130])-B-k(N₃)-NH₂ |
| Ac-PKKKRKV-PEG2-Lys(cyclo[FfΦ-Cit-r-Cit- | SEQ ID NOS 423-424, respectively, in order |
| rQ])-PEG2-k(N₃)-NH₂ | of appearance; Ac- SEQ ID NO: 42-PEG2- |
| | Lys(cyclo[SEQ ID NO: 130])-PEG2-k(N₃)- |
| | NH₂ |
| Ac-PKKKRKV-PEG2-Lys(cyclo[FfΦ-Cit-r-Cit- | SEQ ID NOS 425-426, respectively, in order |
| rQ])-PEG4-k(N₃)-NH₂ | of appearance; Ac- SEQ ID NO: 42-PEG2- |
| | Lys(cyclo[SEQ ID NO: 130])-PEG4-k(N₃)- |
| | NH₂ |
| Ac-PKKKRKV-Lys(cyclo[FfΦ-Cit-r-Cit-rQ])- | SEQ ID NOS 427-428, respectively, in order |
| PEG12-k(N₃)-NH₂ | of appearance; Ac- SEQ ID NO: 42- |
| | Lys(cyclo[SEQ ID NO: 130])-PEG12-k(N₃)- |
| | NH₂ |
| Ac-pkkkrkv-PEG2-Lys(cyclo[FfΦ-Cit-r-Cit- | SEQ ID NOS 429-430, respectively, in order |
| rQ])-PEG12-k(N₃)-NH₂ | of appearance; Ac- SEQ ID NO: 131-PEG2- |
| | Lys(cyclo[SEQ ID NO: 130])-PEG12-k(N₃)- |
| | NH₂ |
| Ac-rrv-PEG2-Lys(cyclo[FfΦ-Cit-r-Cit-rQ])- | SEQ ID NO: 431; Ac-rrv-PEG2- |
| PEG12-OH | Lys(cyclo[SEQ ID NO: 130])-PEG12-OH |
| Ac-PKKKRKV-PEG2-Lys(cyclo[FfΦ-Cit-r-Cit-r- | SEQ ID NOS 432-433, respectively, in order |
| Q])-PEG12-k(N₃)-NH₂ | of appearance; Ac- SEQ ID NO: 42-PEG2- |
| | Lys(cyclo[SEQ ID NO: 130])-PEG12-k(N₃)- |
| | NH₂ |
| Ac-PKKK-Cit-KV-PEG2-Lys(cyclo[FfΦ-Cit-r- | SEQ ID NOS 434-435, respectively, in order |
| Cit-r-Q])-PEG12-k(N₃)-NH₂ | of appearance; Ac- SEQ ID NO: 126-PEG2- |
| | Lys(cyclo[SEQ ID NO: 130])-PEG12-k(N₃)- |
| | NH₂ |
| Ac-PKKKRKV-PEG2-Lys(cyclo[FfΦ-Cit-r-Cit-r- | SEQ ID NOS 436-437, respectively, in order |
| Q]-PEG12-K(N₃)-NH₂ | of appearance; Ac- SEQ ID NO: 42-PEG2- |
| | Lys(cyclo[SEQ ID NO: 130]-PEG12-K(N₃)- |
| | NH₂ |

EEVs comprising a cyclic cell penetrating peptide (cCPP), linker and exocyclic peptide (EP) are provided. An EEV can comprise the structure of Formula (B):

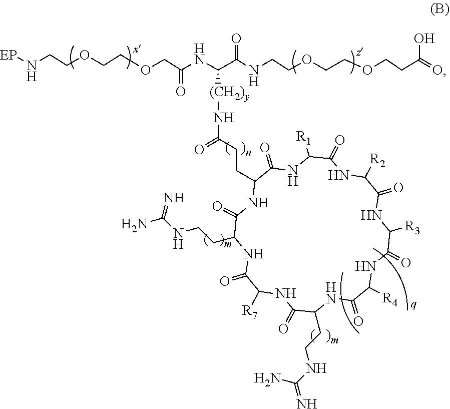

or a protonated form thereof,
wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.
R₁, R₂, R₃, R₄, R₇, EP, m, q, y, x′, z′ are as described herein.
n can be 0. n can be 1. n can be 2.

The EEV can comprise the structure of Formula (B-a) or (B-b):

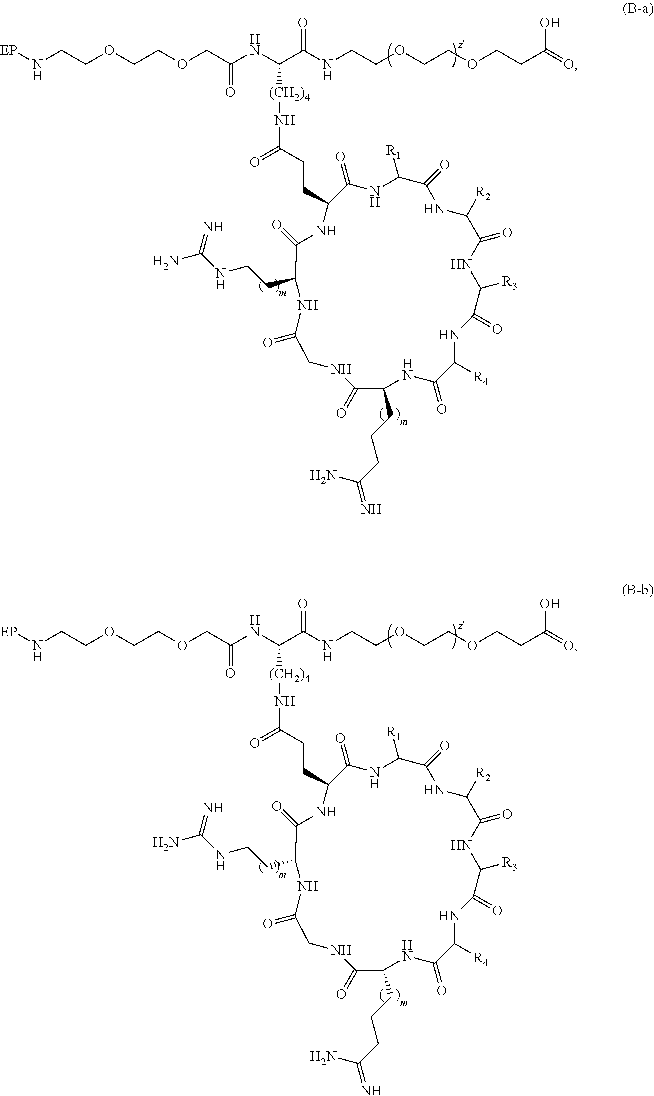

or a protonated form thereof, wherein EP, R₁, R², R³, R⁴, m and z′ are as defined above in Formula (B).

The EEV can comprises the structure of Formula (B-c):

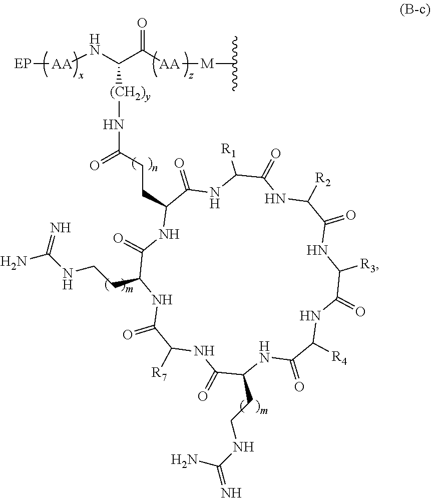

or a protonated form thereof, wherein EP, R₁, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4) (SEQ ID NOS 673-676, respectively, in order of appearance):

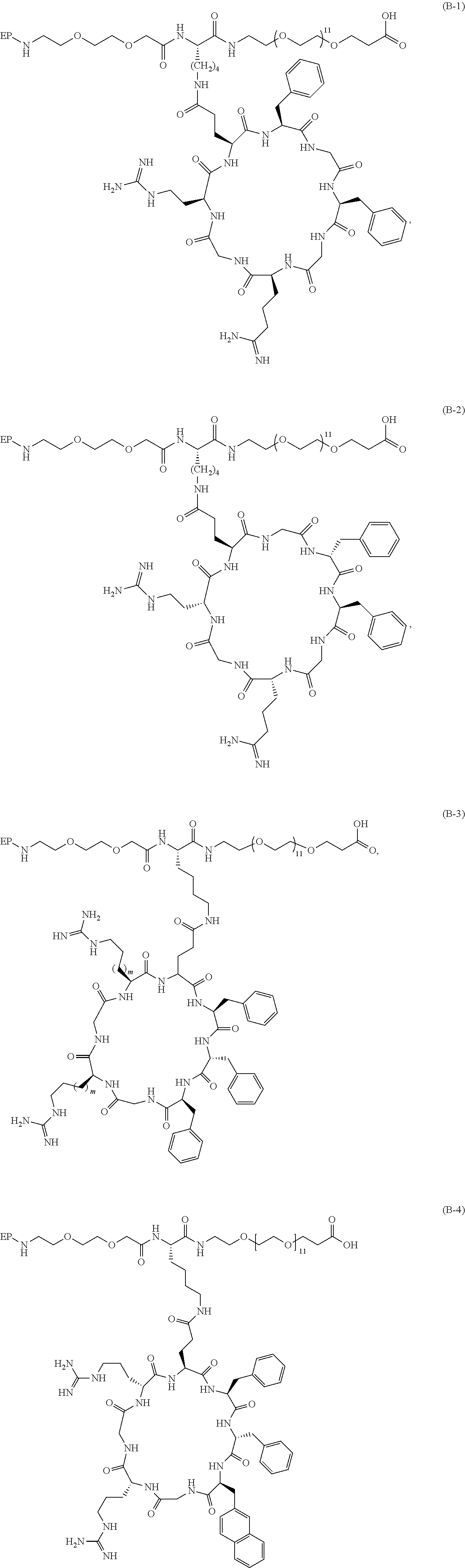

or a protonated form thereof, wherein EP is as defined above in Formula (B).

The EEV can comprise Formula (B) and can have the structure: Ac-PKKKRKVAEEA-K(cyclo[FGFGRGRQ])-PEG12—OH (SEQ ID NOS 438-439, respectively, in order of appearance) (Ac-SEQ ID NO:132-K(cyclo[SEQ ID NO:82])-PEG12—OH) or Ac-PK-KKR-KV-AEEA-K(cyclo[GfFGrGrQ])-PEG12—OH (SEQ ID NOS 440-441, respectively, in order of appearance) (Ac-SEQ ID NO:132-K(cyclo[SEQ ID NO:83])-PEG12—OH).

The EEV can comprise a cCPP of formula (SEQ ID NO: 677):

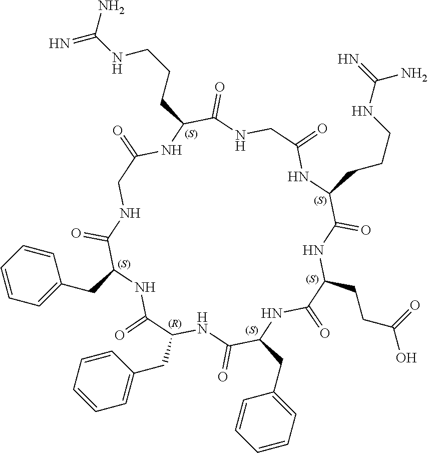

The EEV can comprise formula: Ac-PKKKRKV-miniPEG2-Lys(cyclo(FfFGRGRQ)-PEG2-K(N3) (SEQ ID NOS 442-443, respectively, in order of appearance) (Ac-SEQ ID NO:42-miniPEG2-Lys(cyclo(SEQ ID NO:81)-PEG2-K(N3)).

The EEV can be (SEQ ID NOS 678-679, respectively, in order of appearance):

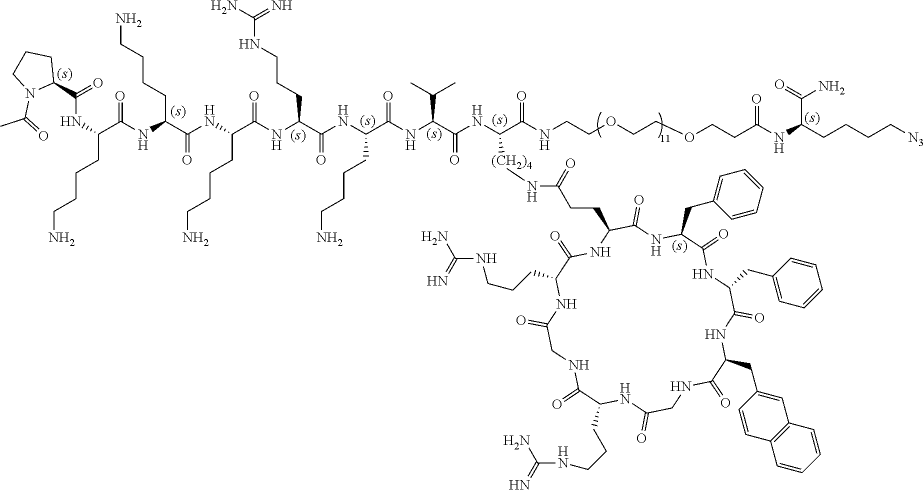

The EEV can be (SEQ ID NOS 680-681, respectively, in order of appearance)

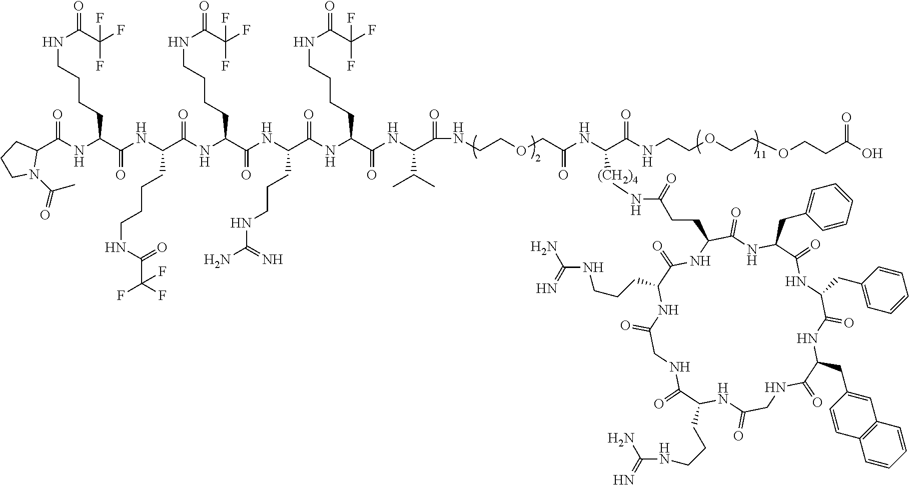

The EEV can be Ac-P-K(Tfa)-K(Tfa)-K(Tfa)-R-K(Tfa)-V-miniPEG2-K(cyclo(Ff-Nal-GrGrQ)-PEG12—OH (SEQ ID NOS 444-445, respectively, in order of appearance) (Ac-SEQ ID NO:134-miniPEG2-K(cyclo(SEQ ID NO:135)-PEG12—OH).

The EEV can be (SEQ ID NOS 682-683, respectively, in order of appearance)

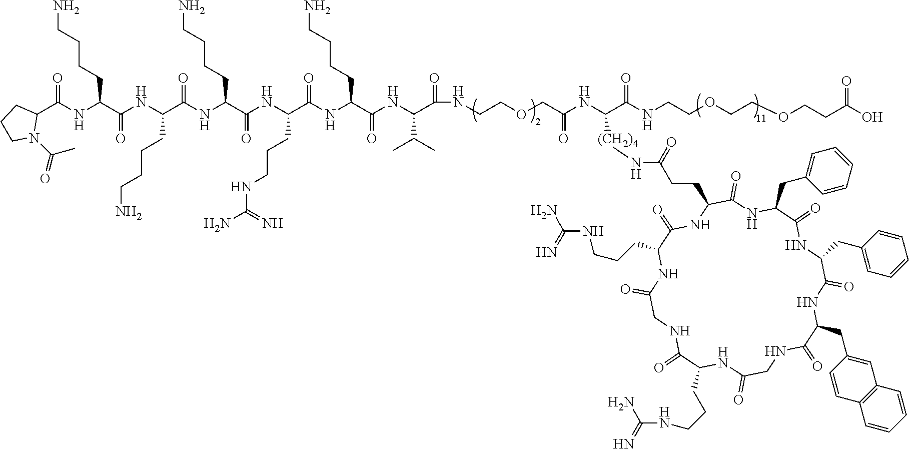

The EEV can be Ac-P-K-K-K-R-K-V-miniPEG2-K(cyclo(Ff-Nal-GrGrQ)-PEG12—OH (SEQ ID NOS 446-447, respectively, in order of appearance) (Ac-SEQ ID NO:42-miniPEG2-K(cyclo(SEQ ID NO:135)-PEG12—OH).

The EEV can be (SEQ ID NOS 684-685, respectively, in order of appearance)

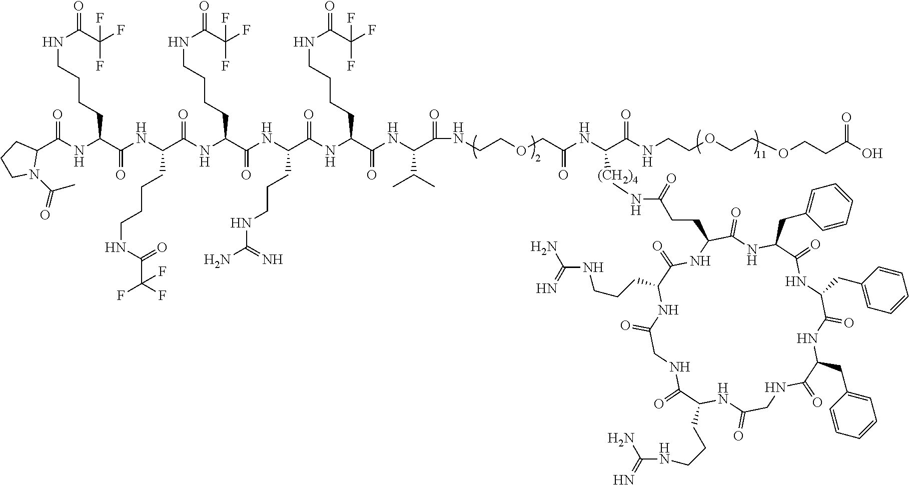

The EEV can be (SEQ ID NOS 686-687, respectively, in order of appearance)

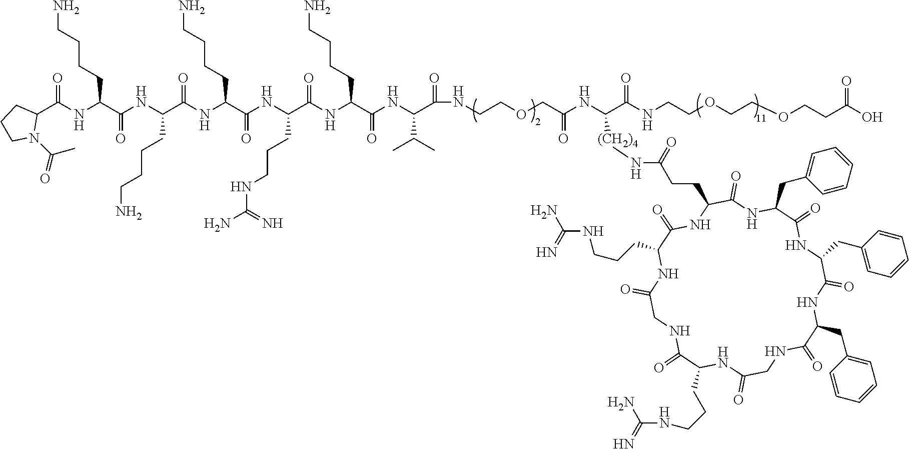

The EEV can be (SEQ ID NOS 688-689, respectively, in order of appearance)

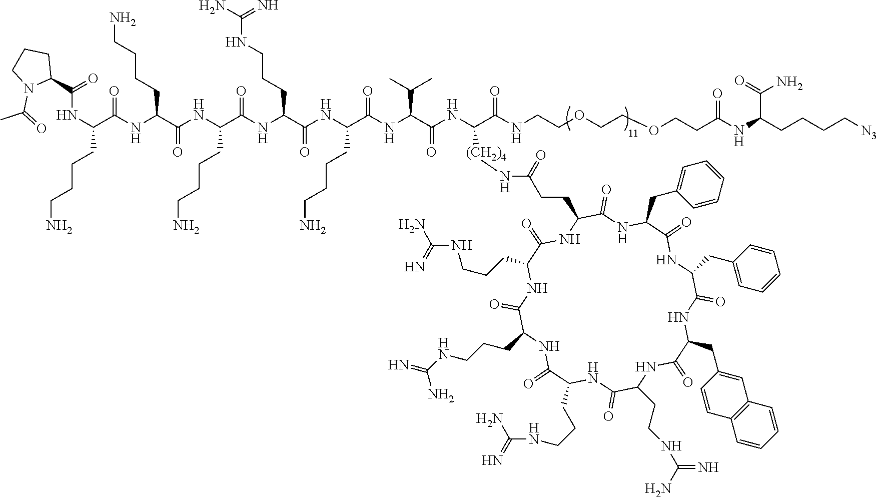

The EEV can be (SEQ ID NOS 690-691, respectively, in order of appearance)

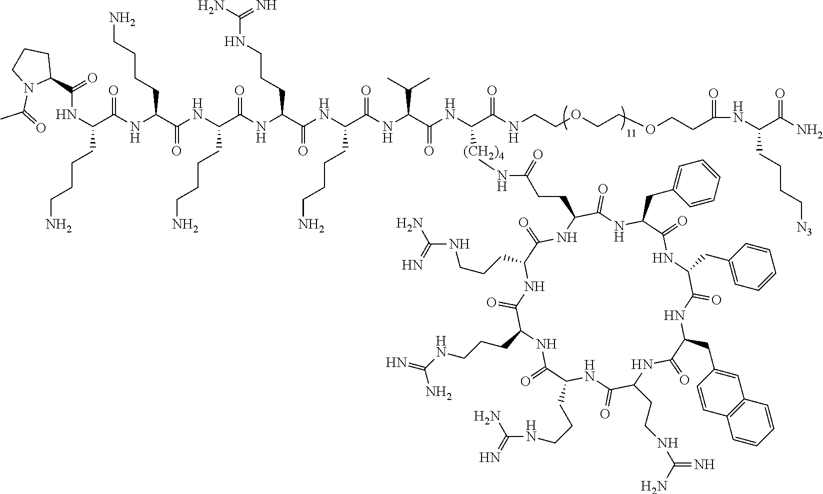

The EEV can be (SEQ ID NOS 692-693, respectively, in order of appearance)

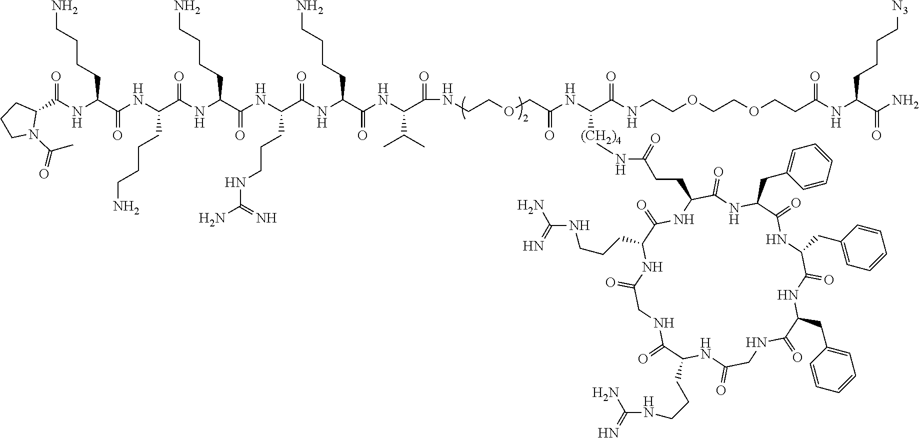

The EEV can be (SEQ ID NOS 694-695, respectively, in order of appearance)

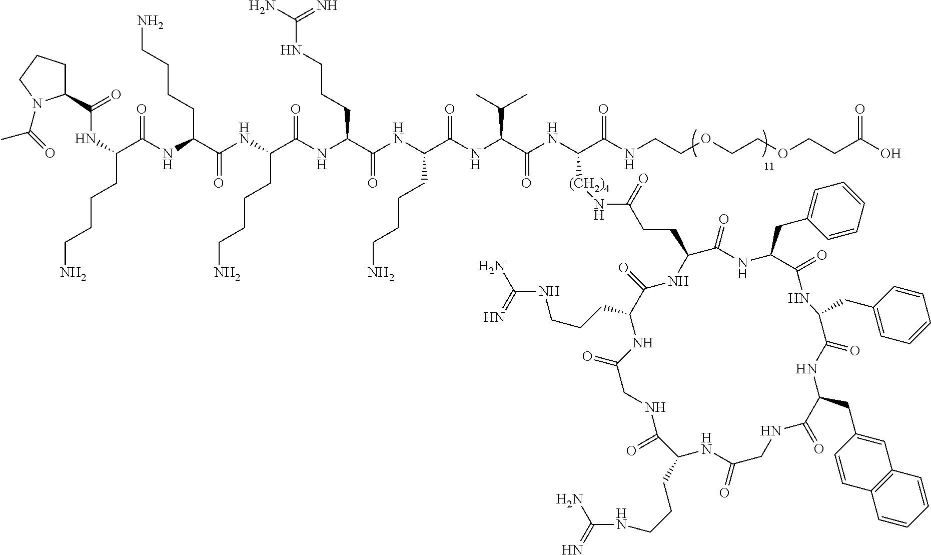

The EEV can be (SEQ ID NOS 696-697, respectively, in order of appearance):

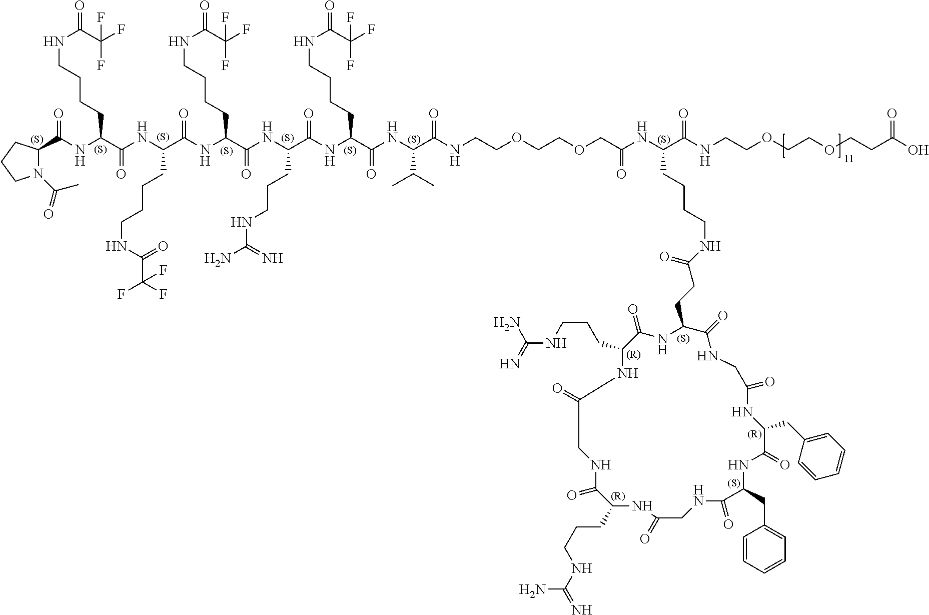

The EEV can be (SEQ ID NOS 698-699, respectively, in order of appearance)

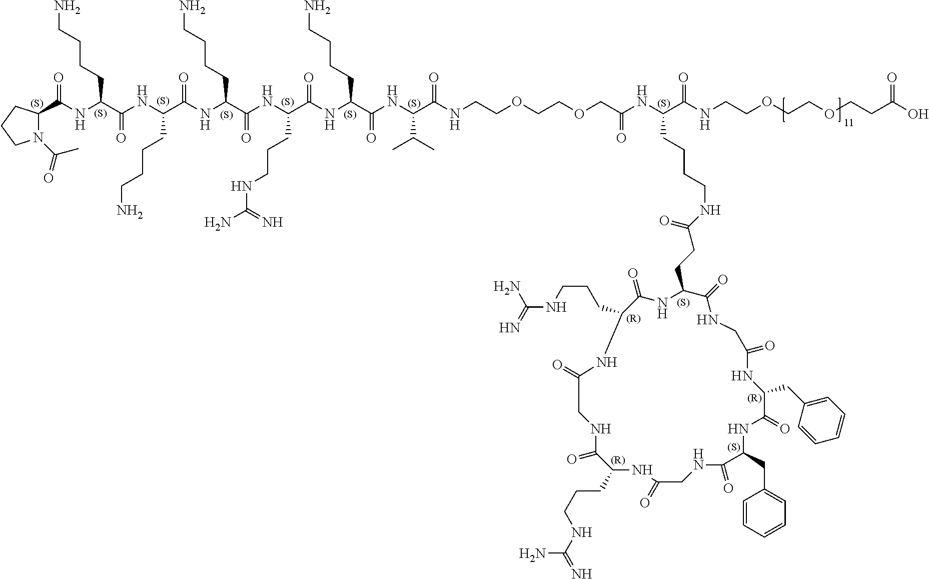

The EEV can be (SEQ ID NOS 700-701, respectively, in order of appearance)

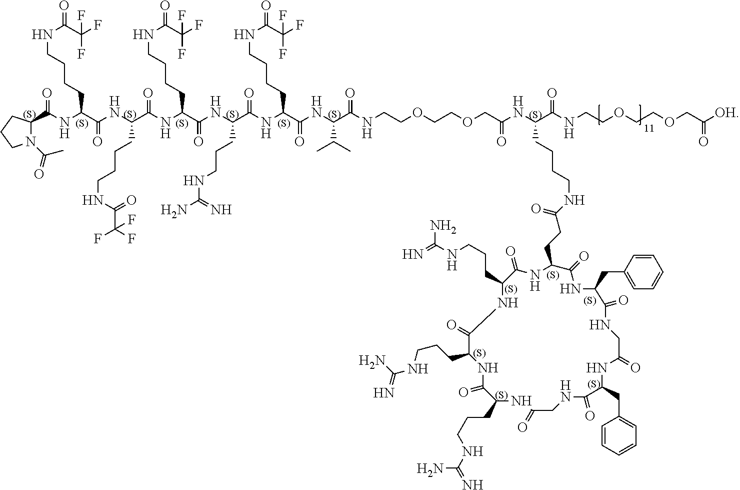

The EEV can be (SEQ ID NOS 702-703, respectively, in order of appearance)

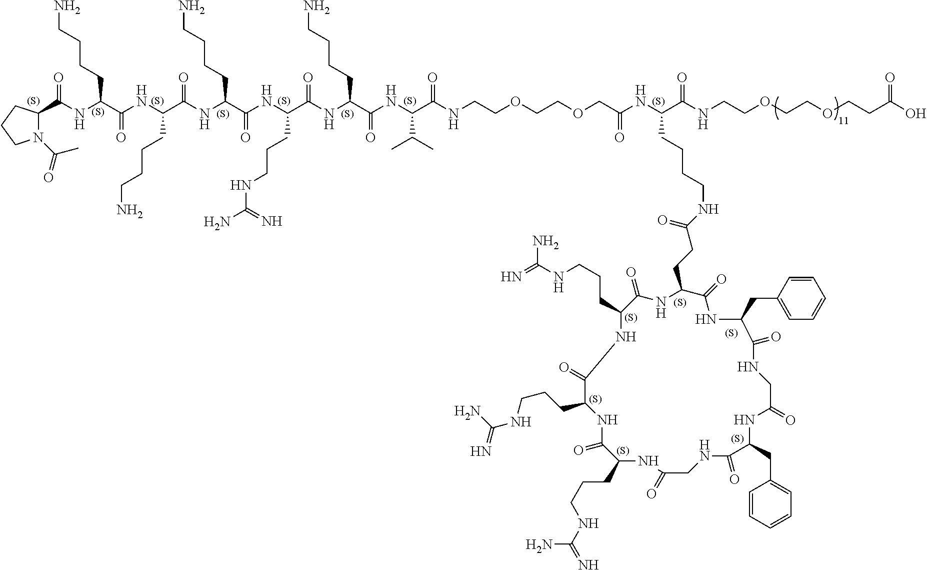

The EEV can be (SEQ ID NOS 704-705, respectively, in order of appearance)

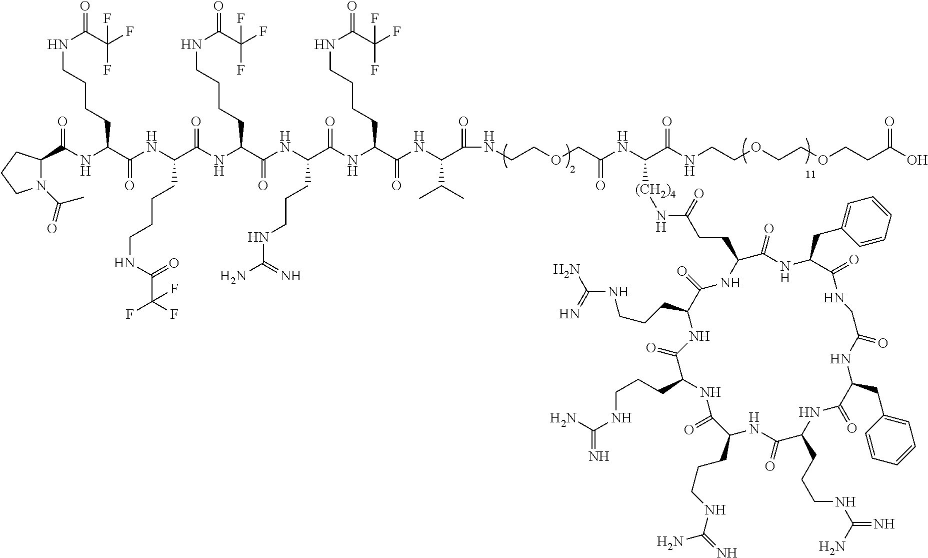

The EEV can be selected from

| | |
|---|---|
| Ac-rr-miniPEG₂-Dap(cyclo[FfΦ-Cit-r-Cit-rQ])-PEG₁₂-OH | SEQ ID NO: 448; Ac-rr- |
| | miniPEG₂-Dap(cyclo[SEQ ID |
| | NO: 136])-PEG₁₂-OH |
| Ac-frr-PEG₂-Dap(cyclo[FfΦ-Cit-r-Cit-rQ])-PEG₁₂-OH | SEQ ID NO: 449; Ac-frr-PEG₂- |
| | Dap(cyclo[SEQ ID NO: 136])- |
| | PEG₁₂-OH |
| Ac-rfr-PEG₂-Dap(cyclo[FfΦ-Cit-r-Cit-rQ])-PEG₁₂-OH | SEQ ID NO: 450; Ac-rfr-PEG₂- |
| | Dap(cyclo[SEQ ID NO: 136])- |
| | PEG₁₂-OH |
| Ac-rbfbr-PEG₂-Dap(cyclo[FfΦ-Cit-r-Cit-rQ])-PEG₁₂-OH | SEQ ID NO: 451; Ac-rbfbr-PEG₂- |
| | Dap(cyclo[SEQ ID NO: 136])- |
| | PEG₁₂-OH |
| Ac-rrr-PEG₂-Dap(cyclo[FfΦ-Cit-r-Cit-rQ])-PEG₁₂-OH | SEQ ID NO: 452; Ac-rrr-PEG₂- |
| | Dap(cyclo[SEQ ID NO: 136])- |
| | PEG₁₂-OH |
| Ac-rbr-PEG₂-Dap(cyclo[FfΦ-Cit-r-Cit-rQ])-PEG₁₂-OH | SEQ ID NO: 453; Ac-rbr-PEG₂- |
| | Dap(cyclo[SEQ ID NO: 136])- |
| | PEG₁₂-OH |
| Ac-rbrbr-PEG₂-Dap(cyclo[FfΦ-Cit-r-Cit-rQ])-PEG₁₂-OH | SEQ ID NO: 454; Ac-rbrbr-PEG₂- |
| | Dap(cyclo[SEQ ID NO: 136])- |
| | PEG₁₂-OH |
| Ac-hh-PEG₂-Dap(cyclo[FfΦ-Cit-r-Cit-rQ])-PEG₁₂-OH | SEQ ID NO: 455; Ac-hh-PEG₂- |
| | Dap(cyclo[SEQ ID NO: 136])- |
| | PEG₁₂-OH |
| Ac-hbh-PEG₂-Dap(cyclo[FfΦ-Cit-r-Cit-rQ])-PEG₁₂-OH | SEQ ID NO: 456; Ac-hbh-PEG₂- |
| | Dap(cyclo[SEQ ID NO: 136])- |
| | PEG₁₂-OH |
| Ac-hbhbh-PEG₂-Dap(cyclo[FfΦ-Cit-r-Cit-rQ])-PEG₁₂-OH | SEQ ID NO: 457; Ac-hbhbh- |
| | PEG₂-Dap(cyclo[SEQ ID |
| | NO: 136])-PEG₁₂-OH |
| Ac-rbhbh-PEG₂-Dap(cyclo[FfΦ-Cit-r-Cit-rQ])-PEG₁₂-OH | SEQ ID NO: 458; Ac-rbhbhPEG₂- |
| | Dap(cyclo[SEQ ID NO: 136])- |
| | PEG₁₂-OH |
| Ac-hbrbh-PEG₂-Dap(cyclo[FfΦ-Cit-r-Cit-rQ])-PEG₁₂-OH | SEQ ID NO: 459; Ac-hbrbh-PEG₂- |
| | Dap(cyclo[SEQ ID NO: 136])- |
| | PEG₁₂-OH |
| Ac-rr-Dap(cyclo[FfΦ-Cit-r-Cit-rQ])-b-OH | SEQ ID NO: 460; Ac-rr- |
| | Dap(cyclo[SEQ ID NO: 136])-b- |
| | OH |
| Ac-frr-Dap(cyclo[FfΦ-Cit-r-Cit-rQ])-b-OH | SEQ ID NO: 461; Ac-frr- |
| | Dap(cyclo[SEQ ID NO: 136])-b- |
| | OH |
| Ac-rfr-Dap(cyclo[FfΦ-Cit-r-Cit-rQ])-b-OH | SEQ ID NO: 462; Ac-rfr- |
| | Dap(cyclo[SEQ ID NO: 136])-b- |
| | OH |
| Ac-rbfbr-Dap(cyclo[FfΦ-Cit-r-Cit-rQ])-b-OH | SEQ ID NO: 463; Ac- rbfbr- |
| | Dap(cyclo[SEQ ID NO: 136])-b- |
| | OH |
| Ac-rrr-Dap(cyclo[FfΦ-Cit-r-Cit-rQ])-b-OH | SEQ ID NO: 464; Ac-rrr- |
| | Dap(cyclo[SEQ ID NO: 136])-b- |
| | OH |
| Ac-rbr-Dap(cyclo[FfΦ-Cit-r-Cit-rQ])-b-OH | SEQ ID NO: 465; Ac-rbr- |
| | Dap(cyclo[SEQ ID NO: 136])-b- |
| | OH |
| Ac-rbrbr-Dap(cyclo[FfΦ-Cit-r-Cit-rQ])-b-OH | SEQ ID NO: 466; Ac-rbrbr- |
| | Dap(cyclo[SEQ ID NO: 136])-b- |
| | OH |
| Ac-hh-Dap(cyclo[FfΦ-Cit-r-Cit-rQ])-b-OH | SEQ ID NO: 467; Ac-hh- |
| | Dap(cyclo[SEQ ID NO: 136])-b- |
| | OH |
| Ac-hbh-Dap(cyclo[FfΦ-Cit-r-Cit-rQ])-b-OH | SEQ ID NO: 468; Ac-hbh- |
| | Dap(cyclo[SEQ ID NO: 136])-b- |
| | OH |
| Ac-hbhbh-Dap(cyclo[FfΦ-Cit-r-Cit-rQ])-b-OH | SEQ ID NO: 469; Ac-hbhbh- |
| | Dap(cyclo[SEQ ID NO: 136])-b- |
| | OH |
| Ac-rbhbh-Dap(cyclo[FfΦ-Cit-r-Cit-rQ])-b-OH | SEQ ID NO: 470; Ac-rbhbh- |
| | Dap(cyclo[SEQ ID NO: 136])-b- |
| | OH |
| Ac-hbrbh-Dap(cyclo[FfΦ-Cit-r-Cit-rQ])-b-OH | SEQ ID NO: 471; Ac-hbrbh- |
| | Dap(cyclo[SEQ ID NO: 136])-b- |
| | OH |
| Ac-KKKK-miniPEG₂-Lys(cyclo[FfΦGrGrQ])-miniPEG₂- | SEQ ID NOS 472-473, |
| K(N₃)-NH₂ | respectively, in order of |
| | appearance; Ac- SEQ ID NO: 7- |
| | miniPEG₂-Lys(cyclo[SEQ ID |
| | NO: 80])-miniPEG₂-K(N₃)-NH₂ |
| Ac-KGKK-miniPEG₂-Lys(cyclo[FfΦGrGrQ])-miniPEG₂- | SEQ ID NOS 474-475, |
| K(N₃)-NH₂ | respectively, in order of |
| | appearance; Ac- SEQ ID NO: 13- |
| | miniPEG₂-Lys(cyclo[SEQ ID |
| | NO: 80])-miniPEG₂-K(N₃)-NH₂ |
| Ac-KKGK-miniPEG₂-Lys(cyclo[FfΦGrGrQ])-miniPEG₂- | SEQ ID NOS 476-477, |
| K(N₃)-NH₂ | respectively, in order of |
| | appearance; Ac- SEQ ID NO: 14- |
| | miniPEG₂-Lys(cyclo[SEQ ID |
| | NO: 80])-miniPEG₂-K(N₃)-NH₂ |
| Ac-KKK-miniPEG₂-Lys(cyclo[FfΦGrGrQ])-miniPEG₂- | SEQ ID NO: 478; Ac-KKK- |
| K(N₃)-NH₂ | miniPEG₂-Lys(cyclo[SEQ ID |
| | NO: 80])-miniPEG₂-K(N₃)-NH₂ |
| Ac-KK-miniPEG₂-Lys(cyclo[FfΦGrGrQ])-miniPEG₂- | SEQ ID NO: 479; Ac-KK- |
| K(N₃)-NH₂ | miniPEG₂-Lys(cyclo[SEQ ID |
| | NO: 80])-miniPEG₂-K(N₃)-NH₂ |
| Ac-KGK-miniPEG₂-Lys(cyclo[FfΦGrGrQ])-miniPEG₂- | SEQ ID NO: 480; Ac-KGK- |
| K(N₃)-NH₂ | miniPEG₂-Lys(cyclo[SEQ ID |
| | NO: 80])-miniPEG₂-K(N₃)-NH₂ |
| Ac-KBK-miniPEG₂-Lys(cyclo[FfΦGrGrQ])-miniPEG₂- | SEQ ID NO: 481; Ac-KBK- |
| K(N₃)-NH₂ | miniPEG₂-Lys(cyclo[SEQ ID |
| | NO: 80])-miniPEG₂-K(N₃)-NH₂ |
| Ac-KBKBK-miniPEG₂-Lys(cyclo[FfΦGrGrQ])-miniPEG₂- | SEQ ID NO: 482; Ac-KBKBK- |
| K(N₃)-NH₂ | miniPEG₂-Lys(cyclo[SEQ ID |
| | NO: 80])-miniPEG₂-K(N₃)-NH₂ |
| Ac-KR-miniPEG₂-Lys(cyclo[FfΦGrGrQ])-miniPEG₂- | SEQ ID NO: 483; Ac-KR- |
| K(N₃)-NH₂ | miniPEG₂-Lys(cyclo[SEQ ID |
| | NO: 80])-miniPEG₂-K(N₃)-NH₂ |
| Ac-KBR-miniPEG₂-Lys(cyclo[FfΦGrGrQ])-miniPEG₂- | SEQ ID NO: 484; Ac-KBR- |
| K(N₃)-NH₂ | miniPEG₂-Lys(cyclo[SEQ ID |
| | NO: 80])-miniPEG₂-K(N₃)-NH₂ |
| Ac-PKKKRKV-miniPEG₂-Lys(cyclo[FfΦGrGrQ])- | SEQ ID NOS 485-486, |
| miniPEG₂-K(N₃)-NH₂ | respectively, in order of |
| | appearance; Ac- SEQ ID NO: 42- |
| | miniPEG₂-Lys(cyclo[SEQ ID |
| | NO: 80])-miniPEG₂-K(N₃)-NH₂ |
| Ac-PKKKRKV-miniPEG₂-Lys(cyclo[FfΦGrGrQ])- | SEQ ID NOS 485-486, |
| miniPEG₂-K(N₃)-NH₂ | respectively, in order of |
| | appearance; Ac- SEQ ID NO: 42- |
| | miniPEG₂-Lys(cyclo[SEQ ID |
| | NO: 80])-miniPEG₂-K(N₃)-NH₂ |
| Ac-PGKKRKV-miniPEG₂-Lys(cyclo[FfΦGrGrQ])- | SEQ ID NOS 487-488, |
| miniPEG₂-K(N₃)-NH₂ | respectively, in order of |
| | appearance; Ac- SEQ ID NO: 43- |
| | miniPEG₂-Lys(cyclo[SEQ ID |
| | NO: 80])-miniPEG₂-K(N₃)-NH₂ |
| Ac-PKGKRKV-miniPEG₂-Lys(cyclo[FfΦGrGrQ])- | SEQ ID NOS 489-490, |
| miniPEG₂-K(N₃)-NH₂ | respectively, in order of |
| | appearance; Ac- SEQ ID NO: 44- |
| | miniPEG₂-Lys(cyclo[SEQ ID |
| | NO: 80])-miniPEG₂-K(N₃)-NH₂ |
| Ac-PKKGRKV-miniPEG₂-Lys(cyclo[FfΦGrGrQ])- | SEQ ID NOS 491-492, |
| miniPEG₂-K(N₃)-NH₂ | respectively, in order of |
| | appearance; Ac- SEQ ID NO: 45- |
| | miniPEG₂-Lys(cyclo[SEQ ID |
| | NO: 80])-miniPEG₂-K(N₃)-NH₂ |
| Ac-PKKKGKV-miniPEG₂-Lys(cyclo[FfΦGrGrQ])- | SEQ ID NOS 493-494, |
| miniPEG₂-K(N₃)-NH₂ | respectively, in order of |
| | appearance; Ac- SEQ ID NO: 46- |
| | miniPEG₂-Lys(cyclo[SEQ ID |
| | NO: 80])-miniPEG₂-K(N₃)-NH₂ |
| Ac-PKKKRGV-miniPEG₂-Lys(cyclo[FfΦGrGrQ])- | SEQ ID NOS 495-496, |
| miniPEG₂-K(N₃)-NH₂ | respectively, in order of |
| | appearance; Ac- SEQ ID NO: 47- |
| | miniPEG₂-Lys(cyclo[SEQ ID |
| | NO: 80])-miniPEG₂-K(N₃)-NH₂ |
| Ac-PKKKRKG-miniPEG₂-Lys(cyclo[FfΦGrGrQ])- | SEQ ID NOS 497-498, |
| miniPEG₂-K(N₃)-NH₂ | respectively, in order of |
| | appearance; Ac- SEQ ID NO: 48- |
| | miniPEG₂-Lys(cyclo[SEQ ID |
| | NO: 80])-miniPEG₂-K(N₃)-NH₂ |
| Ac-KKKRK-miniPEG₂-Lys(cyclo[FfΦGrGrQ])- | SEQ ID NOS 499-500, |
| miniPEG₂-K(N₃)-NH₂ | respectively, in order of |
| | appearance; Ac- SEQ ID NO: 19- |
| | miniPEG₂-Lys(cyclo[SEQ ID |
| | NO: 80])-miniPEG₂-K(N₃)-NH₂ |
| Ac-KKRK-miniPEG₂-Lys(cyclo[FfΦGrGrQ])-miniPEG₂- | SEQ ID NOS 501-502, |
| K(N₃)-NH₂ and | respectively, in order of |
| | appearance; Ac- SEQ ID NO: 8- |
| | miniPEG₂-Lys(cyclo[SEQ ID |
| | NO: 80])-miniPEG₂-K(N₃)-NH₂ |
| | and |
| Ac-KRK-miniPEG₂-Lys(cyclo[FfΦGrGrQ])-miniPEG₂- | SEQ ID NO: 503; Ac-KRK- |
| K(N₃)-NH₂. | miniPEG₂-Lys(cyclo[SEQ ID |
| | NO: 80])-miniPEG₂-K(N₃)-NH₂. |

The EEV can be selected from:
Ac-PKKKRKV-Lys(cyclo[FfΦGrGrQ])-PEG₁₂-K(N₃)—NH₂ (SEQ ID NOS 504-505, respectively, in order of appearance; Ac-SEQ ID NO:42-Lys(cyclo[SEQ ID NO:80])-PEG₁₂-K(N₃)—NH₂)
Ac-PKKKRKV-miniPEG2-Lys(cyclo[FfΦGrGrQ])-miniPEG₂-K(N₃)—NH₂ (SEQ ID NOS 485-486, respectively, in order of appearance; Ac-SEQ ID NO:42-miniPEG₂-Lys(cyclo[SEQ ID NO:80])-miniPEG₂-K(N₃)—NH₂)
Ac-PKKKRKV-miniPEG₂-Lys(cyclo[FGFGRGRQ])-miniPEG₂-K(N₃)—NH₂ (SEQ ID NOS 506-507, respectively, in order of appearance; Ac-SEQ ID NO:42-miniPEG₂-Lys(cyclo[SEQ ID NO:82])-miniPEG₂-K(N₃)—NH₂) Ac-KR-PEG₂-K(cyclo[FGFGRGRQ])-PEG₂-K(N₃)—NH₂ (SEQ ID NO: 508; Ac-KR-PEG₂-K(cyclo[SEQ ID NO:82])-PEG₂-K(N₃)—NH₂)
Ac-PKKKGKV-PEG₂-K(cyclo[FGFGRGRQ])-PEG₂-K(N₃)—NH₂ (SEQ ID NOS 509-510, respectively, in order of appearance; Ac-SEQ ID NO:46-PEG₂-K(cyclo[SEQ ID NO:82])-PEG₂-K(N₃)—NH₂)
Ac-PKKKRKG-PEG₂-K(cyclo[FGFGRGRQ])-PEG₂-K(N₃)—NH₂ (SEQ ID NOS 511-512, respectively, in order of appearance; Ac-SEQ ID NO:48-PEG₂-K(cyclo[SEQ ID NO:82])-PEG₂-K(N₃)—NH₂) Ac-KKKRK-PEG₂-K(cyclo[FGFGRGRQ])-PEG₂-K(N₃)—NH₂ (SEQ ID NOS 513-514, respectively, in order of appearance; Ac-SEQ ID NO:19-PEG₂-K(cyclo[SEQ ID NO:82])-PEG₂-K(N₃)—NH₂)
Ac-PKKKRKV-miniPEG₂-Lys(cyclo[FF(GRGRQ])-miniPEG₂-K(N₃)—NH₂ (SEQ ID NOS 515-516, respectively, in order of appearance; Ac-SEQ ID NO:42-miniPEG₂-Lys(cyclo[SEQ ID NO:80])-miniPEG₂-K(N₃)—NH₂)
Ac-PKKKRKV-miniPEG₂-Lys(cyclo[PhFfΦGrGrQ])-miniPEG₂-K(N₃)—NH₂ (SEQ ID NOS 517-518, respectively, in order of appearance; Ac-SEQ ID NO:42-miniPEG₂-Lys(cyclo[SEQ ID NO:142])-miniPEG₂-K(N₃)—NH₂) Ac-PKKKRKV-miniPEG₂-Lys(cyclo[Ff(SrSrQ])-miniPEG₂-K(N₃)—NH₂ (SEQ ID NOS 519-520, respectively, in order of appearance; Ac-SEQ ID NO:42-miniPEG₂-Lys(cyclo[SEQ ID NO:143])-miniPEG₂-K(N₃)—NH₂).
The EEV can be selected from: Ac-PKKKRKV-miniPEG₂-Lys(cyclo(GfFGrGrQ])-PEG₁₂—OH (SEQ ID NOS 521-522, respectively, in order of appearance; Ac-SEQ ID NO:42-miniPEG₂-Lys(cyclo(SEQ ID NO:133])-PEG₁₂—OH)
Ac-PKKKRKV-miniPEG₂-Lys(cyclo[FGFKRKRQ])-PEG₁₂—OH (SEQ ID NOS 523-524, respectively, in order of appearance; Ac-SEQ ID NO:42-miniPEG₂-Lys(cyclo[SEQ ID NO:144])-PEG₁₂—OH)
Ac-PKKKRKV-miniPEG₂-Lys(cyclo[FGFRGRGQ])-PEG₁₂—OH (SEQ ID NOS 525-526, respectively, in order of appearance; Ac-SEQ ID NO:42-miniPEG₂-Lys(cyclo[SEQ ID NO:145])-PEG₁₂—OH) Ac-PKKKRKV-miniPEG₂-Lys(cyclo[FGFGRGRGRQ])-PEG₁₂—OH (SEQ ID NOS 527-528, respectively, in order of appearance; Ac-SEQ ID NO:42-miniPEG₂-Lys(cyclo[SEQ ID NO:712)-PEG₁₂—OH)
Ac-PKKKRKV-miniPEG₂-Lys(cyclo[FGFGRrRQ])-PEG₁₂—OH (SEQ ID NOS 529-530, respectively, in order of appearance; Ac-SEQ ID NO:42-miniPEG₂-Lys(cyclo[SEQ ID NO:713)-PEG₁₂—OH)
Ac-PKKKRKV-miniPEG₂-Lys(cyclo[FGFGRRRQ])-PEG₁₂—OH (SEQ ID NOS 531-532, respectively, in order of appearance; Ac-SEQ ID NO:42-miniPEG₂-Lys(cyclo[SEQ ID NO:84])-PEG₁₂—OH) and Ac-PKKKRKV-miniPEG₂-Lys(cyclo[FGFRRRRQ])-PEG₁₂—OH (SEQ ID NOS 533-534, respectively, in order of appearance; Ac-SEQ ID NO:42-miniPEG₂-Lys(cyclo[SEQ ID NO:85])-PEG₁₂—OH).
The EEV can be selected from: Ac-K-K-K-R-K-G-miniPEG₂-K(cyclo[FGFGRGRQ])-PEG₁₂—OH (SEQ ID NOS 535-536, respectively, in order of appearance; Ac-SEQ ID NO:714-miniPEG₂-K(cyclo[SEQ ID NO:82])-PEG₁₂—OH)
Ac-K-K-K-R-K-miniPEG₂-K(cyclo[FGFGRGRQ])-PEG₁₂—OH (SEQ ID NOS 537-538, respectively, in order of appearance; Ac-SEQ ID NO:19-miniPEG₂-K(cyclo[SEQ ID NO:82])-PEG₁₂—OH)
Ac-K-K-R-K-K-PEG₄-K(cyclo[FGFGRGRQ])-PEG₁₂—OH (SEQ ID NOS 539-540, respectively, in order of appearance; Ac-SEQ ID NO:22-PEG₄-K(cyclo[SEQ ID NO:82])-PEG₁₂—OH) Ac-K-R-K-K-K-PEG₄-K(cyclo[FGFGRGRQ])-PEG₁₂—OH (SEQ ID NOS 541-542, respectively, in order of appearance; Ac-SEQ ID NO:21-PEG₄-K(cyclo[SEQ ID NO:82])-PEG₁₂—OH)
Ac-K-K-K-K-R-PEG₄-K(cyclo[FGFGRGRQ])-PEG₁₂—OH (SEQ ID NOS 543-544, respectively, in order of appearance; Ac-SEQ ID NO:23-PEG₄-K(cyclo[SEQ ID NO:82])-PEG₁₂—OH)
Ac-R-K-K-K-K-PEG₄-K(cyclo[FGFGRGRQ])-PEG₁₂—OH (SEQ ID NOS 545-546, respectively, in order of appearance; Ac-SEQ ID NO:20-PEG₄-K(cyclo[SEQ ID NO:82])-PEG₁₂—OH) and Ac-K-K-K-R-K-PEG₄-K(cyclo[FGFGRGRQ])-PEG₁₂—OH (SEQ ID NOS 547-548, respectively, in order of appearance; Ac-SEQ ID NO:19-PEG₄-K(cyclo[SEQ ID NO:82])-PEG₁₂—OH).
The EEV can be selected from: Ac-PKKKRKV-PEG₂-K(cyclo[FGFGRGRQ])-PEG₂-K(N₃)—NH₂ (SEQ ID NOS 549-550, respectively, in order of appearance; Ac-SEQ ID NO:42-PEG₂-K(cyclo[SEQ ID NO:82])-PEG₂-K(N₃)—NH₂)
Ac-PKKKRKV-PEG₂-K(cyclo[FGFGRGRQ])-PEG₁₂—OH (SEQ ID NOS 551-552, respectively, in order of appearance; Ac-SEQ ID NO:42-PEG₂-K(cyclo[SEQ ID NO:82])-PEG₁₂—OH)
Ac-PKKKRKV-PEG₂-K(cyclo[GfFGrGrQ])-PEG₂-K(N₃)—NH₂ (SEQ ID NOS 553-554, respectively, in order of appearance; Ac-SEQ ID NO:42-PEG₂-K(cyclo[SEQ ID NO:133])-PEG₂-K(N₃)—NH₂) and Ac-PKKKRKV-PEG₂-K(cyclo[GfFGrGrQ])-PEG₁₂—OH (SEQ ID NOS 555-556, respectively, in order of appearance; Ac-SEQ ID NO:42-PEG₂-K(cyclo[SEQ ID NO:133])-PEG₁₂—OH).
The cargo can be an AC and the EEV can be selected from: Ac-PKKKRKV-PEG₂-K(cyclo[FfΦGrGrQ])-PEG₁₂—OH (SEQ ID NOS 557-558, respectively, in order of appearance; Ac-SEQ ID NO:42-PEG₂-K(cyclo[SEQ ID NO:80])-PEG₁₂—OH)
Ac-PKKKRKV-PEG₂-K(cyclo[Ff(DCit-r-Cit-rQ])-PEG₁₂—OH (SEQ ID NOS 559-560, respectively, in order of appearance; Ac-SEQ ID NO:42-PEG₂-K(cyclo[SEQ ID NO:79])-PEG₁₂—OH)
Ac-PKKKRKV-PEG₂-K(cyclo[FfFGRGRQ])-PEG₁₂—OH (SEQ ID NOS 561-562, respectively, in order of appearance; Ac-SEQ ID NO:42-PEG₂-K(cyclo[SEQ ID NO:81])-PEG₁₂—OH) Ac-PKKKRKV-PEG₂-K(cyclo[FGFGRGRQ])-PEG₁₂—OH (SEQ ID NOS 563-564, respectively, in order of appearance; Ac-SEQ ID NO:42-PEG₂-K(cyclo[SEQ ID NO:82])-PEG₁₂—OH)
Ac-PKKKRKV-PEG₂-K(cyclo[GfFGrGrQ])-PEG₁₂—OH (SEQ ID NOS 565-566, respectively, in order of appearance; Ac-SEQ ID NO:42-PEG₂-K(cyclo[SEQ ID NO:133])-PEG₁₂—OH)
Ac-PKKKRKV-PEG₂-K(cyclo[FGFGRRRQ])-PEG₁₂—OH (SEQ ID NOS 567-568, respectively, in order of appearance; Ac-SEQ ID NO:42-PEG₂-K(cyclo[SEQ ID NO:84])-PEG₁₂—OH) Ac-PKKKRKV-PEG₂-K(cyclo[FGFRRRRQ])-PEG₁₂—OH (SEQ ID NOS 569-570, respectively, in order of appearance; Ac-SEQ ID NO:42-PEG₂-K(cyclo[SEQ ID NO:85])-PEG₁₂—OH)
Ac-rr-PEG₂-K(cyclo[FfΦGrGrQ])-PEG₁₂—OH (SEQ ID NO: 571; Ac-rr-PEG₂-K(cyclo[SEQ ID NO:80])-PEG₁₂—OH)
Ac-rr-PEG₂-K(cyclo[Ff(Cit-r-Cit-rQ])-PEG₁₂—OH (SEQ ID NO: 572; Ac-rr-PEG₂-K(cyclo[SEQ ID NO:79])-PEG₁₂—OH) Ac-rr-PEG₂-K(cyclo[FfF-GRGRQ])-PEG₁₂—OH (SEQ ID NO: 573; Ac-rr-PEG₂-K(cyclo[SEQ ID NO:81])-PEG₁₂—OH)
Ac-rr-PEG₂-K(cyclo[FGFGRGRQ])-PEG₁₂—OH (SEQ ID NO: 574; Ac-rr-PEG₂-K(cyclo[SEQ ID NO:82])-PEG₁₂—OH)
Ac-rr-PEG₂-K(cyclo[GfFGrGrQ])-PEG₁₂—OH (SEQ ID NO: 575; Ac-rr-PEG₂-K(cyclo[SEQ ID NO:133])-PEG₁₂—OH) Ac-rr-PEG₂-K(cyclo[FGFGRRRQ])-PEG₁₂—OH (SEQ ID NO: 576; Ac-rr-PEG₂-K(cyclo[SEQ ID NO:84])-PEG₁₂—OH)
Ac-rr-PEG₂-K(cyclo[FGFRRRRQ])-PEG₁₂—OH (SEQ ID NO: 577; Ac-rr-PEG₂-K(cyclo[SEQ ID NO:85])-PEG₁₂—OH)
Ac-rrr-PEG₂-K(cyclo[FfΦGrGrQ])-PEG₁₂—OH (SEQ ID NO: 578; Ac-rrr-PEG₂-K(cyclo[SEQ ID NO:80])-PEG₁₂—OH) Ac-rrr-PEG₂-K(cyclo[Ff(Cit-r-Cit-rQ])-PEG₁₂—OH (SEQ ID NO: 579; Ac-rrr-PEG₂-K(cyclo[SEQ ID NO:79])-PEG₁₂—OH)
Ac-rrr-PEG₂-K(cyclo[FfFGRGRQ])-PEG₁₂—OH (SEQ ID NO: 580; Ac-rrr-PEG₂-K(cyclo[SEQ ID NO:81])-PEG₁₂—OH)
Ac-rrr-PEG₂-K(cyclo[FGFGRGRQ])-PEG₁₂—OH (SEQ ID NO: 581; Ac-rrr-PEG₂-K(cyclo[SEQ ID NO:82])-PEG₁₂—OH) Ac-rrr-PEG₂-K(cyclo[GfFGrGrQ])-PEG₁₂—OH (SEQ ID NO: 582; Ac-rrr-PEG₂-K(cyclo[SEQ ID NO:133])-PEG₁₂—OH) Ac-rrr-PEG₂-K(cyclo[FGFGRRRQ])-PEG₁₂—OH (SEQ ID NO: 583; Ac-rrr-PEG₂-K(cyclo[SEQ ID NO:84])-PEG₁₂—OH)
Ac-rrr-PEG₂-K(cyclo[FGFRRRRQ])-PEG₁₂—OH (SEQ ID NO: 584; Ac-rrr-PEG₂-K(cyclo[SEQ ID NO:85])-PEG₁₂—OH)
Ac-rhr-PEG₂-K(cyclo[FfΦGrGrQ])-PEG₁₂—OH (SEQ ID NO: 585; Ac-rhr-PEG₂-K(cyclo[SEQ ID NO:80])-PEG₁₂—OH) Ac-rhr-PEG₂-K(cyclo[Ff(Cit-r-Cit-rQ])-PEG₁₂—OH (SEQ ID NO: 586; Ac-rhr-PEG₂-K(cyclo[SEQ ID NO:79])-PEG₁₂—OH)
Ac-rhr-PEG₂-K(cyclo[FfFGRGRQ])-PEG₁₂—OH (SEQ ID NO: 587; Ac-rhr-PEG₂-K(cyclo[SEQ ID NO:81])-PEG₁₂—OH)
Ac-rhr-PEG₂-K(cyclo[FGFGRGRQ])-PEG₁₂—OH (SEQ ID NO: 588; Ac-rhr-PEG₂-K(cyclo[SEQ ID NO:82])-PEG₁₂—OH) Ac-rhr-PEG₂-K(cyclo[GfFGrGrQ])-PEG₁₂—OH (SEQ ID NO: 589; Ac-rhr-PEG₂-K(cyclo[SEQ ID NO:133])-PEG₁₂—OH)
Ac-rhr-PEG₂-K(cyclo[FGFGRRRQ])-PEG₁₂—OH (SEQ ID NO: 590; Ac-rhr-PEG₂-K(cyclo[SEQ ID NO:84])-PEG₁₂—OH)
Ac-rhr-PEG₂-K(cyclo[FGFRRRRQ])-PEG₁₂—OH (SEQ ID NO: 591; Ac-rhr-PEG₂-K(cyclo[SEQ ID NO:85])-PEG₁₂—OH) Ac-rbr-PEG₂-K(cyclo[FfΦGrGrQ])-PEG₁₂—OH (SEQ ID NO: 592; Ac-rbr-PEG₂-K(cyclo[SEQ ID NO:80])-PEG₁₂—OH)
Ac-rbr-PEG₂-K(cyclo[Ff(Cit-r-Cit-rQ])-PEG₁₂—OH (SEQ ID NO: 593; Ac-rbr-PEG₂-K(cyclo[SEQ ID NO:79])-PEG₁₂—OH)
Ac-rbr-PEG₂-K(cyclo[FfFGRGRQ])-PEG₁₂—OH (SEQ ID NO: 594; Ac-rbr-PEG₂-K(cyclo[SEQ ID NO:81])-PEG₁₂—OH) Ac-rbr-PEG₂-K(cyclo[FGFGRGRQ])-PEG₁₂—OH (SEQ ID NO: 595; Ac-rbr-PEG₂-K(cyclo[SEQ ID NO:82])-PEG₁₂—OH)
Ac-rbr-PEG₂-K(cyclo[GfFGrGrQ])-PEG₁₂—OH (SEQ ID NO: 596; Ac-rbr-PEG₂-K(cyclo[SEQ ID NO:133])-PEG₁₂—OH) Ac-rbr-PEG₂-K(cyclo[FGFGRRRQ])-PEG₁₂—OH (SEQ ID NO: 597; Ac-rbr-PEG₂-K(cyclo[SEQ ID NO:84])-PEG₁₂—OH)
Ac-rbr-PEG₂-K(cyclo[FGFRRRRQ])-PEG₁₂—OH (SEQ ID NO: 598; Ac-rbr-PEG₂-K(cyclo[SEQ ID NO:85])-PEG₁₂—OH) Ac-rbrbr-PEG₂-K(cyclo[FfΦGrGrQ])-PEG₁₂—OH (SEQ ID NO: 599; Ac-rbrbr-PEG₂-K(cyclo[SEQ ID NO:80])-PEG₁₂—OH)
Ac-rbrbr-PEG₂-K(cyclo[Ff(Cit-r-Cit-rQ])-PEG₁₂—OH (SEQ ID NO: 600; Ac-rbrbr-PEG₂-K(cyclo[SEQ ID NO:79])-PEG₁₂—OH)
Ac-rbrbr-PEG₂-K(cyclo[FfFGRGRQ])-PEG₁₂—OH (SEQ ID NO: 601; Ac-rbrbr-PEG₂-K(cyclo[SEQ ID NO:81])-PEG₁₂—OH) Ac-rbrbr-PEG₂-K(cyclo[FGFGRGRQ])-PEG₁₂—OH (SEQ ID NO: 602; Ac-rbrbr-PEG₂-K(cyclo[SEQ ID NO:82])-PEG₁₂—OH)
Ac-rbrbr-PEG₂-K(cyclo[GfFGrGrQ])-PEG₁₂—OH (SEQ ID NO: 603; Ac-rbrbr-PEG₂-K(cyclo[SEQ ID NO:133])-PEG₁₂—OH)
Ac-rbrbr-PEG₂-K(cyclo[FGFGRRRQ])-PEG₁₂—OH (SEQ ID NO: 604; Ac-rbrbr-PEG₂-K(cyclo[SEQ ID NO:84])-PEG₁₂—OH) Ac-rbrbr-PEG₂-K(cyclo[FGFRRRRQ])-PEG₁₂—OH (SEQ ID NO: 605; Ac-rbrbr-PEG₂-K(cyclo[SEQ ID NO:85])-PEG₁₂—OH)
Ac-rbhbr-PEG₂-K(cyclo[FfΦGrGrQ])-PEG₁₂—OH (SEQ ID NO: 606; Ac-rbhbr-PEG₂-K(cyclo[SEQ ID NO:80])-PEG₁₂—OH)
Ac-rbhbr-PEG₂-K(cyclo[Ff(Cit-r-Cit-rQ])-PEG₁₂—OH (SEQ ID NO: 607; Ac-rbhbr-PEG₂-K(cyclo[SEQ ID NO:79])-PEG₁₂—OH) Ac-rbhbr-PEG₂-K(cyclo[FfFGRGRQ])-PEG₁₂—OH (SEQ ID NO: 608; Ac-rbhbr-PEG₂-K(cyclo[SEQ ID NO:81])-PEG₁₂—OH)
Ac-rbhbr-PEG₂-K(cyclo[FGFGRGRQ])-PEG₁₂—OH (SEQ ID NO: 609; Ac-rbhbr-PEG₂-K(cyclo[SEQ ID NO:82])-PEG₁₂—OH)
Ac-rbhbr-PEG₂-K(cyclo[GfFGrGrQ])-PEG₁₂—OH (SEQ ID NO: 610; Ac-rbhbr-PEG₂-K(cyclo[SEQ ID NO:133])-PEG₁₂—OH) Ac-rbhbr-PEG₂-K(cyclo[FGFGRRRQ])-PEG₁₂—OH (SEQ ID NO: 611; Ac-rbhbr-PEG₂-K(cyclo[SEQ ID NO:84])-PEG₁₂—OH)
Ac-rbhbr-PEG₂-K(cyclo[FGFRRRRQ])-PEG₁₂—OH (SEQ ID NO: 612; Ac-rbhbr-PEG₂-K(cyclo[SEQ ID NO:85])-PEG₁₂—OH)
Ac-hbrbh-PEG₂-K(cyclo[FfΦGrGrQ])-PEG₁₂—OH (SEQ ID NO: 613; Ac-hbrbh-PEG₂-K(cyclo[SEQ ID NO:80])-PEG₁₂—OH) Ac-hbrbh-PEG₂-K(cyclo[Ff(DCit-r-Cit-rQ])-PEG₁₂—OH (SEQ ID NO: 614; Ac-hbrbh-PEG₂-K(cyclo[SEQ ID NO:79])-PEG₁₂—OH)
Ac-hbrbh-PEG₂-K(cyclo[FfFGRGRQ])-PEG₁₂—OH (SEQ ID NO: 615; Ac-hbrbh-PEG₂-K(cyclo[SEQ ID NO:81])-PEG₁₂—OH)
Ac-hbrbh-PEG₂-K(cyclo[FGFGRGRQ])-PEG₁₂—OH (SEQ ID NO: 616; Ac-hbrbh-PEG₂-K(cyclo[SEQ ID NO:82])-PEG₁₂—OH) Ac-hbrbh-PEG₂-K(cyclo[GfFGrGrQ])-PEG₁₂—OH (SEQ ID NO: 617; Ac-hbrbh-PEG₂-K(cyclo[SEQ ID NO:133])-PEG₁₂—OH)
Ac-hbrbh-PEG₂-K(cyclo[FGFGRRRQ])-PEG₁₂—OH (SEQ ID NO: 618; Ac-hbrbh-PEG₂-K(cyclo[SEQ ID NO:84])-PEG₁₂—OH)
Ac-hbrbh -PEG₂-K(cyclo[FGFRRRRQ])-PEG₁₂—OH (SEQ ID NO: 619; Ac-hbrbh-PEG₂-K(cyclo[SEQ ID NO:85])-PEG₁₂—OH), wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. As used herein, “cargo” is a compound or moiety for which delivery into a cell is desired. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by the cargoty or at least one lone pair of the cCPP forms a bond to the cargo. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

In embodiments, the amino acid side chain comprises a chemically reactive group to which the linker or cargo is conjugated. The chemically reactive group can comprise an amine group, a carboxylic acid, an amide, a hydroxyl group, a sulfhydryl group, a guanidinyl group, a phenolic group, a thioether group, an imidazolyl group, or an indolyl group. In embodiments, the amino acid of the cCPP to which the cargo is conjugated comprises lysine, arginine, aspartic acid, glutamic acid, asparagine, glutamine, homoglutamine, serine, threonine, tyrosine, cysteine, arginine, tyrosine, methionine, histidine or tryptophan.

The cargo can comprise one or more detectable moieties, one or more therapeutic moieties (TMs), one or more targeting moieties, or any combination thereof. In embodiments, the cargo comprises a TM. In embodiments, the cargo comprises an AC.

Cyclic cell penetrating peptides (cCPPs) conjugated to a cargo moiety

The cyclic cell penetrating peptide (cCPP) can be conjugated to a cargo moiety.

The cargo moiety can be conjugated to the linker at the terminal carbonyl group to provide the following structure:

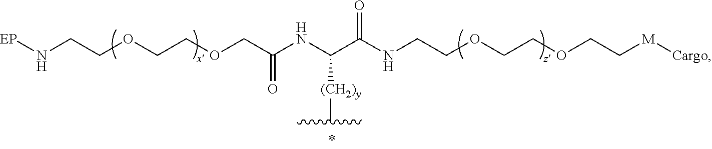

wherein:

EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)x′— and/or —(OCH₂CH₂)_{z}′-can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

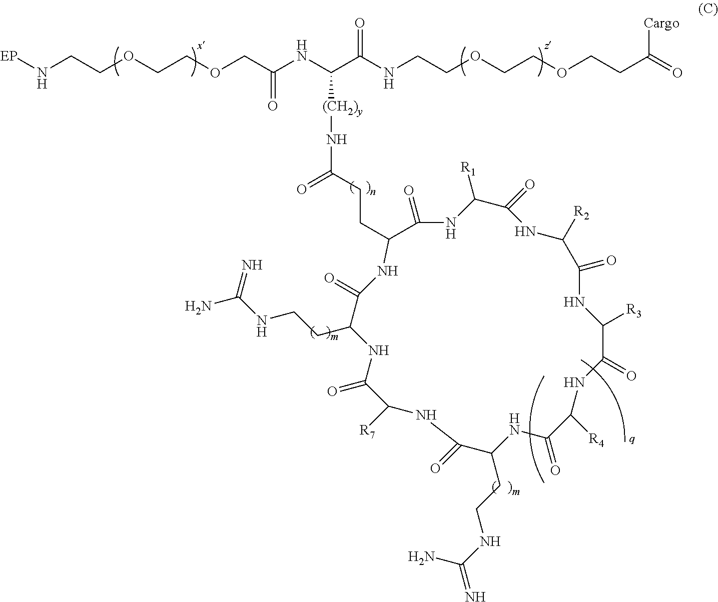

or a protonated form thereof,
wherein.

R₁, R₂, and R₃ can each independently be H or an amino acid residue having a side chain comprising an aromatic group;
R₄ is H or an amino acid side chain;
EP is an exocyclic peptide as defined herein;
Cargo is a moiety as defined herein;
each m is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

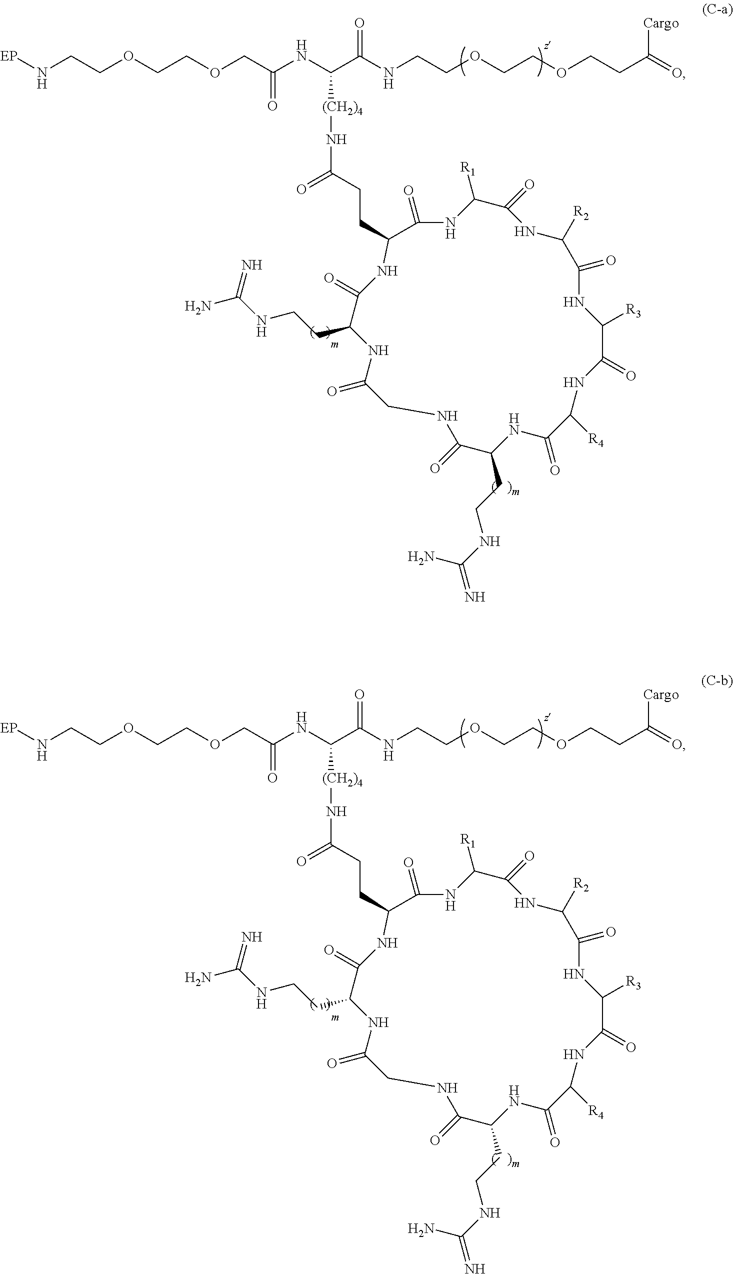

or a protonated form thereof, wherein EP, m and z are as defined above in Formula (C).

The EEV can be conjugated to a cargo and the EEV-conjugate can comprise the structure of Formula (C-c):

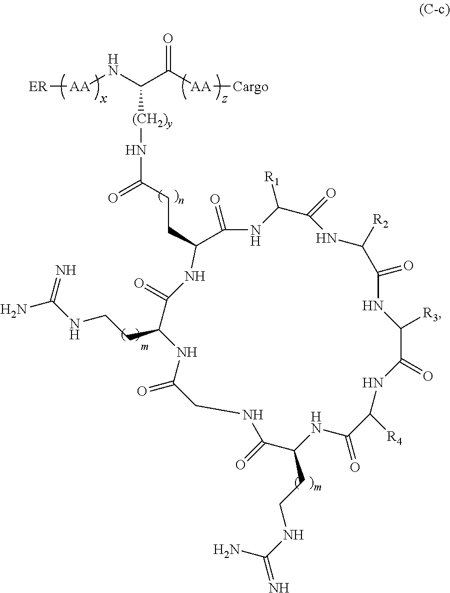

or a protonated form thereof, wherein EP, R₁, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4) (SEQ ID NOS 706-709, respectively, in order of appearance):

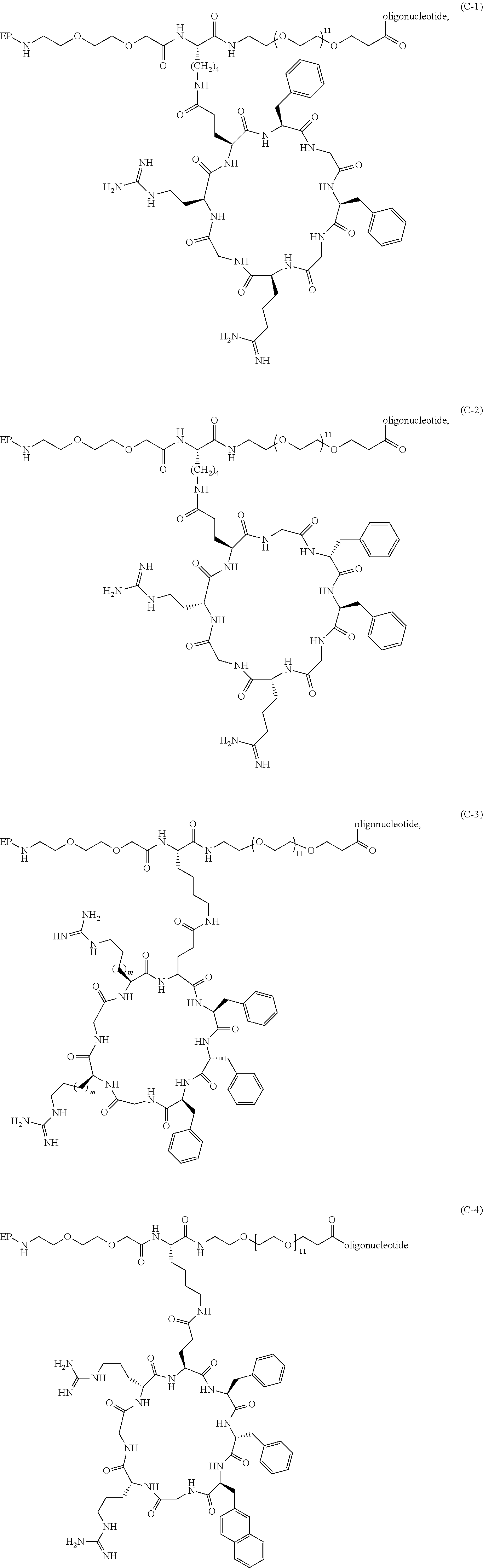

The EEV can be conjugated to an oligonucleotide cargo and the EEV-conjugate can comprise the structure (SEQ ID NOS 710-711 and 658, respectively, in order of appearance):

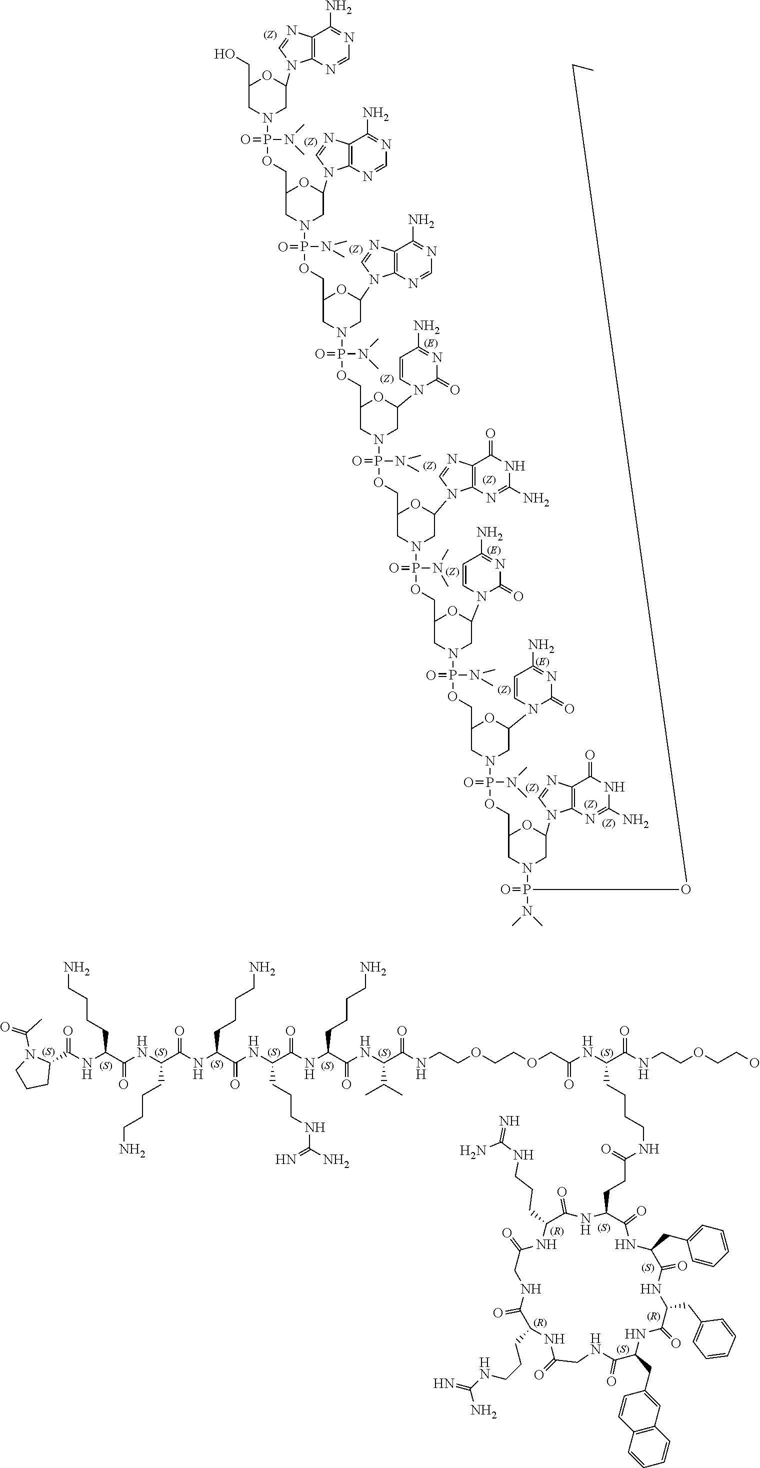

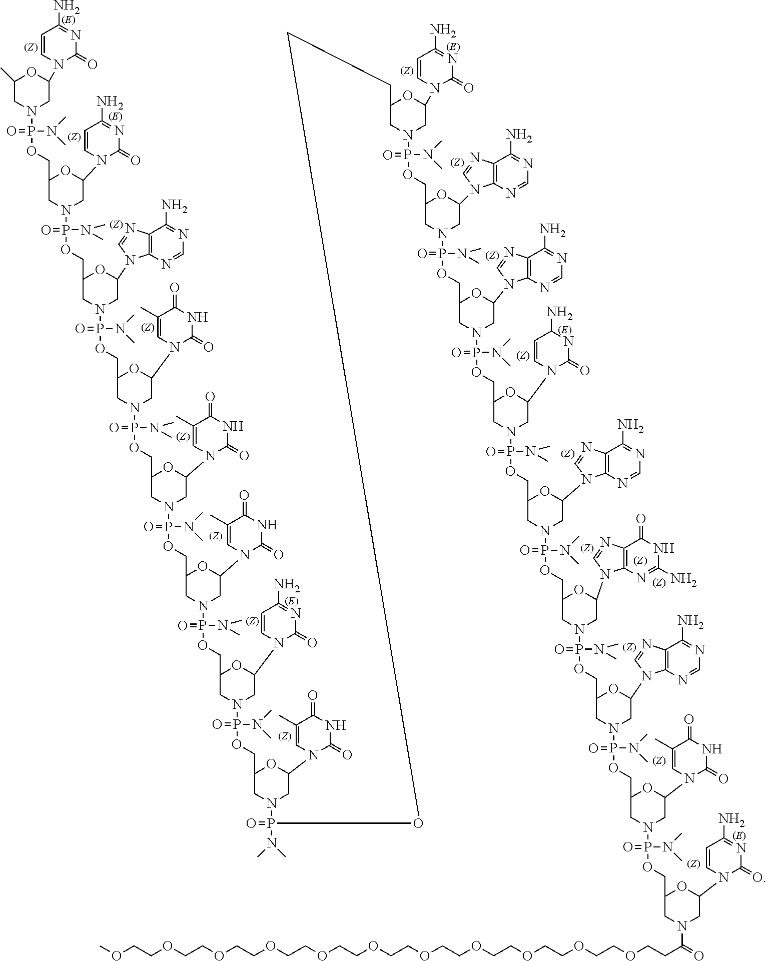

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

The relative cytosolic delivery efficiency of the cCPPs can be in the range of from about 50% to about 450% compared to cyclo(FfΦRrRrQ, SEQ ID NO:150), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 4 10%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween.

The relative cytosolic delivery efficiency of the cCPPs can be improved by greater than about 600% compared to a cyclic peptide comprising cyclo(FfΦRrRrQ, SEQ ID NO:150).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Detectable Moiety

In embodiments, the compound disclosed herein includes a detectable moiety. In embodiments, the detectible moiety is attached to the cell penetrating peptide at the amino group, the carboxylate group, or the side chain of any of the amino acids of the cell penetrating peptide moiety (e.g., at the amino group, the carboxylate group, or the side chain of any amino acid in the CPP). In embodiments, the therapeutic moiety includes a detectable moiety. The detectable moiety can include any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. In embodiments, the label is detectable without the addition of further reagents.

In embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetraphenyl metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (II) tris(4,7-diphenyl-1,10-phenanthroline) (Ru (dpp)3); Ru (II) tris(1,10-phenanthroline) (Ru(phen)3), tris(2,2′-bipyridine)rutheniurn (II) chloride hexahydrate (Ru(bpy)3); erythrosine B; fluorescein; fluorescein isothiocyanate (FITC); eosin; iridium (III) ((N-methyl-benzimidazol-2-yl)-7-(diethylamino)-coumarin));178enzothiazole) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluor‘sc’in; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can include Rhodamine B (Rho), fluorescein isothiocyanate (FITC), 7-amino-4-methylcourmarin (Amc), green fluorescent protein (GFP), or derivatives or combinations thereof.

### Methods of Making

The compounds described herein can be prepared in a variety of ways known to one skilled in the art of organic synthesis or variations thereon as appreciated by those skilled in the art. The compounds described herein can be prepared from readily available starting materials.

Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed.

Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, a,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine.

Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

The above polymers, such as PEG groups, can be attached to an oligonucleotide, such as an AC, under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the AC (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid).

If attached to the AC by reductive alkylation, the polymer selected should have a single reactive aldehyde so that the degree of polymerization is controlled. See, for example, Kinstler et al., Adv. Drug. Delivery Rev. (2002), 54: 477-485; Roberts et al., Adv. Drug Delivery Rev. (2002), 54: 459-476; and Zalipsky et al., Adv. Drug Delivery Rev. (1995), 16: 157-182.

In order to direct covalently link the AC or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making AC and conjugating AC to linear CPP are generally described in US Pub. No. 2018/0298383, which is herein incorporated by reference for all purposes. The methods may be applied to the cyclic CPPs disclosed herein.

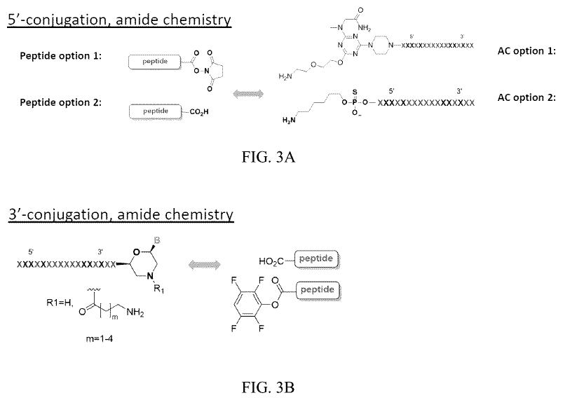

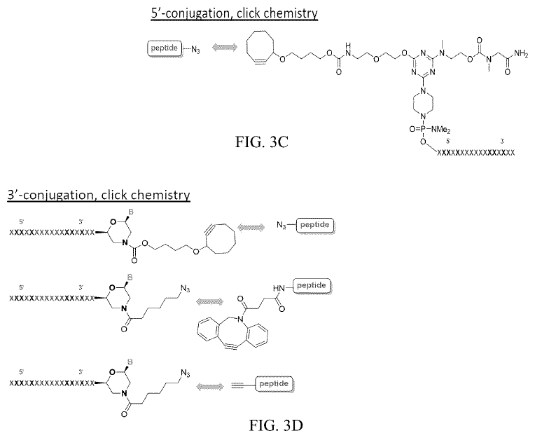

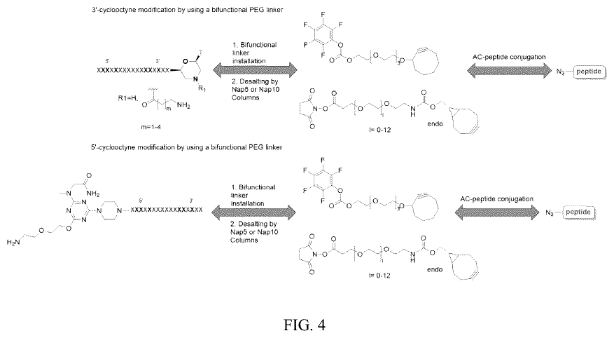

Synthetic schemes are provided in FIG. 3A-3D and FIG. 4.

Non-limiting examples of compounds that include a CPPs and a reactive group useful for conjugation to an AC are shown in Table 9. Example linker groups are also shown. Example reactive groups include tetrafluorophenyl ester (TFP), free carboxylic acid (COOH), and azide (N₃). In Table 9, n is an integer from 0 to 20; Pipa6 is AcRXRRBRRXRYQFLIRXRBRXRB (SEQ ID NO: 620) wherein B is P-Alanine and X is aminohexanoic acid; Dap is 2,3-diaminopropionic acid; NLS is a nuclear localization sequence; PA is beta alanine; -ss-is a disulfide; PABC is poly(A) binding protein C-terminal domain; Cx where x is a number is an alkyl chain of length x; and BCN is bicyclo[6.1.0]nonyne.

TABLE 9
Compounds that include a CPPs and a reactive group

TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N₃)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)-PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N₃)
CPP-PEGₙ-Cys-prodisulfide-K(N₃)
CPP-PEGₙ-K(N₃)
CPP-K(CPP)-PEGₙ-K(N₃)
CPP-PEGₙ-K(CPP)-PEGₙ-TFP
CPP-C₆-TFP
CPP-PEGₙ-K(PEGₙ-CPP)PEGₙ-K(N₃)
Ac-T9-PEGₙ-Lys(CPP-PEGₙ)-K(N₃)
Ac-MSP-PEGₙ-K(CPP-PEGₙ)-K(N₃)
CPP-PEGₙ-TFP (ENTRD 802)
CPP-C₆-TFP (ENTRD 696)
CPP-PEGₙ-K(CPP)-PEGₙ-TFP (ENTRD-344)
CPP-PEGₙ-COOH
CPP-C₁₂-TFP (ENTD-695)
palmitoyl-PEGₙ-K(CPP)-PEGₙ-TFP (ENTD-343)
CPP-PEGₙ-K(N₃) (ENTRD-617)
Ac-T9-PEGₙ-K(CPP)-K(N₃) (ENTRD 673)
Ac-MSP-PEGₙ-K(CPP-PEGₙ)-K(N₃) (ENTRD 675)
Ac-NLS-K(CPP)-PEGₙ-K(N₃) (ENTRD 684)
K(N3)-PEGₙ-NLS-ss-PEGₙ-CPP (ETRD-681)
K(N3)-PEGₙ-NLS-K-βA-βA-CPP (ETRD-682)

In embodiments, the CPPs have free carboxylic acid groups that may be utilized for conjugation to an AC. In embodiments, the EEVs have free carboxylic acid groups that may be utilized for conjugation to an AC.

The structure below is a 3′ cyclooctyne modified PMO used for a click reaction with a

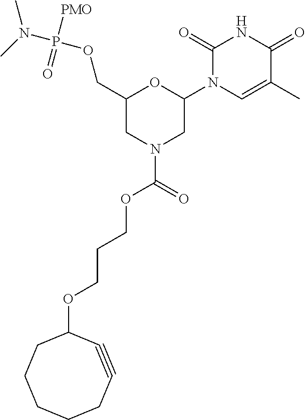

compound that includes an azide:

An example scheme of conjugation of a CPP and linker to the 3′ end of an AC via an amide bond is shown below.

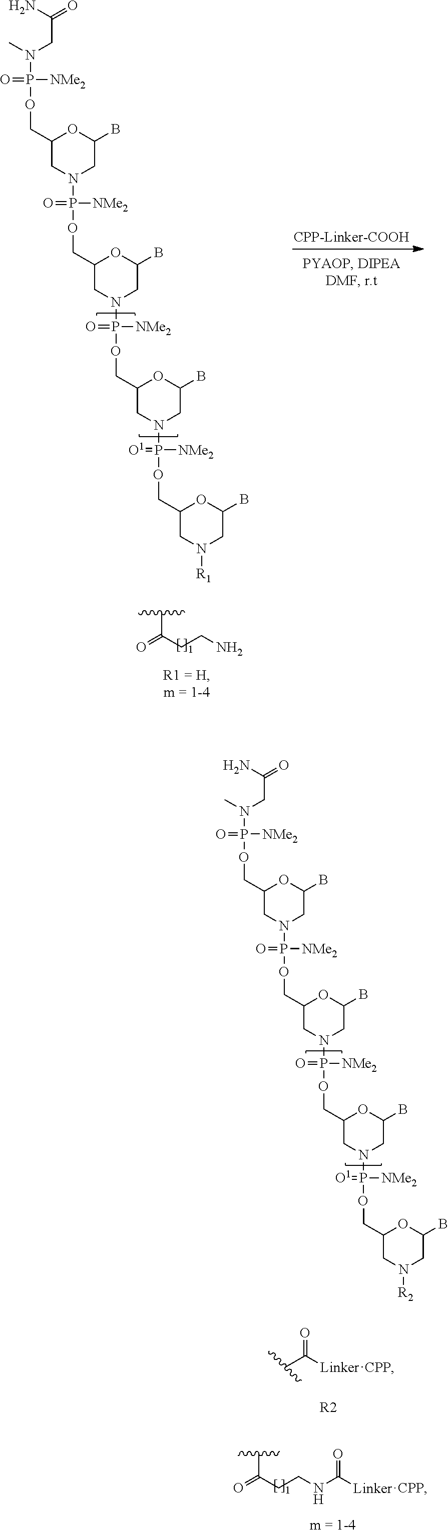

An example scheme of conjugation of a CPP and linker to a 3′-cyclooctyne modified PMO via strain-promoted azide-alkyne cycloaddition is shown below:

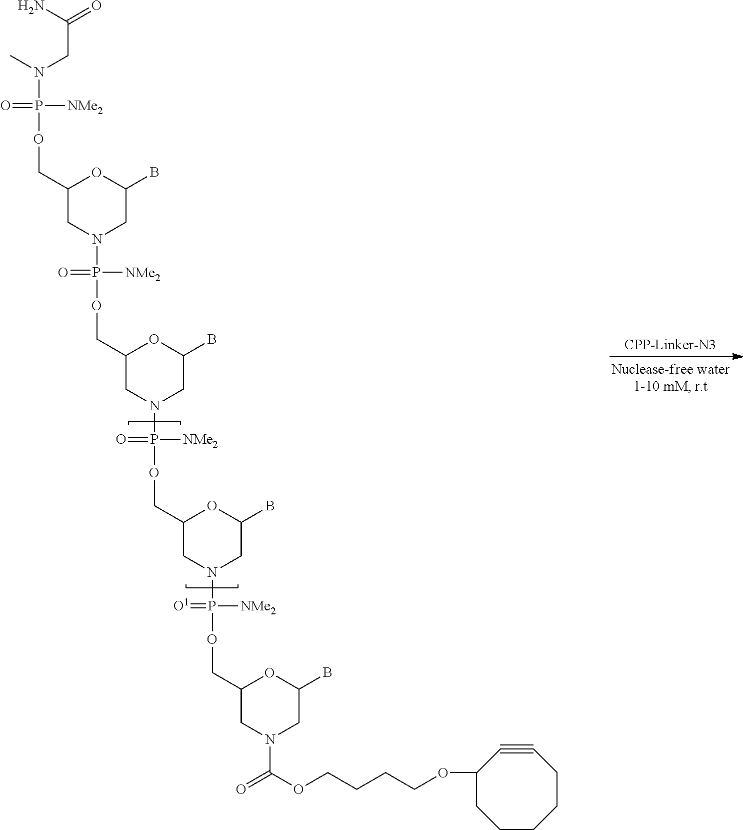

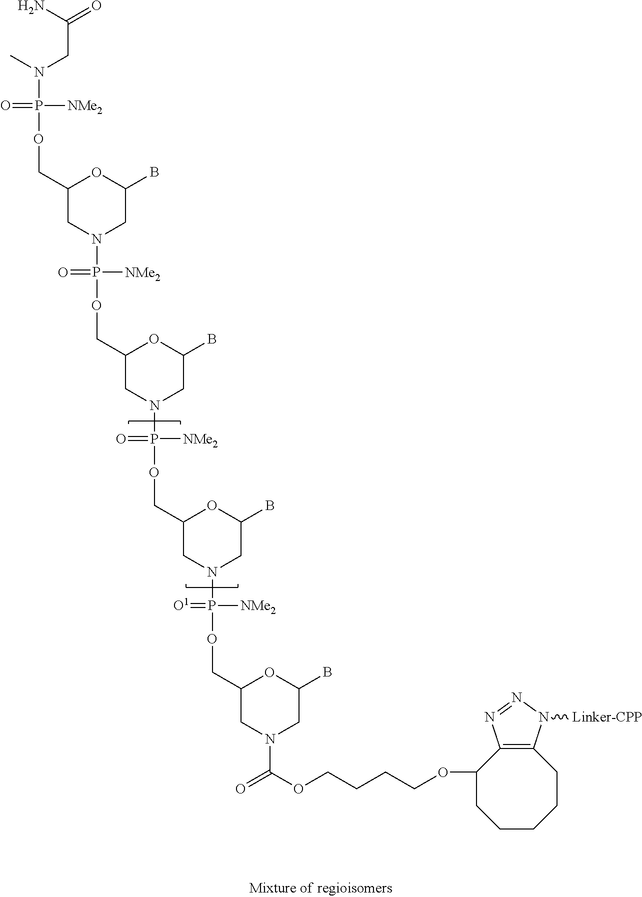

An example of the conjugation chemistry used to connect an AC and CPP with an additional linker containing a polyethylene glycol moiety is shown below:

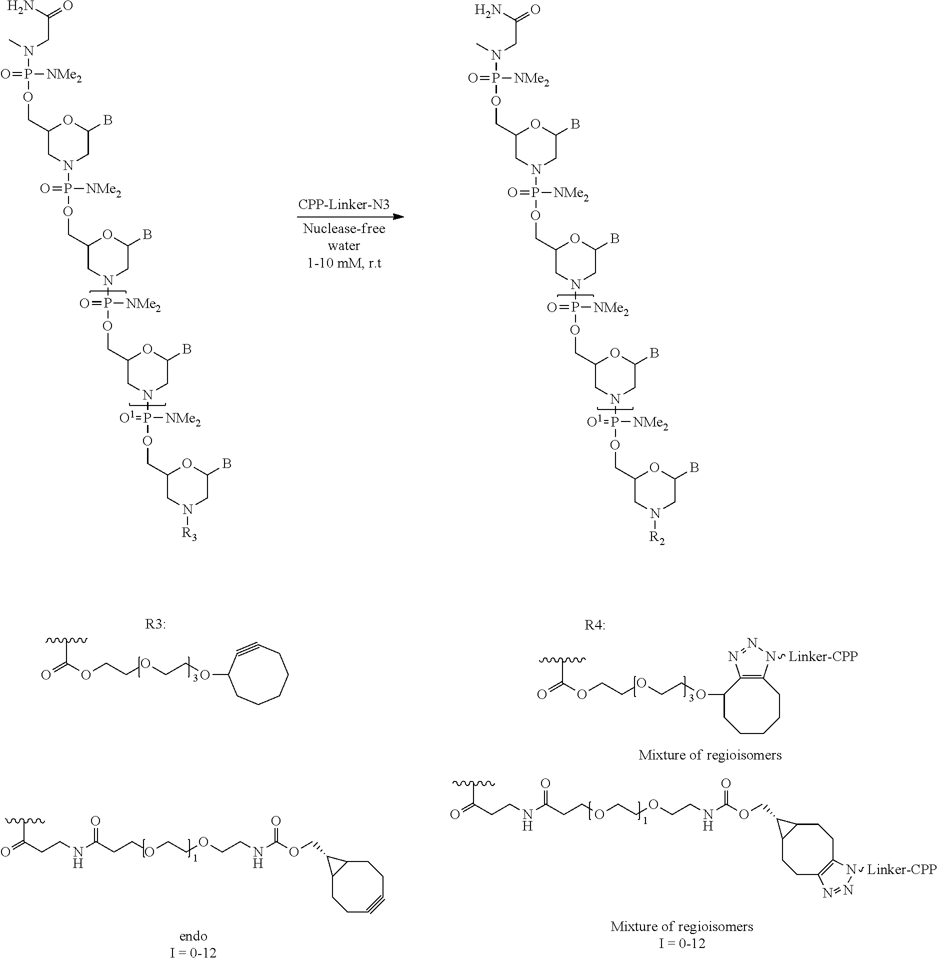

An example of conjugation of a CPP-linker to a 5′-cyclooctyne modified PMO via strain-promoted azide-alkyne cycloaddition (click chemistry) is shown below:

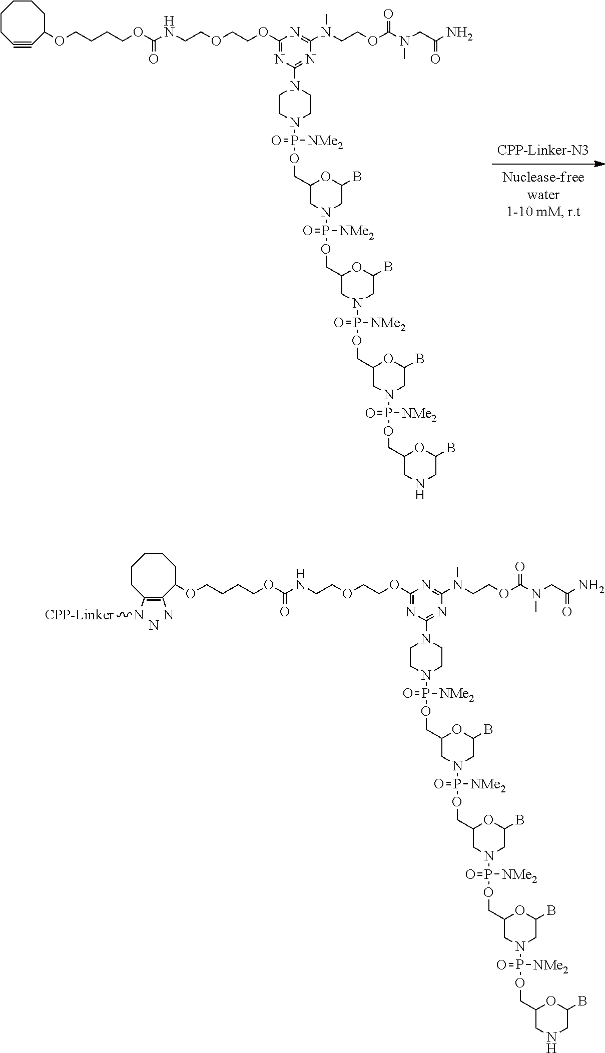

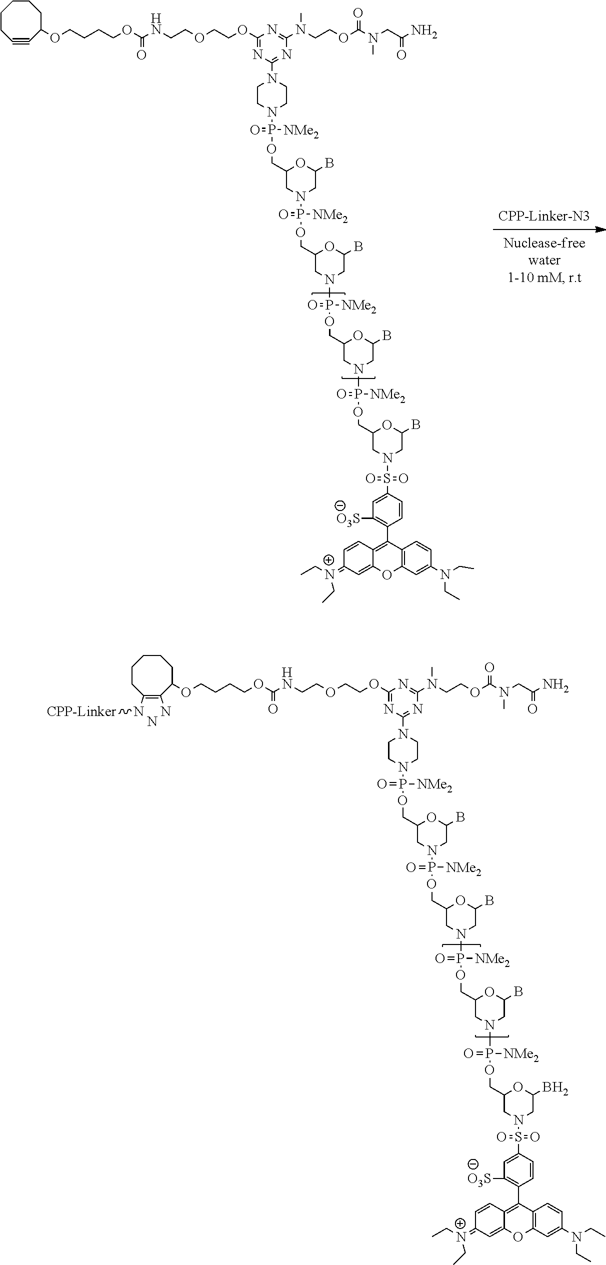

Methods of synthesizing oligomeric antisense compounds are known in the art. The present disclosure is not limited by the method of synthesizing the AC. In embodiments, provided herein are compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds are well known to those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of oligonucleotide purification and analysis are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

In the compounds disclosed herein, the AC is coupled to the CPP (e.g., cyclic peptide). As used herein, “coupled” can refer to a covalent or non-covalent association between the CPP to the AC, including fusion of the CPP to the AC and chemical conjugation of the CPP (e.g., cyclic peptide) to the AC. A non-limiting example of a means to non-covalently attach the CPP to the AC is through the streptavidin/biotin interaction, e.g., by conjugating biotin to CPP and fusing AC to streptavidin. In the resulting compound, the CPP is coupled to the AC via non-covalent association between biotin and streptavidin.

In embodiments, the CPP (e.g., cyclic peptide) is conjugated, directly or indirectly, to the AC to thereby form a CPP-AC conjugate. Conjugation of the AC to the CPP may occur at any appropriate site on these moieties. For example, In embodiments, the 5′ or the 3′ end of the AC may be conjugated to the C-terminus, the N-terminus, or a side chain of an amino acid in the CPP.

In embodiments, the AC is covalently linked to the CPP (e.g., cyclic peptide). Covalent linkage, as used herein, refer to constructs where a CPP moiety is covalently linked to the 5′ and/or 3′ end of the AC moiety. Such conjugates may alternatively be described as having a CPP moiety (e.g., cyclic peptide moiety) and an oligonucleotide moiety. A covalently-linked AC-CPP or CPP-AC conjugate, in accordance with certain embodiments, includes the AC component and the CPP component associated with one another by a linker described herein.

In embodiments, the AC may be conjugated to the CPP (e.g. cyclic peptide) through a side chain of an amino acid on the CPP. Any amino acid side chain on the CPP which is capable of forming a covalent bond, or which may be so modified, can be used to link AC to the CPP. The amino acid on the CPP can be a natural or non-natural amino acid. In embodiments, the amino acid on the CPP used to conjugate the AC is aspartic acid, glutamic acid, glutamine, asparagine, lysine, ornithine, 2,3-diaminopropionic acid, or analogs thereof, wherein the side chain is substituted with a bond to the AC or linker. In embodiments, the amino acid is lysine, or an analog thereof. In embodiments, the amino acid is glutamic acid, or an analog thereof. In embodiments, the amino acid is aspartic acid, or an analog thereof.

In embodiments, the CPP is cyclic. There are numerous possible configurations for the compounds disclosed herein. In embodiments, the compounds of the disclosure include compounds wherein AC is conjugated to the side chain of an amino acid in the cyclic peptide. In embodiments, the compounds disclosed herein have a structure (i.e., exocyclic) according to Formula I-A:

CPP-L-AC (I-A)

wherein the linker is covalently bound to the side chain of an amino acid on the CPP and to the 5′ end of the AC, the backbone of the AC, or the 3′ end of the AC.

### Diseases and Target Genes

In embodiments, compounds and methods are provided for treating a disease or disorder associated with one or more genes having an expanded nucleotide repeats (e.g., expanded trinucleotide repeats such as expanded trinucleotide repeats). In embodiments, compounds and methods are provided for treating a disease or disorder associated with one or more genes having an expanded CTG·CUG trinucleotide repeat. In embodiments, compounds and methods are provided for treating a disease or disorder associated with one or more genes having an expanded CTG-CUG trinucleotide repeat in the 3′-UTR of the gene. In embodiments, compounds and methods are provided for treating a disease or disorder associated with a gene that has an expanded CTG-CUG in the 3′ UTR such as DMPK, ATXN8OS ATXN8, and/or JPH3. In embodiments, compounds and methods are provided for treating a disease or disorder associated with one or more genes having an expanded CTG-CUG trinucleotide repeat in the intron of a gene. In embodiments, compounds and methods are provided for treating a disease or disorder associated with an expanded CTG-CUG trinucleotide repeat in an intron of TCF4. In embodiments, compounds and methods are provided for treating myotonic dystrophy type 1 (DM1), Fuchs' Endothelial Corneal Dystrophy (FECD), Spinocerebellar Ataxia-8 (SCA8), and/or Huntington's Disease-Like (HDL2).

Myotonic dystrophy type 1 (DM1)

In embodiments, compounds, compositions, and methods are provided to treat Myotonic dystrophy (DM1 or Steinert's disease). DM1 is a multisystemic disorder often characterized by muscle degeneration and myotonia or delayed muscle relaxation due to repetitive action potentials in myofibers. Myotonic dystrophy type 1 (DM1) is the most common form of muscular dystrophy, affecting about 1 in 8000 people. DM1 is a paradigm for genetic disorders caused by CTG-CUG expansions. DM1 is a neuromuscular disorder caused by a CTG-CUG repeat expansion in the 3′-untranslated region (UTR) of the dystrophia myotonia protein kinase (DMPK) gene. At the RNA level, the DMPK transcript (e.g., the expanded CUG repeat) sequesters splicing regulator proteins, for example, muscleblind-like (MBNL) protein, which results in incorrect splicing of a number of downstream pre-mRNAs (pre-mRNAs that do not contain an expanded CUG repeat) that are regulated by MBNL1. This gain-of-function is the cause of DM1.

The excessive number of CUG repeats impart toxic activity, referred to as a toxic gain-of-function. Multiple key proteins are misprocessed, and this contributes to the multisystemic nature of the disease, which includes generalized limb weakness, respiratory muscle impairment, cardiac abnormalities, fatigue, gastrointestinal complications, cataracts, incontinence, and excessive daytime sleepiness.

DM1 patients with CTG-CUG expansions within the 3′-untranslated region of DMPK gene are at increased risk for FECD and form CUGexp-MBNL1 foci in corneal endothelium. (Mootha et al., Investigative ophthalmology &visual science, 2017; 58, 4579-4585). Association of MBNL1 with mutant RNA affects the cellular pool of free MBNL1 and triggers mis-splicing of some MBNL1 target genes (e.g., regulated by MBNL 1) in affected brain, muscle, and heart tissues (Jiang et al. Hum Mol Genet. 2004; 13: 3079-3088). Gattey et al. (Cornea. 2014; 33: 96-98) reported FECD in four DM1 subjects including a mother-daughter pair. Thus, the association between DM1 and FECD is likely to be present (FECD is described in more detail elsewhere herein).

Without being bound by theory, there are at least two hypotheses proposed to explain the pathogenesis of DM1. One is that the expanded CTG-CUG repeats inhibit DMPK mRNA or protein production, resulting in DMPK haploinsufficiency. This was supported by studies demonstrating decreased expression of DMPK mRNA and protein in DM1 muscle (Fu, Y. H. et al. (1993) Decreased expression of myotonin-protein kinase messenger RNA and protein in adult form of myotonic dystrophy. Science 260, 235-238). In embodiments, the compounds and methods described herein ameliorate DMPK haploinsufficiency. Another RNA gain-of-function hypothesis proposes that the mutant RNA transcribed from the expanded allele is sufficient to induce symptoms of the disease. This was suggested by observations: (i) the expanded CTG repeats are transcribed into CUG repeats that accumulate in discrete nuclear foci, (ii) expression of only the DMPK 3′-UTR with 200 CTG repeats is sufficient to inhibit myogenesis (Davis, B. M., et al. (1997) Expansion of a CUG trinucleotide repeat in the 31 untranslated region of myotonic dystrophy protein kinase transcripts results in nuclear retention of transcripts. Proc. Natl. Acad. Sci. U.S.A. 94, 7388-7393; Amack, J. D. et al., (1999) Cis and trans effects of the myotonic dystrophy (DM) mutation in a cell culture model. Hum. Mol. Genet. 8, 1975-1984). In embodiments, the compounds and methods described herein reduce transcription of mutant RNA which are associated with the expanded allele.

The expanded CTG-CUG trinucleotide repeats in the 3′ untranslated region of DMPK mRNA form imperfect stable hairpin structures that accumulate in the cell nucleus in small ribonuclear complexes or microscopically visible inclusions, and impair the function of proteins implicated in transcription, splicing or RNA export. Although DMPK genes with CUG repeats are transcribed into mRNA, the mutant transcripts are sequestered in the nucleus as aggregates (foci), which results in a decrease in cytoplasmic DMPK mRNA levels. These aggregations lead to the deregulation of the alternative splicing of many different transcripts due to sequestration of two RNA-binding proteins: MBNL1 (muscleblind-like 1) and CUGBP1 (CUG-binding protein 1), resulting in loss-of-function of MBNL1 and upregulation of CUGBP1 (Lee and Cooper. (2009) “Pathogenic mechanisms of myotonic dystrophy,” Biochem Soc Trans. 37(06): 1281-1286).

In DM1, the RNA-binding protein MBNL1, is sequestered to the double-stranded hairpin structure formed by CUG repeats, depleting it from the nucleoplasm. Then, the CUG repeats to which MBNL1 bound stimulate Protein Kinase C (PKC) activation through an unknown mechanism, which induces CUGBP1 hyperphosphorylation and stabilization. The downstream effects include disruption of alternative splicing, mRNA translation and mRNA decay of downstream genes. An important molecular feature of DM1 is the misregulation of alternative splicing due to sequestration of MBNL1 to CUG repeats with double-stranded hairpin structure. Among more than two dozen splicing events mis-regulated in DM1, the abnormal splicing of the skeletal muscle-specific ClC-1 (chloride channel 1) is known to be one of the causes for myotonia. Increased inclusion of exons containing premature stop codons result in down-regulation of ClC-1 mRNA and protein, which is sufficient to cause myotonia (Charlet-B et al. (2002) Loss of the muscle-specific chloride channel in type 1 myotonic dystrophy due to misregulated alternative splicing. Mol. Cell 10, 45-53; Mankodi, A.et al. (2002) Expanded CUG repeats trigger aberrant splicing of ClC-1 chloride channel pre-mRNA and hyperexcitability of skeletal muscle in myotonic dystrophy. Mol. Cell 10, 35-44). In embodiments, the compounds and methods described herein ameliorate the downstream effects, including disruption of alternative splicing, mRNA translation, and mRNA decay of downstream genes. In embodiments, the compounds and methods described herein reduce the number of splicing events mis-regulated in DM1 compared to a subject with DM1 that is not treated with compounds or methods of the disclosure. For example, in some embodiments, the compounds and methods described herein may reduce the number of mis-regulated splicing events in one or more downstream genes such as 4833439L19Rik, Abcc9, Atp2al, Arhgefl0, Arhgap28, Armcx6, Angell, Best3, Bin1, Brd2, Cacnals, Cacna2dl, Cpd, Cpeb3, Ccpgl, Clasp1, Clcn1, Clk4, Cpeb2, Camk2g, Capzb, Copz2, Coch, cTNT, Ctu2, Cyp2sl, Dctn4, Dnmll, Eya4, Efna3, Efna2, Fbxo31, Fbxo21, Frem2, Fgd4, Fucal, Fnl, Gogla4, Gpr3711, Greb1, Hegl, Insr, Impdh2, IR, Itgav, Jag2, Klcl, Kcan6, Kifl3a, Ldb3, Lrrfip2, Mapt, Macfl, Map3k4, Mapkapl, Mbn11, Mllt3, Mbn12, Mef2c, Mpdz, Mrpll, Mxra7, Mybpcl, Myo9a, Ncapd3, Ngfr, Ndrg3, Ndufv3, Neb, Nfix, Numal, Opal, Pacsin2, Pcolce, Pdlim3, Pla2gl5, Phactr4, Phkal, Phtf2, Ppplrl2b, Ppp3cc, Ppplcc, Ramp2, Rapgefl, Rurn, Ryrl, Sorcs2, Spsb4, Scube2, Sema6c, Sfc8a3, Slain2, Sorbsl, Spag9, Tmem28, Tacc1, Tacc2, Ttc7, Tnik, Tnfrsf22, Tnfrsf25, Trappc9, Trim55, Ttn, Txnl4a, Txlnb, Ube2d3, or Vsp39.

In embodiments, the compounds and methods described herein reduce the number of exons containing premature stop codons which result in down-regulation of ClC-1 mRNA compared to a subject with DM1 that is not treated with compounds or methods of the disclosure.

The levels of MBNL1 and CUGBP1 in the nucleus control a subset of developmentally regulated splicing events that are reversed in DM1. In the embryonic stage, MBNL1 nuclear levels are low and CUGBP1 levels are high. During development, MBNL1 nuclear levels increase while CUGBP1 levels decrease, inducing an embryonic-to-adult transition of downstream splice targets (including IR exon 11, ClC-1 exons containing stop codons and cTNT exon 5). However, in DM1, MBNL1 is sequestered to CUG repeats, resulting in a decrease of functional MBNL1, while CUGBP1 levels are increased due to phosphorylation and stabilization. This simulates the embryonic condition and enhances expression of embryonic isoforms in adults, resulting in multiple disease symptoms (Lee and Cooper; 2009). In embodiments, the compounds and methods described herein reduce amount of MBNL1 sequestered, increase the amount of functional MBNL1, decrease CUGBP1 levels compared to a subject with DM1 that is not treated with compounds or methods of the disclosure.

MBNL1 and CELF1 (also referred to as “CUGBP1”) are developmental regulators of splicing events during fetal to adult transition and modification of their activities in DM1 leads to expression of a fetal splicing pattern in adult tissues. The downstream impact of low MBNL1 and high CELF1 includes disruption of alternative splicing, mRNA translation and mRNA decay in proteins such as cardiac troponin T (cTNT), insulin receptor (INSR), muscle-specific chloride ion channel (CLCN1) and sarcoplasmic/endoplasmic reticulum calcium ATPase 1 (ATP2A1) transcripts, in addition to MBNL1. Konieczny et al. (2017) “Myotonic dystrophy: candidate small molecule therapeutics,” Drug Discovery Today. 22(11):1740-1748.

Compounds and methods for treating myotonic dystrophy using antisense oligomers targeting polyCUG repeats in the 3′-UTR of DMPK gene are described in U.S. Ser. No. 10/106,796B2, U.S. Ser. No. 10/111,962B2, US20150080311A1, each of which is herein incorporated by reference in its entirety for all purposes. However, such PMOs or PPMOs targeting CUG repeats to treat DM1 might have limitations in oligo delivery to muscles, which is the disease affected tissues.

In embodiments, the present disclosure teaches use of diverse cell penetrating peptide (CPP) to deliver the AC (e.g., PMO or ASO) and a degradation sequence described herein e.g., in Tables 2 and 10, to the cytosol of the cell. In embodiments, the CPP or EEV conjugated with the AC delivers the AC of interest to the cellular location where the target sequence on pre-mRNA is located.

In embodiments, the disease is a form of myotonic dystrophy (e.g., myotonic dystrophy type 1 or myotonic dystrophy type 2). In embodiments, the target gene is the DMIPK gene, which encodes myotonic-protein kinase. In embodiments, the compounds provided herein comprise an AC (e.g., ASO) that targets DMPK (e.g., the 3′-untranslated region/polyadenylation of DMPK gene) to degrade DMPK gene. Exemplary oligonucleotides that target DMPK for degradation are provided in Table 10. The degradation sequence may be used in combination with an AC sequence comprising from 10-40 CAG repeats (SEQ TD NO: 414), including but not limited to the AC provided in Table 2.

TABLE 10
Oligonucleotides (AC) targeting
DMPK for degradation.

| Oligo | | SEQ | |
|---|---|---|---|
| (AC) | | ID | |
| ID | Sequence (5′-3′) | NO: | Target |
| | 5′-CAG CAG CAG CAG CAG | | DMPK |
| | CAG CAG-3′-click-K- | | |
| | PEG12-Lys(CPP12)-NLS-AC | | |
| | (all PMO monomers) | | |
| DMPK-A-17 | GGGCCTTTTATTCGCGAGGGTCGGG | 373 | DMPK |
| DMPK-A-18 | GAGGGCCTTTTATTCGCGAGGGTCG | 374 | DMPK |
| DMPK-A-19 | TGGAGGGCCTTTTATTCGCGAGGGT | 375 | DMPK |
| DMPK-A-20 | GATGGAGGGCCTTTTATTCGCGAGG | 376 | DMPK |
| DMPK-A-21 | CAGATGGAGGGCCTTTTATTCGCGA | 377 | DMPK |
| DMPK-A-22 | GGCAGATGGAGGGCCTTTTATTCGC | 378 | DMPK |
| DMPK-A-23 | TGGGCAGATGGAGGGCCTTTTATTC | 379 | DMPK |
| DMPK-A-24 | TTTGGGCAGATGGAGGGCCTTTTAT | 380 | DMPK |
| DMPK-A-25 | GCTTTGGGCAGATGGAGGGCCTTTT | 381 | DMPK |
| DMPK-A-26 | GAGCTTTGGGCAGATGGAGGGCCTT | 382 | DMPK |
| DMPK-A-27 | CAGAGCTTTGGGCAGATGGAGGGCC | 383 | DMPK |
| DMPK-A-28 | TCCAGAGCTTTGGGCAGATGGAGGG | 384 | DMPK |
| DMPK-A-29 | AGTCCAGAGCTTTGGGCAGATGGAG | 385 | DMPK |
| DMPK-A-30 | GGAGTCCAGAGCTTTGGGCAGATGG | 386 | DMPK |
| DMPK-A-31 | GTGGAGTCCAGAGCTTTGGGCAGAT | 387 | DMPK |
| DMPK-A-32 | CTGTGGAGTCCAGAGCTTTGGGCAG | 388 | DMPK |
| DMPK-A-33 | CACTGTGGAGTCCAGAGCTTTGGGC | 389 | DMPK |
| DMPK-A-34 | GACACTGTGGAGTCCAGAGCTTTGG | 390 | DMPK |
| DMPK-A-35 | CGGACACTGTGGAGTCCAGAGCTTT | 391 | DMPK |
| DMPK-A-36 | CGCGGACACTGTGGAGTCCAGAGCT | 392 | DMPK |
| DMPK-A-37 | ACCGCGGACACTGTGGAGTCCAGAG | 393 | DMPK |
| DMPK-A-38 | AAACCGCGGACACTGTGGAGTCCAG | 394 | DMPK |
| DMPK-A-39 | GCAAACCGCGGACACTGTGGAGTCC | 395 | DMPK |
| DMPK-A-40 | ACGCAAACCGCGGACACTGTGGAGT | 396 | DMPK |
| DMPK-A-41 | CAACGCAAACCGCGGACACTGTGGA | 397 | DMPK |

### Spinocerebellar Ataxia-8 (SCA8)

In embodiments, compounds, compositions, and methods are provided to treat Spinocerebellar Ataxia-8 (SCA8). SCA8 is an inherited neurodegenerative condition that is characterized by slowly progressing ataxia. Symptoms normally emerge during the third to fifth decades of life. Symptoms include eye movement abnormalities, sensory neuropathy, dysphagia, cerebellar ataxia, and cognitive impairment.

SCA8 is associated with heterozygous abnormal expanded CTG-CUG repeat in the 3′ UTR of two overlapping genes ATXN8OS and ATXN8. Healthy individuals generally have between 15 and 50 CTG-CUG repeats in the ATXN8OS and ATXN8 genes. Patients with SCA8 have greater than 50 CTG-CUG repeats, sometimes as many as 240 CTG-CUG repeats in the ATXN8OS and ATXN8 genes.

Huntington's disease like-2 (HDL2)

In embodiments, compounds, compositions and methods are provided to treat Huntington's disease like-2 (HDL2) disease. HDL2 is an autosomal dominant neurodegenerative disorder that is phenotypically related to Huntington's disease. HDL2 is characterized by symptoms that include chorea, dystonia, rigidity, bradykinesia, and psychiatric symptoms such as dementia. Symptoms of HDL2 typically occur in mid-life and may lead to a premature death by about 10-15 years.

HDL2 is associated with an expanded CTG-CUG in the 3′ UTR of the junctophilin 3 (JPH3) gene (16q24.3). Healthy individuals generally have between 6 and 27 CTG-CUG repeats in the JPH3 gene. Patients with HDL2 have greater than 40 CTG-CUG repeats, sometimes as many as 60 or more CTG-CUG repeats in the JPH3 gene.

### Fuchs' Endothelial Corneal Dystrophy

In embodiments, compounds, compositions, and methods are provided to treat Fuchs' Endothelial Corneal Dystrophy (FECD). FECD (MIM 136800) is an age-related degenerative disorder of the corneal endothelium. FECD is characterized by progressive loss of corneal endothelial cells, thickening of Descement's membrane, and deposition of extracellular matrix in the form of guttae. When the number of endothelial cells becomes critically low, the cornea swells and causes loss of vision (Elhalis et al. Ocul Surf 2010; 8(4):173-184).

FECD can be inherited as an autosomal dominant trait with genetic heterogeneity. Rare heterozygous mutations in collagen, type VIII, alpha 2 gene (COL8A2, MIM 120252) can give rise to an early-onset corneal endothelial dystrophy. Other genes such as solute carrier family 4, sodium borate transporter, member 11 (SLC4A11, MIM 610206), transcription factor 8 (TCF8, MIM 189909), lipoxygenase homology domains 1 (LOXHDI, MIM 613267), and ATP/GTP binding protein-like 1 (AGBL1, MIM 615523) are collectively associated with a small fraction of adult-onset FECD cases. The genome-wide association studies of adult-onset FECD have suggested that transcription factor 4 (TCF4, MIM 602272) and more recently KN motif- and ankyrin repeat domain-containing protein 4 (KANK4, MIM 614612), laminin gamma-1 (LAMC1, MIM150290), Na⁺/K⁺ transporting ATPase, and beta-1 polypeptide (ATP1B₁, MIM 182330), with the TCF4 locus noted have a predominant effect on FECD (Mootha et al., Investigative ophthalmology &visual science, 2017; 58, 4579-4585).

Expanded trinucleotide repeats at the CTG18.1 locus in intron 2 of TCF4 are associated with FECD (Wieben et al., PLoS One. 2012; 7(11):e49083). Each copy of the expanded CTG18.1 allele of more than 40 CTG-CUG trinucleotide repeats leads to significant risk for development of FECD (Mootha et al., Invest Ophthalmol Vis Sci. 2014; 55: 33-42). RNA nuclear foci, a hallmark of toxic gain of function RNA, has been reported in neurodegenerative disorders caused by simple repeat expansions. Expanded CUG repeat RNA accumulate as nuclear foci in the corneal endothelium of FECD subjects with the CTG18.1 triplet repeat expansion while absent in control samples lacking the triplet expansion (Mootha et al., Invest Ophthalmol Vis Sci. 2015; 56(3):2003-2011). Expanded CUG repeat RNA colocalize with mRNA-splicing factor, muscleblind-like 1 (MBNL1), in nuclear foci in endothelium as a molecular hallmark. The triplet repeat expansion at the CTG18.1 locus may mediate endothelial dysfunction via aberrant gene splicing as a result of the mutant CUG RNA transcripts sequestering the MBNL1 (Du et al., J Biol Chem. 2015; 290: 5979-5990). Thus, two distinct triplet repeats converge on RNA foci and FECD, and it is likely that the foci may play a causal role for FECD.

In embodiments, compounds and methods useful in the treatment of Fuchs' Endothelial Corneal Dystrophy (FECD) that reduce expanded CUG repeat RNA with antisense oligonucleotides are described in WO2018165541A1, U.S. Ser. No. 10/760,076B2, each of which is herein incorporated by reference in its entirety for all purposes. However, such phosphorodiamidate morpholino oligomers (PMOs) or peptide-conjugated PMOs (PPMOs) targeting CUG repeats such as those described in the prior art, to treat DM1 might have limitations in oligo delivery to the target tissue (e.g. endothelial layers in cornea), which is the disease affected tissues.

In embodiments, the present disclosure teaches use of diverse cell penetrating peptide (CPP) or endosomal escape vehicle (EEV) to deliver the AC (e.g. PMO or ASO) described herein, e.g., in Table 6 to the cytosol of the cell. In embodiments, the CPP or EEV conjugated to the AC delivers the AC of interest to the cellular location where the target sequence on pre-mRNA is located.

In embodiments, the disease is Fuchs' Endothelial Corneal Dystrophy (FECD). In embodiments, the target gene is TCF4, which encodes transcription factor 4 (TCF-4), which is also known as immunoglobulin transcription factor 2 (ITF-2). In embodiments, the compounds provided herein comprise an antisense oligonucleotide that targets TCF4. Exemplary oligonucleotides that may be used to target TCF4 are provided in Table 2 and Table 11.

TABLE 11
Exemplary Oligonucleotides targeting
the expanded triplet repeat of TCF4

| Oligo | | |
|---|---|---|
| chemistry | Design | Target |
| 21-mer | 5′-CAG CAG CAG CAG CAG CAG CAG-3′ | TCF4 |
| PMO | (all PMO monomers; SEQ ID NO: 146) | (CUG)n |
| 25-mer | 5′-CAG CAG CAG CAG CAG CAG CAG CAG | TCF4 |
| PMO | C-3′ | (CUG)n |
| | (all PMO monomers: SEQ ID NO: 147) | |
| 21-mer | EEV-NLS_PMO_CAG 21 mer | TCF4 |
| EEV-NLS- | (conjugation from SEQ ID NO: 146) | (CUG)n |
| PMO | | |
| 30-mer | 5′-CAG CAG CAG CAG CAG CAG CAG CAG | TCF4 |
| PMO | CAG CAG-3′ | (CUG)n |
| | (all PMO monomers; SEQ ID NO: 148) | |
| 30-mer | 5′-AGC AGC AGC AGC AGC AGC AGC AGC | TCF4 |
| PMO | AGC AGC-3′ | (CUG)n |
| | (all PMO monomers; SEQ ID NO: 149) | |
| 30-mer | 5′-GCA GCA GCA GCA GCA GCA GCA GCA | TCF4 |
| PMO | GCA GCA-3′ | (CUG)n |
| | (all PMO monomers; SEQ ID NO: 372) | |

PMO: Phosphorodiamidate Morpholino Oligomer

Mootha et al. (2017) reported that DM1 and FECD originate from noncoding CTG expansions even though both are not identical diseases. The DMPK expansion in DM1 results in a multiorgan disease that involves various tissues in the eye including lens, retina, and corneal endothelium. In contrast, the TCF4 repeat expansion appears to affect the corneal endothelium without any clinically apparent sequela to other ocular tissues or bodily organs. Mutant expansions in DMPK and TCF4 share important similarities, such as (i) nuclear foci that contain expanded CUG repeats, (ii) association of foci with MBNL1 protein, and (iii) an ability to cause FECD. It is suggested that the triplet expansions in both DMPK and TCF4 may cause the same corneal endothelial tissue phenotype of FECD through shared molecular mechanisms.

See U.S. patent Ser. No. 10/760,076B2, International Application Publication No.

WO2018165541A1, U.S. Pat. Appl. Publ. No. 2016/0355796 and U.S. Pat. Appl. Publ. No. 2018/0344817, each of which is incorporated by reference herein, and which discloses diseases and corresponding genes prone to forming and/or expanding tandem nucleotide repeats.

### Compositions and Methods of Administration

The compounds of the present disclosure may be formulated into compositions suitable for in vivo applications. The compounds and/or compositions may be administered to a patient that has, or is suspected of having, a disease associated with an expanded trinucleotide repeat.

In vivo application of the disclosed compounds, and compositions containing them, can be accomplished by any suitable method and technique presently or prospectively known to those skilled in the art. For example, the disclosed compounds can be formulated in a physiologically—or pharmaceutically-acceptable composition and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated into pharmaceutical compositions according to known methods for preparing pharmaceutically acceptable compositions.

Formulations are described in detail in a number of sources which are well known and readily available to those skilled in the art. For example, Remington's Pharmaceutical Science by E.W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions also include conventional pharmaceutically acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery.

Compounds can also be incorporated into polymers, examples of which include poly (D-L lactide-co-glycolide) polymer for intracranial tumors; poly[bis(p-carboxyphenoxy) propane:sebacic acid]in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, isotonic agents may be included, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, methods of preparation include vacuum drying and the freeze-drying techniques, which yield a powder of the active ingredient plus any additional desired ingredient present in the previously sterile-filtered solutions.

Useful dosages of the compounds and agents and pharmaceutical compositions disclosed herein can be determined by comparing their in vitro activity, and in vivo activity in animal models.

Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound are disclosed herein. The dose administered to a patient, particularly a human, should be sufficient to achieve a therapeutic response in the patient over a reasonable time frame, without lethal toxicity, and causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein and/or pharmaceutical compositions containing the same, in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form.

In another embodiment, a compound and/or agent disclosed herein is provided in the kit as a liquid or solution. In one embodiment, the kit comprises an ampoule or syringe containing a compound and/or agent disclosed herein in liquid or solution form.

In embodiments, the compound and or composition of the of the disclosure is administered to a patient diagnosed with a disease associated with a nucleotide repeat expansion at a dose of between about 0.1 mg/kg and about 1000 mg/kg, for example, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 210 mg/kg, about 220 mg/kg, about 230 mg/kg, about 240 mg/kg, about 250 mg/kg, about 260 mg/kg, about 270 mg/kg, about 280 mg/kg, about 290 mg/kg, about 300 mg/kg, about 310 mg/kg, about 320 mg/kg, about 330 mg/kg, about 340 mg/kg, about 350 mg/kg, about 360 mg/kg, about 370 mg/kg, about 380 mg/kg, about 390 mg/kg, about 400 mg/kg, about 410 mg/kg, about 420 mg/kg, about 430 mg/kg, about 440 mg/kg, about 450 mg/kg, about 460 mg/kg, about 470 mg/kg, about 480 mg/kg, about 490 mg/kg, about 500 mg/kg, about 510 mg/kg, about 520 mg/kg, about 530 mg/kg, about 540 mg/kg, about 550 mg/kg, about 560 mg/kg, about 570 mg/kg, about 580 mg/kg, about 590 mg/kg, about 600 mg/kg, about 610 mg/kg, about 620 mg/kg, about 630 mg/kg, about 640 mg/kg, about 650 mg/kg, about 660 mg/kg, about 670 mg/kg, about 680 mg/kg, about 690 mg/kg, about 700 mg/kg, about 710 mg/kg, about 720 mg/kg, about 730 mg/kg, about 740 mg/kg, about 750 mg/kg, about 760 mg/kg, about 770 mg/kg, about 780 mg/kg, about 790 mg/kg, about 800 mg/kg, about 810 mg/kg, about 820 mg/kg, about 830 mg/kg, about 840 mg/kg, about 850 mg/kg, about 860 mg/kg, about 870 mg/kg, about 880 mg/kg, about 890 mg/kg, about 900 mg/kg, about 910 mg/kg, about 920 mg/kg, about 930 mg/kg, about 940 mg/kg, about 950 mg/kg, about 960 mg/kg, about 970 mg/kg, about 980 mg/kg, about 990 mg/kg, or about 1000 mg/kg, including all values and ranges therein and in between.

### Methods of Treatment

The present disclosure provides a method of treating disease in a subject in need thereof, comprising administering a compound and/or composition containing the compound disclosed herein. In embodiments, the disease is any of the diseases provided in the present disclosure. In embodiments, the target gene or gene transcript is any of the target genes or gene transcripts provided in the present disclosure.

In embodiments, the patient is identified as having, or at risk of having, any disease as described herein. In embodiments, a method is provided for treating a disease associated with a CTGCUG repeat in a 3′ untranslated region of a gene/transcript. In embodiments, a method is provided for treating myotonic dystrophy. In embodiments, a method is provided for treating myotonic dystrophy type 1 (DM1). In embodiments, a method is provided for treating SCA8. In embodiments, a method is provided for treating HDL2. In embodiments, a method is provided for treating FECD.

In embodiments, treatment refers to partial or complete alleviation, amelioration, relief, inhibition, delaying onset, reducing severity and/or incidence of one or more symptoms in a subject.

Treatment of the disease and/or symptoms of the disease may occur through a variety of molecular mechanisms such as those described herein.

In embodiments, a method is provided for altering the expression and/or activity of a target gene in a subject in need thereof, comprising administering a compound disclosed herein. In embodiments, the treatment results in the lowered expression of a target protein from a target transcript. In embodiments, treatment results in the lowered levels of a target transcript. In embodiments, treatment results in the modulation of splicing of downstream gene transcripts that are regulated by the target transcript and/or proteins that bind to the target transcript. In embodiments, modulation of splicing of downstream gene transcripts results in an increase in downstream transcripts and/or downstream proteins isoforms that are associated with healthy phenotypes. In embodiments, the alternative splicing results in a decrease in downstream transcripts and/or downstream proteins isoforms that are associated with disease phenotypes.

In embodiments, a method is provided for treating DM1 by reducing sequestration of at least one RNA-binding protein to a pre-mRNA comprising at least one expanded CUG repeat. In embodiments, a method is provided for treating DM1 by reducing accumulation of a pre-mRNA comprising at least one expanded CUG repeat. In embodiments, a method is provided for treating DM1 by correcting splicing defects of downstream gene transcripts.

In embodiments, treatment according to the present disclosure results in a decreased level of the target transcript and/or expression of the target transcript (e.g., DMPK, TCF4, JPH3, ATXN8OS and/or ATXN8) genes by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the protein in the subject before the treatment or of one or more control individuals with similar disease without treatment, or compared to treatment with an AC not conjugated to a cyclic CPP disclosed herein. In embodiments, treatment according to the present disclosure results in a decreased level of the target transcript (e.g., DMPK, TCF4, JPH3, ATXN8OS and/or ATXN8) and/or expression of the target transcript by about 5% to about 100%, about 10% to about 100%, about 20% to about 100%, about 50% to about 100%, about 70% to about 100%, about 80% to about 100%, about 90% to about 100%, about 95% to about 100%, about 40% to about 95%, about 50% to about 95%, about 70% to about 95%, or about 90% to about 95% as compared to the average level of the transcript and/or protein in the subject before the treatment or of one or more control individuals with similar disease without treatment, or compared to treatment with an AC not conjugated to a cyclic CPP disclosed herein.

In embodiments, treatment according to the present disclosure results in a decreased number of CUG repeat RNA nuclear foci of a target gene (e.g., DMPK, TCF4, JPH3, ATXN8OS and/or ATXN8) by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level foci in the subject before the treatment or of one or more control individuals with similar disease without treatment, or compared to treatment with an AC not conjugated to a cyclic CPP disclosed herein. In embodiments, treatment according to the present disclosure results in a decreased number of CUG repeat RNA nuclear foci of a target gene (e.g., DMPK, TCF4, JPH3, ATXN8OS and/or ATXN8) by about 5% to about 100%, about 10% to about 100%, about 20% to about 100%, about 50% to about 100%, about 70% to about 100%, about 80% to about 100%, about 90% to about 100%, about 95% to about 100%, about 40% to about 95%, about 50% to about 95%, about 70% to about 95%, or about 90% to about 95% as compared to the average level of foci in the subject before the treatment or of one or more control individuals with similar disease without treatment, or compared to treatment with an AC not conjugated to a cyclic CPP disclosed herein.

In embodiments, treatment according to the present disclosure results in a decreased level of downstream transcript and/or expression of a downstream gene product that is associated with a disease phenotype by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the protein in the subject before the treatment or of one or more control individuals with similar disease without treatment, or compared to treatment with an AC not conjugated to a cyclic CPP disclosed herein. In embodiments, treatment according to the present disclosure results in a decreased level of downstream transcript and/or expression of a downstream gene product that is associated with a disease phenotype by about 5% to about 100%, about 10% to about 100%, about 20% to about 100%, about 50% to about 100%, about 70% to about 100%, about 80% to about 100%, about 90% to about 100%, about 95% to about 100%, about 40% to about 95%, about 50% to about 95%, about 70% to about 95%, or about 90% to about 95% as compared to the average level of the protein in the subject before the treatment or of one or more control individuals with similar disease without treatment, or compared to treatment with an AC not conjugated to a cyclic CPP disclosed herein.

In embodiments, treatment according to the present disclosure results in an increased level of downstream transcript and/or expression of a downstream gene product that is associated with a healthy phenotype by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the protein in the subject before the treatment or of one or more control individuals with similar disease without treatment, or compared to treatment with an AC not conjugated to a cyclic CPP disclosed herein. In embodiments, treatment according to the present disclosure results in an increased level of downstream transcript and/or expression of a downstream gene product that is associated with a healthy phenotype by about 5% to about 100%, about 10% to about 100%, about 20% to about 100%, about 50% to about 100%, about 70% to about 100%, about 80% to about 100%, about 90% to about 100%, about 95% to about 100%, about 40% to about 95%, about 50% to about 95%, about 70% to about 95%, or about 90% to about 95% as compared to the average level of the protein in the subject before the treatment or of one or more control individuals with similar disease without treatment, or compared to treatment with an AC not conjugated to a cyclic CPP disclosed herein.

In embodiments, treatment according to the present disclosure results in decreased expression of a protein isoform associated with a disease phenotype in a subject's corneal tissue, muscle tissue, diaphragm tissue, quadriceps, triceps, tibialis anterior, gastrocnemius, or heart by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the protein in the subject's corneal tissue, muscle tissue, diaphragm tissue, quadriceps, triceps, tibialis anterior, gastrocnemius, or heart before the treatment, compared to one or more control individuals with similar disease without treatment, or compared to treatment with an AC not conjugated to a cyclic CPP disclosed herein. In embodiments, treatment according to the present disclosure results in decreased expression of a protein isoform associated with a disease phenotype in a subject's corneal tissue, muscle tissue, diaphragm tissue, quadriceps, triceps, tibialis anterior, gastrocnemius, or heart by more than about by about 5% to about 100%, about 10% to about 100%, about 20% to about 100%, about 50% to about 100%, about 70% to about 100%, about 80% to about 100%, about 90% to about 100%, about 95% to about 100%, about 40% to about 95%, about 50% to about 95%, about 70% to about 95%, or about 90% to about 95% as compared to the average level of the protein in the subject's corneal tissue, muscle tissue, diaphragm tissue, quadriceps, triceps, tibialis anterior, gastrocnemius, or heart before the treatment, compared to one or more control individuals with similar disease without treatment, or compared to treatment with an AC not conjugated to a cyclic CPP disclosed herein.

In embodiments, treatment according to the present disclosure results in increased expression of an alternately spliced downstream protein in a subject's corneal tissue, muscle tissue, diaphragm tissue, quadriceps, or heart by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 300%, about 350%, about 400%, about 450%, about 500%, about 550%, about 600%, about 650%, about 700%, about 750%, about 800, about 850%, about 900%, about 950%, or about 1000% or more, as compared to the average level of the downstream protein in the subject's corneal tissue, muscle tissue, diaphragm tissue, quadriceps, or heart before the treatment, compared to one or more control individuals with similar disease without treatment, or compared to treatment with an AC not conjugated to a cyclic CPP disclosed herein.

In embodiments, treatment according to the present disclosure results in increased or decreased expression of a wild type protein isomer in a subject's corneal tissue, muscle tissue, diaphragm tissue, quadriceps, or heart by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the wild type protein isomer in the subject's corneal tissue, muscle tissue, diaphragm tissue, quadriceps, or heart before the treatment, compared to one or more control individuals with similar disease without treatment, or compared to treatment with an AC not conjugated to a cyclic CPP disclosed herein.

In embodiments, treatment according to the present disclosure results in decreased expression of a protein in a subject's tissue of interest by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the protein in the subject's tissue of interest before the treatment, compared to one or more control individuals with similar disease without treatment, or compared to treatment with an AC not conjugated to a cyclic CPP disclosed herein.

In embodiments, treatment according to the present disclosure results in increased expression of an alternately spliced downstream protein in a subject's tissue of interest by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 300%, about 350%, about 400%, about 450%, about 500%, about 550%, about 600%, about 650%, about 700%, about 750%, about 800, about 850%, about 900%, about 950%, or about 1000% or more, as compared to the average level of the downstream protein in the subject's tissue of interest before the treatment, compared to one or more control individuals with similar disease without treatment, or compared to treatment with an AC not conjugated to a cyclic CPP disclosed herein.

In embodiments, treatment according to the present disclosure results in increased or decreased expression of a wild type downstream protein isomer in a subject's tissue of interest by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the downsteam protein in the subject's tissue of interest before the treatment, compared to one or more control individuals with similar disease without treatment, or compared to treatment with an AC not conjugated to a cyclic CPP disclosed herein.

In embodiments, the subject's tissue of interest is corneal tissue or muscle tissue.

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have downstream protein expression or activity levels that are less than about 1-99% of normal wild type protein expression or activity levels in an individual not afflicted with the disease. In embodiments, the range includes, but is not limited to less than about 80-99%, less than about 65-80%, less than about 50-65%, less than about 30-50%, less than about 25-30%, less than about 20-25%, less than about 15-20%, less than about 10-15%, less than about 5-10%, less than about 1-5% of normal wild type protein expression or activity levels. In embodiments, the individual may have downstream protein expression or activity levels that are 1-500% higher than normal wild type target protein expression or activity levels in an individual not afflicted with the disease. In embodiments, the range includes, but is not limited to, greater than about 1-10%, about 10-50%, about 50-100%, about 100-200%, about 200-300%, about 300-400%, about 400-500%, or about 500-1000% of normal wild type target protein expression or activity levels.

In embodiments, the individual is an individual who has been recently diagnosed with the disease. Typically, early treatment (treatment commencing as soon as possible after diagnosis) is important to minimize the effects of the disease and to maximize the benefits of treatment.

### Certain Definitions

As used in the description and the appended claims, the singular forms “a,” “an,” and “the” include plural referents unless the context clearly dictates otherwise. Thus, for example, reference to “a composition″ includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 20%, 10%, or 5% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “cell penetrating peptide” or “CPP” refers to a peptide that facilitates delivery of a cargo, e.g., a therapeutic moiety™ into a cell. In embodiments, the CPP is cyclic, and is represented as “cCPP”. In embodiments, the cCPP is capable of directing a therapeutic moiety to penetrate the membrane of a cell. In embodiments, the cCPP delivers the therapeutic moiety to the cytosol of the cell. In embodiments, the cCPP delivers an antisense compound (AC) to a cellular location where a pre-mRNA is located.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide, or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO:42), the nucleoplasmin bipartite NLS with the sequence NLSKRPAAIKKAGQAKKKK(SEQ ID NO:52), the c-myc nuclear localization sequence having the amino acid sequence PAAKRVKLD (SEQ ID NO:53) or RQRRNELKRSF(SEQ ID NO:54), the sequence RMRKFKNKGKDTAELRRRRVEVSVELRKAKKDEQILKRRNV (SEQ ID NO: 371) of the IBB domain from importin-alpha, the sequences VSRKRPRP (SEQ ID NO:57) and PPKKARED (SEQ ID NO:58) of the myoma T protein, the sequence PQPKKKPL (SEQ ID NO:59) of human p53, the sequence SALIKKKKKMAP (SEQ ID NO:60) of mouse c-abl IV, the sequences DRLRR (SEQ ID NO:61) and PKQKKRK (SEQ ID NO:62) of the influenza virus NS1, the sequence RKLKKKIKKL (SEQ ID NO:63) of the Hepatitis virus delta antigen, the sequence REKKKFLKRR (SEQ ID NO:64) of the mouse Mxl protein, the sequence KRKGDEVDGVDEVAKKKSKK (SEQ ID NO:65) of the human poly(ADP-ribose) polymerase, and the sequence RKCLQAGMNLEARKTKK (SEQ ID NO:66) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins, and the like.

As used herein, the term “contiguous” refers to two amino acids, which are connected by a covalent bond. For example, in the context of a representative cyclic cell penetrating peptide (cCPP) such as

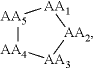

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. For example, arginine or an arginine residue refers to

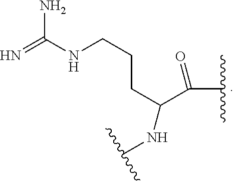 [SMILES: *NC(CCCNC(=N)N)C(*)=O]

The term “protonated form thereof” refers to a protonated form of an amino acid or side chain. For example, the guanidine group on the side chain of arginine may be protonated to form a guanidinium group. The structure of a protonated form of arginine is

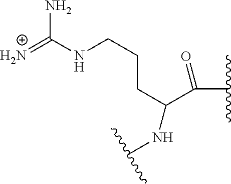 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2 7x electrons, wherein n is any integer. “Heteroaromatic,” defined below, is a subset of aromatic.

Examples of aromatic amino acids include phenylalanine and napthylalinine. The term “non-aromatic” refers to any molecule that does not fall within the definition of aromatic. For example, any linear, branched or cyclic molecule which does not fall within the definition of aromatic is non-aromatic. Examples of non-aromatic amino acids include, but are not limited to, glycine and citrulline.

“Alkyl”, “alkyl chain″ or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄o alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain″ or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls.

Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain″ or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where R^{a} and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where R^{a} and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl.

In addition, each of the foregoing substituents can also be optionally substituted with one or more of the above substituents.

As used herein, the symbol

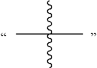

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

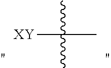

indicates that the chemical entity “XY” is bonded to another chemical entity via the point of attachment bond. Furthermore, the specific point of attachment to the non-depicted chemical entity can be specified by inference. For example, the compound CH₃-R³, wherein R³ is H or

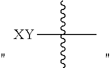

infers that when R³ is “XY”, the point of attachment bond is the same bond as the bond by which R³ is depicted as being bonded to CH₃.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The terms “inhibit”, “inhibiting” or “inhibition″ refer to a decrease in an activity, expression, function or other biological parameter and can include, but does not require complete ablation of the activity, expression, function or other biological parameter. Inhibition can include, for example, at least about a 10% reduction in the activity, response, condition, or disease as compared to a control. In embodiments, expression, activity or function of a gene or protein is decreased by a statistically significant amount. In embodiments, activity or funcion is decreased by at least about 10%, about 20%, about 30%, about 40%, about 50%, and up to about 60%, about 70%, about 80%, about 90% or about 100%.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

As used herein, “treat,” “treating,” “treatment” and variants thereof, refers to any administration of the disclosed compounds that partially or completely alleviates, ameliorates, relieves, inhibits, delays onset of, reduces severity of, and/or reduces incidence of one or more symptoms or features of a disease as described herein. In reference to a patient, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the disclosed compound and/or composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and/or animals without excessive toxicity, irritation, allergic response, or other problems or complications commensurate with a reasonable benefit/risk ratio.

The term “carrier” means a compound, composition, substance, or structure that, when in combination with a compound or composition of the present disclosure, aids or facilitates preparation, storage, administration, delivery, effectiveness, selectivity, or any other feature of the compound or composition for its intended use or purpose, or combinations thereof. For example, a carrier can be selected to minimize any degradation of the active ingredient and to minimize any adverse side effects in the subject.

As used herein, the term “pharmaceutically acceptable carrier” refers to a carrier suitable for administration to a patient. A pharmaceutical carrier may be a substance that aids or facilitates preparation, storage, administration, delivery, effectiveness, selectivity, or any other feature of the compound or composition of the present disclosure for its intended use or purpose, or combinations thereof. For example, a carrier can be selected to reduce degradation of the compound or to reduce adverse side effects in the patient. In embodiments, a pharmaceutically acceptable carrier can be a sterile aqueous or nonaqueous solutions, dispersions, suspensions or emulsions, as well as sterile powders for reconstitution into sterile injectable solutions or dispersions just prior to use.

Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid, and the like. It can also be desirable to include isotonic agents such as sugars, sodium chloride, and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts, and the like.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration″ refers to administration just below the skin. “Intravenous administration″ means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In such embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized compounds or compositions described herein. In embodiments, a dosage unit is a vial that includes reconstituted compounds or compositions described herein.

The term “therapeutic moiety” (TM) refers to a compound that can be used for treating, at least one symptom of a disease or disorder and can include, but is not limited to, therapeutic polypeptides, oligonucleotides, small molecules and other agents that can be used to treat at least one symptom of a disease or disorder. In embodiments, the TM modulates the activity, expression, and/or levels of a target transcript. In embodiments, the TM decreases the levels of the target transcript through decay mechanisms. In embodiments the activity is the ability of the target transcript to bind to (e.g., sequester) one or more proteins. In embodiments, the TM modulates the activity of the target transcript by reducing the affinity between the target transcript and one or more proteins that bind to the target transcript. As a result of reducing the affinity between the target transcript and the one or more proteins the activity of the one or more proteins may be modulated. For example, if the one or more proteins are not bound to the target transcript, they are available to carry out their functions, such as, for example, facilitating the splicing, alternative splicing, and/or exon skipping of other transcripts. As a result of the function of a TM, the activity, expression, and/or levels of the downstream genes that are regulated by the one or more proteins whose interaction with the target transcript is disrupted by the TM may be modulated.

The terms “modulate”, “modulating” and “modulation″ refer to a perturbation of expression, function or activity when compared to the level of expression, function or activity prior to modulation. Modulation can include an increase (stimulation or induction) or a decrease (inhibition or reduction) in expression, function, or activity. In embodiments, the activity of a target transcript is modulated. In embodiments, modulating the activity of the target transcript includes decreasing the ability of the target transcript to bind to one or more proteins. In embodiments, decreasing the affinity between the target transcript and the one or more proteins results in the modulation of the activity of the one or more proteins that interact with the target transcript. For example, if the one or more proteins are not bound to the target transcript, they are available to carry out their functions, such as, for example, facilitating the splicing, alternative splicing, and/or exon skipping of other transcripts (e.g., downstream transcripts). As such, modulating the activity of the target transcript may result in the modulation of the activity, expression, and/or levels of the downstream genes that are regulated by the one or more proteins whose interaction with the target transcript may be disrupted.

“Amino acid” refers to an organic compound that includes an amino group and a carboxylic acid group and has the general formula

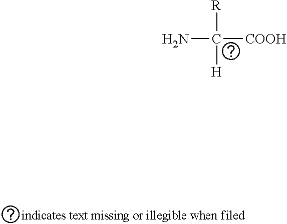

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain″ or “side chain″ refers to the characterizing substituent (“R”) bound to the α-carbon of a natural or non-natural α-amino acid. An amino acid may be incorporated into a polypeptide via a peptide bond.

As used herein, an “uncharged” amino acid is an amino acid having a side chain that has a net neutral charge at pH 7.35 to 7.45. Examples of uncharged amino acids include, but are not limited to, glycine and citrulline.

As used herein, a “charged” amino acid is an amino acid having a side chain having a net charge at a pH of 7.35 to 7.45. An example of a charged amino acid is arginine.

As used herein, the term “sequence identity” refers to the percentage of nucleic acids or amino acids between two oligonucleotide or polypeptide sequences, respectively, that are the same and in the same relative position. As such, one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., Trends Genet.(2000), 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (Identical Residuesx 100)/(Length of Alignment-Total Number of Gaps in Alignment)

In other embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP, gapped BLAST, and PSI-BLAST, in existence as of the date of filing. Such programs are described in Altschul, et al., J. Mol. Biol., (1990),215(3): 403-410; Altschul, et al., Nucleic Acids Res. (1997), 25:3389-3402; Baxevanis et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, “cell targeting moiety” refers to a molecule or macromolecule that specifically binds to a molecule, such as a receptor, on the surface of a target cell. In embodiments, the cell surface molecule is expressed only on the surface of a target cell. In embodiments, the cell surface molecule is also present on the surface of one or more non-target cells, but the amount of cell surface molecule expression is higher on the surface of the target cells. Examples of a cell targeting moiety include, but are not limited to, an antibody, a peptide, a protein, an aptamer, or a small molecule.

As used herein, the terms “antisense compound” and “AC” are used interchangeably to refer to a polymeric nucleic acid structure which is at least partially complementary to a target nucleic acid molecule to which it (the AC) hybridizes. The AC may be a short (in embodiments, less than 50 bases) polynucleotide or polynucleotide homologue that includes at least a portion of a sequence complimentary to a target sequence. In embodiments, the AC is a polynucleotide or polynucleotide homologue that includes a portion that has a sequence complimentary to a target sequence in a target pre-mRNA strand. The AC may be formed of natural nucleic acids, synthetic nucleic acids, nucleic acid homologues, or any combination thereof. In embodiments, the AC includes oligonucleosides. In embodiments, AC includes antisense oligonucleotides. In embodiments, the AC includes conjugate groups. Nonlimiting examples of ACs include, but are not limited to, primers, probes, antisense oligonucleotides, external guide sequence (EGS) oligonucleotides, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self-complementarity to allow for hybridization and formation of a fully or partially double-stranded compound. In embodiments, an AC modulates (increases, decreases, or changes) the expression, levels, and/or activity of a target transcript (e.g., target nucleic acid). In embodiments, the AC decreases the level of the target transcript through inducing decay mechanisms. In embodiments, the AC modulates the activity of the target transcript. In embodiments, the AC modulates the activity of the target transcript by decreasing the ability of the target transcript to bind one or more proteins. In embodiments, decreasing the affinity between the target transcript and the one or more proteins may result in the modulation of the activity of the one or more proteins. For example, if the one or more proteins are not bound to the target transcript, they are available to carry out their functions, such as, for example, facilitating the splicing, alternative splicing, and/or exon skipping of other transcripts (downstream transcripts).

As such, AC mediated modulation of the activity of the target transcript may result in modulation of the activity, expression, and/or levels of the downstream genes that are regulated by the one or more proteins whose interaction with the target transcript may be disrupted.

As used herein, the terms “targeting” or “targeted to” refer to the association of a therapeutic moiety, for example, an antisense compound with a target nucleic acid molecule or a region of a target nucleic acid molecule. In embodiments, the therapeutic moiety includes an antisense compound that is capable of hybridizing to a target nucleic acid under physiological conditions. In embodiments, the antisense conpound targets a specific portion or site within the target nucleic acid, for example, a portion of the target nucleic acid having at least one ideniifiable structure, function, or characteristic such as a particular exon or intron, or selected nucleobases or motifs within an exon or intron.

As used herein, the terms “target nucleic acid sequence,” “target nucleotide sequence,” and “target sequence” refer to the nucleic acid sequence or the nucleotide sequence to which a therapeutic moiety, such as an antisense compound, binds or hybridizes. Target nucleic acids include, but are not limited, to a portion of a target transcript, target RNA (including, but not limited to pre-mRNA and mRNA or portions thereof), a portion of target cDNA derived from such RNA, as well as a portion of target non-translated RNA, such as miRNA. For example, in embodiments, a target nucleic acid can be a portion of a target cellular gene (or mRNA transcribed from such gene) whose expression or transcription is associated with a particular disorder or disease state. The term “portion″ refers to a defined number of contiguous (i.e., linked) nucleotides of a nucleic acid.

As used herein, the term “transcript” or “gene transcript” refers to an RNA molecule transcribed from DNA and includes, but is not limited to mRNA, pre-mRNA, and partially processed RNA.

The terms “target transcript” and “target RNA” refer to the pre-mRNA or mRNA transcript that is bound by the therapeutic moiety. The target transcript may include a target nucleotide sequence. In embodiments, the target transcript includes a target nucleotide sequence that includes an expanded CUG trinucleotide repeat.

The term “target gene” and “gene of interest” refer to the gene of which modulation of the expression and/or activity is desired or intended. The target gene may be transcribed into a target transcript that includes a target nucleotide sequence. The target transcript may be translated into a protein of interest.

The term “target protein″ refers to the polypeptide or protein encoded by the target transcript (e.g., target mRNA).

As used herein, the term “mRNA” refers to an RNA molecule that encodes a protein and includes pre-mRNA and mature mRNA. “Pre-mRNA” refers to a newly synthesized eukaryotic mRNA molecule directly after DNA transcription. In embodiments, a pre-mRNA is capped with a 5′ cap, modified with a 3′ poly-A tail, and/or spliced to produce a mature mRNA sequence. In embodiments, pre-mRNA includes one or more introns. In embodiments, the pre-mRNA undergoes a process known as splicing to remove introns and join exons. In embodiments, pre-mRNA includes one or more splicing elements or splice regulatory elements. In embodiments, pre-mRNA includes a polyadenylation site.

As used herein, the term “expression,” “gene expression,” “expression of a gene,” or the like refers to all the functions and steps by which information encoded in a gene is converted into a functional gene product, such as a polypeptide or a non-coding RNA, in a cell. Examples of non-coding RNA include transfer RNA (tRNA) and ribosomal RNA. Gene expression of a polypeptide includes transcription of the gene to form a pre-mRNA, processing of the pre-mRNA to form a mature mRNA, translocating the mature mRNA from the nucleus to the cytoplasm, translation of the mature mRNA into the polypeptide, and assembly of the encoded polypeptide. Expression includes partial expression. For example, expression of a gene may be referred to as generation of a gene transcript. Translation of a mature mRNA may be referred to as expression of the mature mRNA.

As used herein, “modulation of gene expression″ or the like refers to modulation of one or more of the processes associated with gene expression. For example, modification of gene expression may include modification of one or more of gene transcription, RNA processing, RNA translocation from the nucleus to the cytoplasm, and translation of mRNA into a protein.

As used herein, the term “gene” refers to a nucleic acid sequence that encompasses a 5′ prornoter region associated with the expression of the gene product, and any intron and exon regions and 3′ untranslated regions (“UTR”) associated with the expression of the gene product.

The term “immune cell” refers to a cell of hematopoietic origin and that plays a role in the immune response. Immune cells include, but are not limited to, lymphocytes (e.g., B cells and T cells), natural killer (NK) cells, and myeloid cells. The term “myeloid cells” includes monocytes, macrophages and granulocytes (e.g., basophils, neutrophils, eosinophils and mast cells).

Monocytes are lymphocytes that circulate through the blood for 1-3 days, after which time, they either migrate into tissues and differentiate into macrophages or inflammatory dendritic cells or die. The term “macrophage” as used herein includes fetal-derived macrophages (which also can be referred to as resident tissue macrophages) and macrophages derived from monocytes that have migrated from the bloodstream into a tissue in the body (which can be referred to as monocyte-derived macrophages). Depending on which tissue the macrophage is located, it be referred to as a Kupffer cell (liver), an intraglomular mesangial cell (kidney), an alveolar macrophage (lungs), a sinus histiocyte (lymph nodes), a hofbauer cell (placenta), microglia (brain and spinal cord), or langerhans (skin), among others.

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

As used herein, the term “nucleoside” refers to a glycosylamine that includes a nucleobase and a sugar. Nucleosides include, but are not limited to, natural nucleosides, abasic nucleosides, modified nucleosides, and nucleosides having mimetic bases and/or sugar groups. A “natural nucleoside” or “unmodified nucleoside” is a nucleoside that includes a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside having a phosphate group covalently linked to the sugar. Nucleotides may be modified with any of a variety of substituents.

As used herein, the term “nucleobase” refers to the base portion of a nucleoside or nucleotide. A nucleobase may include any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase that includes a heterocycle.

As used herein “internucleoside linkage” refers to a covalent linkage between adjacent nucleosides.

As used herein “natural internucleoside linkage” refers to a 3′ to 5′ phosphodiester linkage.

As used herein, the term “modified internucleoside linkage” refers to any linkage between nucleosides or nucleotides other than a naturally occurring internucleoside linkage.

As used herein “oligonucleoside” refers to an oligonucleotide in which the internucleoside linkages do not contain a phosphorus atom.

As used herein the term “chimeric antisense compound” refers to an antisense compound, having at least one sugar, nucleobase, and/or internucleoside linkage that is differentially modified as compared to the other sugars, nucleobases, and internucleoside linkages within the same oligomeric compound. The remainder of the sugars, nucleobases, and internucleoside linkages can be independently modified or unmodified. In general, a chimeric oligomeric compound will have modified nucleosides that can be in isolated positions or grouped together in regions that will define a particular motif. Any combination of modifications and or mimetic groups can include a chimeric oligomeric compound as described herein.

As used herein, the term “mixed-backbone antisense oligonucleotide” refers to an antisense oligonucleotide wherein at least one internucleoside linkage of the antisense oligonucleotide is different from at least one other internucleoside linkage of the antisense oligonucleotide.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U).

In embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association.

Therefore, described herein are antisense compounds that may include up to about 20% nucleotides that are mismatched (i.e., are not nucleobase complementary to the corresponding nucleotides of the target). In embodiments, the antisense compounds contain no more than about 15%, for example, not more than about 10%, for example, not more than 5%, or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99%, or 100% nucleobase complementary to a target nucleic acid.

As used herein, “hybridization″ means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In embodiments, an antisense oligonucleotide specifically hybridizes to more than one target site. In embodiments, an oligomeric compound specifically hybridizes with its target under stringent hybridization conditions.

“Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

As used herein, the term “2′-modified” or “2′-substituted” means a sugar that includes substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O-C1-C10 alkyl, —OCF3, 0—(CH2)2—O—CH3, 2′-O(CH₂)2SCH3, O—(CH2)2—O—N(Rm)(Rn), or O—CH₂—C(═O)—N(Rm)(Rn), where each Rm and Rn is, independently, H or substituted or unsubstituted C₁-C₁₀ alkyl.

As used herein, the term “MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of an antisense compound that includes the modified nucleotide to a target nucleic acid. High-affinity modifications include, but are not limited to, BNAs, LNAs and 2′-MOE.

As used herein the term “mimetic” refers to groups that are substituted for a sugar, a nucleobase, and/or internucleoside linkage in an AC. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside, and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, P-D-LNA, ENA, Oxyamino BNA (2′-O—N(CH3)—CH2—4′) and Aminooxy BNA (2′—N(CH₃)—O—CH₂—4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene group, thereby forming a 2′-C,4′-C-oxymethylene linkage. LNAs include, but are not limited to, α-L-LNA, and R-D-LNA.

As used herein, the term “cap structure” or “terminal cap moiety” refers to chemical modifications, which have been incorporated at either end of an AC.The term “therapeutic polypeptide” refers to a naturally occurring or recombinantly produced macromolecule that includes two or more amino acids and has therapeutic, prophylactic or other biological activity.

The term “small molecule” refers to an organic compound with pharmacological activity and a molecular weight of less than about 2000 Daltons, or less than about 1000 Daltons, or less than about 500 Daltons. Small molecule therapeutics are typically manufactured by chemical synthesis.

“Wild type target protein″ refers to a native, functional protein isomer produced by a wild type, normal, or unmutated version of the target gene. The wild type target protein also refers to a protein resulting from a target pre-mRNA that has been re-spliced.

A “re-spliced target protein″, as used herein, refers to the protein encoded by the mRNA resulting from the splicing of the target pre-mRNA to which the AC hybridizes. Re-spliced target protein may be identical to a wild type target protein, may be homologous to a wild type target protein, may be a functional variant of a wild type target protein, may be an isoform of a wild type target protein, or may be an active fragment of a wild type target protein.

As used herein, an “expanded trinucleotide repeat,” such as an “expanded” CUG or and “expanded” CTG repeat, means a gene containing or encoding the trinucleotide repeat contains a number of repeated consecutive trinucleotides that is greater than present in a wild type gene.

Expanded nucleotide repeats may be written as XXX-NNN or (XXX-NNN) where XXX refers to the DNA repeat and NNN refers to the RNA repeat that is transcribed from the DNA repeat. For example, the CTG·CUG repeat, refers to a gene having a CTG DNA repeat from which a RNA having a CUG repeat is transcribed. In embodiments, the number of repeats in an expanded trinucleotide repeat is 5 or more, 10 or more 15, or more or 20 or more than the wild type gene. In embodiments, the expanded trinucleotide repeat includes 2x, 3x, 4x, 5x, 10x, 20x, 50x or more trinucleotide repeats than the wild type gene. The expanded trinucleotide repeat may result in a disease in a subject having a gene that contains the expanded trinucleotide repeat. For example, a subject having an expanded CTG repeat in a gene may suffer from DM1 or FECD. In DM1, the DPMK gene contains an expanded CTG repeat. Subjects that suffer from DM1 may have 50 or more CTG repeats in the 3′ untranslated region (UTR) of the DPMK gene, while non-disease subjects typically have 5 to 34 CTG repeats in the 3′ UTR of the DPMK gene. In FECD, the TCF4 gene contains an expanded CTG repeat. Subjects that suffer from FECD may have 40 or more CTG repeats in a CTG18.1 locus of the TCF4 gene, while non-disease subjects typically have 30 or less CTG repeats in the CTG18.1 locus of the TCF4 gene. mRNA transcribed from a gene having an expanded CTG repeat will have an expanded CUG repeat.

The term “downstream” in the present disclosure, as it relates to a gene, mRNA, or protein, refers to a gene, mRNA, or protein that is affected by binding of AC to the target nucleotide (e.g., target transcript) but is not the gene, mRNA, or protein corresponding to the target nucleotide.

Binding of the AC to the target nucleotide may reduce aggregation or sequestration of RNA binding protein such as MBNL1 or CUGBP1 on accumulated mRNA having CUG repeats, which may make available such RNA binding proteins for proper transcription, RNA processing, and/or expression of downstream gene products.

As used herein, “functional fragment” or “active fragment” refers to a portion of a eukaryotic wild type target protein that exhibits an activity, such as one or more activities of a full-length wild type target protein, or that possesses another activity. In embodiments, a re-spliced target protein that shares at least one biological activity of wild type target protein is considered to be an active fragment of the wild type target protein. Activity can be any percentage of activity (i.e., more or less) of the full-length wild type target protein, including but not limited to, about 1% of the activity, about 2%, about 3%, about 4%, about 5%, about 10%, about 20%, about 30%, about 40%, about 50%, about 60%, about 70%, about 80%, about 90%, about 95%, about 96%, about 97%, about 98%, about 99%, about 100%, about 200%, about 300%, about 400%, about 500%, or more (including all values and ranges in between these values) activity compared to the wild type target protein. Thus, in embodiments, the active fragment may retain at least a portion of one or more biological activities of wild type target protein. In embodiments, the active fragment may enhance one or more biological activities of wild type target protein.

“Wild type target protein″ refers to a native, functional protein isomer produced by a wild type, normal, or unmutated version of the target gene. The wild type target protein also refers to the protein resulting from a target pre-mRNA that has been properly spliced.

As used herein, the terms “splicing” and “processing” refer to the modification of a pre-mRNA following transcription, in which introns are removed and exons are joined. Splicing occurs in a series of reactions that are catalyzed by a large RNA-protein complex composed of five small nuclear ribonucleoproteins (snRNPs) referred to as a spliceosome. Within an intron, a 3′ splice site, a 5′ splice site, and a branch site are required for splicing. The RNA components of snRNPs interact with the intron and may be involved in catalysis.

As used herein, alternative splicing refers to the splicing of different combinations of exons present in a gene, which results in the generation of different mRNA transcripts from a single gene.

A “re-spliced target protein″, as used herein, refers to the protein encoded by the mRNA resulting from the splicing of the target pre-mRNA to which the AC hybridizes. Re-spliced target protein may be identical to a wild type target protein, may be homologous to a wild type target protein, may be a functional variant of a wild type target protein, or may be an active fragment of a wild type target protein.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

### Example 1. Assessment of PMOs and PMO-EEV compounds for impacts on RNA foci formation and splicing rescue in DM1-related cell lines

The effect of a (CUG)7 (SEQ ID NO: 413) repeat PMO and a (CUG)7 (SEQ ID NO: 413) repeat PMO-EEV compound (A and D in Table 12) on RNA foci formation and splicing rescue was evaluated in DM1-related cell lines. A PMO-EEV compound having a mismatched PMO sequence and a PMO having a scrambled sequence were also included (B and C in Table 12).

### Experimental

Cells. The PMOs and PMO-EEVs were evaluated in a DM1 HeLa cell model (HeLa-480), which is a stable cell line with high CUG repeat load and downstream splicing defects, and DM1 myoblasts derived from a DM1 patient with about 2600 CTG repeats and downstream splicing defects. HeLa-480 recapitulates pathogenic hallmarks of DM1, including CUG ribonuclear foci and mis-splicing of pre-mRNA targets of the muscleblind (MBNL) alternative splicing factors. It was noted that DM1 myoblasts grow quite slowly (doubling time of about 7 days) and do not transfect well. Control HeLa and HeLa-480 cells were treated with ENDOPORTER without additional compounds. HeLa-480 cells represent the disease state and HeLa cells represent the un-diseased state.

RNA foci analysis. HeLa-480 cells or DM1 myoblasts were treated with 1 μM, 3 μM, or 10 μM of compounds A-D (Table 12). All compounds were transfected without a transfection reagent or using the ENDOPORTER (available from GENETOOLS LLC in Philomath, Oregon) transfection agent designed to deliver naturally charged PMOs into cells. Cells were incubated for 24 hours and then fixed for qualitative RNA foci analysis via microscopy. Upon qualitative visual inspection of the treated Hela-480 cells, compound A (PMO-EEV) showed the least amount of RNA foci. No conclusion was drawn from the DM1 myoblasts.

Splicing analysis and RT-PCR. HeLa-480 cells treated as described above were harvested after 24 or 48 hours of the incubation and the total RNA was extracted. RT-PCR was performed to measure and/or quantify splicing patterns of DM-affected exons in target RNAs such as MBNL1 and CLASP1. The percentage of exon inclusion of interest was evaluated.

TABLE 12
PMOs and PMO-EEVs tested in Example 1.

| ID | Sequence (all PMO) | Purity | Solvent |
|---|---|---|---|
| A | CAGCAGCAGCAGCAGCAGCAG-click-K- | 91 | Saline |
| | PEG12-K(FfΦRrRrQ)-PKKKRKV-Ac | | (0.9% |
| | (SEQ ID NOS 622-623 and 643, | | NaCl) |
| | respectively, in order of | | |
| | appearance; SEQ ID NO: 154)- | | |
| | click-K-PEG12-(SEQ ID NO: 78)- | | |
| | (SEQ ID NO: 42)-Ac | | |
| B | GTAACTGTATTTGGTACTTCC-C3-NH2- | 99 | PBSX1 |
| | PEG4-COT-PEG12-PKKKRKV-(FfΦRrRrQ) | | |
| | (SEQ ID NOS 624-625 and 644, | | |
| | respectively, in order of | | |
| | appearance; SEQ ID NO: 317)- | | |
| | C3-NH2-PEG4-COT-PEG12-(SEQ | | |
| | ID NO: 78)-(SEQ ID NO: 42)-Ac | | |
| C | AGCCAGAGCACCGCAACCGGACGAG | 81 | Saline |
| | (SEQ ID NO: 318) | | (0.9% |
| | | | NaCl) |
| D | CAGCAGCAGCAGCAGCAGCAG | 75 | Saline |
| | (SEQ ID NO: 638) | | (0.9% |
| | | | NaCl) |

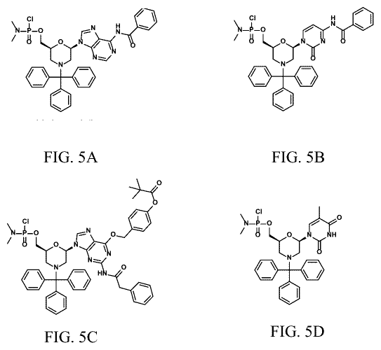

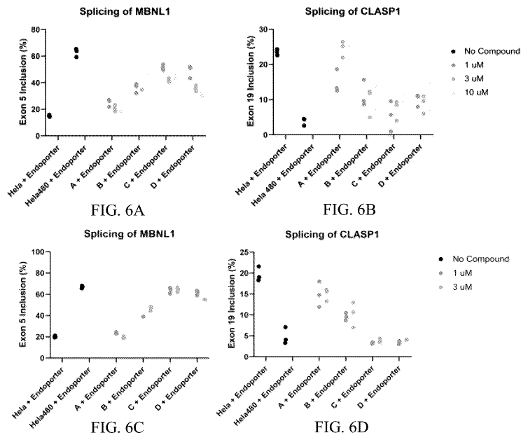

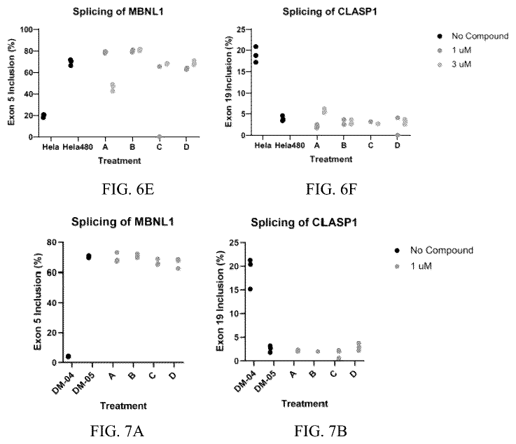

Results. FIG. 6A-6D show the RT-PCR results of the alternative splicing events of MBNL1 (6A and 6C, exon 5 inclusion) and CLASP1 (6B and 6D, exon 19 inclusion) 24 hours (6A and 6B) and 48 hours (6C and 6D) after HeLa-480 cells were treated with compounds A-D and the ENDOPORTER transfection agent. A reduction of exon 5 inclusion in MBNL1 was observed for cells treated with each of compounds A-D at both the 24— and 48-hour time points (FIGS. 6A and 6C). Cells treated with compound A showed the largest rescue (decrease in exon 5 inclusion). An increase in exon 19 inclusion in CLASP1 was observed for cells treated with each of compounds A-D at both the 24— and 48-hour time points (FIGS. 6B and 6D). Cells treated with compound A showed the largest rescue (increase in exon 19 inclusion). In contrast, when HeLa-480 cells were treated with A-D without the ENDOPORTER transfection reagent no change in the splicing events of MBNL1 (FIG. 6E) or CLASP1 (FIG. 6F) were observed, except for compound A.

FIG. 7A-7B show the RT-PCR results of alternative splicing events of MBNL1 and CLASP1 after DM1 myoblasts were treated with compound A-D, the negative control DM-04, or the positive control DM-05. Treatment with all the compounds resulted in the rescue of the splicing events of MBNL1 (FIG. 7A) and CLASP1 (FIG. 7B).

### Example 2. Assessment of EEV-PMO 221-1106 for impacts on splicing rescue in DM1-mouse model

The effect of a PMO only and a PMO-EEV (Table 13) on splicing rescue was evaluated in vivo using an HSA-LR DM1-mouse model.

### Experimental

Mouse Model. HSA-LR is a transgenic mouse model having expanded long CUG repeats (LR) in the 3′-UTR of a human skeletal actin (HSA) transgene and expresses CUGexp RNA (e.g., expanded CUG RNA) at high levels in skeletal muscle (Mankodi et al., Science 2000, 289(5485):1769-1773). The HSA-LR mouse shows myotonic phenotype along with splicing defects. A Friend Virus B NIH Jackson (FVB/NJ) mouse model was used as a control and to make the HSA-LR transgenic mice.

Experimental design. Compounds A-D were administered to the mice via Retro-orbital injection or intravenous (IV) injection at a single dose of 100 μL compound solution per 20 g body weight. The scale was proportional to body weight of each mouse (e.g., 150 μL per 30 g body weight).

TABLE 13
PMOs and PMO-EEVs tested in Example 2.

| | | | Con. | Sol- |
|---|---|---|---|---|
| ID | Compound | Sequence (all PMOs) | (mg/ml) | vent |
| (A) | Saline | Vehicle control | NA | Saline |
| | | | | (0.9% |
| | | | | NaCl) |
| (B) | 221 | 5′-CAG CAG CAG CAG | 6 | Saline |
| | | CAG CAG CAG-3′ | | (0.9% |
| | | (SEQ ID NO: 638) | | NaCl) |
| (C) | 221-1106 | 5′-CAG CAG CAG CAG | 4 | Saline |
| | at 20 mpk | CAG CAG CAG-3′-PEG4COT- | | (0.9% |
| | | click-K-miniPEG2- | | NaCl) |
| | | Lys(FfFGRGRE)-miniPEG2- | | |
| | | VKRKKKP-Ac | | |
| | | (SEQ ID NOS 626-627 and | | |
| | | 645, respectively, | | |
| | | in order of appearance; | | |
| | | SEQ ID NO: 154)- | | |
| | | PEG4COT- | | |
| | | click-K-miniPEG2-K(SEQ | | |
| | | ID NO: 76)-miniPEG2- | | |
| | | (SEQ ID NO: 42)-Ac | | |
| (D) | 221-1106 | 5′-CAG CAG CAG CAG CAG | 8 | Saline |
| | at 40 mpk | CAG CAG-3′-PEG4COT- | | (0.9% |
| | | click-K-miniPEG2- | | NaCl) |
| | | Lys(FfFGRGRE)-miniPEG2- | | |
| | | VKRKKKP-AC | | |
| | | (SEQ ID NOS 626-627 and | | |
| | | 645, respectively, in | | |
| | | order of appearance; | | |
| | | SEQ ID NO: 154)- | | |
| | | PEG4COT- | | |
| | | click-K-miniPEG2-K(SEQ | | |
| | | ID NO: 76)-miniPEG2- | | |
| | | (SEQ ID NO: 42)-Ac | | |

Animals were age matched and assigned into six treatment groups. Two control groups were used; Group 1: FVB/NJ mice (FVB/NJ; un-diseased control) and Group 2: HSA-LR (diseased control) mice, each of which were injected with saline. Four treatment groups (Groups A-D) were used; HSA-LR mice injected with compounds A, B, C, or D. The FVB/NJ mice were 5 weeks and 4 days old when injected, while the HSA-LR mice were 6 weeks and 1 or 2 days old when injected. Four mice (two males and two females) per group were utilized for this experiment.

Mice were sacrificed 1 week post treatment. Tissues (Gastrocnemius, Quadriceps, Tibialis Anterior (TA)) were harvested and flash frozen in liquid nitrogen and stored at −80° C. for further evaluation of splicing rescue analysis.

Total RNA was extracted from tissue samples and analyzed by RT-PCR to assess AC-induced alternative RNA splicing rescue events on (i) Atp2al exon 22, (ii) Nfix exon 7, (iii) Clcn1 exon 7a, and (iv) Mbn11 exon 5. The percentage of exon inclusion of interest was evaluated.

Results. Prior to treatment all HSA-LR mice had myotonia. After injection of compounds, all mice were disoriented. Mice injected with compounds A and B (Groups A and B) recovered within 15 minutes, while mice injected with compounds C and D (Groups C and D) took several hours to recover. All treated mice completely recovered by the next day. At time of sacrifice, Groups A and B had myotonia; however, Groups C and D clearly did not have myotonia.

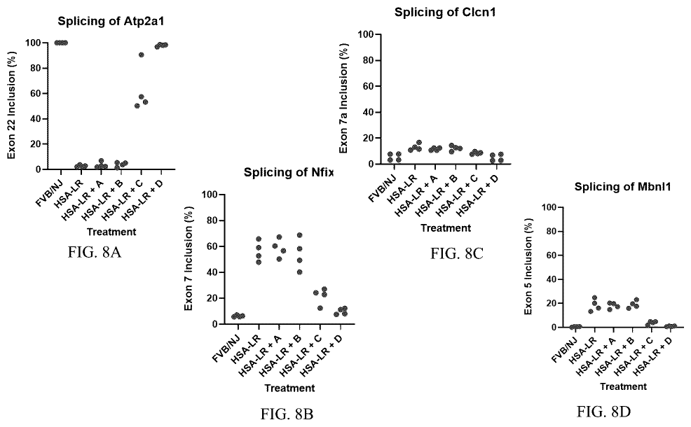

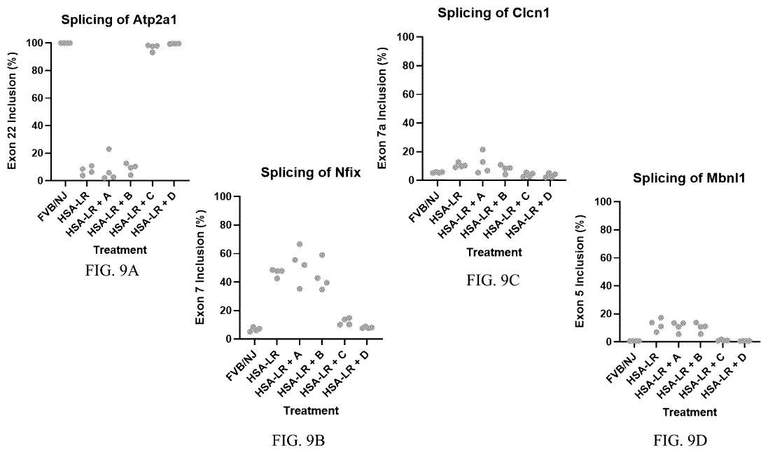

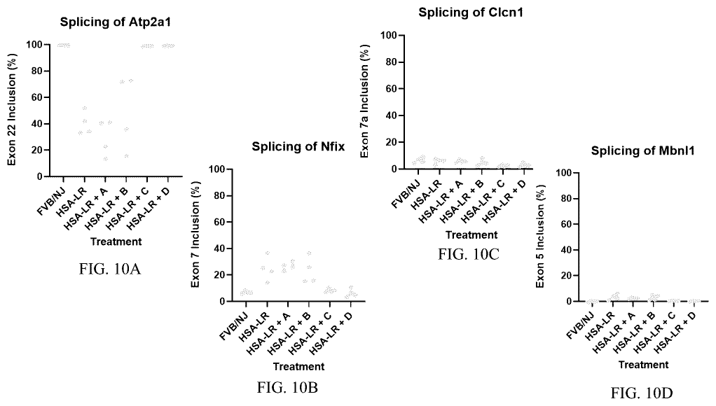

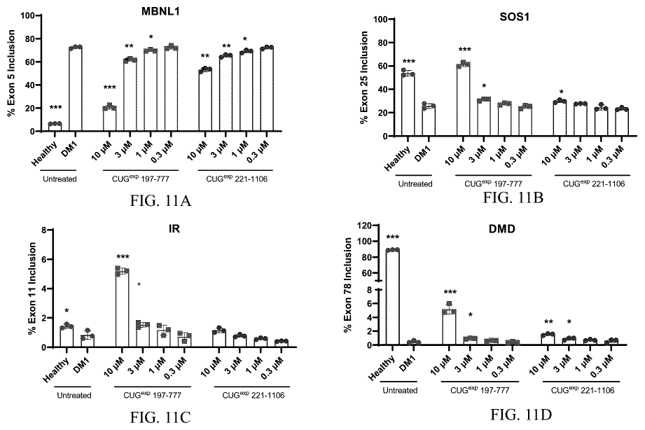

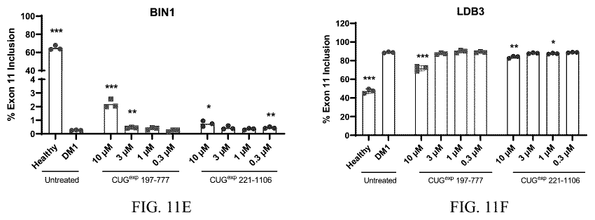

FIGS. 8A-10D show the RNA splicing measurements for Atp2al (for exon 22 inclusion; FIGS. 8A, 9A, and 10A), Nfix (for exon 7 inclusion; FIGS. 8B, 9B, and 10B), Clcn1 (for exon 7a inclusion; FIGS. 8C, 9C, and 10C) and Mbn11 (for exon 5 inclusion; FIGS. 8D, 9D, and 10D) in gastrocnemius (FIGS. 8A-8D), quadricep (FIG. 9A-9D), and tibialis anterior (FIG. 10A-10D) muscle tissue of treated mice. Mice treated with compounds C and D (PMO-EEV) showed a rescue of Atp2al and Nfix splicing events in gastrocnemius, quadriceps, and tibialis anterior tissue while the PMO and saline groups did not show rescue of splicing events (FIGS. 8A, 8B, 9A, 9B, 10A, and 10B. Similarly, in the gastrocnemius and quadricep muscle tissue, mice treated with compounds C and D showed a rescue of Clcn1 (FIGS. 8C and 9C) and Mbn11 (FIGS. 8D and 9D) splicing events while the PMO and saline groups did not show rescue of splicing events. Regarding the splicing of Clcn1 and Mbn11 in the tibialis tissue (FIG. 10C-10D), no alternative splicing defects were detected in the control mouse line (FVB/NJ) and the DM1 mouse model (HSA-LR), As such, treatment with compounds A-D did not result in splicing rescue in the Clcn1 and Mbn11 genes in the tibialis tissue.

These results demonstrate that positive impacts of PMO-EEV treatment on splicing rescue in vivo study using DM1 mouse model as well as potential use of PMO-EEV compounds to treat myotonic dystrophy (DM).

### Example 3. Assessment of various PMO-EEV compounds for correcting mis-splicing events in Immortalized Myoblasts from DM1 patients

The effect of two DMPK CUG-targeting PMO-EEVs (197-777 and 221-1106; Table 14) on splicing rescue of DM1-related genes was evaluated in vitro using DM1 patient derived muscle myoblasts and myotubes.

### Experimental

Cell culture. Immortalized myoblasts from DM1 patients (ASA308DM1), and unaffected individuals (KM1421; AB 1190) were obtained. DM1 patient myoblasts harbor 2600 CTG repeats in the 3′-UTR of DMPK. Myoblasts were cultured in a growth medium of Skeletal Muscle Cell Growth Medium (available from PromoCell in Heidelberg, Germany), 2% horse serum (available from Gibco in Bristol, RI), 1% chick embryo extract (available from USB Corp in Cleveland, OH), and 0.5 mg/mL penicillin/streptomycin (Gibco). For myogenic differentiation, confluent cultures were switched to differentiation medium of DMEM supplemented with 2% horse serum and cultured for four days.

TABLE 14
Compounds tested in Example 3

| Compound | | PMO sequence | PMO | Conjugation |
|---|---|---|---|---|
| ID | EEV Sequence (N to C) | (5′-3′) | modifications | Chemistry |
| PMO only | NA | CAGCAGCA | 5′OH | NA |
| | | GCAGCAGC | | |
| | | AGCAG | | |
| | | (SEQ ID NO: | | |
| | | 639) | | |
| 197-777 | Ac-PKKKRKV- | CAGCAGCA | 5′-sarcosine | Click |
| (DM1-1) | Lys(cyclo[Ff-Nal-RrRrQ])- | GCAGCAGC | amide; 3′-C4- | |
| | PEG₁₂-K(N3)-NH2 (SEQ | AGCAG | cyclooctyne | |
| | ID NOS 628-629, | (SEQ ID NO: | | |
| | respectively, in order of | 640) | | |
| | appearance) | | | |
| | Ac-(SEQ ID NO: 42)-- | | | |
| | Lys(SEQ ID NO: 78)- | | | |
| | PEG₁₂-K(N3)-NH2 | | | |
| 221-1106 | Ac-PKKKRKV-miniPEG2- | CAGCAGCA | 5′-OH; 3′- | ?Click with |
| (DM1-2) | Lys(cyclo[FfFGRGRQ]- | GCAGCAGC | secondary amine | a PEG4- |
| | miniPEG2-K(N3)-NH2 | AGCAG | morpholino | COT linker |
| | (SEQ ID NOS 630-631, | (SEQ ID NO: | | |
| | respectively, in order of | 641) | | |
| | appearance) | | | |
| | Ac-(SEQ ID NO: 42)- | | | |
| | miniPEG2-Lys(SEQ ID | | | |
| | NO: 76)-miniPEG2- | | | |
| | K(N3)NH2 | | | |

Treatment. DM1 patient muscle cells were treated with 10 μm, 3 μm, 1 μm, or 0.3 μm of the compounds using two different treatment conditions. In the first condition, myoblasts were plated at 75-80% confluence, the compounds were serially diluted in growth medium, and cells were bathed for 24 hours to allow for free-uptake of compound. The compound-containing media was removed, myoblasts washed with 1X DPBS (Gibco), and differentiated for four days prior to harvest. For the second condition run in parallel, myoblasts were differentiated three days prior to treatment, compounds were serially diluted in differentiation medium, and myotubes were harvested for analysis 24 hours later.

RNA isolation and PCR. Total RNA was isolated with the RNEASY Mini Kit (available from Qiagen in Germantown, MD) according to the manufacturer's instructions. For exon inclusion, 100 ng RNA was reverse transcribed and used for PCR (OneStep RT-PCR Kit, Qiagen).

Samples were analyzed by LabChip (available from PerkinElmer in Waltham, MD) with the HT DNA High Sensitivity Assay Kit.

Results. DMPK CUG targeting PMO (not conjugated with EEV) improved mis-splicing of MBNL1 exon 5 (data not shown). FIG. 11A-11F shows mixed rescue of splicing defect of MBNL1 (FIG. 11A) and its targets (SOS1, IR, DMD, BIN1, LDB3; FIG. 11B-11F) in DM1 patient derived muscle cells treated with various concentrations of DMPK CUG-targeting EEV-PMOs (CUG^{exp} 197-777 and CUG° P 221-1106). EEV-PMO 197-777 elicited moderate correction of mis-splicing events in DM1 patient muscle cells. MBNL1 and SOS1 showed the best response of mis-splicing correction. EEV-PMO 197-777 chosen as tool compound for follow-up experiments described below.

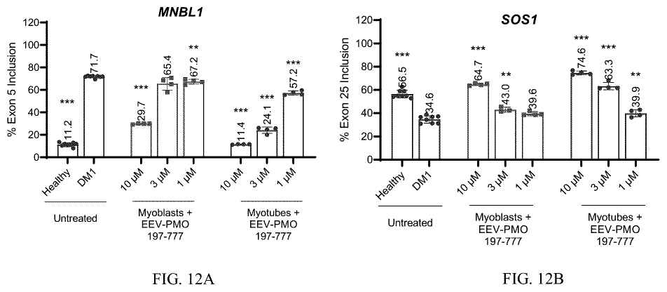

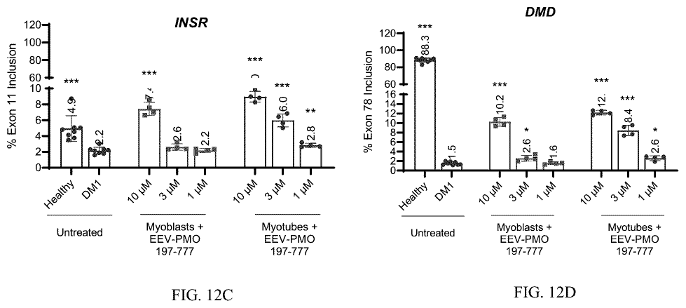

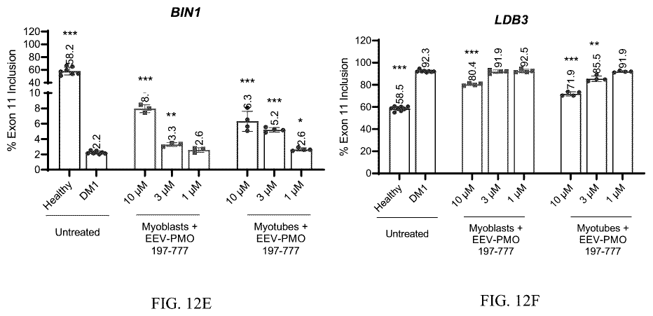

DM1 patient derived myoblasts and myotubes were treated with 10 μm, 3 μm, and 1 μm of DMPK CUG-targeting EEV-PMO 197-777 using methods similar to those described above. Rescue of alternative RNA splicing events of MNBL1 and MNBL1 targets was evaluated. Various extendts of splicing correction was observed for MNBL1 (exon 5 exclusion; FIG. 12A), SOS1 (exon 25 inclusion; FIG. 12B), INSR (exon 11 inclusion; FIG. 12C), DMD (exon 78 inclusion, FIG. 12D), BIN1(exon 11 inclusion; FIG. 12E), and LDB3 (exon 11 exclusion; FIG. 12F) after myoblasts and myotubes were treated with EEV-PMO.

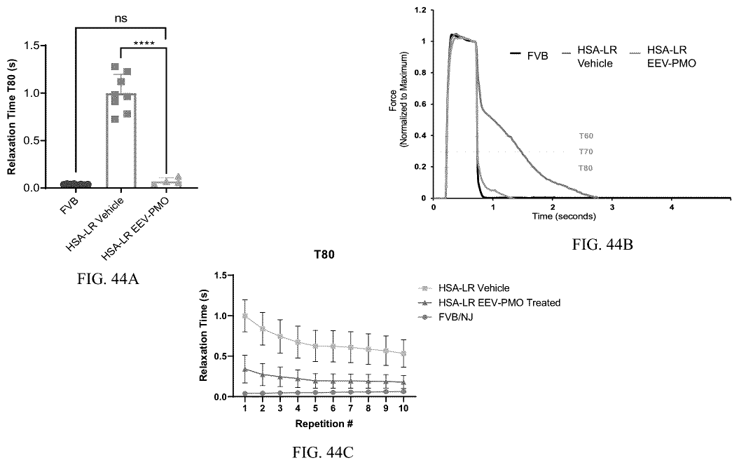

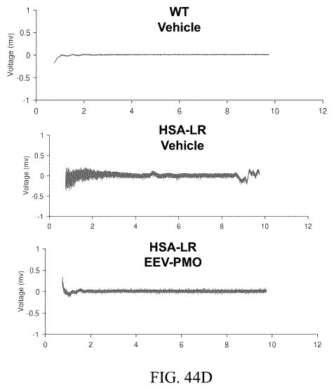

FIGS. 44A-44D show the reversal of myotonia phenotypes in HSA-LR mice treated with 20 mpk 221-1106 quantified by muscle relaxation assay. FIGS. 44A and 44C show plots of relaxation time to 80% of peak isometric force and FIG. 44B shows the force trace raw data. FIG. 44D shows the reversal of myotonia phenotypes in HSA-LR mice treated with 20 mpk 221-1106 quantified by representative electromyography (EMG) traces.

### Example 4. Evaluation of PMO-EEV 221-1120 in a DM1 mouse model

A DM1 mouse model was done to study the effect of EEV-PMO 221-1120 (also referred to as EEV-PMO-DM1-3 or DM1-3; PMO 221=5′-CAG CAG CAG CAG CAG CAG CAG-3′ (SEQ ID NO: 638; all PMO monomers); EEV 1120=Ac-PKKKRKV-AEEA-Lys(cyclo[FGFGRGRQ]-PEG12—OH (SEQ ID NOS 632-633, respectively, in order of appearance; Ac-(SEQ ID NO: 42)-AEEA-Lys(SEQ ID NO: 82)-PEG12—OH)) on the splicing and mRNA levels of downstream genes in the same HSA-LR transgenic mouse model as described in Example 2. The PMO and EEV were conjugated using amide chemistry.

Experimental. There were two general treatment groups: 1) wild-type mice; and 2) HSA-LR mice (DM1 disease model). Within the HSA-LR treatment group there were two sub-treatment groups: 1) HSA-LR treated with saline (control); and 2) HSA-LR+EEV-PMO 221-1120. Mice were treated with 15 mpk, 30 mpk, 60 mpk, or 90 mpk (based on the PMO) of the PMO-EEV or saline via tail intravenous injection. Seven days after treatment, mice were sacrificed, and tissues were collected for analysis.

RT-PCR assays (correction of splicing). Tissues were homogenized by OMNI BEAD MILL HOMOGENIZER and the RNA was extracted by QIACUBEQ. RT-PCR assays were performed using one-step RT-PCT kit (Qiagen) following the manufacturer's protocols with 35 PCR cycles of 940° C. for 30 seconds; 600° C. for 30 seconds and 720° C. for 30 seconds. Sequence of gene specific primers are as follows: Clcn1 exon 7a inclusion Forward primer=5′-TTCACATCGCCAGCATCTGTGC-3′ (SEQ ID NO: 319), Reverse primer=5′-CACGGAACACAAAGGCACTGAATGT-3′ (SEQ ID NO: 320); Mbn11 exon5 inclusion forward primer=5′-GCTGCCCAATACCAGGTCAAC-3′ (SEQ ID NO: 321), reverse primer=5′-TGGTGGGAGAAATGCTGTATGC-3′ (SEQ ID NO: 322); Atp2al exon 22 inclusion forward primer=5′-GCTCATGGTCCTCAAGATCTCAC-3′ (SEQ ID NO: 323), reverse primer: 5′-GGGTCAGTGCCTCAGCTTTG-3′ (SEQ ID NO: 324); Nfix exon 7 inclusion forward primer=5′-TCGACGACAGTGAGATGGAG-3′ (SEQ ID NO: 325), reverse primer 5′ CAAACTCCTTCAGCGAGTCC-3′ (SEQ ID NO: 326). Primers for Clcn1, Mbn11, and Atp2al were from Klein et al., The Journal of Clinical Investigation. 2019, 129 (11), pg. 4739; and primers for Nfix were from Chen et al., Scientific Reports. 2016, 6(1), pg. 1. The cDNA products were separated on 2% agarose E-gel with SYBR SAFE dye. The percentage exon inclusion was calculated by the ratio of the un-skipped band/(un-skipped band+skipped band).

Mouse DM1 splicing index (mDSI) calculation. The mDSI was calculated following the literature protocol from Tanner et. al. (Nucleic acids research. 2021, 49 (4), pg. 2240-54). For each sample i, normalized splicing values were calculated for each splice event j as (PSIi,j -PSI_{wildtype,j})/(PSI_{HSALR,j}-PSI_{wildtype,j}), where PSI_{wildtype,j} is the average PSI for event j across the wildtype mice, and PSI_{HSALR,j} is the average PSI for event j across the HSALR mice. mDSI is then calculated as the mean of all normalized splicing values, which are Atp2al, Nfix, Mbn11 and Clcn1 in the studies.

qRT-PCR assays (HSA mRNA knockdown). Reverse transcription was performed using the High-Capacity cDNA Reverse Transcription Kit from Life Technologies Corporation following the manufacturer's protocols. Quantitative real-time PCR were performed using Bio-Rad SyBr Green Supermix and QuantStudio3 qPCR machine with gene specific primers: HSA mRNA forward primer=5′-TTCCATCGTCCACCGCAAAT-3′ (SEQ ID NO: 327), reverse primer=5′-AGTTTACGATGGCAGCAACG-3′ (SEQ ID NO; 328), both primers from Klein et al., The Journal of Clinical Investigation. 2019, 129 (11), pg. 4739; and mouse GAPDH forward primer=5′-AGGTCGGTGTGAACGGATTTG-3′ (SEQ ID NO: 329), reverse primer=5′-TGTAGACCATGTAGTTGAGGTCA-3′ (SEQ ID NO: 330).

RNAseq. PolyA RNAseq using Next Generation Sequencing was done for transcriptome profiling. The Z-score for each gene was calculated as the (sample value -the mean)/(the standard deviation). Differential splicing analysis was done on the RNAseq data to calculate the percent spliced (PSI) of individual exons for each gene. The PSI is a ratio of normalized read counts indicating the inclusion of a transcript element over the total normalized reads for that event (inclusion and exclusion reads). For example, if an exon is included in the reads 100% of the time, the PSI is 1. Additionally, if an exon is excluded from the reads 100% of the time, the PSI is 0.

Twenty-two genes of interest known to be predictive of DM1 were analyzed. Additionally, the genes studied in Wagner et al. (PLOS Gen 2016 (47)) and Tanner et al. (NAR 2021 (48), 4, 2240-2254) were analyzed. The mouse exons were mapped to the human location. In some cases, the boundary of the exons in mice and/or the human genome was not completely known. As such, the data was analyzed using different boundaries. The correct boundaries were verified using the RNAseq data.

RNA CUG Foci analysis. Tibialis anterior muscle sections were stained for CUG foci (FISH, red) and nuclei (Hoechst, blue). TA muscle sections were imaged and the number of nuclei having a CUG RNA foci were quantified.

### Results

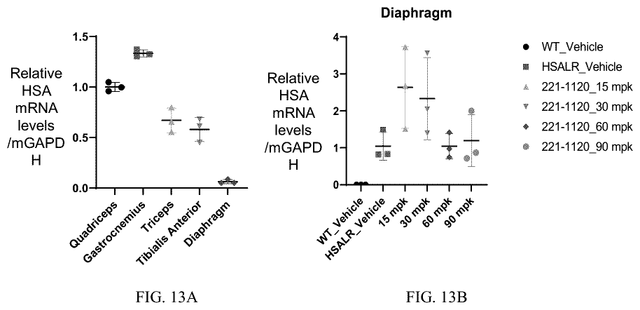

HSA mRNA knockdown. The diaphragm only expressed 5-10% of the HSA mRNA levels compared to quadriceps, tibialis anterior, and triceps (FIG. 13A). EEV-PMO treatment did not seem to change the HSA mRNA levels in the diaphragm (FIG. 13B). The expression level of the HSA 220 CUG repeats may not be sufficient for the mis-splicing phenotype in DM1 in the diaphragm.

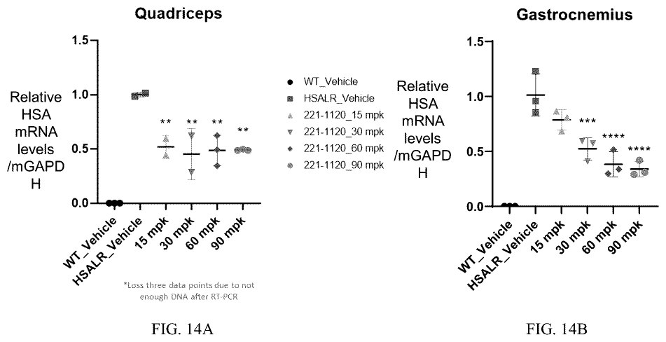

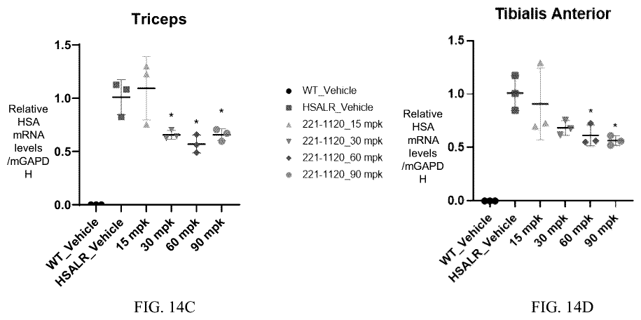

The EEV-PMO knock downed HSA mRNA in a dose-dependent manner, confirming target engagement in quadricep (FIG. 14A), gastrocnemius (FIG. 14B), tricep (FIG. 14C), and tibialis anterior (FIG. 14D) tissue. Additionally, the Ct (cycle threshold) value of HSA mRNA is similar to the level of mouse GAPDH (˜15), suggesting high expression of HSA transgene in HSA-LR mice in the quadriceps.

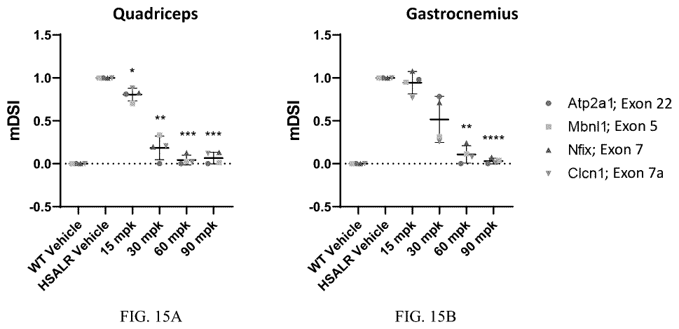

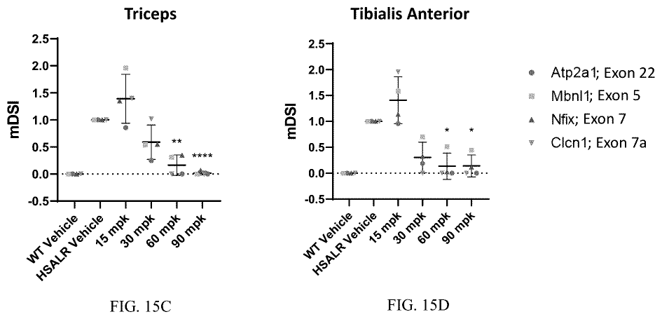

mDSI (correction of splicing). The mouse DM1 splicing index (mDSI) for the quadriceps, the gastrocnemius, the triceps, and the tibialis anterior are shown in FIG. 15A-15D. Treatment with EEV-PMO corrected DM1 relevant splicing defects (Atp2al exon 22, Nfix exon 7, Clcn1 exon 7a, Mbn11 exon 5) at 1-week post injection in the quadriceps (FIG. 15A), gastrocnemius (FIG. 15B), triceps (FIG. 15C), and tibialis anterior (FIG. 15D) in a dose dependent manner with higher doses approaching or equivalent to wild type (full correction). Approximately 50%-60% human skeletal actin RNA knockdown in HSA-LR mice was achieved at drug concentrations that achieve near complete splicing correction.

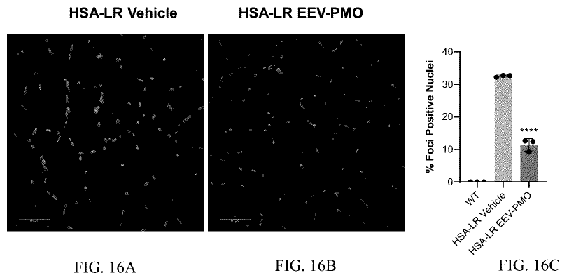

FIGS. 16A-B show images of tibialis anterior tissue of HSA-LR mice (FIG. 16A) and HSA-LR mice treated with EEV-PMO (FIG. 16B) stained for CUG foci (red) and nuclei (blue). Qualitative and quantitative assessment (FIG. 16C) showed that EEV-PMO treatment reduced number of nuclei had CUG foci.

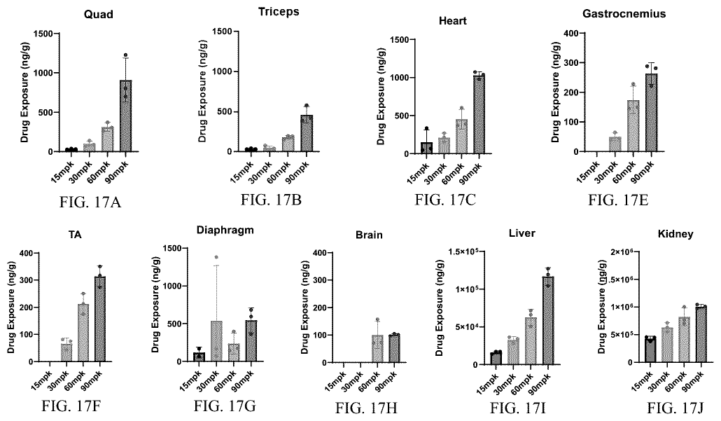

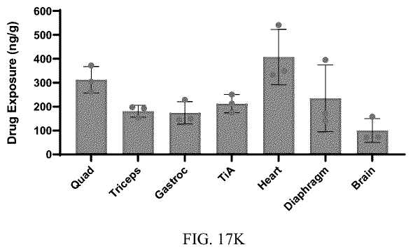

Drug Exposure. Drug exposure was studied using LC-MS. FIGS. 17A-17C and 17E-17J show a dose dependent response for PMO-EEV exposure in the quadriceps (FIG. 17A), triceps (FIG. 17B), heart (FIG. 17C), gastrocnemius (FIG. 17E), tibialis anterior (TA; FIG. 17F), liver (FIG. 17I), and kidney (FIG. 17J). No dose-dependent response was observed in the diaphragm (FIG. 17G). The EEV-PMO was not detected in the brain except at the 60 mpk and 90 mpk dosage levels. FIG. 17K shows drug exposure of various tissues at the 60 mpk dosage level.

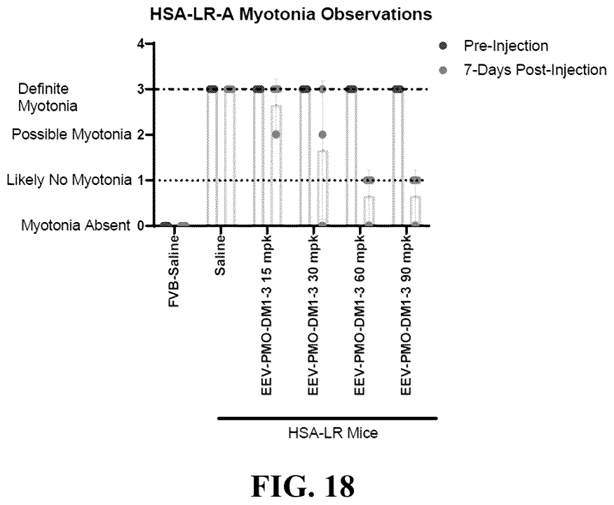

Myotonia Response: A dose dependent myotonia reduction in HSA-LR mice 7 days after treatment with EEV-PMO-DM1-3at 15, 30, 60 and 90 mpk was observed (FIG. 18). Myotonia is likely ameliorated one week after treatment with EEV-PMO-DM1-3. HSA-LR mice treated with a single dose of 90 mpk EEV-PMO-DM1-3 did not exhibit obvious signs of hind limb myotonia after induction.

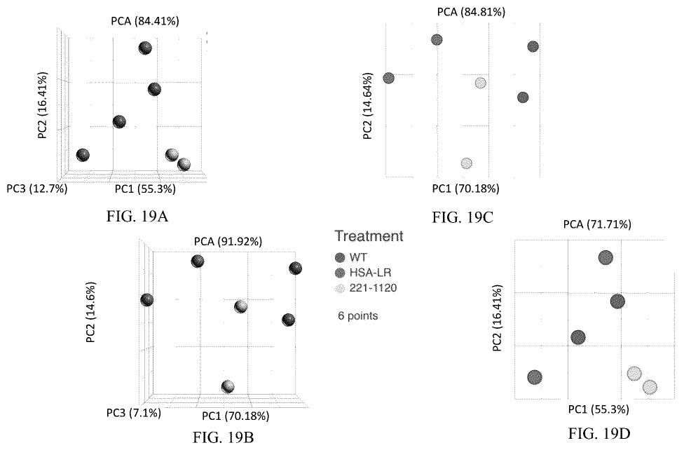

RNAseq Data Analysis. FIG. 19A-19D show the results of a principal component analysis. Principal component analysis can be used to reveal the similarity between samples based on the distance matrix. This type of plot is useful for visualizing the overall effect of experimental covariates and batch effects. The x-axis is the direction that explains the most variance and the y-axis is the second most. The percentage of the total variance per direction is shown as PCA. The wild type and HSA-LR mice are in distinct groups. Gene expression in the gastrocnemius muscle of HSA-LR mice treated with PMO-EEV was shifted toward that of wild type mice.

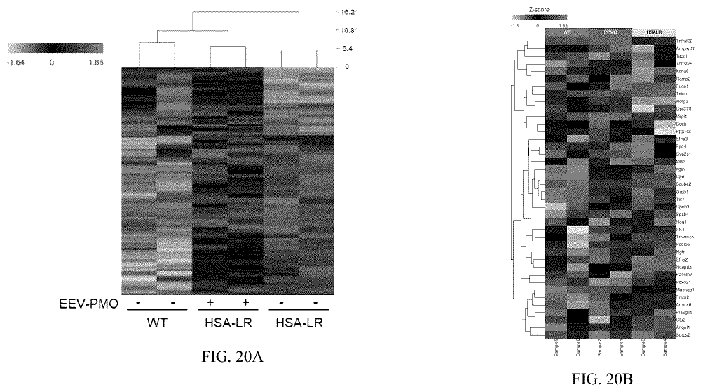

FIG. 20A is a heatmap showing differentially expressed genes (by Z-score) from three treatment groups: 1) WT mice; 2) HSA-LR mice; and 3) HSA-LR+EEV-PMO 221-1120 (60 mpk). A total of 956 (p<0.5) genes differentially expressed between the treatment groups, indicating a difference between the wild type mice (WT) and the disease model mice (HSA-LR).

Treatment with EEV-PMO (HSA-LR (+,+)) resulted in global gene expression correction, shifting away from a disease profile (in red, HSA-LR (−,−)) and toward that of wild type mice (WT above).

FIG. 20B is a heatmap used to visualize the expression profile of 40 of the 43 genes found to have more than 7 CTG repeats from a BLAST analysis. Three of the CTG repeat gene found in the Blast analysis (Crb2, Hsd3b6, and Inhbe) were not included due to low reads number. This analysis is useful to identify co-regulated genes across the treatment conditions.

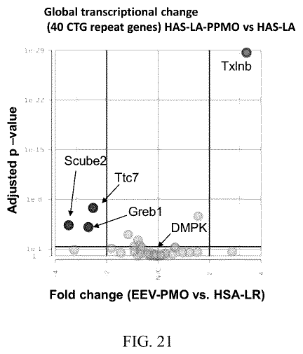

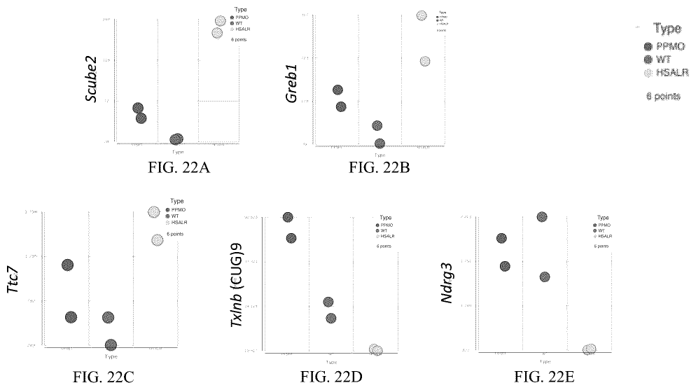

FIG. 21 shows a volcano plot of the global transcriptional change across the EEV-PMO treated group and the HSA-LA group. Each data point in the scatter plot represents a gene. The fold change of each gene is represented on the x-axis and the log 10 of its adjusted p-value is on the y-axis. Genes with an adjusted p-value less than 0.05 and a fold change greater than 2 are indicated by red dots. These represent up-regulated genes. Genes with an adjusted p-value less than 0.05 and a fold change less than −2 are indicated by blue dots. These represent down-regulated genes. Three genes were found to be significantly downregulated (Txlnb, Scube2 and Greb1) and one gene was significantly upregulated (Txlnb). Most transcripts containing at least (CUG)7 (SEQ ID NO: 413) were not significantly influenced. PCA Analysis of these genes showed that Scube2 (FIG. 22A), Greb1 (FIG. 22B), Ttc7 (FIG. 22C), Txlnb(CUG)9, and Ndrg3 (FIG. 22E) showed correction when treated with EEV-PMO. Txlnb is overcorrected by treatment (FIG. 22D).

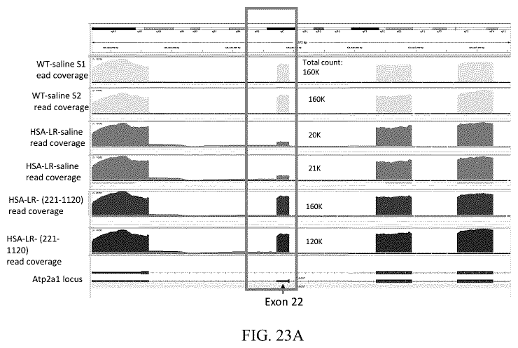

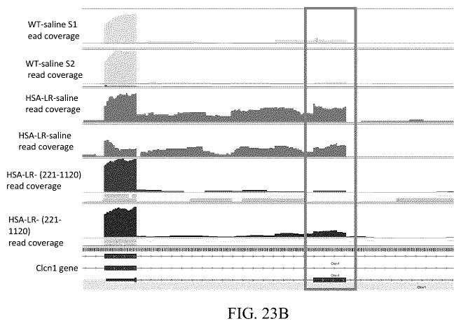

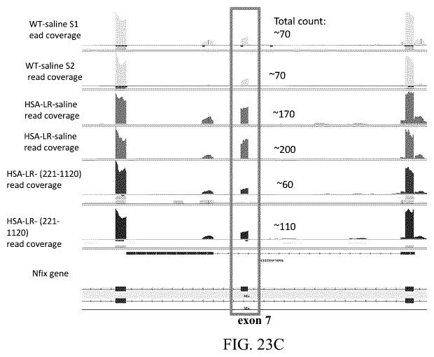

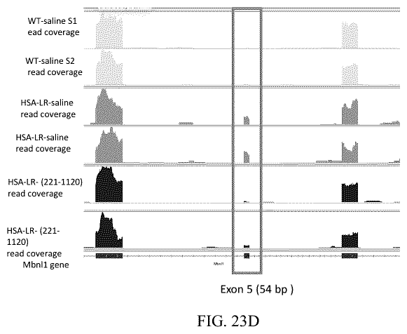

FIGS. 23A-23D show the transcriptome data for various genes and various treatment groups. The HSA-LR+EEV-PMO 221-1120 treatment group showed correction of the inclusion of exon 22 of Atp2al (FIG. 23A), exclusion of exon 7 of Clcn1(FIG. 23B), exclusion of exon 7 of Nfix (FIG. 23C), and the exclusion of exon 7 of Mbn11 (FIG. 23D).

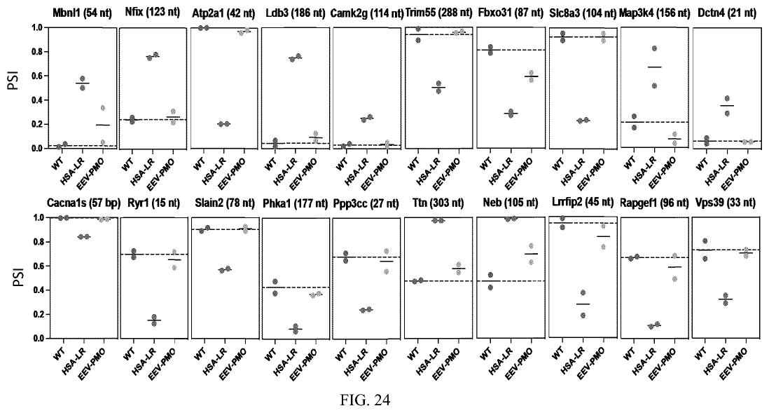

FIG. 24 show the percent spliced (PSI) of individual exons for various genes. The genes are MBNL-1 responsive splicing biomarkers (e.g., downstream genes). The choice of MBNL-1 dependent biomarkers was selected based on the dynamic range between the wildtype and disease groups as described in the literature. The HSA-LR+EEV-PMO 221-1120 treatment group showed correction for exon inclusion/exclusion for all of the 20 genes of interest including Mbn11, Nfix, Atp2al, Ldb3, Camk2g, Trim55, Fbox31, Slc8a3, Map3k4, Dctn4, Cacnals, Ryrl, Slain2, Phkal, Ppp3cc, Ttn, Neb, lrrfip2, Rapgefl, and Vsp39.

### Example 5. Evaluation of PMO-EEV 221-1120 in a DM1 mouse model second DM1 mouse model

PMO-EEV DM1-3 (221-1120; see Example 4 for the sequence) was evaluated in a second DM1 mouse model using method similar to those described in Example 4.

Experimental. Seven-week-old HSA-LR mice were administered a single dose 80 mpk of EEV-PMO-DM1-3 or a 20 mpk dose EEV-PMO-DM1-3 every other week for six weeks (total of 80 mpk over 4 doses) intravenously and tissues were harvested after 1 week to 12 weeks post the single dose or two weeks after the final dose. RT-PCR was used to determine alternative splicing for specific genes (Atp2al, Clcn1, Nfix, MBNL1). Q-PCR was used to determine the reduction of mRNA level of actin-HSA after treatment. LC-mass was used to determine drug level in quadricep, gastrocnemius, tibialis anterior, triceps, diaphragm, heart, kidney, liver, brain, and plasma. Myotonia reduction was recorded 7 days after treatment with the EEV-PMO-DM1-3 compound.

Results. Similar trends to those observed in Example 4 were observed for the rescue of splicing for Atp2al, Clcn1, Nfix, and MBNL1 in the tibialis anterior, gastrocnemius, tricep, and quadricep tissues (data not shown). Additionally, similar trends to those observed in Example 4 were observed for the HSA mRNA knockdown tibialis anterior, gastrocnemius, tricep, and quadricep tissues both 1-week and 4-weeks post treatment (data not shown).

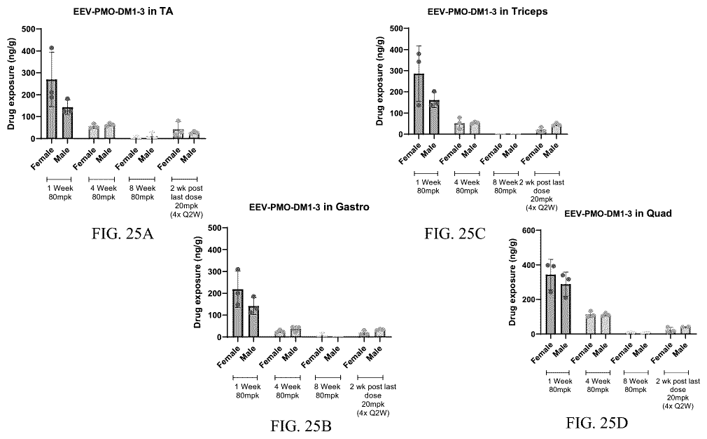

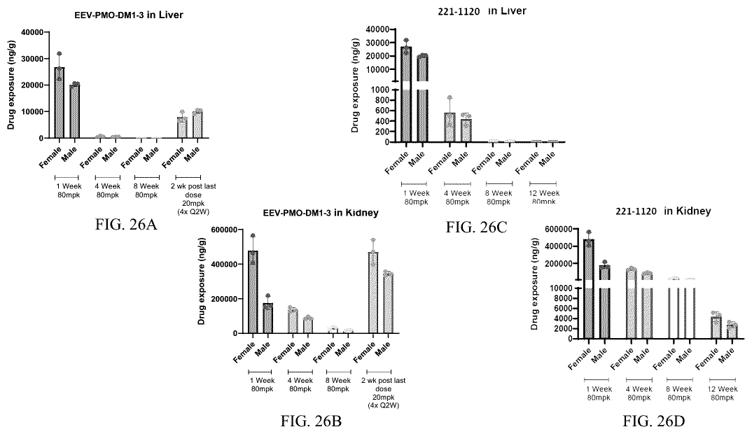

FIG. 25A-25D are plots showing a decrease in drug level with 80 mpk EEV-PMO-DM1-3 after 1 week to 8 weeks in the tibialis anterior (FIG. 25A), gastrocnemius (FIG. 25B), triceps (FIG. 25C), and quadricep (FIG. 25D) tissues. EEV-PMO-DM1-3 (60 mpk oligo, 80 mpk whole drug) fully correct mis-splicing in gastrocnemius, triceps, tibialis anterior and quadricep post 1 week treatment. FIG. 26A-26B are plots showing a decrease in drug levels was observed with the single 80 mpk dose of EEV-PMO-DM1-3 after 1 week to 4 weeks, to 8 weeks, and to 12 weeks in the liver. A relatively higher amount of EEV-PMO-DM1-3 in the liver was observed 2 weeks post the last dose of the 6-week dosing regime when compared to 4 weeks post the single dose regime.

FIG. 26C-26D shows a decrease in drug levels was observed with the single 80 mpk dose of EEV-PMO-DM1-3 after 1 week to 4 weeks, to 8 weeks, and to 12 weeks in the kidney. A relatively low amount of EEV-PMO-DM1-3 in the kidney was also observed from 2 weeks post the last dose of the 6-week dosing regime when compared to 4 weeks post the single dose regime. At 12 weeks post the single dose of EEV-PMO-DM1-3, the drug was still present in the kidney but not in the liver.

Subjective myotonia observations were made and shown in Table 15. The multi-dosing regime (Q2W) showed no signs of myotonia rescue after two weeks post treatment. There was a mixed effect on myotonia in mice treated with the single 80 mpk dose that disappeared by 12

TABLE 15
Myotonia Observations

| | Group | | | | | | | | | |
|---|---|---|---|---|---|---|---|---|---|---|
| | Pre- | | 1 week | | 4-week | | 8-week | | 12-week | |
| | dosing | | post dose | | post dose | | post dose | | post dose | |
| | Gender | | | | | | | | | |
| | F | M | F | M | F | M | F | M | F | M |
| FVB | 0 | 0 | 0 | 0 | 0 | 0 | 0 | 0 | 0 | 0 |
| HSA-LR | + + | + + | + + | + + | + + | + + | + + | + + | + + | + + |
| 221-112 80- mpk | + + | + + | + | + | + | + | 0 | + | + + | + + |
| 221-1120 20 mpk | + + | + + | NA | NA | NA | NA | + + | + + | NA | NA |
| Q2W | | | | | | | | | | |

F = female;
M = male;
0 = no mice displayed myotonia;
+ = mixed myotonia, some mice displayed reduced myotonia;
+ + = all mice displayed myotonia

A similar experiment was performed to evaluate intravenous administration of EEV-PMO-DM1-3 for a longer duration and at a higher dose. Eight-week-old HSA-LR mice were treated with 40 mpk, 60 mpk, 80 mpk, or 120 mpk of EEV-PMO-DM1-3 intravenously and tissues were harvested after 4 weeks to 12 weeks. RT-PCR was used to determine alternative splicing for specific genes (Atp2al, Clcn1, Nfix, MBNL1). Myotonia reduction was recorded 7 days after treatment with EEV-PMO-DM1-3. Similar trends to those observed in Example 4 were observed for the rescue of splicing for Atp2al, Clcn1, Nfix, and MBNL1 in the tibialis anterior gastrocnemius tissues (data not shown).

Subjective myotonia observations were made and shown in Table 16. Females displayed more myotonia than males. There are no signs of myotonia in both male and female mice dosed with 120 mpk after 8 weeks post treatment.

TABLE 16
Myotonia Observations

| | Group | | | | | | | | | |
|---|---|---|---|---|---|---|---|---|---|---|
| | Pre- | | 1 week | | 4-week | | 8-week | | 12-week | |
| | dosing | | post dose | | post dose | | post dose | | post dose | |
| | Gender | | | | | | | | | |
| | F | M | F | M | F | M | F | M | F | M |
| FVB | 0 | 0 | 0 | 0 | 0 | 0 | 0 | 0 | 1 0 | 0 |
| HSA-LR | + + | + + | + + | + + | + + | + + | + + | + + | + + | + + |
| 221-1120 40 mpk | + + | + + | + | + + | + + | + + | NA | NA | NA | NA |
| 221-1120 60 mpk | + + | + + | + | 0 | + | + | NA | NA | NA | NA |
| 221-1120 80 mpk | + + | + + | 0 | 0 | + | + | + | 0 | + | 0 |
| 221-1120 120 mpk | + + | + + | 0 | 0 | 0 | 0 | 0 | 0 | 0 | 0 |

F = female;
M = male;
0 = no mice displayed myotonia;
+ = mixed myotonia, some mice displayed reduced myotonia;
+ + = all mice displayed myotonia

### Example 6. Treatment of Patient derived DM1 cells with EEV-PMO-DM1-3

PMO-EEV DM1-3 (221-1120; see Example 4 for the sequence) was evaluated in DM1 patient derived myoblasts.

Experimental. Patient myoblasts were treated with 30 micromolar of DM1-3 throughout four days of differentiation. Splicing correction was assessed by one-step RT-PCR and Labpchip (Plotted mean±SD; n=4). HCR—FISH and sequestered MBNL 1 protein detection assays were used to detect RNA CUG foci. Results: EEV-PMO-DM1-3promotes significant biomarker splicing correction and a reduction in nuclear foci in DM1 patient-derived muscle cells.

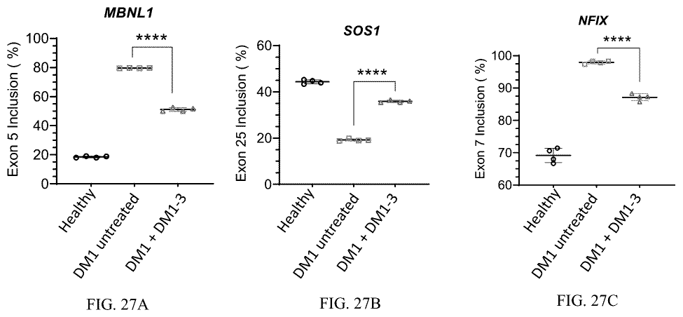

Results. FIG. 27A-27C are plots showing that EEV-PMO-DM1-3 promotes significant biomarker splicing correction (MBLN1, SOS1, and NFIX) in DM1 patient-derived muscle cells.

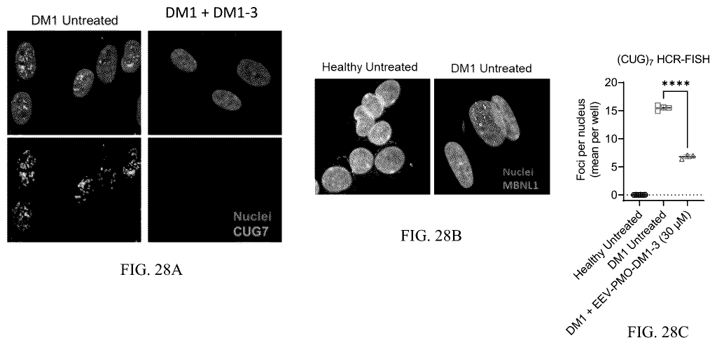

Additionally, treatment with DM1-3 resulted in the reduction of nuclear foci in DM1 patient-derived muscle cells (FIG. 28A-28C).

### Example 7. Cytotoxicity Screening of EEV-PMO-DM1-3 in Renal Cells

PMO-EEV DM1-3 (221-1120; see Example 4 for the sequence) was evaluated in human renal cells.

Experimental. Human Primary Renal Proximal Tubular Epithelial Cells (RPTECs) were exposed to varying concentrations (1:2 serial dilution in saline with a final dilution factor of 4x from about 6 μM to about 800 μM) of PMO-DM1 and EEV-PMO-DM1-3 for 24 hours and screened for viability using a CELLTITER-GLO luminescent viability assay. Melittin was used as a positive control at 16.6 μM.

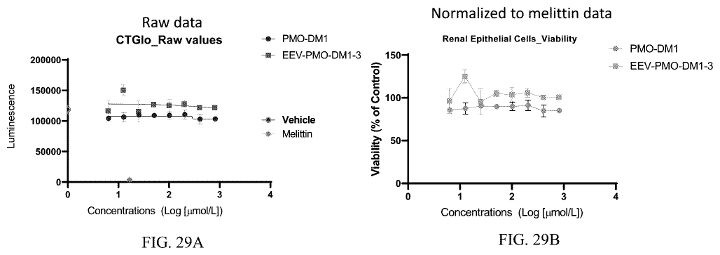

Results. FIG. 29A-29B show that PMO-DM1 or its conjugated EEV-PMO-DM1-3 did not show any toxicity even with the highest concentration of 817 μM or 797 μM, respectively.

### Example 8. Assessment of PMO-EEV 221-1113 for ability to correct mis-splicing events and downstream splicing in immortalized cells DM1 patients and HeLa-480 cells

Immortalized DM1 patient-derived (2,600 CUG repeats) muscle cells and HeLa-480 (DM1 model cell line, see Example 1) were treated with the EEV-PMO construct 221-1113 and analyzed for correction of aberrant splicing and foci quantification. EEV 1113 is Ac-PKKKRKV-miniPEG-K(cyclo(Ff-Nal-GrGrQ)-PEG₁₂—OH (SEQ ID NOS 634-635, respectively, in order of appearance; Ac-(SEQ ID NO: 42)-miniPEG-K(cyclo(SEQ ID NO: 80)-PEG₁₂—OH). EEV-PMO 221-1113 is EEV 1113 conjugated to PMO sequence 221 (5′-CAG CAG CAG CAG CAG CAG CAG-3′ (SEQ ID NO: 638; all PMO monomers) via amide bond chemistry.

Experimental. Methods similar to those described in Example 1 and Example 3 were used.

### Results.

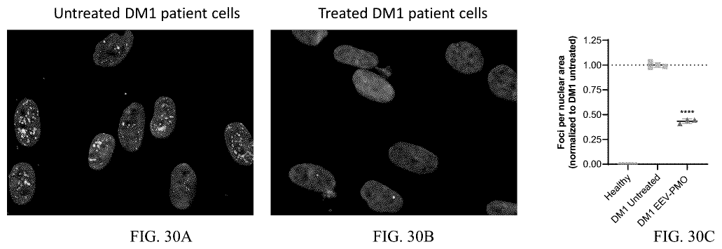

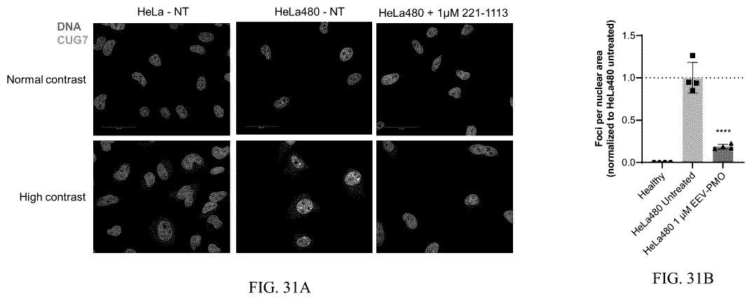

RNA CUG Foci analysis. Cells were stained for nuclei (Hoeschet, blue) and for RNA CUG repeat foci (green) and imaged. A reduction in RNA CUG foci was observed between untreated DM1 patient cells and EEV-PMO treated DM1 cells (FIG. 30A-30C). Similarly, a reduction in RNA CUG foci was observed between untreated HeLa-480 cells and EEV-PMO HeLa-480 cells (FIG. 31A-31B).

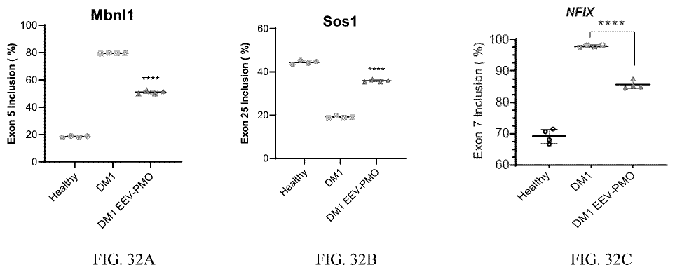

Correction of downstream splicing. PMO-EEV treated DM1 patient derived cells and HeLa-480 cells were analyzed for percent exon 5 inclusion for MBLN1, percent exon 25 inclusion for SOS1, and percent inclusion of exon 7 for NFIX. Treatment with EEV-PMO resulted in a rescue of splicing events for Mbn11 (FIG. 32A), SosI (FIG. 32B), and NFIX (FIG. 32C).

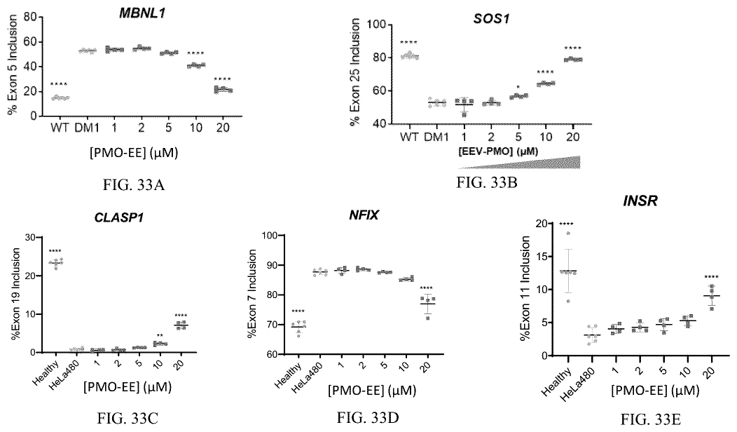

Additionally, HeLa-480 cell treated EEV-PMO showed a dose dependent correction of MBNL1 (FIG. 33A) splicing and the downstream missplicings of SOS1 (FIG. 33B), CLASP1 (FIG. 33C), NFIX (FIG. 33D), and INSR (FIG. 33E) in a dose dependent manner.

### Example 9. Evaluation of EEV-PMO 221-1106 in a second DM1 mouse model

A DM1 mouse model was done to study the effect of EEV-PMO 221-1106 on the splicing and mRNA levels of downstream genes.

Experimental. Human skeletal actin long repeat (HSA-LR) transgenic mice were used as the DM1 disease model. Similar methods to those described in Example 5 were used.

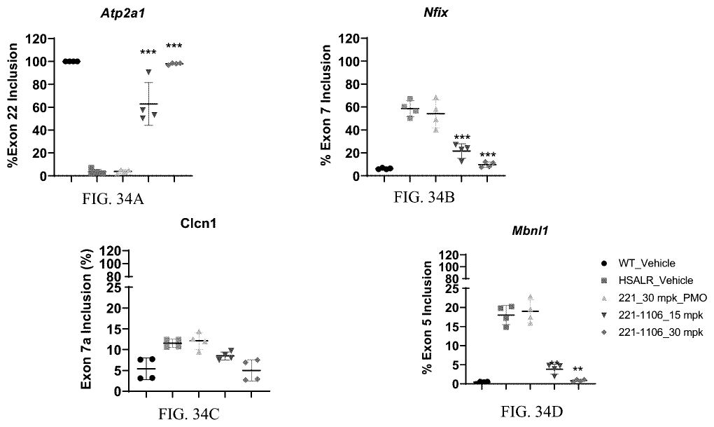

Results. FIGS. 34A-34D show a dose dependent correction of the inclusion of exon 22 in Atp2al (FIG. 34A), exon 7 in Nfix (FIG. 34B), exon 7A in Clcn1 (FIG. 34C), and Mbn11 (FIG. 34D) in the gastroenemius of mice treated with various concentrations of EEV-PMO 221-1106.

Treatment with PMO 221 alone did not result in correction of splicing.

### Example 10. DM1 mouse model to study effect of different lengths of CUG repeats in PMOs

A DM1 mouse model was done to study the effect of PMO-EEV 221-1121 (PMO has7 CAG repeats (SEQ ID NO: 146), 21-mer) and PMO-EEV 0325-1121 (PMO has 8 CAG repeats (SEQ ID NO: 642), 24-mer) on the splicing and mRNA levels of downstream genes. PMO-EEV 221-1121 is PMO 221 (5′-CAG CAG CAG CAG CAG CAG CAG-3′; SEQ ID NO: 638; all PMO monomers) conjugated to EEV 1121 (Ac-PKKKRKV-miniPEG₂-Lys(cyclo[GfFGrGrQ])-PEG₁₂—OH (SEQ ID NOS 636-637, respectively, in order of appearance; Ac-(SEQ ID NO: 42)-miniPEG₂-Lys(SEQ ID NO:74)-PEG₁₂—OH ) via amide chemistry. PMO-EEV 0325-1121 is PMO 0325 (5′-CAG CAG CAG CAG CAG CAG CAG-CAG-3′; SEQ ID NO: 642; all PMO monomers) conjugated to EEV 1121 via amide chemistry.

Experimental. Human skeletal actin long repeat (HSA-LR) transgenic mice were used as the DM1 disease model. Briefly, HSA-LR mice were dosed with 20 mpk, 40 mpk, or 60 mpk of either 0221-1121 or 0325-1121 via intravenous injection into the tail vein. One week post injection, mice were sacrificed, and tissue was collected. Other experimental methods are similar to those described in Example 5 were used.

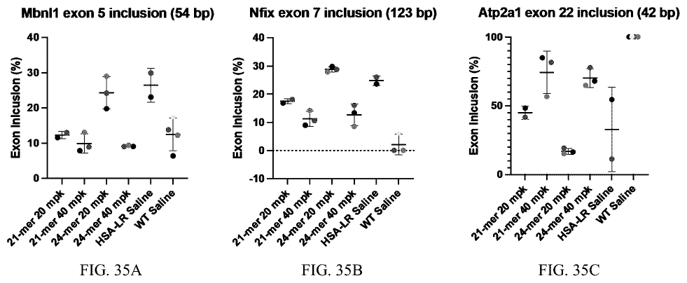

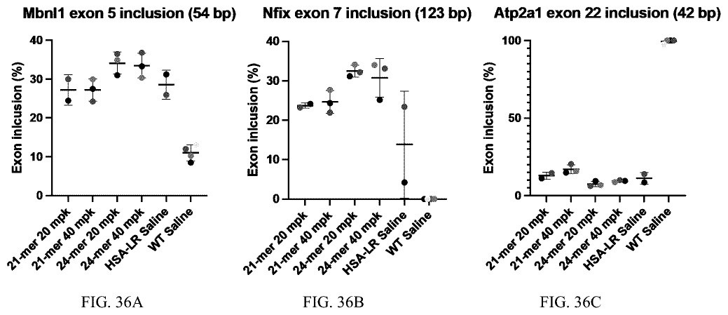

Results. 0221-1121 (21-mer) was more effective in correcting exon splicing in Mbn11 (FIG. 35A), Nfix (FIG. 35B), and Atp2al (FIG. 35C) than 0325-1121 (24-mer) in the tibialis anterior tissue. This result was unexpected. It was expected that the 24-mer would be more effective as it would have a higher hybridization efficiency and higher thermal melting temperature. In the gastrocnemius tissue, the differences were less pronounced as shown in FIGS. 36A-36C. Subjective myotonia observations were made using the male mice (Table 17). Mixed myotonia was observed at 1 week post treatment for the 21-mer at 40 mpk, similar to the results in Table 11.

TABLE 17
Myotonia Observations

| | Pre- | 1 week post | 4 week post |
|---|---|---|---|
| Group | dosing | dose | dose |
| Gender | M | M | M |
| FVB | 0 | 0 | NA |
| HSA-LR | + + | + + | NA |
| 221-112 20 mpk | + + | + + | NA |
| 221-1120 40 mpk | + + | + | |
| 325-1120 20 mpk | + + | + + | NA |
| 325-1120 40 mpk | + + | + + | |

M = male;
0 = no mice displayed myotonia;
+ = mixed myotonia, some mice displayed reduced myotonia;
++ = all mice displayed myotonia

### Example 11. Pharmacokinetic studies of the EEV-PMO 221-1120 in CD1 mice

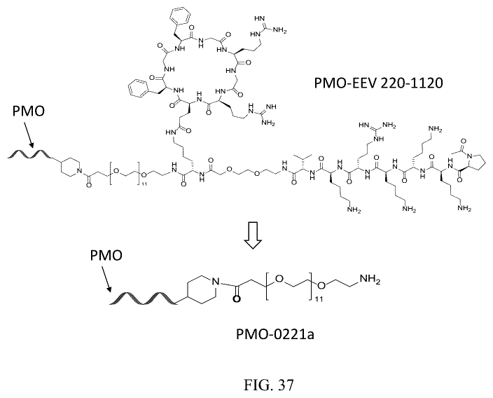

A CD1 mouse model was used to study the plasma, kidney, and tibialis anterior drug exposure (AUC) to the EEV-PMO construct 221-1120 (see Example 4 for the sequence) and PMO-0221a, the major metabolite of 221-1120 (see FIG. 37) was also measured.

Experimental. Five- to seven-week-old CD1 mice were treated with 80 mpk of the EEV-PMO construct 221-1120 via intravenous injection. Mice were bled and/or scarified at various time points.

Results. Table 18, Table 19, and Table 20 show the pharmacokinetic properties observed in the plasma, kidney, and tibialis anterior, respectively. For the tables: AUCₗₐₛₜ=area under the curve from zero to last quantifiable concentration; D=dose; Cₘₐₓ=maximum serum or plasma concentration; Tₘₐₓ=time to reach Cₘₐₓ; CL=total plasma, serum, or blood clearance; ti/2=elimination half-life; Vₛₛ=apparent volume of distribution at equilibrium; Qₕ=hepatic blood flow (ml/min/kg).

The AUC values for the metabolite is ˜1000-fold lower in the tibias anterior compared to the kidney. The metabolite mean residence time (MRT) values in plasma may be directly related to tissue MRT values as a result of moving from tissues to plasma before urinary excretion.

TABLE 18
Plasma pharmacokinetic properties

| | 221-1120 | PMO-0221a |
|---|---|---|
| AUCₗₐₛₜ (nM * hr) | 9290 | 953 |
| AUCₗₐₛₜ/D | 1121 | 115 |
| Cₘₐₓ (nM) | 16217 | 12 |
| Cₘₐₓ/D | 1956 | 1.4 |
| Tₘₐₓ (hr) | 0.1 | 24 |
| CL (mL/min/kg) | 15 | — |
| Qₕ (%) | 16 | — |
| t_{1/2} (hr) | 19 | 68 |
| MRTₗₐₛₜ (hr) | 1.2 | 60.0 |
| Vₛₛ (mL/kg) | 1325 | — |

TABLE 19
Kidney pharmacokinetic properties

| | 221-1120 | PMO-0221a |
|---|---|---|
| AUCₗₐₛₜ (pmol/g * hr) | 51609 | 10113709 |
| AUCₗₐₛₜ/D | 6225 | 1219865 |
| Cₘₐₓ (pmol/g) | 8754 | 105053 |
| Cₘₐₓ/D | 1056 | 12671 |
| Tₘₐₓ (hr) | 4 | 24 |
| t_{1/2} (hr) | 5 | 87 |
| MRTₗₐₛₜ (hr) | 8 | 68 |

TABLE 20
Tibialis anterior pharmacokinetic properties

| | 221-1120 | PMO-0221a |
|---|---|---|
| AUCₗₐₛₜ (pmol/g * hr) | — | 9140 |
| AUCₗₐₛₜ/D | — | 1102 |
| Cₘₐₓ (pmol/g) | 34 | 162 |
| Cₘₐₓ/D | 4 | 20 |
| Tₘₐₓ (hr) | 4 | 24 |
| t_{1/2} (hr) | — | 48 |
| MRTₗₐₛₜ (hr) | — | 50 |

### Example 12. Evaluation of PMO-EEV 221-1120 in a third DM1 mouse model

A DM1 mouse model study similar to Examples 5 and 9 was conducted to evaluate the effect of various doses of PMO-EEV 221-220 (see Example 4 for the sequence) in HSA-LR mice.

Experimental. Eight-week-old HSA-LR mice were administered 40, 60, 80 or 120 mpk of PMO-EEV 221-1120 intravenously and tissues were harvested after 4 to 12 weeks. RT-PCR was used to determine alternative splicing for specific genes (Atp2al, Clcn1, Nfix, MBNL1). LC-mass was used to determine drug level in Quad, gastro, TA, Triceps, diaphragm, heart, kidney, liver, brain, plasma. RNA-seq was used to determine the transcription level change between a treated disease model, an untreated disease model and wild-type. Q-PCR was used to determine the reduction of mRNA level of actin-HSA after treatment.

Results. Fluorescence imaging was used to determine RNA Foci reduction after treatment with the EEV-oligo compound (data no shown). Myotonia reduction was recorded 7 days after treatment with the EEV-oligo compound (data not shown). The results of these experiments show similar trends to the mice treated with PMO-EEV 221-1120 in Example 5 and Example 14.

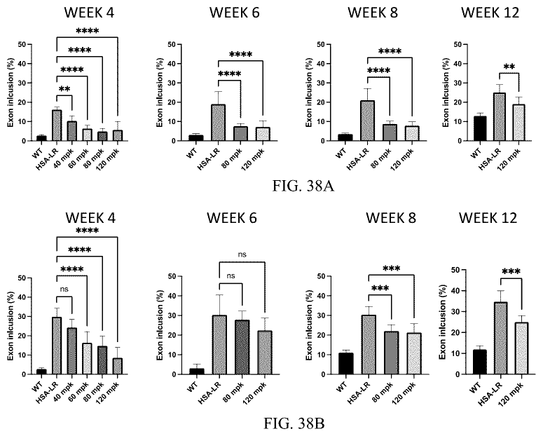

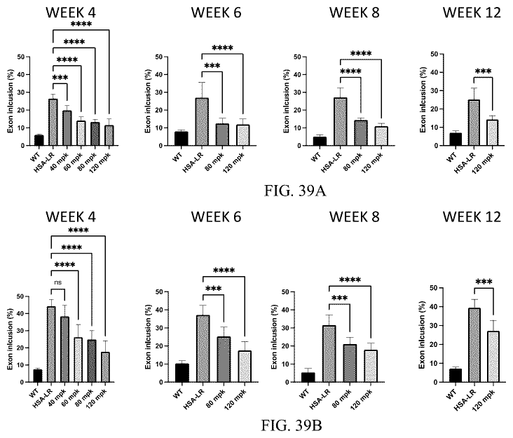

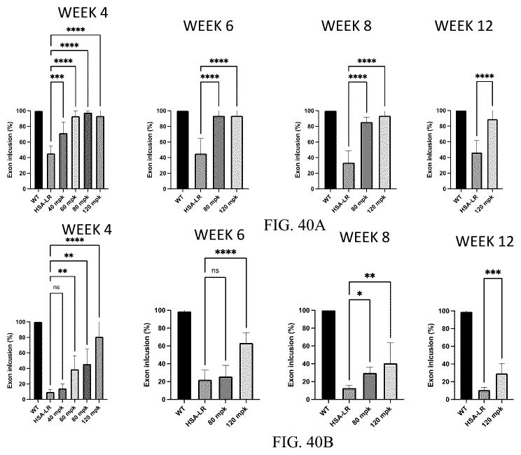

MBNL1, NFIX, and ATP2A1 splicing correction was observed in the tibialis anterior (FIGS. 38A, 39A, 40A) and gastrocnemius (FIGS. 38B, 39B, 40B) at various doses of the EEV-PMO. MBNL1, NFIX, and ATP2Alsplicing correction was observed in both the tibialis anterior and gastrocnemius 12 weeks post treatment with 120 mpk EEV-PMO.

### Example 13. Evaluation of EEV-PMO 221-1120 in HeLa480 cells

HeLa480 cells were treated with various concentrations of EEV-PMO 221-1120 (see Example 4 for the sequence) and analyzed for CUG repeat foci, selective r(CUG) reduction, and downstream splicing correction of MBNL1 and SOS1.

Experimental. Hela480 cells were constructed as described in earlier Examples. RT-PCR and foci staining was performed similar to other Examples described herein.

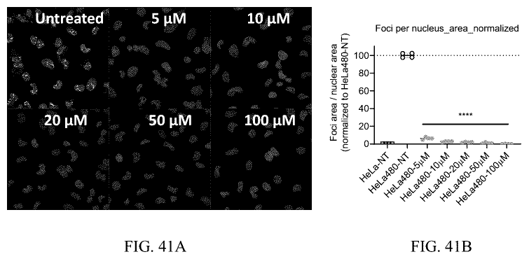

Results. FIG. 41A-41B shows example images of control cells (untreated) and cells treated with 5 μM, 10 μM, 20 μM, 50 μM, and 100 μM of EEV-PMO 221-1120. There is a reduction in CUG foci (green) in the treated HeLa480 group when compared to the untreated HeLa480 cells.

FIG. 41B is a plot quantifying the foci per nuclear area. FIG. 41A-41B show that EEV-PMO 221-1120 can reduce nuclear CUG RNA foci. Almost a complete reduction is observed in the 5 μM dose.

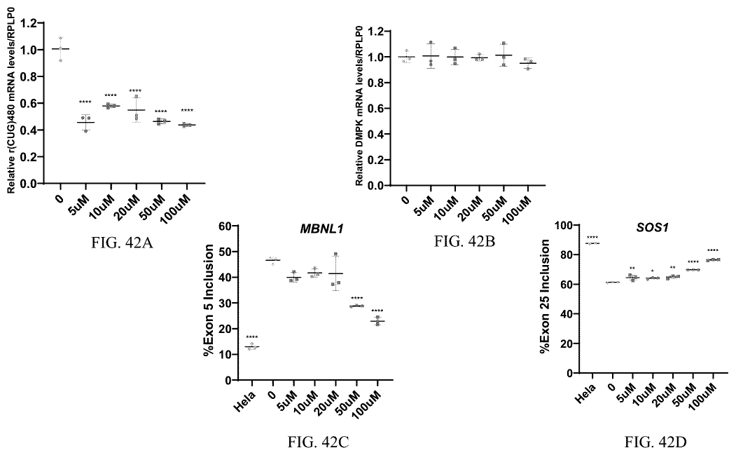

FIG. 42A-42B indicate the EEV-PMO 221-1120 treatment can selectively knockdown repeat expansion-containing DMPK transcript in the HeLa480 cell line.

FIG. 42C-42D show that treatment with EEV-PMO 221-1120 corrected MBNL1 (FIG. 42C) and SOCS1 (FIG. 42D) splicing in a dose dependent manner.

A number of embodiments of the invention have been described. Nevertheless, it will be understood that various modifications may be made without departing from the spirit and scope of the invention. Accordingly, other embodiments are within the scope of the following claims.

1-81. (canceled)

82. A compound comprising:
(a) an antisense compound that is complementary to at least a portion of an expanded CUG repeat in a target mRNA sequence;
(b) a cyclic peptide of the formula: 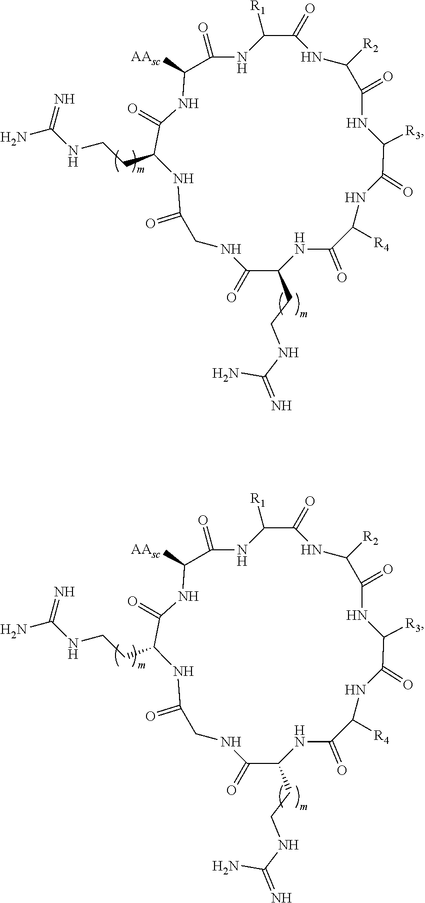
or a protonated form thereof, wherein:
R₁, R₂, and R₃ are each independently H or an amino acid residue having a side chain comprising an aromatic group;
at least two of R₁, R₂, and R₃ are the side chain of phenylalanine;
R₄ is H or an amino acid side chain;
AA_{SC} is an amino acid side chain; and
each m is independently an integer of 0, 1, 2, or 3.

83. The compound of claim 82, wherein one of R₁, R₂, and R₃ is naphthylalanine.

84. The compound of claim 82, wherein one of R₁, R₂, and R₃ is H.

85. The compound of claim 82, wherein R₄ is H.

86. The compound of claim 82, wherein the cyclic cell penetrating peptide is 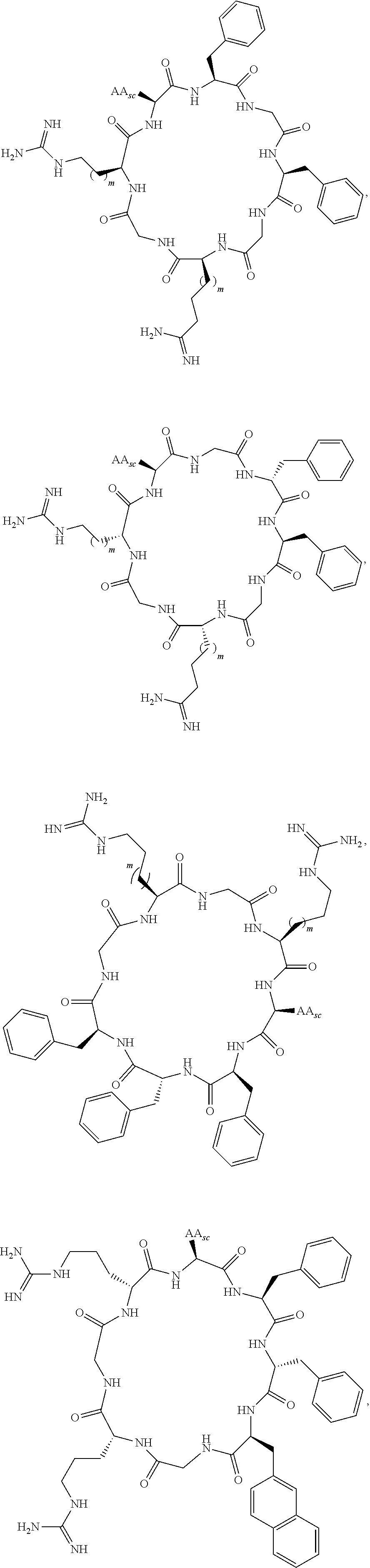
or a protonated form thereof.

87. The compound of claim 82, wherein the compound further comprises an exocyclic peptide.

88. The compound of claim 87, wherein the exocyclic peptide comprises from 2 to 10 amino acids.

89. The compound of claim 87, wherein the exocyclic peptide comprises 2, 3, or 4 lysine residues.

90. The compound of claim 87, wherein the exocyclic peptide comprises at least 2 amino acid residues with a hydrophobic side chain.

91. The compound of claim 87, wherein the exocyclic peptide comprises PGKKRKV.

92. The compound of claim 87, wherein the exocyclic peptide comprises PKKKRKV.

93. The compound of claim 87, wherein the exocyclic peptide comprises PKKKGKV.

94. The compound of claim 87, wherein the exocyclic peptide comprises PKKKRKG.

95. The compound of claim 82, further comprising a linker, wherein the linker conjugates the antisense compound and the exocyclic peptide to the cyclic peptide.

96. The compound of claim 95, wherein the linker has the following structure: 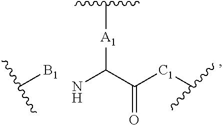
wherein A₁, B₁, and C₁, each independently comprise a hydrocarbon linker, a polyethylene glycol (PEG) linker, or one or more amino acid residue.

97. The compound of claim 95, wherein the linker has the following formula: 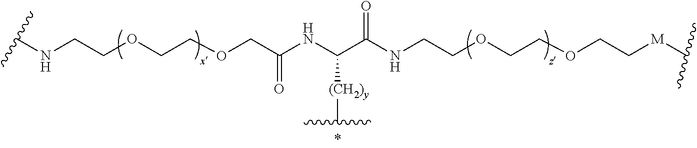
wherein:
x′ is an integer from 1-23;
y is an integer from 1-5;
z′ is an integer from 1-23;
* is the point of attachment to the AA_{SC} of the cyclic peptide;
and M is a bonding group.

98. The compound of claim 97, wherein z′ is 11.

99. The compound of claim 97, wherein x′ is 1.

100. The compound of claim 97, wherein the exocyclic peptide is conjugated to the linker at the amino end of the linker and the antisense compound is conjugated to M.

101. The compound of claim 97, wherein M is —C(O)—.

102. The compound of claim 97, wherein the compound is 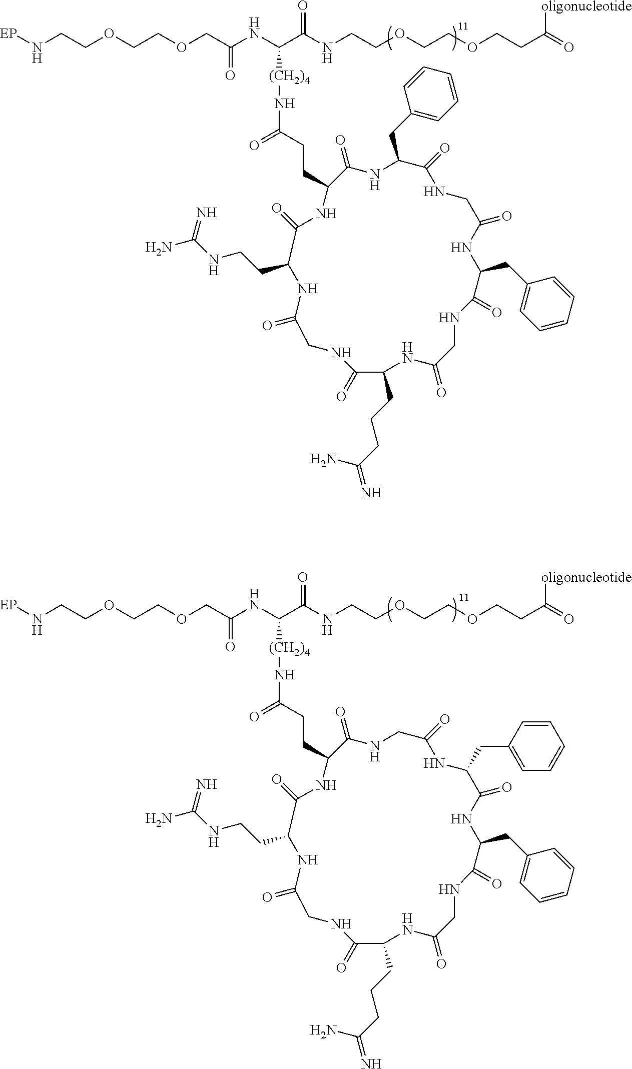 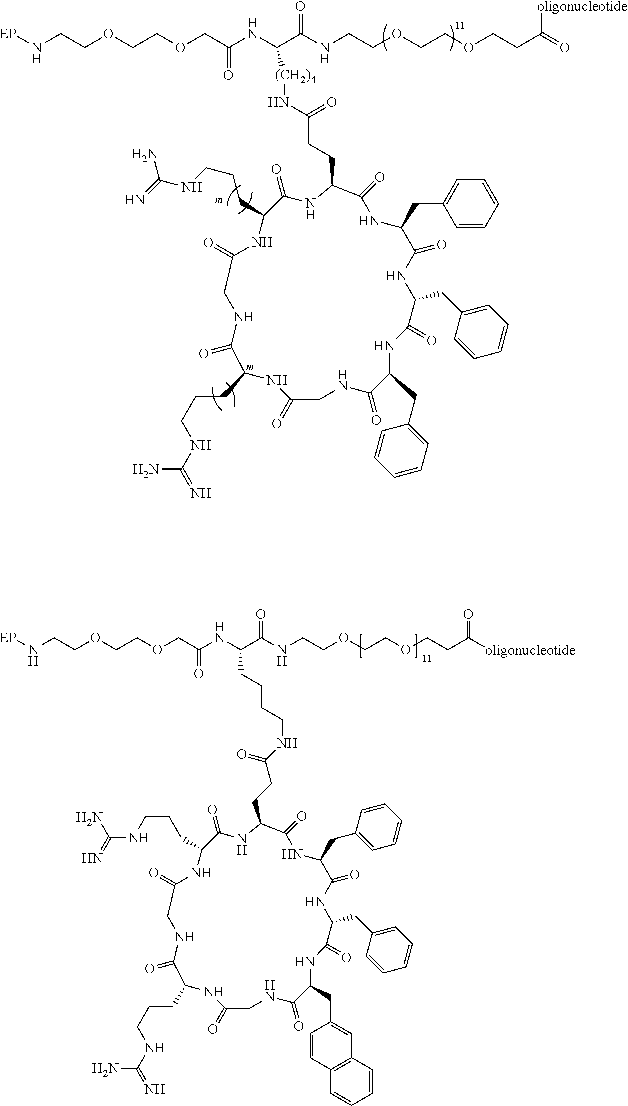
or a protonated form or salt thereof,
wherein EP is the exocyclic peptide, and
oligonucleotide is the antisense compound.

103. The compound of claim 82, wherein the antisense compound comprises AG(CAG)ₙ, G(CAG)ₙ, (CAG)nAG, or (CAG)nA, wherein n is an integer from 1 to 50.

104. The compound of claim 82, wherein the antisense compound comprises 5 to 10 CAG repeats.

105. The compound of claim 82, wherein the antisense compound comprises 5′-CAG CAG CAG CAG CAG CAG CAG CAG-3′.

106. The compound of claim 82, wherein the antisense compound comprises 5′-CAG CAG CAG CAG CAG CAG CAG CAG CAG-3′.

107. The compound of claim 100, wherein the antisense compound comprises 5′-CAG CAG CAG CAG CAG CAG CAG-3′.

108. The compound of claim 82, wherein the antisense compound further comprises any one of
GGGCCTTTTATTCGCGAGGGTCGGG;
GAGGGCCTTTTATTCGCGAGGGTCG;
TGGAGGGCCTTTTATTCGCGAGGGT;
GATGGAGGGCCTTTTATTCGCGAGG;
CAGATGGAGGGCCTTTTATTCGCGA;
GGCAGATGGAGGGCCTTTTATTCGC;
TGGGCAGATGGAGGGCCTTTTATTC;
TTTGGGCAGATGGAGGGCCTTTTAT;
GCTTTGGGCAGATGGAGGGCCTTTT;
GAGCTTTGGGCAGATGGAGGGCCTT;
CAGAGCTTTGGGCAGATGGAGGGCC;
TCCAGAGCTTTGGGCAGATGGAGGG;
AGTCCAGAGCTTTGGGCAGATGGAG;
GGAGTCCAGAGCTTTGGGCAGATGG;
GTGGAGTCCAGAGCTTTGGGCAGAT;
CTGTGGAGTCCAGAGCTTTGGGCAG;
CACTGTGGAGTCCAGAGCTTTGGGC;
GACACTGTGGAGTCCAGAGCTTTGG;
CGGACACTGTGGAGTCCAGAGCTTT;
CGCGGACACTGTGGAGTCCAGAGCT;
ACCGCGGACACTGTGGAGTCCAGAG;
AAACCGCGGACACTGTGGAGTCCAG;
GCAAACCGCGGACACTGTGGAGTCC;
ACGCAAACCGCGGACACTGTGGAGT;
or
CAACGCAAACCGCGGACACTGTGGA.

109. A compound comprising:
(a) an antisense compound that is complementary to at least a portion of an expanded CUG repeat in a target mRNA sequence;
(b) a cyclic peptide of the formula: 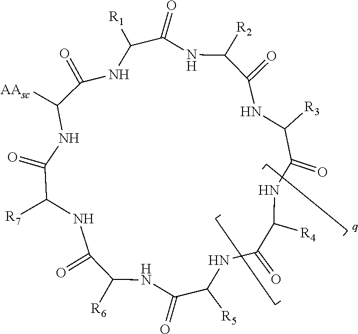
O Rₛ or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
AA_{SC} is an amino acid side chain; and
q is 1, 2, 3 or 4,
wherein at least two amino acids of the cyclic peptide are charged amino acids, at least two amino acids of the cyclic peptide are aromatic hydrophobic amino acids, and at least two amino acids of the cyclic peptide are uncharged, non-aromatic amino acids.

110. The compound of claim 109, wherein at least two of R, R₂, and R₃ are side chains of aromatic hydrophobic amino acids.

111. The compound of claim 109, wherein at least two of R₁, R₂, and R₃ are independently phenylalanine or naphthylalanine.

112. The compound of claim 109, wherein at least two of R₅, R₆, and R₇ are side chains of charged amino acids.

113. The compound of claim 109, wherein at least two of R₅, R₆, and R₇ are independently arginine, homoarginine, N-methylarginine, and N,N-dimethylarginine.

114. The compound of claim 109, wherein q is 1.

115. The compound of claim 109, wherein R₄ is H.

116. The compound of claim 109, wherein the compound further comprises an exocyclic peptide comprising from 2 to 10 amino acids; wherein the exocyclic peptide comprises 2, 3, or 4 lysine residues; and wherein the exocyclic peptide comprises at least 2 amino acid residues with a hydrophobic side chain.

117. The compound of claim 116, wherein the exocyclic peptide comprises PGKKRKV, PKKKRKV, PKKKGKV, or PKKKRKG.

118. The compound of claim 116, further comprising a linker, wherein the linker conjugates the antisense compound and the exocyclic peptide to the cyclic peptide.

119. The compound of claim 118 wherein the linker has the following structure: 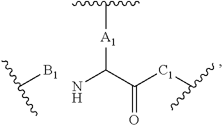
wherein A₁, B₁, and C₁, each independently comprise a hydrocarbon linker, a polyethylene glycol (PEG) linker, or one or more amino acid residue.

120. The compound of claim 118, wherein the linker has the following formula: 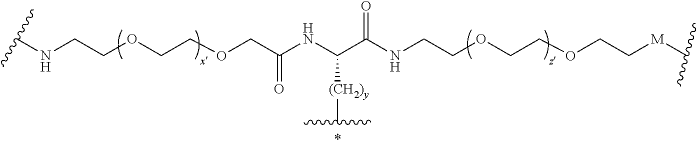
wherein:
x′ is an integer from 1-23;
y is an integer from 1-5;
z′ is an integer from 1-23;
* is the point of attachment to the AA_{SC} of the cyclic peptide;
and M is a bonding group; and
wherein the exocyclic peptide is conjugated to the linker at the amino end of the linker and the antisense compound is conjugated to M.

121. The compound of claim 120, wherein M is —C(O)—.

122. The compound of claim 120, wherein z′ is 11.

123. The compound of claim 120, wherein x′ is 1.

124. The compound of claim 109, wherein the antisense compound comprises 5 to 10 CAG repeats.

125. The compound of claim 109, wherein the antisense compound comprises
5′-CAG CAG CAG CAG CAG CAG CAG CAG-3′;
5′-CAG CAG CAG CAG CAG CAG CAG CAG CAG-3′;
or
5′-CAG CAG CAG CAG CAG CAG CAG-3′.

126. A pharmaceutical composition comprising a carrier and a compound comprising:
(a) an antisense compound that is complementary to at least a portion of an expanded CUG repeat in a target mRNA sequence;
(b) a cyclic peptide of the formula: 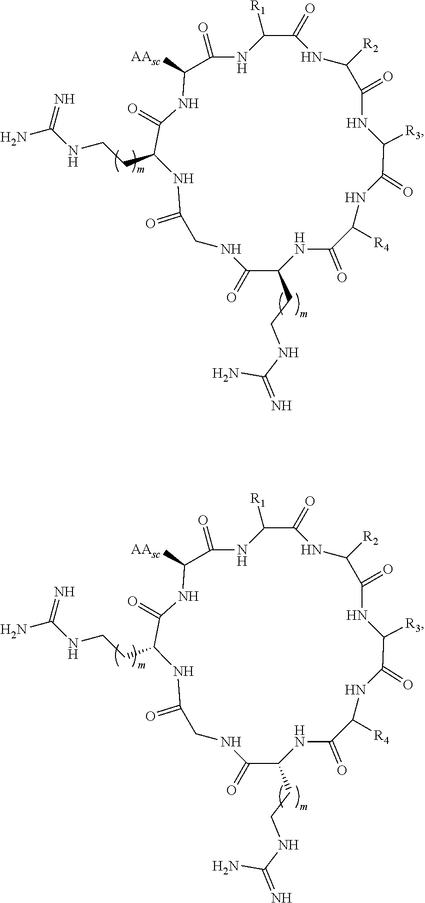
or a protonated form thereof, wherein:
R₁, R₂, and R₃ are each independently H or an amino acid residue having a side chain comprising an aromatic group;
at least two of R₁, R₂, and R₃ are the side chain of phenylalanine;
R₄ is H or an amino acid side chain;
AA_{SC} is an amino acid side chain; and
each m is independently an integer of 0, 1, 2, or 3.

127. The pharmaceutical composition of claim 126, wherein the compound further comprises an exocyclic peptide and the compound is 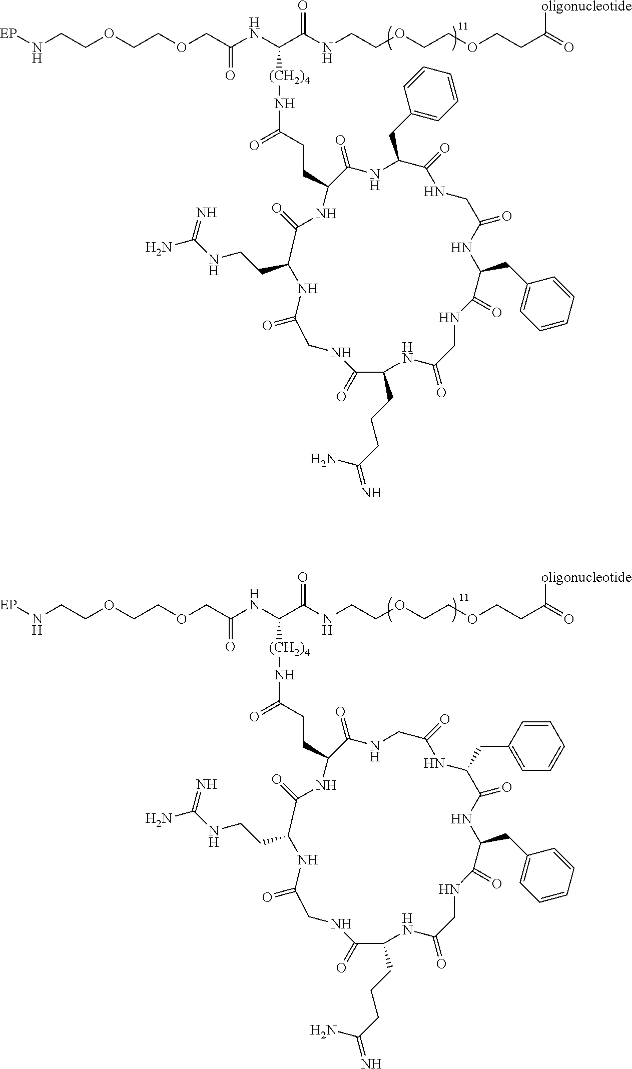 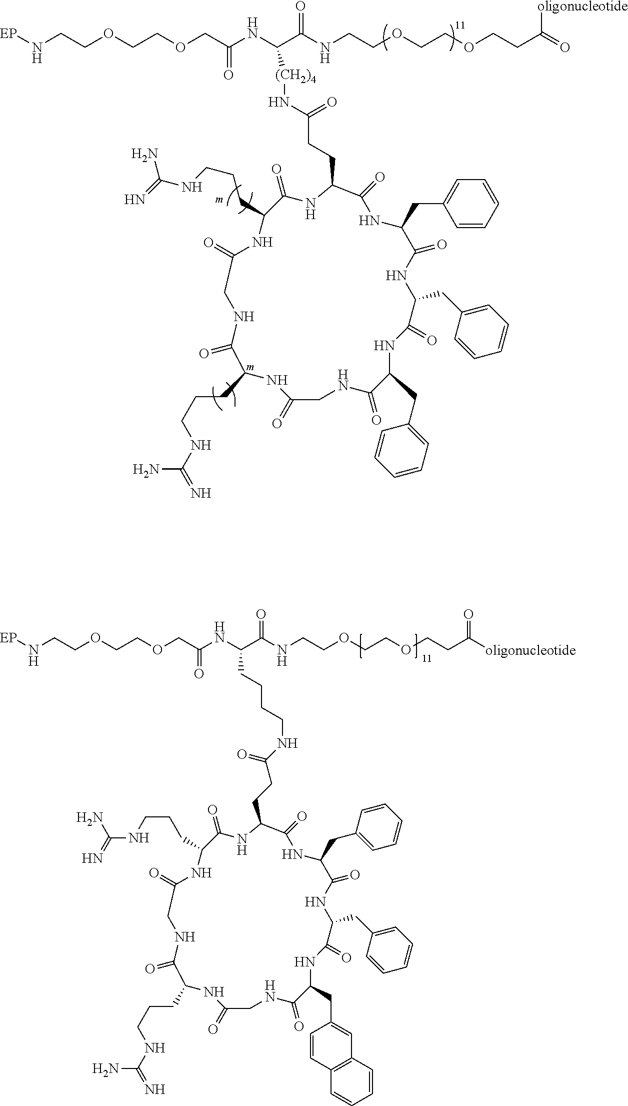
or a protonated form or salt thereof,
wherein EP is the exocyclic peptide, and
oligonucleotide is the antisense compound.

128. The pharmaceutical composition of claim 126, wherein the compound further comprises an exocyclic comprising the sequence comprises PGKKRKV, PKKKRKV, PKKKGKV, or PKKKRKG.

129. The compound of claim 126, wherein the antisense compound comprises
5′-CAG CAG CAG CAG CAG CAG CAG CAG-3′;
5′-CAG CAG CAG CAG CAG CAG CAG CAG CAG-3′;
or
5′-CAG CAG CAG CAG CAG CAG CAG-3′.

130. A pharmaceutical composition comprising a carrier and a compound comprising: an antisense compound that is complementary to at least a portion of an expanded CUG repeat in a target mRNA sequence;
(a) a cyclic peptide of the formula: 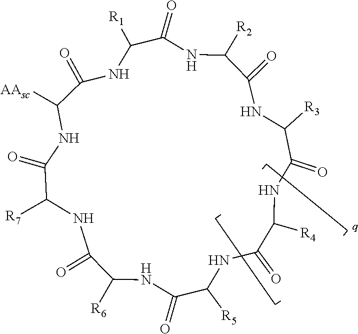
or a protonated form thereof, wherein:
R₁, R₂, and P3 are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
AA_{SC} is an amino acid side chain; and
q is 1, 2, 3 or 4,
wherein at least two amino acids of the cyclic peptide are charged amino acids, at least two amino acids of the cyclic peptide are aromatic hydrophobic amino acids, and at least two amino acids of the cyclic peptide are uncharged, non-aromatic amino acids.

131. The composition of claim 130, wherein at least two of R₁, R₂, and R₃ are independently phenylalanine or naphthylalanine and wherein at least two of R₅, R₆, and R₇ are independently arginine, homoarginine, N-methylarginine, and N,N-dimethylarginine.

132. The pharmaceutical composition of claim 131, wherein the compound further comprises an exocyclic comprising the sequence comprises PGKKRKV, PKKKRKV, PKKKGKV, or PKKKRKG.

133. The pharmaceutical composition of claim 131, wherein comprising a linker, wherein the linker conjugates the antisense compound and the exocyclic peptide to the cyclic peptide, the linker the following formula: 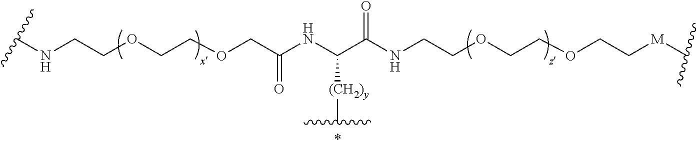
wherein:
x′ is an integer from 1-23;
y is an integer from 1-5;
z′ is an integer from 1-23;
* is the point of attachment to the AA_{SC} of the cyclic peptide;
and M is a bonding group; and
wherein the exocyclic peptide is conjugated to the linker at the amino end of the linker and the antisense compound is conjugated to M.

134. The compound of claim 131, wherein the antisense compound comprises
5′-CAG CAG CAG CAG CAG CAG CAG CAG-3′;
5′-CAG CAG CAG CAG CAG CAG CAG CAG CAG-3′;
or
5′-CAG CAG CAG CAG CAG CAG CAG-3′.

135. A method of treating myotonic dystrophy (DM) in a subject in need thereof, comprising administering a pharmaceutical composition to the subject, the pharmaceutical composition comprising a carrier and a compound comprising:
(a) an antisense compound that is complementary to at least a portion of an expanded CUG repeat in a target mRNA sequence;
(b) a cyclic peptide of the formula: 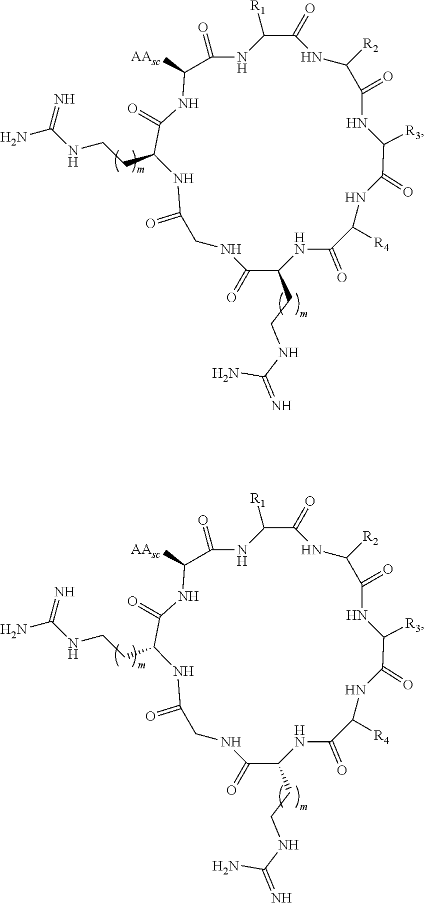
or a protonated form thereof, wherein:
R₁, R₂, and R₃ are each independently H or an amino acid residue having a side chain comprising an aromatic group;
at least two of R₁, R₂, and R₃ are the side chain of phenylalanine;
R₄ is H or an amino acid side chain;
AA_{SC} is an amino acid side chain; and
each m is independently an integer of 0, 1, 2, or 3.

136. The method of claim 135, wherein the compound further comprises an exocyclic peptide comprising the sequence comprises PGKKRKV, PKKKRKV, PKKKGKV, or PKKKRKG, and wherein the compound is 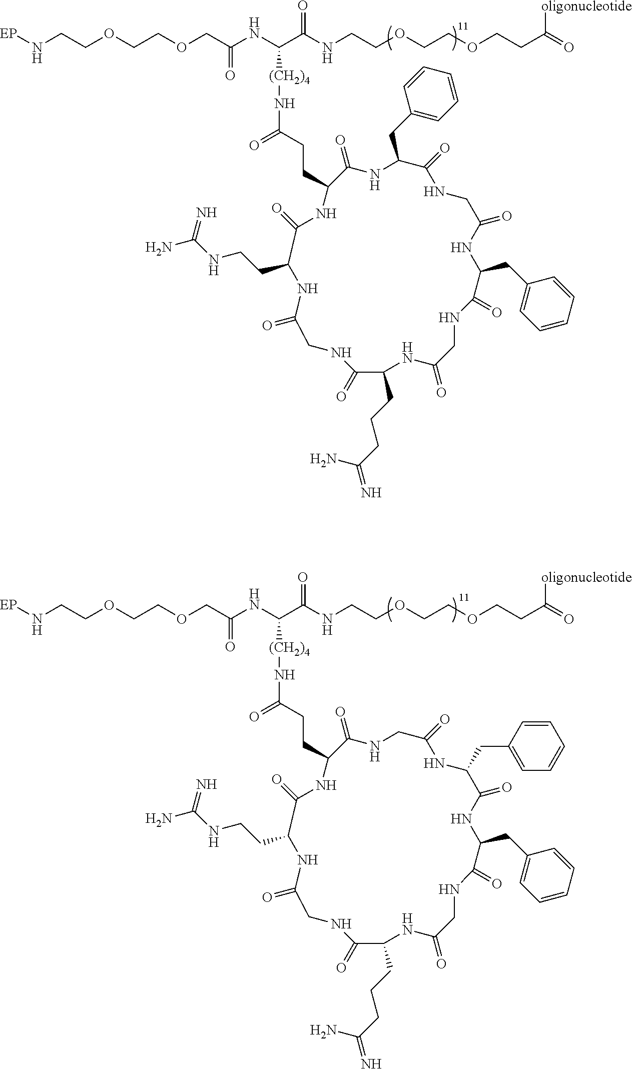 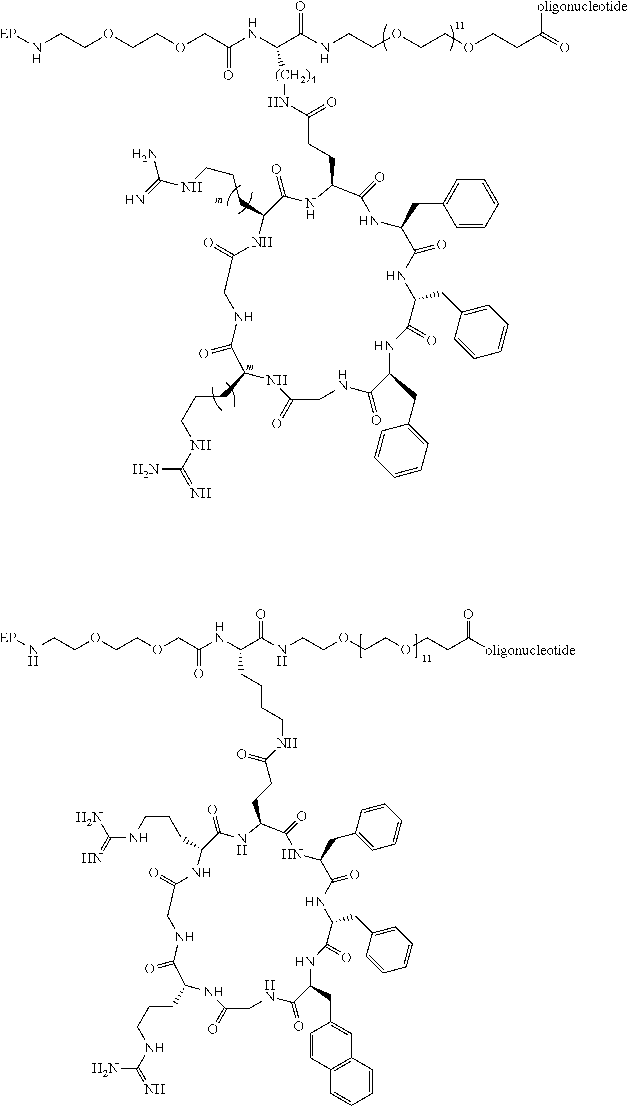
or a protonated form or salt thereof,
wherein EP is the exocyclic peptide, and
oligonucleotide is the antisense compound.

137. The method of claim 135, wherein the administering results in an increase in the expression of a wild-type protein in muscle tissue, wherein the wild-type protein is expressed from a gene that does not have an expanded CUG repeat.

138. The method of claim 135, wherein the muscle tissue is diaphragm tissue, quadricep tissues, heat tissue, or any combination thereof.

139. The method of claim 135, wherein the administration prevents or reduces foci formation.

140. A method of treating myotonic dystrophy (DM) in a subject in need thereof, comprising administering a pharmaceutical composition to the subject, the pharmaceutical composition comprising a carrier and a compound comprising:
(a) an antisense compound that is complementary to at least a portion of an expanded CUG repeat in a target mRNA sequence;
(b) a cyclic peptide of the formula: 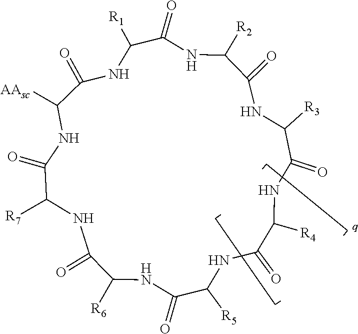
or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
AA_{SC} is an amino acid side chain; and
q is 1, 2, 3 or 4,
wherein at least two amino acids of the cyclic peptide are charged amino acids, at least two amino acids of the cyclic peptide are aromatic hydrophobic amino acids, and at least two amino acids of the cyclic peptide are uncharged, non-aromatic amino acids.

141. The method of claim 140, wherein the compound further comprises an exocyclic peptide of the sequence PGKKRKV, PKKKRKV, PKKKGKV, or PKKKRKG, and wherein the compound further comprises a linker of the formula 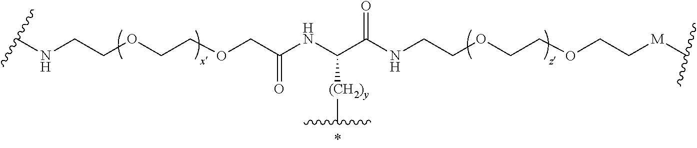
wherein,
x′ is an integer from 1-23;
y is an integer from 1-5;
z′ is an integer from 1-23;
* is the point of attachment to the AA_{SC} of the cyclic peptide;
and M is a bonding group; and
wherein the exocyclic peptide is conjugated to the linker at the amino end of the linker and the antisense compound is conjugated to M.

142. The method of claim 140, wherein the antisense compound comprises
5′-CAG CAG CAG CAG CAG CAG CAG CAG-3′;
5′-CAG CAG CAG CAG CAG CAG CAG CAG CAG-3′;
or
5′-CAG CAG CAG CAG CAG CAG CAG-3′.

143. The method of claim 140, wherein the administering results in an increase in the expression of a wild-type protein in muscle tissue, wherein the wild-type protein is expressed from a gene that does not have an expanded CUG repeat.

144. The method of claim 143, wherein the muscle tissue is diaphragm tissue, quadricep tissues, heat tissue, or any combination thereof.

145. The method of claim 140, wherein the administration prevents or reduces foci formation.


SEQUENCE LISTING (713 sequences)

SEQ ID NO: 1 - protein, 4 aa, synthetic construct
  REGION 1..4: note = Description of Artificial Sequence: Synthetic peptide
  VARIANT 1..4: note = May have D or L stereochemistry
HHHH

SEQ ID NO: 2 - protein, 4 aa, synthetic construct
  REGION 1..4: note = Description of Artificial Sequence: Synthetic peptide
  VARIANT 1..4: note = May have D or L stereochemistry
KHKK

SEQ ID NO: 3 - protein, 4 aa, synthetic construct
  REGION 1..4: note = Description of Artificial Sequence: Synthetic peptide
  VARIANT 1..4: note = May have D or L stereochemistry
KKHK

SEQ ID NO: 4 - protein, 4 aa, synthetic construct
  REGION 1..4: note = Description of Artificial Sequence: Synthetic peptide
  VARIANT 1..4: note = May have D or L stereochemistry
KKKH

SEQ ID NO: 5 - protein, 4 aa, synthetic construct
  REGION 1..4: note = Description of Artificial Sequence: Synthetic peptide
  VARIANT 1..4: note = May have D or L stereochemistry
KHKH

SEQ ID NO: 6 - protein, 4 aa, synthetic construct
  REGION 1..4: note = Description of Artificial Sequence: Synthetic peptide
  VARIANT 1..4: note = May have D or L stereochemistry
HKHK

SEQ ID NO: 7 - protein, 4 aa, synthetic construct
  REGION 1..4: note = Description of Artificial Sequence: Synthetic peptide
  VARIANT 1..4: note = May have D or L stereochemistry
KKKK

SEQ ID NO: 8 - protein, 4 aa, synthetic construct
  REGION 1..4: note = Description of Artificial Sequence: Synthetic peptide
  VARIANT 1..4: note = May have D or L stereochemistry
KKRK

SEQ ID NO: 9 - protein, 4 aa, synthetic construct
  REGION 1..4: note = Description of Artificial Sequence: Synthetic peptide
  VARIANT 1..4: note = May have D or L stereochemistry
KRKK

SEQ ID NO: 10 - protein, 4 aa, synthetic construct
  REGION 1..4: note = Description of Artificial Sequence: Synthetic peptide
  VARIANT 1..4: note = May have D or L stereochemistry
KRRK

SEQ ID NO: 11 - protein, 4 aa, synthetic construct
  REGION 1..4: note = Description of Artificial Sequence: Synthetic peptide
  VARIANT 1..4: note = May have D or L stereochemistry
RKKR

SEQ ID NO: 12 - protein, 4 aa, synthetic construct
  REGION 1..4: note = Description of Artificial Sequence: Synthetic peptide
  VARIANT 1..4: note = May have D or L stereochemistry
RRRR

SEQ ID NO: 13 - protein, 4 aa, synthetic construct
  REGION 1..4: note = Description of Artificial Sequence: Synthetic peptide
  VARIANT 1..4: note = May have D or L stereochemistry
KGKK

SEQ ID NO: 14 - protein, 4 aa, synthetic construct
  REGION 1..4: note = Description of Artificial Sequence: Synthetic peptide
  VARIANT 1..4: note = May have D or L stereochemistry
KKGK

SEQ ID NO: 15-16 - skipped (2 entries)

SEQ ID NO: 17 - protein, 5 aa, synthetic construct
  REGION 1..5: note = Description of Artificial Sequence: Synthetic peptide
  VARIANT 1..5: note = May have D or L stereochemistry
RRRRR

SEQ ID NO: 18 - protein, 5 aa, synthetic construct
  REGION 1..5: note = Description of Artificial Sequence: Synthetic peptide
  VARIANT 1..5: note = May have D or L stereochemistry
KKKKK

SEQ ID NO: 19 - protein, 5 aa, synthetic construct
  REGION 1..5: note = Description of Artificial Sequence: Synthetic peptide
  VARIANT 1..5: note = May have D or L stereochemistry
KKKRK

SEQ ID NO: 20 - protein, 5 aa, synthetic construct
  REGION 1..5: note = Description of Artificial Sequence: Synthetic peptide
  VARIANT 1..5: note = May have D or L stereochemistry
RKKKK

SEQ ID NO: 21 - protein, 5 aa, synthetic construct
  REGION 1..5: note = Description of Artificial Sequence: Synthetic peptide
  VARIANT 1..5: note = May have D or L stereochemistry
KRKKK

SEQ ID NO: 22 - protein, 5 aa, synthetic construct
  REGION 1..5: note = Description of Artificial Sequence: Synthetic peptide
  VARIANT 1..5: note = May have D or L stereochemistry
KKRKK

SEQ ID NO: 23 - protein, 5 aa, synthetic construct
  REGION 1..5: note = Description of Artificial Sequence: Synthetic peptide
  VARIANT 1..5: note = May have D or L stereochemistry
KKKKR

SEQ ID NO: 24 - skipped

SEQ ID NO: 25 - protein, 6 aa, synthetic construct
  REGION 1..6: note = Description of Artificial Sequence: Synthetic peptide
  VARIANT 1..6: note = May have D or L stereochemistry
RKKKKG

SEQ ID NO: 26 - protein, 6 aa, synthetic construct
  REGION 1..6: note = Description of Artificial Sequence: Synthetic peptide
  VARIANT 1..6: note = May have D or L stereochemistry
KRKKKG

SEQ ID NO: 27 - protein, 6 aa, synthetic construct
  REGION 1..6: note = Description of Artificial Sequence: Synthetic peptide
  VARIANT 1..6: note = May have D or L stereochemistry
KKRKKG

SEQ ID NO: 28 - protein, 6 aa, synthetic construct
  REGION 1..6: note = Description of Artificial Sequence: Synthetic peptide
  VARIANT 1..6: note = May have D or L stereochemistry
KKKKRG

SEQ ID NO: 29 - protein, 6 aa, synthetic construct
  REGION 1..6: note = Description of Artificial Sequence: Synthetic peptide
  SITE 6: note = MOD_RES - Beta-alanine
  VARIANT 1..6: note = May have D or L stereochemistry
RKKKKX

SEQ ID NO: 30 - protein, 6 aa, synthetic construct
  REGION 1..6: note = Description of Artificial Sequence: Synthetic peptide
  SITE 6: note = MOD_RES - Beta-alanine
  VARIANT 1..6: note = May have D or L stereochemistry
KRKKKX

SEQ ID NO: 31 - protein, 6 aa, synthetic construct
  REGION 1..6: note = Description of Artificial Sequence: Synthetic peptide
  SITE 6: note = MOD_RES - Beta-alanine
  VARIANT 1..6: note = May have D or L stereochemistry
KKRKKX

SEQ ID NO: 32 - protein, 6 aa, synthetic construct
  REGION 1..6: note = Description of Artificial Sequence: Synthetic peptide
  SITE 6: note = MOD_RES - Beta-alanine
  VARIANT 1..6: note = May have D or L stereochemistry
KKKKRX

SEQ ID NO: 33 - protein, 6 aa, synthetic construct
  REGION 1..6: note = Description of Artificial Sequence: Synthetic peptide
  VARIANT 1..6: note = May have D or L stereochemistry
KKKRKV

SEQ ID NO: 34 - protein, 6 aa, synthetic construct
  REGION 1..6: note = Description of Artificial Sequence: Synthetic peptide
  VARIANT 1..6: note = May have D or L stereochemistry
RRRRRR

SEQ ID NO: 35 - protein, 6 aa, synthetic construct
  REGION 1..6: note = Description of Artificial Sequence: Synthetic peptide
  VARIANT 1..6: note = May have D or L stereochemistry
HHHHHH

SEQ ID NO: 36 - protein, 6 aa, synthetic construct
  REGION 1..6: note = Description of Artificial Sequence: Synthetic peptide
  VARIANT 1..6: note = May have D or L stereochemistry
RHRHRH

SEQ ID NO: 37 - protein, 6 aa, synthetic construct
  REGION 1..6: note = Description of Artificial Sequence: Synthetic peptide
  VARIANT 1..6: note = May have D or L stereochemistry
HRHRHR

SEQ ID NO: 38 - protein, 6 aa, synthetic construct
  REGION 1..6: note = Description of Artificial Sequence: Synthetic peptide
  VARIANT 1..6: note = May have D or L stereochemistry
KRKRKR

SEQ ID NO: 39 - protein, 6 aa, synthetic construct
  REGION 1..6: note = Description of Artificial Sequence: Synthetic peptide
  VARIANT 1..6: note = May have D or L stereochemistry
RKRKRK

SEQ ID NO: 40-41 - skipped (2 entries)

SEQ ID NO: 42 - protein, 7 aa, synthetic construct
  REGION 1..7: note = Description of Artificial Sequence: Synthetic peptide
  VARIANT 1..7: note = May have D or L stereochemistry
PKKKRKV

SEQ ID NO: 43 - protein, 7 aa, synthetic construct
  REGION 1..7: note = Description of Artificial Sequence: Synthetic peptide
  VARIANT 1..7: note = May have D or L stereochemistry
PGKKRKV

SEQ ID NO: 44 - protein, 7 aa, synthetic construct
  REGION 1..7: note = Description of Artificial Sequence: Synthetic peptide
  VARIANT 1..7: note = May have D or L stereochemistry
PKGKRKV

SEQ ID NO: 45 - protein, 7 aa, synthetic construct
  REGION 1..7: note = Description of Artificial Sequence: Synthetic peptide
  VARIANT 1..7: note = May have D or L stereochemistry
PKKGRKV

SEQ ID NO: 46 - protein, 7 aa, synthetic construct
  REGION 1..7: note = Description of Artificial Sequence: Synthetic peptide
  VARIANT 1..7: note = May have D or L stereochemistry
PKKKGKV

SEQ ID NO: 47 - protein, 7 aa, synthetic construct
  REGION 1..7: note = Description of Artificial Sequence: Synthetic peptide
  VARIANT 1..7: note = May have D or L stereochemistry
PKKKRGV

SEQ ID NO: 48 - protein, 7 aa, synthetic construct
  REGION 1..7: note = Description of Artificial Sequence: Synthetic peptide
  VARIANT 1..7: note = May have D or L stereochemistry
PKKKRKG

SEQ ID NO: 49-51 - skipped (3 entries)

SEQ ID NO: 52 - protein, 19 aa, synthetic construct
  REGION 1..19: note = Description of Artificial Sequence: Synthetic peptide
NLSKRPAAIKKAGQAKKKK

SEQ ID NO: 53 - protein, 9 aa, synthetic construct
  REGION 1..9: note = Description of Artificial Sequence: Synthetic peptide
PAAKRVKLD

SEQ ID NO: 54 - protein, 11 aa, synthetic construct
  REGION 1..11: note = Description of Artificial Sequence: Synthetic peptide
RQRRNELKRSF

SEQ ID NO: 55 - protein, 27 aa, synthetic construct
  REGION 1..27: note = Description of Artificial Sequence: Synthetic peptide
RMRKFKNKGKDTAELRRRRVEVSVELR

SEQ ID NO: 56 - protein, 14 aa, synthetic construct
  REGION 1..14: note = Description of Artificial Sequence: Synthetic peptide
KAKKDEQILKRRNV

SEQ ID NO: 57 - protein, 8 aa, synthetic construct
  REGION 1..8: note = Description of Artificial Sequence: Synthetic peptide
VSRKRPRP

SEQ ID NO: 58 - protein, 8 aa, synthetic construct
  REGION 1..8: note = Description of Artificial Sequence: Synthetic peptide
PPKKARED

SEQ ID NO: 59 - protein, 8 aa, synthetic construct
  REGION 1..8: note = Description of Artificial Sequence: Synthetic peptide
PQPKKKPL

SEQ ID NO: 60 - protein, 12 aa, synthetic construct
  REGION 1..12: note = Description of Artificial Sequence: Synthetic peptide
SALIKKKKKMAP

SEQ ID NO: 61 - protein, 5 aa, synthetic construct
  REGION 1..5: note = Description of Artificial Sequence: Synthetic peptide
DRLRR

SEQ ID NO: 62 - protein, 7 aa, synthetic construct
  REGION 1..7: note = Description of Artificial Sequence: Synthetic peptide
PKQKKRK

SEQ ID NO: 63 - protein, 10 aa, synthetic construct
  REGION 1..10: note = Description of Artificial Sequence: Synthetic peptide
RKLKKKIKKL

SEQ ID NO: 64 - protein, 10 aa, synthetic construct
  REGION 1..10: note = Description of Artificial Sequence: Synthetic peptide
REKKKFLKRR

SEQ ID NO: 65 - protein, 20 aa, synthetic construct
  REGION 1..20: note = Description of Artificial Sequence: Synthetic peptide
KRKGDEVDGVDEVAKKKSKK

SEQ ID NO: 66 - protein, 17 aa, synthetic construct
  REGION 1..17: note = Description of Artificial Sequence: Synthetic peptide
RKCLQAGMNLEARKTKK

SEQ ID NO: 67 - skipped

SEQ ID NO: 68 - protein, 7 aa, synthetic construct
  REGION 1..7: note = Description of Artificial Sequence: Synthetic peptide
FGFGRGR

SEQ ID NO: 69 - protein, 7 aa, synthetic construct
  REGION 1..7: note = Description of Artificial Sequence: Synthetic peptide
  SITE 2: note = MOD_RES - D-phenylalanine
  SITE 5: note = MOD_RES - D-arginine
  SITE 7: note = MOD_RES - D-arginine
GFFGRGR

SEQ ID NO: 70 - protein, 7 aa, synthetic construct
  REGION 1..7: note = Description of Artificial Sequence: Synthetic peptide
  SITE 2: note = MOD_RES - D-phenylalanine
  SITE 3: note = MOD_RES - L-naphthylalanine
FFXGRGR

SEQ ID NO: 71 - protein, 7 aa, synthetic construct
  REGION 1..7: note = Description of Artificial Sequence: Synthetic peptide
  SITE 2: note = MOD_RES - D-phenylalanine
FFFGRGR

SEQ ID NO: 72 - protein, 7 aa, synthetic construct
  REGION 1..7: note = Description of Artificial Sequence: Synthetic peptide
  SITE 2: note = MOD_RES - D-phenylalanine
  SITE 3: note = MOD_RES - L-naphthylalanine
  SITE 5: note = MOD_RES - D-arginine
  SITE 7: note = MOD_RES - D-arginine
FFXGRGR

SEQ ID NO: 73 - skipped

SEQ ID NO: 74 - protein, 8 aa, synthetic construct
  REGION 1..8: note = Description of Artificial Sequence: Synthetic peptide
  SITE 2: note = MOD_RES - D-phenylalanine
  SITE 5: note = MOD_RES - D-arginine
  SITE 7: note = MOD_RES - D-arginine
GFFGRGRQ

SEQ ID NO: 75 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
FFXGRGRQ

SEQ ID NO: 76 - protein, 8 aa, synthetic construct
  REGION 1..8: note = Description of Artificial Sequence: Synthetic peptide
  SITE 2: note = MOD_RES - D-phenylalanine
FFFGRGRQ

SEQ ID NO: 77 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
  SITE 5: note = MOD_RES - D-arginine
  SITE 7: note = MOD_RES - D-arginine
FFXGRGRQ

SEQ ID NO: 78 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
  SITE 5: note = MOD_RES - D-arginine
  SITE 7: note = MOD_RES - D-arginine
FFXRRRRQ

SEQ ID NO: 79 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
  SITE 4: note = MOD_RES - Citrulline
  SITE 5: note = MOD_RES - D-arginine
  SITE 6: note = MOD_RES - Citrulline
  SITE 7: note = MOD_RES - D-arginine
FFXXRXRQ

SEQ ID NO: 80 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
  SITE 5: note = MOD_RES - D-arginine
  SITE 7: note = MOD_RES - D-arginine
FFXGRGRQ

SEQ ID NO: 81 - protein, 8 aa, synthetic construct
  REGION 1..8: note = Description of Artificial Sequence: Synthetic peptide
  SITE 2: note = MOD_RES - D-phenylalanine
FFFGRGRQ

SEQ ID NO: 82 - protein, 8 aa, synthetic construct
  REGION 1..8: note = Description of Artificial Sequence: Synthetic peptide
FGFGRGRQ

SEQ ID NO: 83 - protein, 8 aa, synthetic construct
  REGION 1..8: note = Description of Artificial Sequence: Synthetic peptide
  SITE 2: note = MOD_RES - D-phenylalanine
  SITE 5: note = MOD_RES - D-arginine
  SITE 7: note = MOD_RES - D-arginine
GFFGRGRQ

SEQ ID NO: 84 - protein, 8 aa, synthetic construct
  REGION 1..8: note = Description of Artificial Sequence: Synthetic peptide
FGFGRRRQ

SEQ ID NO: 85 - protein, 8 aa, synthetic construct
  REGION 1..8: note = Description of Artificial Sequence: Synthetic peptide
FGFRRRRQ

SEQ ID NO: 86 - protein, 7 aa, synthetic construct
  REGION 1..7: note = Description of Artificial Sequence: Synthetic peptide
  SITE 2: note = MOD_RES - L-naphthylalanine
FXRRRRQ

SEQ ID NO: 87 - protein, 7 aa, synthetic construct
  REGION 1..7: note = Description of Artificial Sequence: Synthetic peptide
  SITE 1: note = MOD_RES - D-phenylalanine
  SITE 2: note = MOD_RES - L-naphthylalanine
  SITE 4: note = MOD_RES - D-arginine
  SITE 6: note = MOD_RES - D-arginine
FXRRRRQ

SEQ ID NO: 88 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
FFXRGRGQ

SEQ ID NO: 89 - protein, 6 aa, synthetic construct
  REGION 1..6: note = Description of Artificial Sequence: Synthetic peptide
  SITE 2: note = MOD_RES - L-naphthylalanine
FXRRRQ

SEQ ID NO: 90 - protein, 6 aa, synthetic construct
  REGION 1..6: note = Description of Artificial Sequence: Synthetic peptide
  SITE 2: note = MOD_RES - L-naphthylalanine
FXRRRC

SEQ ID NO: 91 - protein, 6 aa, synthetic construct
  REGION 1..6: note = Description of Artificial Sequence: Synthetic peptide
  SITE 2: note = MOD_RES - L-naphthylalanine
  SITE 6: note = MOD_RES - Selenocysteine
FXRRRU

SEQ ID NO: 92 - protein, 6 aa, synthetic construct
  REGION 1..6: note = Description of Artificial Sequence: Synthetic peptide
  SITE 4: note = MOD_RES - L-naphthylalanine
RRRXFQ

SEQ ID NO: 93 - protein, 6 aa, synthetic construct
  REGION 1..6: note = Description of Artificial Sequence: Synthetic peptide
  SITE 5: note = MOD_RES - L-naphthylalanine
RRRRXF

SEQ ID NO: 94 - protein, 6 aa, synthetic construct
  REGION 1..6: note = Description of Artificial Sequence: Synthetic peptide
  SITE 2: note = MOD_RES - L-naphthylalanine
FXRRRR

SEQ ID NO: 95 - protein, 7 aa, synthetic construct
  REGION 1..7: note = Description of Artificial Sequence: Synthetic peptide
  SITE 2: note = MOD_RES - D-naphthylalanine
  SITE 3: note = MOD_RES - D-arginine
  SITE 5: note = MOD_RES - D-arginine
  SITE 7: note = MOD_RES - D-glutamine
FXRRRRQ

SEQ ID NO: 96 - protein, 7 aa, synthetic construct
  REGION 1..7: note = Description of Artificial Sequence: Synthetic peptide
  SITE 2: note = MOD_RES - D-naphthylalanine
  SITE 3: note = MOD_RES - D-arginine
  SITE 5: note = MOD_RES - D-arginine
FXRRRRQ

SEQ ID NO: 97 - protein, 7 aa, synthetic construct
  REGION 1..7: note = Description of Artificial Sequence: Synthetic peptide
  SITE 2: note = MOD_RES - L-naphthylalanine
FXRRRRQ

SEQ ID NO: 98 - protein, 7 aa, synthetic construct
  REGION 1..7: note = Description of Artificial Sequence: Synthetic peptide
  SITE 1: note = MOD_RES - D-phenylalanine
  SITE 2: note = MOD_RES - L-naphthylalanine
  SITE 4: note = MOD_RES - D-arginine
  SITE 6: note = MOD_RES - D-arginine
FXRRRRQ

SEQ ID NO: 99 - protein, 7 aa, synthetic construct
  REGION 1..7: note = Description of Artificial Sequence: Synthetic peptide
  SITE 5: note = MOD_RES - L-naphthylalanine
RRFRXRQ

SEQ ID NO: 100 - protein, 7 aa, synthetic construct
  REGION 1..7: note = Description of Artificial Sequence: Synthetic peptide
  SITE 6: note = MOD_RES - L-naphthylalanine
FRRRRXQ

SEQ ID NO: 101 - protein, 7 aa, synthetic construct
  REGION 1..7: note = Description of Artificial Sequence: Synthetic peptide
  SITE 1: note = MOD_RES - D-arginine
  SITE 5: note = MOD_RES - L-naphthylalanine
RRFRXRQ

SEQ ID NO: 102 - protein, 7 aa, synthetic construct
  REGION 1..7: note = Description of Artificial Sequence: Synthetic peptide
  SITE 3: note = MOD_RES - L-naphthylalanine
RRXFRRQ

SEQ ID NO: 103 - protein, 8 aa, synthetic construct
  REGION 1..8: note = Description of Artificial Sequence: Synthetic peptide
CRRRRFWQ

SEQ ID NO: 104 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
  SITE 5: note = MOD_RES - D-arginine
  SITE 7: note = MOD_RES - D-arginine
FFXRRRRQ

SEQ ID NO: 105 - protein, 8 aa, synthetic construct
  REGION 1..8: note = Description of Artificial Sequence: Synthetic peptide
  SITE 3: note = MOD_RES - L-naphthylalanine
FFXRRRRQ

SEQ ID NO: 106 - protein, 8 aa, synthetic construct
  REGION 1..8: note = Description of Artificial Sequence: Synthetic peptide
  SITE 6: note = MOD_RES - L-naphthylalanine
RFRFRXRQ

SEQ ID NO: 107 - protein, 8 aa, synthetic construct
  REGION 1..8: note = Description of Artificial Sequence: Synthetic peptide
  SITE 1: note = MOD_RES - Selenocysteine
URRRRFWQ

SEQ ID NO: 108 - protein, 8 aa, synthetic construct
  REGION 1..8: note = Description of Artificial Sequence: Synthetic peptide
CRRRRFWQ

SEQ ID NO: 109 - protein, 8 aa, synthetic construct
  REGION 1..8: note = Description of Artificial Sequence: Synthetic peptide
  SITE 2: note = MOD_RES - L-naphthylalanine
FXRRRRQK

SEQ ID NO: 110 - protein, 8 aa, synthetic construct
  REGION 1..8: note = Description of Artificial Sequence: Synthetic peptide
  SITE 2: note = MOD_RES - L-naphthylalanine
FXRRRRQC

SEQ ID NO: 111 - protein, 8 aa, synthetic construct
  REGION 1..8: note = Description of Artificial Sequence: Synthetic peptide
  SITE 1: note = MOD_RES - D-phenylalanine
  SITE 2: note = MOD_RES - L-naphthylalanine
  SITE 4: note = MOD_RES - D-arginine
  SITE 6: note = MOD_RES - D-arginine
FXRRRRRQ

SEQ ID NO: 112 - protein, 8 aa, synthetic construct
  REGION 1..8: note = Description of Artificial Sequence: Synthetic peptide
  SITE 2: note = MOD_RES - L-naphthylalanine
FXRRRRRQ

SEQ ID NO: 113 - protein, 9 aa, synthetic construct
  REGION 1..9: note = Description of Artificial Sequence: Synthetic peptide
  SITE 5: note = MOD_RES - L-naphthylalanine
  SITE 8: note = MOD_RES - Norleucine
RRRRXFDXC

SEQ ID NO: 114 - protein, 5 aa, synthetic construct
  REGION 1..5: note = Description of Artificial Sequence: Synthetic peptide
  SITE 2: note = MOD_RES - L-naphthylalanine
FXRRR

SEQ ID NO: 115 - protein, 5 aa, synthetic construct
  REGION 1..5: note = Description of Artificial Sequence: Synthetic peptide
FWRRR

SEQ ID NO: 116 - protein, 5 aa, synthetic construct
  REGION 1..5: note = Description of Artificial Sequence: Synthetic peptide
  SITE 4: note = MOD_RES - L-naphthylalanine
RRRXF

SEQ ID NO: 117 - protein, 5 aa, synthetic construct
  REGION 1..5: note = Description of Artificial Sequence: Synthetic peptide
RRRWF

SEQ ID NO: 118 - protein, 8 aa, synthetic construct
  SITE 2: note = D-amino acid
  SITE 3: note = 3-(2-naphthyl)-alanine
  SITE 4: note = 4-Guanidinophenylalanine
  SITE 5: note = D-amino acid
  SITE 6: note = 4-Guanidinophenylalanine
  SITE 7: note = D-amino acid
FFXXRXRQ

SEQ ID NO: 119 - protein, 8 aa, synthetic construct
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 120 - protein, 8 aa, synthetic construct
  SITE 2: note = D-amino acid
  SITE 3: note = 3-(2-naphthyl)-alanine
  SITE 4: note = 3-(1-piperidinyl)-l-alanine
  SITE 5: note = D-amino acid
  SITE 6: note = 3-(1-piperidinyl)-l-alanine
  SITE 7: note = D-amino acid
FFXXRXRQ

SEQ ID NO: 121 - protein, 8 aa, synthetic construct
  SITE 2: note = D-amino acid
  SITE 3: note = 3-(2-naphthyl)-alanine
  SITE 4: note = N-epsilon,N-epsilon-dimethyl-l-lysine
  SITE 5: note = D-amino acid
  SITE 6: note = N-epsilon,N-epsilon-dimethyl-l-lysine
  SITE 7: note = D-amino acid
FFXXRXRQ

SEQ ID NO: 122 - protein, 8 aa, synthetic construct
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 123 - protein, 7 aa, synthetic construct
  SITE 1: note = D-amino acid
  SITE 2: note = 3-(2-naphthyl)-alanine
  SITE 4: note = Citrulline
  SITE 6: note = Citrulline
FXRXRXQ

SEQ ID NO: 124-126 - skipped (3 entries)

SEQ ID NO: 127 - protein, 8 aa, synthetic construct
  SITE 2: note = D-amino acid
  SITE 3: note = 3-(2-naphthyl)-alanine
  SITE 5: note = D-amino acid
  SITE 6: note = Citrulline
  SITE 7: note = D-amino acid
FFXRRXRQ

SEQ ID NO: 128 - protein, 8 aa, synthetic construct
  SITE 2: note = D-amino acid
  SITE 3: note = 3-(2-naphthyl)-alanine
  SITE 4: note = Citrulline
  SITE 5: note = D-amino acid
  SITE 7: note = D-amino acid
FFXXRRRQ

SEQ ID NO: 129 - protein, 8 aa, synthetic construct
  SITE 2: note = D-amino acid
  SITE 3: note = 3-(2-naphthyl)-alanine
  SITE 5: note = D-Citrulline
  SITE 7: note = D-Citrulline
FFXRXRXQ

SEQ ID NO: 130 - protein, 8 aa, synthetic construct
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 131 - protein, 7 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
PKKKRKV

SEQ ID NO: 132 - protein, 7 aa, synthetic construct
PKKKRKV

SEQ ID NO: 133 - protein, 8 aa, synthetic construct
  SITE 2: note = D-amino acid
  SITE 5: note = D-amino acid
  SITE 7: note = D-amino acid
GFFGRGRQ

SEQ ID NO: 134 - protein, 7 aa, synthetic construct
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
PKKKRKV

SEQ ID NO: 135 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 136 - protein, 8 aa, synthetic construct
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 137-141 - skipped (5 entries)

SEQ ID NO: 142 - protein, 8 aa, synthetic construct
  SITE 1: note = Beta-homophenylalanine
  SITE 2: note = D-amino acid
  SITE 3: note = 3-(2-naphthyl)-alanine
  SITE 5: note = D-amino acid
  SITE 7: note = D-amino acid
XFXGRGRQ

SEQ ID NO: 143 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXSRSRQ

SEQ ID NO: 144 - protein, 8 aa, synthetic construct
FGFKRKRQ

SEQ ID NO: 145 - protein, 8 aa, synthetic construct
FGFRGRGQ

SEQ ID NO: 146 - DNA, 21 bp, synthetic construct
  misc_feature 1..21: note = Description of Artificial Sequence: Synthetic oligonucleotide
  source 1..21: mol_type = other DNA
  misc_feature 1..21: note = Phosphorodiamidate morpholino backbone
cagcagcagcagcagcagcag

SEQ ID NO: 147 - DNA, 25 bp, synthetic construct
  misc_feature 1..25: note = Description of Artificial Sequence: Synthetic oligonucleotide
  source 1..25: mol_type = other DNA
  misc_feature 1..25: note = Phosphorodiamidate morpholino backbone
cagcagcagcagcagcagcagcagc

SEQ ID NO: 148 - DNA, 30 bp, synthetic construct
  misc_feature 1..30: note = Description of Artificial Sequence: Synthetic oligonucleotide
  source 1..30: mol_type = other DNA
  misc_feature 1..30: note = Phosphorodiamidate morpholino backbone
cagcagcagcagcagcagcagcagcagcag

SEQ ID NO: 149 - DNA, 30 bp, synthetic construct
  misc_feature 1..30: note = Description of Artificial Sequence: Synthetic oligonucleotide
  source 1..30: mol_type = other DNA
  misc_feature 1..30: note = Phosphorodiamidate morpholino backbone
agcagcagcagcagcagcagcagcagcagc

SEQ ID NO: 150 - protein, 8 aa, synthetic construct
  REGION 1..8: note = Description of Artificial Sequence: Synthetic peptide
  SITE 2: note = MOD_RES - D-phenylalanine
  SITE 3: note = MOD_RES - L-naphthylalanine
  SITE 5: note = MOD_RES - D-arginine
  SITE 7: note = MOD_RES - D-arginine
FFXRRRRQ

SEQ ID NO: 151 - DNA, 12 bp, synthetic construct
  misc_feature 1..12: note = Description of Artificial Sequence: Synthetic oligonucleotide
  source 1..12: mol_type = other DNA
cagcagcagcag

SEQ ID NO: 152 - DNA, 15 bp, synthetic construct
  misc_feature 1..15: note = Description of Artificial Sequence: Synthetic oligonucleotide
  source 1..15: mol_type = other DNA
cagcagcagcagcag

SEQ ID NO: 153 - DNA, 18 bp, synthetic construct
  misc_feature 1..18: note = Description of Artificial Sequence: Synthetic oligonucleotide
  source 1..18: mol_type = other DNA
cagcagcagcagcagcag

SEQ ID NO: 154 - DNA, 21 bp, synthetic construct
  misc_feature 1..21: note = Description of Artificial Sequence: Synthetic oligonucleotide
  source 1..21: mol_type = other DNA
cagcagcagcagcagcagcag

SEQ ID NO: 155 - DNA, 24 bp, synthetic construct
  misc_feature 1..24: note = Description of Artificial Sequence: Synthetic oligonucleotide
  source 1..24: mol_type = other DNA
cagcagcagcagcagcagcagcag

SEQ ID NO: 156 - DNA, 27 bp, synthetic construct
  misc_feature 1..27: note = Description of Artificial Sequence: Synthetic oligonucleotide
  source 1..27: mol_type = other DNA
cagcagcagcagcagcagcagcagcag

SEQ ID NO: 157 - DNA, 30 bp, synthetic construct; identical to SEQ ID NO: 148
  misc_feature 1..30: note = Description of Artificial Sequence: Synthetic oligonucleotide
  source 1..30: mol_type = other DNA

SEQ ID NO: 158 - DNA, 33 bp, synthetic construct
  misc_feature 1..33: note = Description of Artificial Sequence: Synthetic oligonucleotide
  source 1..33: mol_type = other DNA
cagcagcagcagcagcagcagcagcagcagcag

SEQ ID NO: 159 - DNA, 36 bp, synthetic construct
  misc_feature 1..36: note = Description of Artificial Sequence: Synthetic oligonucleotide
  source 1..36: mol_type = other DNA
cagcagcagcagcagcagcagcagcagcagcagcag

SEQ ID NO: 160 - DNA, 39 bp, synthetic construct
  misc_feature 1..39: note = Description of Artificial Sequence: Synthetic oligonucleotide
  source 1..39: mol_type = other DNA
cagcagcagcagcagcagcagcagcagcagcagcagcag

SEQ ID NO: 161 - DNA, 42 bp, synthetic construct
  misc_feature 1..42: note = Description of Artificial Sequence: Synthetic oligonucleotide
  source 1..42: mol_type = other DNA
cagcagcagcagcagcagcagcagcagcagcagcagcagcag

SEQ ID NO: 162 - DNA, 45 bp, synthetic construct
  misc_feature 1..45: note = Description of Artificial Sequence: Synthetic oligonucleotide
  source 1..45: mol_type = other DNA
cagcagcagcagcagcagcagcagcagcagcagcagcagcagcag

SEQ ID NO: 163 - DNA, 48 bp, synthetic construct
  misc_feature 1..48: note = Description of Artificial Sequence: Synthetic oligonucleotide
  source 1..48: mol_type = other DNA
cagcagcagcagcagcagcagcagcagcagcagcagcagcagcagcag

SEQ ID NO: 164 - DNA, 51 bp, synthetic construct
  misc_feature 1..51: note = Description of Artificial Sequence: Synthetic oligonucleotide
  source 1..51: mol_type = other DNA
cagcagcagcagcagcagcagcagcagcagcagcagcagcagcagcagcag

SEQ ID NO: 165 - DNA, 54 bp, synthetic construct
  misc_feature 1..54: note = Description of Artificial Sequence: Synthetic oligonucleotide
  source 1..54: mol_type = other DNA
cagcagcagcagcagcagcagcagcagcagcagcagcagcagcagcagcagcag

SEQ ID NO: 166 - DNA, 57 bp, synthetic construct
  misc_feature 1..57: note = Description of Artificial Sequence: Synthetic oligonucleotide
  source 1..57: mol_type = other DNA
cagcagcagcagcagcagcagcagcagcagcagcagcagcagcagcagcagcagcag

SEQ ID NO: 167 - DNA, 60 bp, synthetic construct
  misc_feature 1..60: note = Description of Artificial Sequence: Synthetic oligonucleotide
  source 1..60: mol_type = other DNA
cagcagcagcagcagcagcagcagcagcagcagcagcagcagcagcagcagcagcagcag

SEQ ID NO: 168 - DNA, 63 bp, synthetic construct
  misc_feature 1..63: note = Description of Artificial Sequence: Synthetic oligonucleotide
  source 1..63: mol_type = other DNA
cagcagcagcagcagcagcagcagcagcagcagcagcagcagcagcagcagcagcagcagcag

SEQ ID NO: 169 - DNA, 66 bp, synthetic construct
  misc_feature 1..66: note = Description of Artificial Sequence: Synthetic oligonucleotide
  source 1..66: mol_type = other DNA
cagcagcagcagcagcagcagcagcagcagcagcagcagcagcagcagcagcagcagcagcagcag

SEQ ID NO: 170 - DNA, 69 bp, synthetic construct
  misc_feature 1..69: note = Description of Artificial Sequence: Synthetic oligonucleotide
  source 1..69: mol_type = other DNA
cagcagcagcagcagcagcagcagcagcagcagcagcagcagcagcagcagcagcagcagcagcagcag

SEQ ID NO: 171 - DNA, 72 bp, synthetic construct
  misc_feature 1..72: note = Description of Artificial Sequence: Synthetic oligonucleotide
  source 1..72: mol_type = other DNA
cagcagcagcagcagcagcagcagcagcagcagcagcagcagcagcagcagcagcagcagcagcagcagcag

SEQ ID NO: 172 - DNA, 75 bp, synthetic construct
  misc_feature 1..75: note = Description of Artificial Sequence: Synthetic oligonucleotide
  source 1..75: mol_type = other DNA
cagcagcagcagcagcagcagcagcagcagcagcagcagcagcagcagcagcagcagcagcagcagcagcagcag

SEQ ID NO: 173 - DNA, 78 bp, synthetic construct
  misc_feature 1..78: note = Description of Artificial Sequence: Synthetic oligonucleotide
  source 1..78: mol_type = other DNA
cagcagcagcagcagcagcagcagcagcagcagcagcagcagcagcagcagcagcagcagcagcagcagcagcagcag

SEQ ID NO: 174 - DNA, 81 bp, synthetic construct
  misc_feature 1..81: note = Description of Artificial Sequence: Synthetic oligonucleotide
  source 1..81: mol_type = other DNA
cagcagcagcagcagcagcagcagcagcagcagcagcagcagcagcagcagcagcagcagcagcagcagcagcagcagcag

SEQ ID NO: 175 - DNA, 84 bp, synthetic construct
  misc_feature 1..84: note = Description of Artificial Sequence: Synthetic oligonucleotide
  source 1..84: mol_type = other DNA
cagcagcagcagcagcagcagcagcagcagcagcagcagcagcagcagcagcagcagcagcagcagcagcagcagcagcagcag

SEQ ID NO: 176 - DNA, 87 bp, synthetic construct
  misc_feature 1..87: note = Description of Artificial Sequence: Synthetic oligonucleotide
  source 1..87: mol_type = other DNA
cagcagcagcagcagcagcagcagcagcagcagcagcagcagcagcagcagcagcagcagcagcagcagcagcagcagcagcagcag

SEQ ID NO: 177 - DNA, 90 bp, synthetic construct
  misc_feature 1..90: note = Description of Artificial Sequence: Synthetic oligonucleotide
  source 1..90: mol_type = other DNA
cagcagcagcagcagcagcagcagcagcagcagcagcagcagcagcagcagcagcagcagcagcagcagcagcagcagcagcagcagcag

SEQ ID NO: 178 - DNA, 93 bp, synthetic construct
  misc_feature 1..93: note = Description of Artificial Sequence: Synthetic oligonucleotide
  source 1..93: mol_type = other DNA
cagcagcagcagcagcagcagcagcagcagcagcagcagcagcagcagcagcagcagcagcagcagcagcagcagcagcagcagcagcagcag

SEQ ID NO: 179 - DNA, 96 bp, synthetic construct
  misc_feature 1..96: note = Description of Artificial Sequence: Synthetic oligonucleotide
  source 1..96: mol_type = other DNA
cagcagcagcagcagcagcagcagcagcagcagcagcagcagcagcagcagcagcagcagcagcagcagcagcagcagcagcagcagcagcagcag

SEQ ID NO: 180 - DNA, 99 bp, synthetic construct
  misc_feature 1..99: note = Description of Artificial Sequence: Synthetic oligonucleotide
  source 1..99: mol_type = other DNA
cagcagcagcagcagcagcagcagcagcagcagcagcagcagcagcagcagcagcagcagcagcagcagcagcagcagcagcagcagcagcagcagcag

SEQ ID NO: 181 - DNA, 102 bp, synthetic construct
  misc_feature 1..102: note = Description of Artificial Sequence: Synthetic polynucleotide
  source 1..102: mol_type = other DNA
cagcagcagcagcagcagcagcagcagcagcagcagcagcagcagcagcagcagcagcagcagcagcagcagcagcagcagcagcagcagcagcagcagcag

SEQ ID NO: 182 - DNA, 105 bp, synthetic construct
  misc_feature 1..105: note = Description of Artificial Sequence: Synthetic polynucleotide
  source 1..105: mol_type = other DNA
cagcagcagcagcagcagcagcagcagcagcagcagcagcagcagcagcagcagcagcagcagcagcagcagcagcagcagcagcagcagcagcagcagcagcag

SEQ ID NO: 183 - DNA, 108 bp, synthetic construct
  misc_feature 1..108: note = Description of Artificial Sequence: Synthetic polynucleotide
  source 1..108: mol_type = other DNA
cagcagcagcagcagcagcagcagcagcagcagcagcagcagcagcagcagcagcagcagcagcagcagcagcagcagcagcagcagcagcagcagcagcagcagcag

SEQ ID NO: 184 - DNA, 111 bp, synthetic construct
  misc_feature 1..111: note = Description of Artificial Sequence: Synthetic polynucleotide
  source 1..111: mol_type = other DNA
cagcagcagcagcagcagcagcagcagcagcagcagcagcagcagcagcagcagcagcagcagcagcagcagcagcagcagcagcagcagcagcagcagcagcagcagcag

SEQ ID NO: 185 - DNA, 114 bp, synthetic construct
  misc_feature 1..114: note = Description of Artificial Sequence: Synthetic polynucleotide
  source 1..114: mol_type = other DNA
cagcagcagcagcagcagcagcagcagcagcagcagcagcagcagcagcagcagcagcagcagcagcagcagcagcagcagcagcagcagcagcagcagcagcagcagcagcag

SEQ ID NO: 186 - DNA, 117 bp, synthetic construct
  misc_feature 1..117: note = Description of Artificial Sequence: Synthetic polynucleotide
  source 1..117: mol_type = other DNA
cagcagcagcagcagcagcagcagcagcagcagcagcagcagcagcagcagcagcagcagcagcagcagcagcagcagcagcagcagcagcagcagcagcagcagcagcagcagcag

SEQ ID NO: 187 - DNA, 120 bp, synthetic construct
  misc_feature 1..120: note = Description of Artificial Sequence: Synthetic polynucleotide
  source 1..120: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 188 - DNA, 123 bp, synthetic construct
  misc_feature 1..123: note = Description of Artificial Sequence: Synthetic polynucleotide
  source 1..123: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 189 - DNA, 126 bp, synthetic construct
  misc_feature 1..126: note = Description of Artificial Sequence: Synthetic polynucleotide
  source 1..126: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 190 - DNA, 129 bp, synthetic construct
  misc_feature 1..129: note = Description of Artificial Sequence: Synthetic polynucleotide
  source 1..129: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 191 - DNA, 132 bp, synthetic construct
  misc_feature 1..132: note = Description of Artificial Sequence: Synthetic polynucleotide
  source 1..132: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 192 - DNA, 135 bp, synthetic construct
  misc_feature 1..135: note = Description of Artificial Sequence: Synthetic polynucleotide
  source 1..135: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 193 - DNA, 138 bp, synthetic construct
  misc_feature 1..138: note = Description of Artificial Sequence: Synthetic polynucleotide
  source 1..138: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 194 - DNA, 141 bp, synthetic construct
  misc_feature 1..141: note = Description of Artificial Sequence: Synthetic polynucleotide
  source 1..141: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 195 - DNA, 144 bp, synthetic construct
  misc_feature 1..144: note = Description of Artificial Sequence: Synthetic polynucleotide
  source 1..144: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 196 - DNA, 147 bp, synthetic construct
  misc_feature 1..147: note = Description of Artificial Sequence: Synthetic polynucleotide
  source 1..147: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 197 - DNA, 150 bp, synthetic construct
  misc_feature 1..150: note = Description of Artificial Sequence: Synthetic polynucleotide
  source 1..150: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 198 - DNA, 12 bp, synthetic construct
  misc_feature 1..12: note = Description of Artificial Sequence: Synthetic oligonucleotide
  source 1..12: mol_type = other DNA
agcagcagcagc

SEQ ID NO: 199 - DNA, 15 bp, synthetic construct
  misc_feature 1..15: note = Description of Artificial Sequence: Synthetic oligonucleotide
  source 1..15: mol_type = other DNA
agcagcagcagcagc

SEQ ID NO: 200 - DNA, 18 bp, synthetic construct
  misc_feature 1..18: note = Description of Artificial Sequence: Synthetic oligonucleotide
  source 1..18: mol_type = other DNA
agcagcagcagcagcagc

SEQ ID NO: 201 - DNA, 21 bp, synthetic construct
  misc_feature 1..21: note = Description of Artificial Sequence: Synthetic oligonucleotide
  source 1..21: mol_type = other DNA
agcagcagcagcagcagcagc

SEQ ID NO: 202 - DNA, 24 bp, synthetic construct
  misc_feature 1..24: note = Description of Artificial Sequence: Synthetic oligonucleotide
  source 1..24: mol_type = other DNA
agcagcagcagcagcagcagcagc

SEQ ID NO: 203 - DNA, 27 bp, synthetic construct
  misc_feature 1..27: note = Description of Artificial Sequence: Synthetic oligonucleotide
  source 1..27: mol_type = other DNA
agcagcagcagcagcagcagcagcagc

SEQ ID NO: 204 - DNA, 30 bp, synthetic construct; identical to SEQ ID NO: 149
  misc_feature 1..30: note = Description of Artificial Sequence: Synthetic oligonucleotide
  source 1..30: mol_type = other DNA

SEQ ID NO: 205 - DNA, 33 bp, synthetic construct
  misc_feature 1..33: note = Description of Artificial Sequence: Synthetic oligonucleotide
  source 1..33: mol_type = other DNA
agcagcagcagcagcagcagcagcagcagcagc

SEQ ID NO: 206 - DNA, 36 bp, synthetic construct
  misc_feature 1..36: note = Description of Artificial Sequence: Synthetic oligonucleotide
  source 1..36: mol_type = other DNA
agcagcagcagcagcagcagcagcagcagcagcagc

SEQ ID NO: 207 - DNA, 39 bp, synthetic construct
  misc_feature 1..39: note = Description of Artificial Sequence: Synthetic oligonucleotide
  source 1..39: mol_type = other DNA
agcagcagcagcagcagcagcagcagcagcagcagcagc

SEQ ID NO: 208 - DNA, 42 bp, synthetic construct
  misc_feature 1..42: note = Description of Artificial Sequence: Synthetic oligonucleotide
  source 1..42: mol_type = other DNA
agcagcagcagcagcagcagcagcagcagcagcagcagcagc

SEQ ID NO: 209 - DNA, 45 bp, synthetic construct
  misc_feature 1..45: note = Description of Artificial Sequence: Synthetic oligonucleotide
  source 1..45: mol_type = other DNA
agcagcagcagcagcagcagcagcagcagcagcagcagcagcagc

SEQ ID NO: 210 - DNA, 48 bp, synthetic construct
  misc_feature 1..48: note = Description of Artificial Sequence: Synthetic oligonucleotide
  source 1..48: mol_type = other DNA
agcagcagcagcagcagcagcagcagcagcagcagcagcagcagcagc

SEQ ID NO: 211 - DNA, 51 bp, synthetic construct
  misc_feature 1..51: note = Description of Artificial Sequence: Synthetic oligonucleotide
  source 1..51: mol_type = other DNA
agcagcagcagcagcagcagcagcagcagcagcagcagcagcagcagcagc

SEQ ID NO: 212 - DNA, 54 bp, synthetic construct
  misc_feature 1..54: note = Description of Artificial Sequence: Synthetic oligonucleotide
  source 1..54: mol_type = other DNA
agcagcagcagcagcagcagcagcagcagcagcagcagcagcagcagcagcagc

SEQ ID NO: 213 - DNA, 57 bp, synthetic construct
  misc_feature 1..57: note = Description of Artificial Sequence: Synthetic oligonucleotide
  source 1..57: mol_type = other DNA
agcagcagcagcagcagcagcagcagcagcagcagcagcagcagcagcagcagcagc

SEQ ID NO: 214 - DNA, 60 bp, synthetic construct
  misc_feature 1..60: note = Description of Artificial Sequence: Synthetic oligonucleotide
  source 1..60: mol_type = other DNA
agcagcagcagcagcagcagcagcagcagcagcagcagcagcagcagcagcagcagcagc

SEQ ID NO: 215 - DNA, 63 bp, synthetic construct
  misc_feature 1..63: note = Description of Artificial Sequence: Synthetic oligonucleotide
  source 1..63: mol_type = other DNA
agcagcagcagcagcagcagcagcagcagcagcagcagcagcagcagcagcagcagcagcagc

SEQ ID NO: 216 - DNA, 66 bp, synthetic construct
  misc_feature 1..66: note = Description of Artificial Sequence: Synthetic oligonucleotide
  source 1..66: mol_type = other DNA
agcagcagcagcagcagcagcagcagcagcagcagcagcagcagcagcagcagcagcagcagcagc

SEQ ID NO: 217 - DNA, 69 bp, synthetic construct
  misc_feature 1..69: note = Description of Artificial Sequence: Synthetic oligonucleotide
  source 1..69: mol_type = other DNA
agcagcagcagcagcagcagcagcagcagcagcagcagcagcagcagcagcagcagcagcagcagcagc

SEQ ID NO: 218 - DNA, 72 bp, synthetic construct
  misc_feature 1..72: note = Description of Artificial Sequence: Synthetic oligonucleotide
  source 1..72: mol_type = other DNA
agcagcagcagcagcagcagcagcagcagcagcagcagcagcagcagcagcagcagcagcagcagcagcagc

SEQ ID NO: 219 - DNA, 75 bp, synthetic construct
  misc_feature 1..75: note = Description of Artificial Sequence: Synthetic oligonucleotide
  source 1..75: mol_type = other DNA
agcagcagcagcagcagcagcagcagcagcagcagcagcagcagcagcagcagcagcagcagcagcagcagcagc

SEQ ID NO: 220 - DNA, 78 bp, synthetic construct
  misc_feature 1..78: note = Description of Artificial Sequence: Synthetic oligonucleotide
  source 1..78: mol_type = other DNA
agcagcagcagcagcagcagcagcagcagcagcagcagcagcagcagcagcagcagcagcagcagcagcagcagcagc

SEQ ID NO: 221 - DNA, 81 bp, synthetic construct
  misc_feature 1..81: note = Description of Artificial Sequence: Synthetic oligonucleotide
  source 1..81: mol_type = other DNA
agcagcagcagcagcagcagcagcagcagcagcagcagcagcagcagcagcagcagcagcagcagcagcagcagcagcagc

SEQ ID NO: 222 - DNA, 84 bp, synthetic construct
  misc_feature 1..84: note = Description of Artificial Sequence: Synthetic oligonucleotide
  source 1..84: mol_type = other DNA
agcagcagcagcagcagcagcagcagcagcagcagcagcagcagcagcagcagcagcagcagcagcagcagcagcagcagcagc

SEQ ID NO: 223 - DNA, 87 bp, synthetic construct
  misc_feature 1..87: note = Description of Artificial Sequence: Synthetic oligonucleotide
  source 1..87: mol_type = other DNA
agcagcagcagcagcagcagcagcagcagcagcagcagcagcagcagcagcagcagcagcagcagcagcagcagcagcagcagcagc

SEQ ID NO: 224 - DNA, 90 bp, synthetic construct
  misc_feature 1..90: note = Description of Artificial Sequence: Synthetic oligonucleotide
  source 1..90: mol_type = other DNA
agcagcagcagcagcagcagcagcagcagcagcagcagcagcagcagcagcagcagcagcagcagcagcagcagcagcagcagcagcagc

SEQ ID NO: 225 - DNA, 93 bp, synthetic construct
  misc_feature 1..93: note = Description of Artificial Sequence: Synthetic oligonucleotide
  source 1..93: mol_type = other DNA
agcagcagcagcagcagcagcagcagcagcagcagcagcagcagcagcagcagcagcagcagcagcagcagcagcagcagcagcagcagcagc

SEQ ID NO: 226 - DNA, 96 bp, synthetic construct
  misc_feature 1..96: note = Description of Artificial Sequence: Synthetic oligonucleotide
  source 1..96: mol_type = other DNA
agcagcagcagcagcagcagcagcagcagcagcagcagcagcagcagcagcagcagcagcagcagcagcagcagcagcagcagcagcagcagcagc

SEQ ID NO: 227 - DNA, 99 bp, synthetic construct
  misc_feature 1..99: note = Description of Artificial Sequence: Synthetic oligonucleotide
  source 1..99: mol_type = other DNA
agcagcagcagcagcagcagcagcagcagcagcagcagcagcagcagcagcagcagcagcagcagcagcagcagcagcagcagcagcagcagcagcagc

SEQ ID NO: 228 - DNA, 102 bp, synthetic construct
  misc_feature 1..102: note = Description of Artificial Sequence: Synthetic polynucleotide
  source 1..102: mol_type = other DNA
agcagcagcagcagcagcagcagcagcagcagcagcagcagcagcagcagcagcagcagcagcagcagcagcagcagcagcagcagcagcagcagcagcagc

SEQ ID NO: 229 - DNA, 105 bp, synthetic construct
  misc_feature 1..105: note = Description of Artificial Sequence: Synthetic polynucleotide
  source 1..105: mol_type = other DNA
agcagcagcagcagcagcagcagcagcagcagcagcagcagcagcagcagcagcagcagcagcagcagcagcagcagcagcagcagcagcagcagcagcagcagc

SEQ ID NO: 230 - DNA, 108 bp, synthetic construct
  misc_feature 1..108: note = Description of Artificial Sequence: Synthetic polynucleotide
  source 1..108: mol_type = other DNA
agcagcagcagcagcagcagcagcagcagcagcagcagcagcagcagcagcagcagcagcagcagcagcagcagcagcagcagcagcagcagcagcagcagcagcagc

SEQ ID NO: 231 - DNA, 111 bp, synthetic construct
  misc_feature 1..111: note = Description of Artificial Sequence: Synthetic polynucleotide
  source 1..111: mol_type = other DNA
agcagcagcagcagcagcagcagcagcagcagcagcagcagcagcagcagcagcagcagcagcagcagcagcagcagcagcagcagcagcagcagcagcagcagcagcagc

SEQ ID NO: 232 - DNA, 114 bp, synthetic construct
  misc_feature 1..114: note = Description of Artificial Sequence: Synthetic polynucleotide
  source 1..114: mol_type = other DNA
agcagcagcagcagcagcagcagcagcagcagcagcagcagcagcagcagcagcagcagcagcagcagcagcagcagcagcagcagcagcagcagcagcagcagcagcagcagc

SEQ ID NO: 233 - DNA, 117 bp, synthetic construct
  misc_feature 1..117: note = Description of Artificial Sequence: Synthetic polynucleotide
  source 1..117: mol_type = other DNA
agcagcagcagcagcagcagcagcagcagcagcagcagcagcagcagcagcagcagcagcagcagcagcagcagcagcagcagcagcagcagcagcagcagcagcagcagcagcagc

SEQ ID NO: 234 - DNA, 120 bp, synthetic construct
  misc_feature 1..120: note = Description of Artificial Sequence: Synthetic polynucleotide
  source 1..120: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 235 - DNA, 123 bp, synthetic construct
  misc_feature 1..123: note = Description of Artificial Sequence: Synthetic polynucleotide
  source 1..123: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 236 - DNA, 126 bp, synthetic construct
  misc_feature 1..126: note = Description of Artificial Sequence: Synthetic polynucleotide
  source 1..126: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 237 - DNA, 129 bp, synthetic construct
  misc_feature 1..129: note = Description of Artificial Sequence: Synthetic polynucleotide
  source 1..129: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 238 - DNA, 132 bp, synthetic construct
  misc_feature 1..132: note = Description of Artificial Sequence: Synthetic polynucleotide
  source 1..132: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 239 - DNA, 135 bp, synthetic construct
  misc_feature 1..135: note = Description of Artificial Sequence: Synthetic polynucleotide
  source 1..135: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 240 - DNA, 138 bp, synthetic construct
  misc_feature 1..138: note = Description of Artificial Sequence: Synthetic polynucleotide
  source 1..138: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 241 - DNA, 141 bp, synthetic construct
  misc_feature 1..141: note = Description of Artificial Sequence: Synthetic polynucleotide
  source 1..141: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 242 - DNA, 144 bp, synthetic construct
  misc_feature 1..144: note = Description of Artificial Sequence: Synthetic polynucleotide
  source 1..144: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 243 - DNA, 147 bp, synthetic construct
  misc_feature 1..147: note = Description of Artificial Sequence: Synthetic polynucleotide
  source 1..147: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 244 - DNA, 150 bp, synthetic construct
  misc_feature 1..150: note = Description of Artificial Sequence: Synthetic polynucleotide
  source 1..150: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagc

SEQ ID NO: 245 - DNA, 12 bp, synthetic construct
  misc_feature 1..12: note = Description of Artificial Sequence: Synthetic oligonucleotide
  source 1..12: mol_type = other DNA
gcagcagcagca

SEQ ID NO: 246 - DNA, 15 bp, synthetic construct
  misc_feature 1..15: note = Description of Artificial Sequence: Synthetic oligonucleotide
  source 1..15: mol_type = other DNA
gcagcagcagcagca

SEQ ID NO: 247 - DNA, 18 bp, synthetic construct
  misc_feature 1..18: note = Description of Artificial Sequence: Synthetic oligonucleotide
  source 1..18: mol_type = other DNA
gcagcagcagcagcagca

SEQ ID NO: 248 - DNA, 21 bp, synthetic construct
  misc_feature 1..21: note = Description of Artificial Sequence: Synthetic oligonucleotide
  source 1..21: mol_type = other DNA
gcagcagcagcagcagcagca

SEQ ID NO: 249 - DNA, 24 bp, synthetic construct
  misc_feature 1..24: note = Description of Artificial Sequence: Synthetic oligonucleotide
  source 1..24: mol_type = other DNA
gcagcagcagcagcagcagcagca

SEQ ID NO: 250 - DNA, 27 bp, synthetic construct
  misc_feature 1..27: note = Description of Artificial Sequence: Synthetic oligonucleotide
  source 1..27: mol_type = other DNA
gcagcagcagcagcagcagcagcagca

SEQ ID NO: 251 - DNA, 30 bp, synthetic construct
  misc_feature 1..30: note = Description of Artificial Sequence: Synthetic oligonucleotide
  source 1..30: mol_type = other DNA
gcagcagcagcagcagcagcagcagcagca

SEQ ID NO: 252 - DNA, 33 bp, synthetic construct
  misc_feature 1..33: note = Description of Artificial Sequence: Synthetic oligonucleotide
  source 1..33: mol_type = other DNA
gcagcagcagcagcagcagcagcagcagcagca

SEQ ID NO: 253 - DNA, 36 bp, synthetic construct
  misc_feature 1..36: note = Description of Artificial Sequence: Synthetic oligonucleotide
  source 1..36: mol_type = other DNA
gcagcagcagcagcagcagcagcagcagcagcagca

SEQ ID NO: 254 - DNA, 39 bp, synthetic construct
  misc_feature 1..39: note = Description of Artificial Sequence: Synthetic oligonucleotide
  source 1..39: mol_type = other DNA
gcagcagcagcagcagcagcagcagcagcagcagcagca

SEQ ID NO: 255 - DNA, 42 bp, synthetic construct
  misc_feature 1..42: note = Description of Artificial Sequence: Synthetic oligonucleotide
  source 1..42: mol_type = other DNA
gcagcagcagcagcagcagcagcagcagcagcagcagcagca

SEQ ID NO: 256 - DNA, 45 bp, synthetic construct
  misc_feature 1..45: note = Description of Artificial Sequence: Synthetic oligonucleotide
  source 1..45: mol_type = other DNA
gcagcagcagcagcagcagcagcagcagcagcagcagcagcagca

SEQ ID NO: 257 - DNA, 48 bp, synthetic construct
  misc_feature 1..48: note = Description of Artificial Sequence: Synthetic oligonucleotide
  source 1..48: mol_type = other DNA
gcagcagcagcagcagcagcagcagcagcagcagcagcagcagcagca

SEQ ID NO: 258 - DNA, 51 bp, synthetic construct
  misc_feature 1..51: note = Description of Artificial Sequence: Synthetic oligonucleotide
  source 1..51: mol_type = other DNA
gcagcagcagcagcagcagcagcagcagcagcagcagcagcagcagcagca

SEQ ID NO: 259 - DNA, 54 bp, synthetic construct
  misc_feature 1..54: note = Description of Artificial Sequence: Synthetic oligonucleotide
  source 1..54: mol_type = other DNA
gcagcagcagcagcagcagcagcagcagcagcagcagcagcagcagcagcagca

SEQ ID NO: 260 - DNA, 57 bp, synthetic construct
  misc_feature 1..57: note = Description of Artificial Sequence: Synthetic oligonucleotide
  source 1..57: mol_type = other DNA
gcagcagcagcagcagcagcagcagcagcagcagcagcagcagcagcagcagcagca

SEQ ID NO: 261 - DNA, 60 bp, synthetic construct
  misc_feature 1..60: note = Description of Artificial Sequence: Synthetic oligonucleotide
  source 1..60: mol_type = other DNA
gcagcagcagcagcagcagcagcagcagcagcagcagcagcagcagcagcagcagcagca

SEQ ID NO: 262 - DNA, 63 bp, synthetic construct
  misc_feature 1..63: note = Description of Artificial Sequence: Synthetic oligonucleotide
  source 1..63: mol_type = other DNA
gcagcagcagcagcagcagcagcagcagcagcagcagcagcagcagcagcagcagcagcagca

SEQ ID NO: 263 - DNA, 66 bp, synthetic construct
  misc_feature 1..66: note = Description of Artificial Sequence: Synthetic oligonucleotide
  source 1..66: mol_type = other DNA
gcagcagcagcagcagcagcagcagcagcagcagcagcagcagcagcagcagcagcagcagcagca

SEQ ID NO: 264 - DNA, 69 bp, synthetic construct
  misc_feature 1..69: note = Description of Artificial Sequence: Synthetic oligonucleotide
  source 1..69: mol_type = other DNA
gcagcagcagcagcagcagcagcagcagcagcagcagcagcagcagcagcagcagcagcagcagcagca

SEQ ID NO: 265 - DNA, 72 bp, synthetic construct
  misc_feature 1..72: note = Description of Artificial Sequence: Synthetic oligonucleotide
  source 1..72: mol_type = other DNA
gcagcagcagcagcagcagcagcagcagcagcagcagcagcagcagcagcagcagcagcagcagcagcagca

SEQ ID NO: 266 - DNA, 75 bp, synthetic construct
  misc_feature 1..75: note = Description of Artificial Sequence: Synthetic oligonucleotide
  source 1..75: mol_type = other DNA
gcagcagcagcagcagcagcagcagcagcagcagcagcagcagcagcagcagcagcagcagcagcagcagcagca

SEQ ID NO: 267 - DNA, 78 bp, synthetic construct
  misc_feature 1..78: note = Description of Artificial Sequence: Synthetic oligonucleotide
  source 1..78: mol_type = other DNA
gcagcagcagcagcagcagcagcagcagcagcagcagcagcagcagcagcagcagcagcagcagcagcagcagcagca

SEQ ID NO: 268 - DNA, 81 bp, synthetic construct
  misc_feature 1..81: note = Description of Artificial Sequence: Synthetic oligonucleotide
  source 1..81: mol_type = other DNA
gcagcagcagcagcagcagcagcagcagcagcagcagcagcagcagcagcagcagcagcagcagcagcagcagcagcagca

SEQ ID NO: 269 - DNA, 84 bp, synthetic construct
  misc_feature 1..84: note = Description of Artificial Sequence: Synthetic oligonucleotide
  source 1..84: mol_type = other DNA
gcagcagcagcagcagcagcagcagcagcagcagcagcagcagcagcagcagcagcagcagcagcagcagcagcagcagcagca

SEQ ID NO: 270 - DNA, 87 bp, synthetic construct
  misc_feature 1..87: note = Description of Artificial Sequence: Synthetic oligonucleotide
  source 1..87: mol_type = other DNA
gcagcagcagcagcagcagcagcagcagcagcagcagcagcagcagcagcagcagcagcagcagcagcagcagcagcagcagcagca

SEQ ID NO: 271 - DNA, 90 bp, synthetic construct
  misc_feature 1..90: note = Description of Artificial Sequence: Synthetic oligonucleotide
  source 1..90: mol_type = other DNA
gcagcagcagcagcagcagcagcagcagcagcagcagcagcagcagcagcagcagcagcagcagcagcagcagcagcagcagcagcagca

SEQ ID NO: 272 - DNA, 93 bp, synthetic construct
  misc_feature 1..93: note = Description of Artificial Sequence: Synthetic oligonucleotide
  source 1..93: mol_type = other DNA
gcagcagcagcagcagcagcagcagcagcagcagcagcagcagcagcagcagcagcagcagcagcagcagcagcagcagcagcagcagcagca

SEQ ID NO: 273 - DNA, 96 bp, synthetic construct
  misc_feature 1..96: note = Description of Artificial Sequence: Synthetic oligonucleotide
  source 1..96: mol_type = other DNA
gcagcagcagcagcagcagcagcagcagcagcagcagcagcagcagcagcagcagcagcagcagcagcagcagcagcagcagcagcagcagcagca

SEQ ID NO: 274 - DNA, 99 bp, synthetic construct
  misc_feature 1..99: note = Description of Artificial Sequence: Synthetic oligonucleotide
  source 1..99: mol_type = other DNA
gcagcagcagcagcagcagcagcagcagcagcagcagcagcagcagcagcagcagcagcagcagcagcagcagcagcagcagcagcagcagcagcagca

SEQ ID NO: 275 - DNA, 102 bp, synthetic construct
  misc_feature 1..102: note = Description of Artificial Sequence: Synthetic polynucleotide
  source 1..102: mol_type = other DNA
gcagcagcagcagcagcagcagcagcagcagcagcagcagcagcagcagcagcagcagcagcagcagcagcagcagcagcagcagcagcagcagcagcagca

SEQ ID NO: 276 - DNA, 105 bp, synthetic construct
  misc_feature 1..105: note = Description of Artificial Sequence: Synthetic polynucleotide
  source 1..105: mol_type = other DNA
gcagcagcagcagcagcagcagcagcagcagcagcagcagcagcagcagcagcagcagcagcagcagcagcagcagcagcagcagcagcagcagcagcagcagca

SEQ ID NO: 277 - DNA, 108 bp, synthetic construct
  misc_feature 1..108: note = Description of Artificial Sequence: Synthetic polynucleotide
  source 1..108: mol_type = other DNA
gcagcagcagcagcagcagcagcagcagcagcagcagcagcagcagcagcagcagcagcagcagcagcagcagcagcagcagcagcagcagcagcagcagcagcagca

SEQ ID NO: 278 - DNA, 111 bp, synthetic construct
  misc_feature 1..111: note = Description of Artificial Sequence: Synthetic polynucleotide
  source 1..111: mol_type = other DNA
gcagcagcagcagcagcagcagcagcagcagcagcagcagcagcagcagcagcagcagcagcagcagcagcagcagcagcagcagcagcagcagcagcagcagcagcagca

SEQ ID NO: 279 - DNA, 114 bp, synthetic construct
  misc_feature 1..114: note = Description of Artificial Sequence: Synthetic polynucleotide
  source 1..114: mol_type = other DNA
gcagcagcagcagcagcagcagcagcagcagcagcagcagcagcagcagcagcagcagcagcagcagcagcagcagcagcagcagcagcagcagcagcagcagcagcagcagca

SEQ ID NO: 280 - DNA, 117 bp, synthetic construct
  misc_feature 1..117: note = Description of Artificial Sequence: Synthetic polynucleotide
  source 1..117: mol_type = other DNA
gcagcagcagcagcagcagcagcagcagcagcagcagcagcagcagcagcagcagcagcagcagcagcagcagcagcagcagcagcagcagcagcagcagcagcagcagcagcagca

SEQ ID NO: 281 - DNA, 120 bp, synthetic construct
  misc_feature 1..120: note = Description of Artificial Sequence: Synthetic polynucleotide
  source 1..120: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 282 - DNA, 123 bp, synthetic construct
  misc_feature 1..123: note = Description of Artificial Sequence: Synthetic polynucleotide
  source 1..123: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 283 - DNA, 126 bp, synthetic construct
  misc_feature 1..126: note = Description of Artificial Sequence: Synthetic polynucleotide
  source 1..126: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 284 - DNA, 129 bp, synthetic construct
  misc_feature 1..129: note = Description of Artificial Sequence: Synthetic polynucleotide
  source 1..129: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 285 - DNA, 132 bp, synthetic construct
  misc_feature 1..132: note = Description of Artificial Sequence: Synthetic polynucleotide
  source 1..132: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 286 - DNA, 135 bp, synthetic construct
  misc_feature 1..135: note = Description of Artificial Sequence: Synthetic polynucleotide
  source 1..135: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 287 - DNA, 138 bp, synthetic construct
  misc_feature 1..138: note = Description of Artificial Sequence: Synthetic polynucleotide
  source 1..138: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 288 - DNA, 141 bp, synthetic construct
  misc_feature 1..141: note = Description of Artificial Sequence: Synthetic polynucleotide
  source 1..141: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 289 - DNA, 144 bp, synthetic construct
  misc_feature 1..144: note = Description of Artificial Sequence: Synthetic polynucleotide
  source 1..144: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 290 - DNA, 147 bp, synthetic construct
  misc_feature 1..147: note = Description of Artificial Sequence: Synthetic polynucleotide
  source 1..147: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 291 - DNA, 150 bp, synthetic construct
  misc_feature 1..150: note = Description of Artificial Sequence: Synthetic polynucleotide
  source 1..150: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagca

SEQ ID NO: 292-317 - skipped (26 entries)

SEQ ID NO: 318 - DNA, 25 bp, synthetic construct
  misc_feature 1..25: note = Description of Artificial Sequence: Synthetic oligonucleotide
  source 1..25: mol_type = other DNA
  misc_feature 1..25: note = Phosphorodiamidate morpholino backbone
agccagagcaccgcaaccggacgag

SEQ ID NO: 319 - DNA, 22 bp, synthetic construct
  misc_feature 1..22: note = Description of Artificial Sequence: Synthetic oligonucleotide
  source 1..22: mol_type = other DNA
ttcacatcgccagcatctgtgc

SEQ ID NO: 320 - DNA, 25 bp, synthetic construct
  misc_feature 1..25: note = Description of Artificial Sequence: Synthetic oligonucleotide
  source 1..25: mol_type = other DNA
cacggaacacaaaggcactgaatgt

SEQ ID NO: 321 - DNA, 21 bp, synthetic construct
  misc_feature 1..21: note = Description of Artificial Sequence: Synthetic oligonucleotide
  source 1..21: mol_type = other DNA
gctgcccaataccaggtcaac

SEQ ID NO: 322 - DNA, 22 bp, synthetic construct
  misc_feature 1..22: note = Description of Artificial Sequence: Synthetic oligonucleotide
  source 1..22: mol_type = other DNA
tggtgggagaaatgctgtatgc

SEQ ID NO: 323 - DNA, 23 bp, synthetic construct
  misc_feature 1..23: note = Description of Artificial Sequence: Synthetic oligonucleotide
  source 1..23: mol_type = other DNA
gctcatggtcctcaagatctcac

SEQ ID NO: 324 - DNA, 20 bp, synthetic construct
  misc_feature 1..20: note = Description of Artificial Sequence: Synthetic oligonucleotide
  source 1..20: mol_type = other DNA
gggtcagtgcctcagctttg

SEQ ID NO: 325 - DNA, 20 bp, synthetic construct
  misc_feature 1..20: note = Description of Artificial Sequence: Synthetic oligonucleotide
  source 1..20: mol_type = other DNA
tcgacgacagtgagatggag

SEQ ID NO: 326 - DNA, 20 bp, synthetic construct
  misc_feature 1..20: note = Description of Artificial Sequence: Synthetic oligonucleotide
  source 1..20: mol_type = other DNA
caaactccttcagcgagtcc

SEQ ID NO: 327 - DNA, 20 bp, synthetic construct
  misc_feature 1..20: note = Description of Artificial Sequence: Synthetic oligonucleotide
  source 1..20: mol_type = other DNA
ttccatcgtccaccgcaaat

SEQ ID NO: 328 - DNA, 20 bp, synthetic construct
  misc_feature 1..20: note = Description of Artificial Sequence: Synthetic oligonucleotide
  source 1..20: mol_type = other DNA
agtttacgatggcagcaacg

SEQ ID NO: 329 - DNA, 21 bp, synthetic construct
  misc_feature 1..21: note = Description of Artificial Sequence: Synthetic oligonucleotide
  source 1..21: mol_type = other DNA
aggtcggtgtgaacggatttg

SEQ ID NO: 330 - DNA, 23 bp, synthetic construct
  misc_feature 1..23: note = Description of Artificial Sequence: Synthetic oligonucleotide
  source 1..23: mol_type = other DNA
tgtagaccatgtagttgaggtca

SEQ ID NO: 331-368 - skipped (38 entries)

SEQ ID NO: 369 - DNA, 15 bp, synthetic construct
  misc_feature 1..15: note = Description of Artificial Sequence: Synthetic oligonucleotide
  source 1..15: mol_type = other DNA
taacgttgagggcat

SEQ ID NO: 370 - protein, 7 aa, synthetic construct
  REGION 1..7: note = Description of Artificial Sequence: Synthetic peptide
  SITE 1: note = Acetylated Residue
PKKKRKV

SEQ ID NO: 371 - protein, 41 aa, synthetic construct
  REGION 1..41: note = Description of Artificial Sequence: Synthetic polypeptide
RMRKFKNKGKDTAELRRRRVEVSVELRKAKKDEQILKRRNV

SEQ ID NO: 372 - DNA, 30 bp, synthetic construct; identical to SEQ ID NO: 251
  misc_feature 1..30: note = Description of Artificial Sequence: Synthetic oligonucleotide
  source 1..30: mol_type = other DNA
  misc_feature 1..30: note = Phosphorodiamidate morpholino backbone

SEQ ID NO: 373 - DNA, 25 bp, synthetic construct
  misc_feature 1..25: note = Description of Artificial Sequence: Synthetic oligonucleotide
  source 1..25: mol_type = other DNA
gggccttttattcgcgagggtcggg

SEQ ID NO: 374 - DNA, 25 bp, synthetic construct
  misc_feature 1..25: note = Description of Artificial Sequence: Synthetic oligonucleotide
  source 1..25: mol_type = other DNA
gagggccttttattcgcgagggtcg

SEQ ID NO: 375 - DNA, 25 bp, synthetic construct
  misc_feature 1..25: note = Description of Artificial Sequence: Synthetic oligonucleotide
  source 1..25: mol_type = other DNA
tggagggccttttattcgcgagggt

SEQ ID NO: 376 - DNA, 25 bp, synthetic construct
  misc_feature 1..25: note = Description of Artificial Sequence: Synthetic oligonucleotide
  source 1..25: mol_type = other DNA
gatggagggccttttattcgcgagg

SEQ ID NO: 377 - DNA, 25 bp, synthetic construct
  misc_feature 1..25: note = Description of Artificial Sequence: Synthetic oligonucleotide
  source 1..25: mol_type = other DNA
cagatggagggccttttattcgcga

SEQ ID NO: 378 - DNA, 25 bp, synthetic construct
  misc_feature 1..25: note = Description of Artificial Sequence: Synthetic oligonucleotide
  source 1..25: mol_type = other DNA
ggcagatggagggccttttattcgc

SEQ ID NO: 379 - DNA, 25 bp, synthetic construct
  misc_feature 1..25: note = Description of Artificial Sequence: Synthetic oligonucleotide
  source 1..25: mol_type = other DNA
tgggcagatggagggccttttattc

SEQ ID NO: 380 - DNA, 25 bp, synthetic construct
  misc_feature 1..25: note = Description of Artificial Sequence: Synthetic oligonucleotide
  source 1..25: mol_type = other DNA
tttgggcagatggagggccttttat

SEQ ID NO: 381 - DNA, 25 bp, synthetic construct
  misc_feature 1..25: note = Description of Artificial Sequence: Synthetic oligonucleotide
  source 1..25: mol_type = other DNA
gctttgggcagatggagggcctttt

SEQ ID NO: 382 - DNA, 25 bp, synthetic construct
  misc_feature 1..25: note = Description of Artificial Sequence: Synthetic oligonucleotide
  source 1..25: mol_type = other DNA
gagctttgggcagatggagggcctt

SEQ ID NO: 383 - DNA, 25 bp, synthetic construct
  misc_feature 1..25: note = Description of Artificial Sequence: Synthetic oligonucleotide
  source 1..25: mol_type = other DNA
cagagctttgggcagatggagggcc

SEQ ID NO: 384 - DNA, 25 bp, synthetic construct
  misc_feature 1..25: note = Description of Artificial Sequence: Synthetic oligonucleotide
  source 1..25: mol_type = other DNA
tccagagctttgggcagatggaggg

SEQ ID NO: 385 - DNA, 25 bp, synthetic construct
  misc_feature 1..25: note = Description of Artificial Sequence: Synthetic oligonucleotide
  source 1..25: mol_type = other DNA
agtccagagctttgggcagatggag

SEQ ID NO: 386 - DNA, 25 bp, synthetic construct
  misc_feature 1..25: note = Description of Artificial Sequence: Synthetic oligonucleotide
  source 1..25: mol_type = other DNA
ggagtccagagctttgggcagatgg

SEQ ID NO: 387 - DNA, 25 bp, synthetic construct
  misc_feature 1..25: note = Description of Artificial Sequence: Synthetic oligonucleotide
  source 1..25: mol_type = other DNA
gtggagtccagagctttgggcagat

SEQ ID NO: 388 - DNA, 25 bp, synthetic construct
  misc_feature 1..25: note = Description of Artificial Sequence: Synthetic oligonucleotide
  source 1..25: mol_type = other DNA
ctgtggagtccagagctttgggcag

SEQ ID NO: 389 - DNA, 25 bp, synthetic construct
  misc_feature 1..25: note = Description of Artificial Sequence: Synthetic oligonucleotide
  source 1..25: mol_type = other DNA
cactgtggagtccagagctttgggc

SEQ ID NO: 390 - DNA, 25 bp, synthetic construct
  misc_feature 1..25: note = Description of Artificial Sequence: Synthetic oligonucleotide
  source 1..25: mol_type = other DNA
gacactgtggagtccagagctttgg

SEQ ID NO: 391 - DNA, 25 bp, synthetic construct
  misc_feature 1..25: note = Description of Artificial Sequence: Synthetic oligonucleotide
  source 1..25: mol_type = other DNA
cggacactgtggagtccagagcttt

SEQ ID NO: 392 - DNA, 25 bp, synthetic construct
  misc_feature 1..25: note = Description of Artificial Sequence: Synthetic oligonucleotide
  source 1..25: mol_type = other DNA
cgcggacactgtggagtccagagct

SEQ ID NO: 393 - DNA, 25 bp, synthetic construct
  misc_feature 1..25: note = Description of Artificial Sequence: Synthetic oligonucleotide
  source 1..25: mol_type = other DNA
accgcggacactgtggagtccagag

SEQ ID NO: 394 - DNA, 25 bp, synthetic construct
  misc_feature 1..25: note = Description of Artificial Sequence: Synthetic oligonucleotide
  source 1..25: mol_type = other DNA
aaaccgcggacactgtggagtccag

SEQ ID NO: 395 - DNA, 25 bp, synthetic construct
  misc_feature 1..25: note = Description of Artificial Sequence: Synthetic oligonucleotide
  source 1..25: mol_type = other DNA
gcaaaccgcggacactgtggagtcc

SEQ ID NO: 396 - DNA, 25 bp, synthetic construct
  misc_feature 1..25: note = Description of Artificial Sequence: Synthetic oligonucleotide
  source 1..25: mol_type = other DNA
acgcaaaccgcggacactgtggagt

SEQ ID NO: 397 - DNA, 25 bp, synthetic construct
  misc_feature 1..25: note = Description of Artificial Sequence: Synthetic oligonucleotide
  source 1..25: mol_type = other DNA
caacgcaaaccgcggacactgtgga

SEQ ID NO: 398 - DNA, 120 bp, synthetic construct; identical to SEQ ID NO: 187
  misc_feature 1..120: note = Description of Artificial Sequence: Synthetic polynucleotide
  misc_difference 1..120: note = misc_feature - This sequence may encompass 5-40 "cag" repeating units
  source 1..120: mol_type = other DNA

SEQ ID NO: 399 - DNA, 150 bp, synthetic construct; identical to SEQ ID NO: 197
  misc_feature 1..150: note = Description of Artificial Sequence: Synthetic polynucleotide
  misc_difference 1..150: note = misc_feature - This sequence may encompass 1-50 "cag" repeating units
  source 1..150: mol_type = other DNA

SEQ ID NO: 400 - DNA, 152 bp, synthetic construct
  misc_feature 1..152: note = Description of Artificial Sequence: Synthetic polynucleotide
  misc_difference 3..152: note = misc_feature - This region may encompass 1-50 "cag" repeating units
  source 1..152: mol_type = other DNA
agcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 401 - DNA, 151 bp, synthetic construct
  misc_feature 1..151: note = Description of Artificial Sequence: Synthetic polynucleotide
  misc_difference 2..151: note = misc_feature - This region may encompass 1-50 "cag" repeating units
  source 1..151: mol_type = other DNA
gcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 402 - DNA, 152 bp, synthetic construct
  misc_feature 1..152: note = Description of Artificial Sequence: Synthetic polynucleotide
  misc_difference 1..150: note = misc_feature - This region may encompass 1-50 "cag" repeating units
  source 1..152: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagag

SEQ ID NO: 403 - DNA, 151 bp, synthetic construct
  misc_feature 1..151: note = Description of Artificial Sequence: Synthetic polynucleotide
  misc_difference 1..150: note = misc_feature - This region may encompass 1-50 "cag" repeating units
  source 1..151: mol_type = other DNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaga

SEQ ID NO: 404 - DNA, 150 bp, synthetic construct; identical to SEQ ID NO: 197
  misc_feature 1..150: note = Description of Artificial Sequence: Synthetic polynucleotide
  misc_difference 1..150: note = misc_feature - This sequence may encompass 2-50 "cag" repeating units
  source 1..150: mol_type = other DNA

SEQ ID NO: 405 - DNA, 60 bp, synthetic construct; identical to SEQ ID NO: 167
  misc_feature 1..60: note = Description of Artificial Sequence: Synthetic polynucleotide
  misc_difference 1..60: note = misc_feature - This sequence may encompass 2-20 "cag" repeating units
  source 1..60: mol_type = other DNA

SEQ ID NO: 406 - DNA, 30 bp, synthetic construct; identical to SEQ ID NO: 148
  misc_feature 1..30: note = Description of Artificial Sequence: Synthetic polynucleotide
  misc_difference 1..30: note = misc_feature - This sequence may encompass 2-10 "cag" repeating units
  source 1..30: mol_type = other DNA

SEQ ID NO: 407 - DNA, 30 bp, synthetic construct; identical to SEQ ID NO: 148
  misc_feature 1..30: note = Description of Artificial Sequence: Synthetic polynucleotide
  misc_difference 1..30: note = misc_feature - This sequence may encompass 4-10 "cag" repeating units
  source 1..30: mol_type = other DNA

SEQ ID NO: 408 - DNA, 30 bp, synthetic construct; identical to SEQ ID NO: 148
  misc_feature 1..30: note = Description of Artificial Sequence: Synthetic polynucleotide
  misc_difference 1..30: note = misc_feature - This sequence may encompass 5-10 "cag" repeating units
  source 1..30: mol_type = other DNA

SEQ ID NO: 409 - DNA, 30 bp, synthetic construct; identical to SEQ ID NO: 148
  misc_feature 1..30: note = Description of Artificial Sequence: Synthetic polynucleotide
  misc_difference 1..30: note = misc_feature - This sequence may encompass 6-10 "cag" repeating units
  source 1..30: mol_type = other DNA

SEQ ID NO: 410 - DNA, 27 bp, synthetic construct
  misc_feature 1..27: note = Description of Artificial Sequence: Synthetic oligonucleotide
  misc_difference 1..27: note = misc_feature - This sequence may encompass 6-9 "cag" repeating units
  source 1..27: mol_type = other DNA
cagcagcagcagcagcagcagcagcag

SEQ ID NO: 411 - DNA, 24 bp, synthetic construct
  misc_feature 1..24: note = Description of Artificial Sequence: Synthetic oligonucleotide
  misc_difference 1..24: note = misc_feature - This sequence may encompass 6-8 "cag" repeating units
  source 1..24: mol_type = other DNA
cagcagcagcagcagcagcagcag

SEQ ID NO: 412 - DNA, 21 bp, synthetic construct
  misc_feature 1..21: note = Description of Artificial Sequence: Synthetic oligonucleotide
  misc_difference 1..21: note = misc_feature - This sequence may encompass 6-7 "cag" repeating units
  source 1..21: mol_type = other DNA
cagcagcagcagcagcagcag

SEQ ID NO: 413 - RNA, 21 nt, synthetic construct
  misc_feature 1..21: note = Description of Artificial Sequence: Synthetic oligonucleotide
  source 1..21: mol_type = other RNA
  misc_feature 1..21: note = Phosphorodiamidate morpholino backbone
ctgctgctgctgctgctgctg

SEQ ID NO: 414 - DNA, 120 bp, synthetic construct; identical to SEQ ID NO: 187
  misc_feature 1..120: note = Description of Artificial Sequence: Synthetic polynucleotide
  misc_difference 1..120: note = misc_feature - This sequence may encompass 10-40 "cag" repeating units
  source 1..120: mol_type = other DNA

SEQ ID NO: 415 - protein, 8 aa, synthetic construct
  SITE 1: note = Acetylated Residue
  SITE 8: note = Residue attached to another sequence via the side chain
PKKKRKVK

SEQ ID NO: 416 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 5: note = D-amino acid
  SITE 6: note = Citrulline
  SITE 7: note = D-amino acid
FFXRRXRQ

SEQ ID NO: 417 - protein, 8 aa, synthetic construct
  SITE 1: note = Acetylated Residue
  SITE 8: note = Residue attached to another sequence via the side chain
PKKKRKVK

SEQ ID NO: 418 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 7: note = D-amino acid
FFXXRRRQ

SEQ ID NO: 419 - protein, 8 aa, synthetic construct
  SITE 1: note = Acetylated Residue
  SITE 8: note = Residue attached to another sequence via the side chain
PKKKRKVK

SEQ ID NO: 420 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 5: note = D-Citrulline
  SITE 7: note = D-Citrulline
FFXRXRXQ

SEQ ID NO: 421 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 422 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 423 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 424 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 425 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 426 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 427 - protein, 8 aa, synthetic construct
  SITE 1: note = Acetylated Residue
  SITE 8: note = Residue attached to another sequence via the side chain
PKKKRKVK

SEQ ID NO: 428 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 429 - protein, 7 aa, synthetic construct
  SITE 1: note = Acetylated D-Pro
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-Valine attached to another sequence via a 2x polyethylene glycol lysine linker
PKKKRKV

SEQ ID NO: 430 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 431 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 432 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 433 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 434 - protein, 7 aa, synthetic construct
  SITE 1: note = Acetylated Residue
  SITE 5: note = Citrulline
  SITE 7: note = Residue attached to another sequence via a 2x polyethylene glycol Lysine linker
PKKKXKV

SEQ ID NO: 435 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 436 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 437 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 438 - protein, 9 aa, synthetic construct
  SITE 1: note = Acetylated Residue
  SITE 8: note = 2-[2-[2-aminoethoxy]ethoxy]acetic acid
  SITE 9: note = Residue attached to another sequence via the side chain
PKKKRKVXK

SEQ ID NO: 439 - protein, 8 aa, synthetic construct
  SITE 1: note = Residue attached to the side chain of another sequence
FGFGRGRQ

SEQ ID NO: 440 - protein, 9 aa, synthetic construct
  SITE 1: note = Acetylated Residue
  SITE 8: note = 2-[2-[2-aminoethoxy]ethoxy]acetic acid
  SITE 9: note = Residue attached to another sequence via the side chain
PKKKRKVXK

SEQ ID NO: 441 - protein, 8 aa, synthetic construct
  SITE 1: note = Residue attached to the side chain of another sequence
  SITE 2: note = D-amino acid
  SITE 5: note = D-amino acid
  SITE 7: note = D-amino acid
GFFGRGRQ

SEQ ID NO: 442 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 443 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
FFFGRGRQ

SEQ ID NO: 444 - protein, 7 aa, synthetic construct
  SITE 1: note = Acetylated Residue
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
  SITE 7: note = Residue attached to another sequence via a 2-[2-[2-aminoethoxy]ethoxy]acetic acid Lysine linker
PKKKRKV

SEQ ID NO: 445 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 446 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 447 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 448 - protein, 8 aa, synthetic construct
  SITE 1: note = Residue attached to another sequence via a 2-[2-[2-aminoethoxy]ethoxy]acetic acid 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 449 - protein, 8 aa, synthetic construct
  SITE 1: note = Residue attached to another sequence via a 2-[2-[2-aminoethoxy]ethoxy]acetic acid 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 450 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 451 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 452 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 453 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 454 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 455 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 456 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 457 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 458 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 459 - protein, 8 aa, synthetic construct
  SITE 1: note = Residue attached to another sequence via a 2x polyethylene glycol 2,3-diaminopropionic acid linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 460 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 461 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 462 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 463 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 464 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 465 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 466 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 467 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 468 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 469 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 470 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 471 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 472 - protein, 4 aa, synthetic construct
  SITE 1: note = Acetylated Residue
  SITE 4: note = Residue attached to another sequence via a 2-[2-[2-aminoethoxy]ethoxy]acetic acid Lysine linker
KKKK

SEQ ID NO: 473 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 474 - protein, 4 aa, synthetic construct
  SITE 1: note = Acetylated Residue
  SITE 4: note = Residue attached to another sequence via a 2-[2-[2-aminoethoxy]ethoxy]acetic acid Lysine linker
KGKK

SEQ ID NO: 475 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 476 - protein, 4 aa, synthetic construct
  SITE 1: note = Acetylated Residue
  SITE 4: note = Residue attached to another sequence via a 2-[2-[2-aminoethoxy]ethoxy]acetic acid Lysine linker
KKGK

SEQ ID NO: 477 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 478 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 479 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 480 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 481 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 482 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 483 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 484 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 485 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 486 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 487 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PGKKRKV

SEQ ID NO: 488 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 489 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKGKRKV

SEQ ID NO: 490 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 491 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKGRKV

SEQ ID NO: 492 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 493 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKGKV

SEQ ID NO: 494 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 495 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRGV

SEQ ID NO: 496 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 497 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKG

SEQ ID NO: 498 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 499 - protein, 5 aa, synthetic construct
  SITE 1: note = Acetylated Residue
  SITE 5: note = Residue attached to another sequence via a 2-[2-[2-aminoethoxy]ethoxy]acetic acid Lysine linker
KKKRK

SEQ ID NO: 500 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 501 - protein, 4 aa, synthetic construct
  SITE 1: note = Acetylated Residue
  SITE 4: note = Residue attached to another sequence via a 2-[2-[2-aminoethoxy]ethoxy]acetic acid Lysine linker
KKRK

SEQ ID NO: 502 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 503 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 504 - protein, 8 aa, synthetic construct
  SITE 1: note = Acetylated Residue
  SITE 7: note = Residue attached to another sequence via the side chain
PKKKRKVK

SEQ ID NO: 505 - protein, 8 aa, synthetic construct
  SITE 1: note = Residue attached to another sequence via the side chain
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 506 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 507 - protein, 8 aa, synthetic construct
  SITE 1: note = Residue attached to another sequence via a 2-[2-[2-aminoethoxy]ethoxy]acetic acid Lysine linker
FGFGRGRQ

SEQ ID NO: 508 - protein, 8 aa, synthetic construct
  SITE 1: note = Residue attached to another sequence via a 2x polyethylene glycol Lysine linker
FGFGRGRQ

SEQ ID NO: 509 - protein, 7 aa, synthetic construct
  SITE 1: note = Acetylated Residue
  SITE 7: note = Residue attached to another sequence via a 2x polyethylene glycol Lysine linker
PKKKGKV

SEQ ID NO: 510 - protein, 8 aa, synthetic construct
  SITE 1: note = Residue attached to another sequence via a 2x polyethylene glycol Lysine linker
FGFGRGRQ

SEQ ID NO: 511 - protein, 7 aa, synthetic construct
  SITE 1: note = Acetylated Residue
  SITE 7: note = Residue attached to another sequence via a 2x polyethylene glycol Lysine linker
PKKKRKG

SEQ ID NO: 512 - protein, 8 aa, synthetic construct
  SITE 1: note = Residue attached to another sequence via a 2x polyethylene glycol Lysine linker
FGFGRGRQ

SEQ ID NO: 513 - protein, 5 aa, synthetic construct
  SITE 1: note = Acetylated Residue
  SITE 5: note = Residue attached to another sequence via a 2x polyethylene glycol Lysine linker
KKKRK

SEQ ID NO: 514 - protein, 8 aa, synthetic construct
  SITE 1: note = Residue attached to another sequence via a 2x polyethylene glycol Lysine linker
FGFGRGRQ

SEQ ID NO: 515 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 516 - protein, 8 aa, synthetic construct
  SITE 1: note = Residue attached to another sequence via a 2-[2-[2-aminoethoxy]ethoxy]acetic acid Lysine linker
  SITE 3: note = 3-(2-naphthyl)-alanine
FFXGRGRQ

SEQ ID NO: 517 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 518 - protein, 8 aa, synthetic construct
  SITE 1: note = Beta-homophenylalanine attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
XFXGRGRQ

SEQ ID NO: 519 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 520 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXSRSRQ

SEQ ID NO: 521 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 522 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 5: note = D-amino acid
  SITE 7: note = D-amino acid
GFFGRGRQ

SEQ ID NO: 523 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 524 - protein, 8 aa, synthetic construct
  SITE 1: note = Residue attached to another sequence via a 2-[2-[2-aminoethoxy]ethoxy]acetic acid Lysine linker
FGFKRKRQ

SEQ ID NO: 525 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 526 - protein, 8 aa, synthetic construct
  SITE 1: note = Residue attached to another sequence via a 2-[2-[2-aminoethoxy]ethoxy]acetic acid Lysine linker
FGFRGRGQ

SEQ ID NO: 527 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 528 - protein, 10 aa, synthetic construct
  SITE 1: note = Residue attached to another sequence via a 2-[2-[2-aminoethoxy]ethoxy]acetic acid Lysine linker
FGFGRGRGRQ

SEQ ID NO: 529 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 530 - protein, 8 aa, synthetic construct
  SITE 1: note = Residue attached to another sequence via a 2-[2-[2-aminoethoxy]ethoxy]acetic acid Lysine linker
  SITE 6: note = D-amino acid
FGFGRRRQ

SEQ ID NO: 531 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 532 - protein, 8 aa, synthetic construct
  SITE 1: note = Residue attached to another sequence via a 2-[2-[2-aminoethoxy]ethoxy]acetic acid Lysine linker
FGFGRRRQ

SEQ ID NO: 533 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 534 - protein, 8 aa, synthetic construct
  SITE 1: note = Residue attached to another sequence via a 2-[2-[2-aminoethoxy]ethoxy]acetic acid Lysine linker
FGFRRRRQ

SEQ ID NO: 535 - protein, 6 aa, synthetic construct
  SITE 1: note = Acetylated Residue
  SITE 6: note = Residue attached to another sequence via a 2-[2-[2-aminoethoxy]ethoxy]acetic acid Lysine linker
KKKRKG

SEQ ID NO: 536 - protein, 8 aa, synthetic construct
  SITE 1: note = Residue attached to another sequence via a 2-[2-[2-aminoethoxy]ethoxy]acetic acid Lysine linker
FGFGRGRQ

SEQ ID NO: 537 - protein, 5 aa, synthetic construct
  SITE 1: note = Acetylated Residue
  SITE 5: note = Residue attached to another sequence via a 2-[2-[2-aminoethoxy]ethoxy]acetic acid Lysine linker
KKKRK

SEQ ID NO: 538 - protein, 8 aa, synthetic construct
  SITE 1: note = Residue attached to another sequence via a 2-[2-[2-aminoethoxy]ethoxy]acetic acid Lysine linker
FGFGRGRQ

SEQ ID NO: 539 - protein, 5 aa, synthetic construct
  SITE 1: note = Acetylated Residue
  SITE 5: note = Residue attached to another sequence via a 4x polyethylene glycol Lysine linker
KKRKK

SEQ ID NO: 540 - protein, 8 aa, synthetic construct
  SITE 1: note = Residue attached to another sequence via a 4x polyethylene glycol Lysine linker
FGFGRGRQ

SEQ ID NO: 541 - protein, 5 aa, synthetic construct
  SITE 1: note = Acetylated Residue
  SITE 5: note = Residue attached to another sequence via a 4x polyethylene glycol Lysine linker
KRKKK

SEQ ID NO: 542 - protein, 8 aa, synthetic construct
  SITE 1: note = Residue attached to another sequence via a 4x polyethylene glycol Lysine linker
FGFGRGRQ

SEQ ID NO: 543 - protein, 5 aa, synthetic construct
  SITE 1: note = Acetylated Residue
  SITE 5: note = Residue attached to another sequence via a 4x polyethylene glycol Lysine linker
KKKKR

SEQ ID NO: 544 - protein, 8 aa, synthetic construct
  SITE 1: note = Residue attached to another sequence via a 4x polyethylene glycol Lysine linker
FGFGRGRQ

SEQ ID NO: 545 - protein, 5 aa, synthetic construct
  SITE 1: note = Acetylated Residue
  SITE 5: note = Residue attached to another sequence via a 4x polyethylene glycol Lysine linker
RKKKK

SEQ ID NO: 546 - protein, 8 aa, synthetic construct
  SITE 1: note = Residue attached to another sequence via a 4x polyethylene glycol Lysine linker
FGFGRGRQ

SEQ ID NO: 547 - protein, 5 aa, synthetic construct
  SITE 1: note = Acetylated Residue
  SITE 5: note = Residue attached to another sequence via a 4x polyethylene glycol Lysine linker
KKKRK

SEQ ID NO: 548 - protein, 8 aa, synthetic construct
  SITE 1: note = Residue attached to another sequence via a 4x polyethylene glycol Lysine linker
FGFGRGRQ

SEQ ID NO: 549 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 550 - protein, 8 aa, synthetic construct
  SITE 1: note = Residue attached to another sequence via a 2x polyethylene glycol Lysine linker
FGFGRGRQ

SEQ ID NO: 551 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 552 - protein, 8 aa, synthetic construct
  SITE 1: note = Residue attached to another sequence via a 2x polyethylene glycol Lysine linker
FGFGRGRQ

SEQ ID NO: 553 - protein, 7 aa, synthetic construct
  SITE 1: note = Acetylated Residue
  SITE 7: note = Residue attached to another sequence via a 2x polyethylene glycol Lysine linker
PKKKRKV

SEQ ID NO: 554 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 555 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 556 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 557 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 558 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 559 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 560 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 561 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 562 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 563 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 564 - protein, 8 aa, synthetic construct
  SITE 1: note = Residue attached to another sequence via a 2x polyethylene glycol Lysine linker
FGFGRGRQ

SEQ ID NO: 565 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 566 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 567 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 568 - protein, 8 aa, synthetic construct
  SITE 1: note = Residue attached to another sequence via a 2x polyethylene glycol Lysine linker
FGFGRRRQ

SEQ ID NO: 569 - protein, 7 aa, synthetic construct
  SITE 1: note = Acetylated Residue
  SITE 7: note = Residue attached to another sequence via a 2x polyethylene glycol Lysine linker
  VARIANT 1..7: note = May have D or L stereochemistry
PKKKRKV

SEQ ID NO: 570 - protein, 8 aa, synthetic construct
  SITE 1: note = Residue attached to another sequence via a 2x polyethylene glycol Lysine linker
FGFRRRRQ

SEQ ID NO: 571 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 572 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 573 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 574 - protein, 8 aa, synthetic construct
  SITE 1: note = Residue attached to another sequence via a 2x polyethylene glycol Lysine linker
FGFGRGRQ

SEQ ID NO: 575 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 576 - protein, 8 aa, synthetic construct
  SITE 1: note = Residue attached to another sequence via a 2x polyethylene glycol Lysine linker
FGFGRRRQ

SEQ ID NO: 577 - protein, 8 aa, synthetic construct
  SITE 1: note = Residue attached to another sequence via a 2x polyethylene glycol Lysine linker
FGFRRRRQ

SEQ ID NO: 578 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 579 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 580 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 581 - protein, 8 aa, synthetic construct
  SITE 1: note = Residue attached to another sequence via a 2x polyethylene glycol Lysine linker
FGFGRGRQ

SEQ ID NO: 582 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 583 - protein, 8 aa, synthetic construct
  SITE 1: note = Residue attached to another sequence via a 2x polyethylene glycol Lysine linker
FGFGRRRQ

SEQ ID NO: 584 - protein, 8 aa, synthetic construct
  SITE 1: note = Residue attached to another sequence via a 2x polyethylene glycol Lysine linker
FGFRRRRQ

SEQ ID NO: 585 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 586 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 587 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 588 - protein, 8 aa, synthetic construct
  SITE 1: note = Residue attached to another sequence via a 2x polyethylene glycol Lysine linker
FGFGRGRQ

SEQ ID NO: 589 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 590 - protein, 8 aa, synthetic construct
  SITE 1: note = Residue attached to another sequence via a 2x polyethylene glycol Lysine linker
FGFGRRRQ

SEQ ID NO: 591 - protein, 8 aa, synthetic construct
  SITE 1: note = Residue attached to another sequence via a 2x polyethylene glycol Lysine linker
FGFRRRRQ

SEQ ID NO: 592 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 593 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 594 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 595 - protein, 8 aa, synthetic construct
  SITE 1: note = Residue attached to another sequence via a 2x polyethylene glycol Lysine linker
FGFGRGRQ

SEQ ID NO: 596 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 597 - protein, 8 aa, synthetic construct
  SITE 1: note = Residue attached to another sequence via a 2x polyethylene glycol Lysine linker
FGFGRRRQ

SEQ ID NO: 598 - protein, 8 aa, synthetic construct
  SITE 1: note = Residue attached to another sequence via a 2x polyethylene glycol Lysine linker
FGFRRRRQ

SEQ ID NO: 599 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 600 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 601 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 602 - protein, 8 aa, synthetic construct
  SITE 1: note = Residue attached to another sequence via a 2x polyethylene glycol Lysine linker
FGFGRGRQ

SEQ ID NO: 603 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 604 - protein, 8 aa, synthetic construct
  SITE 1: note = Residue attached to another sequence via a 2x polyethylene glycol Lysine linker
FGFGRRRQ

SEQ ID NO: 605 - protein, 8 aa, synthetic construct
  SITE 1: note = Residue attached to another sequence via a 2x polyethylene glycol Lysine linker
FGFRRRRQ

SEQ ID NO: 606 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 607 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 608 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 609 - protein, 8 aa, synthetic construct
  SITE 1: note = Residue attached to another sequence via a 2x polyethylene glycol Lysine linker
FGFGRGRQ

SEQ ID NO: 610 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 611 - protein, 8 aa, synthetic construct
  SITE 1: note = Residue attached to another sequence via a 2x polyethylene glycol Lysine linker
FGFGRRRQ

SEQ ID NO: 612 - protein, 8 aa, synthetic construct
  SITE 1: note = Residue attached to another sequence via a 2x polyethylene glycol Lysine linker
FGFRRRRQ

SEQ ID NO: 613 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 614 - protein, 8 aa, synthetic construct
  SITE 1: note = Residue attached to another sequence via a 2x polyethylene glycol Lysine linker
  SITE 2: note = D-amino acid
  SITE 3: note = 3-(2-naphthyl)-alanine
  SITE 4: note = Citrulline
  SITE 5: note = D-amino acid
  SITE 6: note = Citrulline
  SITE 7: note = D-amino acid
FFXXRXRQ

SEQ ID NO: 615 - protein, 8 aa, synthetic construct
  SITE 1: note = Residue attached to another sequence via a 2x polyethylene glycol Lysine linker
  SITE 2: note = D-amino acid
FFFGRGRQ

SEQ ID NO: 616 - protein, 8 aa, synthetic construct
  SITE 1: note = Residue attached to another sequence via a 2x polyethylene glycol Lysine linker
FGFGRGRQ

SEQ ID NO: 617 - protein, 8 aa, synthetic construct
  SITE 1: note = Residue attached to another sequence via a 2x polyethylene glycol Lysine linker
  SITE 2: note = D-amino acid
  SITE 5: note = D-amino acid
  SITE 7: note = D-amino acid
GFFGRGRQ

SEQ ID NO: 618 - protein, 8 aa, synthetic construct
  SITE 1: note = Residue attached to another sequence via a 2x polyethylene glycol Lysine linker
FGFGRRRQ

SEQ ID NO: 619 - protein, 8 aa, synthetic construct
  SITE 1: note = Residue attached to another sequence via a 2x polyethylene glycol Lysine linker
FGFRRRRQ

SEQ ID NO: 620 - protein, 22 aa, synthetic construct
  SITE 1: note = Acetylated Residue
  SITE 2: note = aminohexanoic acid
  SITE 5: note = Beta-alanine
  SITE 8: note = aminohexanoic acid
  SITE 16: note = aminohexanoic acid
  SITE 18: note = Beta-alanine
  SITE 20: note = aminohexanoic acid
  SITE 22: note = Beta-alanine
RXRRXRRXRYQFLIRXRXRXRX

SEQ ID NO: 621 - DNA, 21 bp, synthetic construct
  source 1..21: mol_type = other DNA
  misc_feature 21: note = nucleotide attached to another sequence via a linker
  misc_feature 1..21: note = Phosphorodiamidate morpholino backbone
cagcagcagcagcagcagcag

SEQ ID NO: 622 - DNA, 21 bp, synthetic construct
  source 1..21: mol_type = other DNA
  misc_feature 21: note = nucleotide attached to another sequence via a linker
cagcagcagcagcagcagcag

SEQ ID NO: 623 - protein, 8 aa, synthetic construct
  SITE 1: note = Residue attached to another sequence via a Lysine 12x polyethylene glycol lysine linker
  SITE 2: note = D-amino acid
  SITE 3: note = 3-(2-naphthyl)-alanine
  SITE 5: note = D-amino acid
  SITE 7: note = D-amino acid
  SITE 8: note = Residue attached to another sequence via the side chain
FFXRRRRQ

SEQ ID NO: 624 - DNA, 21 bp, synthetic construct
  source 1..21: mol_type = other DNA
  misc_feature 21: note = nucleotide attached to another sequence via a linker
gtaactgtatttggtacttcc

SEQ ID NO: 625 - protein, 7 aa, synthetic construct
  SITE 1: note = Residue attached to another sequence via a linker
  SITE 7: note = Residue attached to another sequence via a linker
PKKKRKV

SEQ ID NO: 626 - DNA, 21 bp, synthetic construct
  source 1..21: mol_type = other DNA
  misc_feature 21: note = nucleotide attached to another sequence via a linker
cagcagcagcagcagcagcag

SEQ ID NO: 627 - protein, 8 aa, synthetic construct
  SITE 1: note = Residue attached to another sequence via a Lysine 2-[2-[2-aminoethoxy]ethoxy]acetic acid Lysine linker
  SITE 2: note = D-amino acid
  SITE 8: note = Residue attached to another sequence via a 2-[2-[2-aminoethoxy]ethoxy]acetic acid linker
FFFGRGRE

SEQ ID NO: 628 - protein, 8 aa, synthetic construct
  SITE 1: note = Acetylated Residue
  SITE 7: note = Residue attached to another sequence via the side chain
PKKKRKVK

SEQ ID NO: 629 - protein, 8 aa, synthetic construct
  SITE 1: note = Residue attached to the side chain of another sequence
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 630 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 631 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
FFFGRGRQ

SEQ ID NO: 632 - protein, 9 aa, synthetic construct
  SITE 1: note = Acetylated Residue
  SITE 8: note = 2-[2-[2-aminoethoxy]ethoxy]acetic acid
  SITE 9: note = Residue attached to another sequence via the side chain
PKKKRKVXK

SEQ ID NO: 633 - protein, 8 aa, synthetic construct
  SITE 1: note = Residue attached to the side chain of another sequence
FGFGRGRQ

SEQ ID NO: 634 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 635 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 636 - protein, 7 aa, synthetic construct
  SITE 1: note = Acetylated Residue
  SITE 7: note = Residue attached to another sequence via a 2-[2-[2-aminoethoxy]ethoxy]acetic acid Lysine linker
PKKKRKV

SEQ ID NO: 637 - protein, 8 aa, synthetic construct
  SITE 1: note = Residue attached to another sequence via a 2-[2-[2-aminoethoxy]ethoxy]acetic acid Lysine linker
  SITE 2: note = D-amino acid
  SITE 5: note = D-amino acid
  SITE 7: note = D-amino acid
GFFGRGRQ

SEQ ID NO: 638 - DNA, 21 bp, synthetic construct
  source 1..21: mol_type = other DNA
  misc_feature 1..21: note = Phosphorodiamidate morpholino backbone
cagcagcagcagcagcagcag

SEQ ID NO: 639 - DNA, 21 bp, synthetic construct
  source 1..21: mol_type = other DNA
  misc_feature 1..21: note = Phosphorodiamidate morpholino backbone
cagcagcagcagcagcagcag

SEQ ID NO: 640 - DNA, 21 bp, synthetic construct
  source 1..21: mol_type = other DNA
  modified_base 1: mod_base = OTHER; note = Sarcosine amide modified nucleotide
  modified_base 21: mod_base = OTHER; note = C4-cyclooctyne modified nucleotide
  misc_feature 1..21: note = Phosphorodiamidate morpholino backbone
cagcagcagcagcagcagcag

SEQ ID NO: 641 - DNA, 21 bp, synthetic construct
  source 1..21: mol_type = other DNA
  modified_base 21: mod_base = OTHER; note = Secondary amine morpholino modified nucleotide
  misc_feature 1..21: note = Phosphorodiamidate morpholino backbone
cagcagcagcagcagcagcag

SEQ ID NO: 642 - DNA, 24 bp, synthetic construct
  source 1..24: mol_type = other DNA
  misc_feature 1..24: note = Phosphorodiamidate morpholino backbone
cagcagcagcagcagcagcagcag

SEQ ID NO: 643 - protein, 7 aa, synthetic construct
  SITE 1: note = Acetylated Residue
  SITE 7: note = Residue attached to the side chain of another sequence
VKRKKKP

SEQ ID NO: 644 - protein, 8 aa, synthetic construct
  SITE 1: note = Residue attached to another sequence via a linker
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 645 - protein, 7 aa, synthetic construct
  SITE 1: note = Acetylated Residue
  SITE 7: note = Residue attached to another sequence via a 2-[2-[2-aminoethoxy]ethoxy]acetic acid linker
PKKKRKV

SEQ ID NO: 646 - protein, 7 aa, synthetic construct
  SITE 1: note = Any amino acid
  VARIANT 5: note = The number of carbons in the side chain may vary
  VARIANT 7: note = The number of carbons in the side chain may vary
XFGFRGR

SEQ ID NO: 647 - protein, 8 aa, synthetic construct
  VARIANT 1: note = Any amino acid
  SITE 3: note = D-amino acid
  VARIANT 6: note = The number of carbons in the D-arginine side chain may vary
  VARIANT 8: note = The number of carbons in the D-arginine side chain may vary
XGFFGRGR

SEQ ID NO: 648 - protein, 8 aa, synthetic construct
  VARIANT 1: note = Any amino acid
  SITE 3: note = D-amino acid
  VARIANT 6: note = The number of carbons in the side chain may vary
  VARIANT 8: note = The number of carbons in the side chain may vary
XFFFGRGR

SEQ ID NO: 649 - protein, 8 aa, synthetic construct
  VARIANT 1: note = Any amino acid
  SITE 3: note = D-amino acid
  SITE 4: note = 3-(2-naphthyl)-alanine
  SITE 6: note = D-amino acid
  SITE 7: note = D-amino acid
  SITE 8: note = D-amino acid
XFFXGRGR

SEQ ID NO: 650 - protein, 8 aa, synthetic construct
  VARIANT 1: note = Any amino acid
XFGFGRRR

SEQ ID NO: 651 - protein, 8 aa, synthetic construct
  VARIANT 1: note = Any amino acid
XFGFRRRR

SEQ ID NO: 652 - protein, 7 aa, synthetic construct
QFGFRGR

SEQ ID NO: 653 - protein, 8 aa, synthetic construct
  SITE 3: note = D-amino acid
  SITE 6: note = D-amino acid
  SITE 8: note = D-amino acid
QGFFGRGR

SEQ ID NO: 654 - protein, 8 aa, synthetic construct
  VARIANT 1: note = Any amino acid
  SITE 3: note = D-amino acid
  VARIANT 6: note = The number of carbons in the side chain may vary
  VARIANT 8: note = The number of carbons in the side chain may vary
XFFFGRGR

SEQ ID NO: 655 - protein, 8 aa, synthetic construct
  SITE 3: note = D-amino acid
  SITE 4: note = 3-(2-naphthyl)-alanine
  SITE 6: note = D-amino acid
  SITE 7: note = D-amino acid
  SITE 8: note = D-amino acid
QFFXGRGR

SEQ ID NO: 656 - protein, 8 aa, synthetic construct
  VARIANT 1: note = Any amino acid
  SITE 3: note = D-amino acid
  SITE 4: note = 3-(2-naphthyl)-alanine
  VARIANT 5: note = Citrulline where the number of carbons in the side chain may vary
  VARIANT 6: note = D-amino acid where the number of carbons in the side chain may vary
  VARIANT 7: note = Citrulline where the number of carbons in the side chain may vary
  VARIANT 8: note = D-amino acid where the number of carbons in the side chain may vary
XFFXXRXR

SEQ ID NO: 657 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 658 - protein, 7 aa, synthetic construct
  SITE 1: note = Acetylated Residue
PKKKRKV

SEQ ID NO: 659 - protein, 8 aa, synthetic construct
FGFGRGRQ

SEQ ID NO: 660 - protein, 8 aa, synthetic construct
  VARIANT 1: note = Any amino acid
  SITE 3: note = D-amino acid
  SITE 4: note = 3-(2-naphthyl)-alanine
  VARIANT 5: note = Citrulline where the number of carbons in the side chain may vary
  VARIANT 6: note = D-amino acid where the number of carbons in the side chain may vary
  VARIANT 7: note = Citrulline where the number of carbons in the side chain may vary
  VARIANT 8: note = D-amino acid where the number of carbons in the side chain may vary
XFFXXRXR

SEQ ID NO: 661 - protein, 8 aa, synthetic construct
  VARIANT 1: note = Any amino acid
  SITE 3: note = D-amino acid
  SITE 4: note = 3-(2-naphthyl)-alanine
  VARIANT 5: note = Citrulline where the number of carbons in the side chain may vary
  VARIANT 6: note = D-Citrulline where the number of carbons in the side chain may vary
  VARIANT 7: note = The number of carbons in the side chain may vary
  VARIANT 8: note = D-amino acid where the number of carbons in the side chain may vary
XFFXXXRR

SEQ ID NO: 662 - protein, 8 aa, synthetic construct
  VARIANT 1: note = Any amino acid
  SITE 3: note = D-amino acid
  SITE 4: note = 3-(2-naphthyl)-alanine
  VARIANT 5: note = The number of carbons in the side chain may vary
  VARIANT 6: note = D-amino acid where the number of carbons in the side chain may vary
  VARIANT 7: note = Citrulline where the number of carbons in the side chain may vary
  VARIANT 8: note = D-Citrulline where the number of carbons in the side chain may vary
XFFXRRXX

SEQ ID NO: 663 - protein, 8 aa, synthetic construct
  VARIANT 1: note = Any amino acid
  SITE 3: note = D-amino acid
  SITE 4: note = 3-(2-naphthyl)-alanine
  VARIANT 5: note = The number of carbons in the side chain may vary
  VARIANT 6: note = D-Citrulline where the number of carbons in the side chain may vary
  VARIANT 7: note = The number of carbons in the side chain may vary
  VARIANT 8: note = D-Citrulline where the number of carbons in the side chain may vary
XFFXRXRX

SEQ ID NO: 664 - protein, 8 aa, synthetic construct
  VARIANT 1: note = Any amino acid
  SITE 3: note = D-amino acid
  SITE 4: note = 3-(2-naphthyl)-alanine
  VARIANT 5: note = Citrulline where the number of carbons in the side chain may vary
  VARIANT 6: note = D-amino acid where the number of carbons in the side chain may vary
  VARIANT 7: note = The number of carbons in the side chain may vary
  VARIANT 8: note = D-Arginine where the number of carbons in the side chain may vary
XFFXXRRR

SEQ ID NO: 665 - protein, 8 aa, synthetic construct
  VARIANT 1: note = Any amino acid
  SITE 3: note = D-amino acid
  SITE 4: note = 3-(2-naphthyl)-alanine
  VARIANT 5: note = The number of carbons in the side chain may vary
  VARIANT 6: note = D-Citrulline where the number of carbons in the side chain may vary
  VARIANT 7: note = The number of carbons in the side chain may vary
  VARIANT 8: note = D-Arginine where the number of carbons in the side chain may vary
XFFXRXRR

SEQ ID NO: 666 - protein, 8 aa, synthetic construct
  VARIANT 1: note = Any amino acid
  SITE 3: note = D-amino acid
  SITE 4: note = 3-(2-naphthyl)-alanine
  VARIANT 5: note = The number of carbons in the side chain may vary
  VARIANT 6: note = D-amino acid where the number of carbons in the side chain may vary
  VARIANT 7: note = Citrulline where the number of carbons in the side chain may vary
  VARIANT 8: note = D-Arginine where the number of carbons in the side chain may vary
XFFXRRXR

SEQ ID NO: 667 - protein, 8 aa, synthetic construct
  VARIANT 1: note = Any amino acid
  SITE 3: note = D-amino acid
  SITE 4: note = 3-(2-naphthyl)-alanine
  VARIANT 5: note = The number of carbons in the side chain may vary
  VARIANT 6: note = D-amino acid where the number of carbons in the side chain may vary
  VARIANT 7: note = The number of carbons in the side chain may vary
  VARIANT 8: note = D-Citrulline where the number of carbons in the side chain may vary
XFFXRRRX

SEQ ID NO: 668 - protein, 8 aa, synthetic construct
  SITE 3: note = D-amino acid
  SITE 4: note = 3-(2-naphthyl)-alanine
  SITE 6: note = D-amino acid
  SITE 8: note = D-amino acid
  SITE 7: note = L-citrulline
EFFXRRXR

SEQ ID NO: 669 - protein, 8 aa, synthetic construct
  SITE 2: note = D-amino acid
  SITE 3: note = 3-(2-naphthyl)-alanine
  SITE 4: note = 3-(4-piperidinyl)alanine
  SITE 5: note = D-amino acid
  SITE 6: note = 3-(4-piperidinyl)alanine
  SITE 7: note = D-amino acid
FFXXRXRE

SEQ ID NO: 670 - protein, 8 aa, synthetic construct
  SITE 2: note = D-amino acid
  SITE 3: note = 3-(2-naphthyl)-alanine
  SITE 4: note = N,N-dimethyl lysine
  SITE 5: note = D-amino acid
  SITE 6: note = N,N-dimethyl lysine
  SITE 7: note = D-amino acid
FFXKRKRE

SEQ ID NO: 671 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRE

SEQ ID NO: 672 - protein, 8 aa, synthetic construct
QFGFGRGR

SEQ ID NO: 673 - protein, 8 aa, synthetic construct
  SITE 3: note = D-amino acid
  SITE 6: note = D-amino acid
  SITE 8: note = D-amino acid
QGFFGRGR

SEQ ID NO: 674 - protein, 8 aa, synthetic construct
  SITE 3: note = D-amino acid
  VARIANT 6: note = The number of carbons in the side chain may vary
  VARIANT 8: note = The number of carbons in the side chain may vary
QFFFGRGR

SEQ ID NO: 675 - protein, 8 aa, synthetic construct
  SITE 3: note = D-amino acid
  SITE 4: note = 3-(2-naphthyl)-alanine
  SITE 6: note = D-amino acid
  SITE 8: note = D-amino acid
QFFXGRGR

SEQ ID NO: 676 - protein, 8 aa, synthetic construct
  SITE 2: note = D-amino acid
FFFGRGRE

SEQ ID NO: 677 - protein, 7 aa, synthetic construct
  SITE 1: note = Acetylated Residue
PKKKRKV

SEQ ID NO: 678 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 679 - protein, 7 aa, synthetic construct
  SITE 1: note = Acetylated Residue
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
PKKKRKV

SEQ ID NO: 680 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 681 - protein, 7 aa, synthetic construct
  SITE 1: note = Acetylated Residue
PKKKRKV

SEQ ID NO: 682 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 683 - protein, 7 aa, synthetic construct
  SITE 1: note = Acetylated Residue
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
PKKKRKV

SEQ ID NO: 684 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 685 - protein, 7 aa, synthetic construct
  SITE 1: note = Acetylated Residue
PKKKRKV

SEQ ID NO: 686 - protein, 8 aa, synthetic construct
  SITE 5: note = D-amino acid
  SITE 7: note = D-amino acid
FGFGRGRQ

SEQ ID NO: 687 - protein, 7 aa, synthetic construct
  SITE 1: note = Acetylated Residue
PKKKRKV

SEQ ID NO: 688 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 689 - protein, 7 aa, synthetic construct
  SITE 1: note = Acetylated Residue
PKKKRKV

SEQ ID NO: 690 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXRRRRQ

SEQ ID NO: 691 - protein, 7 aa, synthetic construct
  SITE 1: note = Acetylated Residue
PKKKRKV

SEQ ID NO: 692 - protein, 8 aa, synthetic construct
  SITE 2: note = D-amino acid
FFFGRGRQ

SEQ ID NO: 693 - protein, 7 aa, synthetic construct
  SITE 1: note = Acetylated Residue
PKKKRKV

SEQ ID NO: 694 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 695 - protein, 7 aa, synthetic construct
  SITE 1: note = Acetylated Residue
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
PKKKRKV

SEQ ID NO: 696 - protein, 8 aa, synthetic construct
  SITE 2: note = D-amino acid
  SITE 5: note = D-amino acid
  SITE 7: note = D-amino acid
GFFGRGRQ

SEQ ID NO: 697 - protein, 7 aa, synthetic construct
  SITE 1: note = Acetylated Residue
PKKKRKV

SEQ ID NO: 698 - protein, 8 aa, synthetic construct
  SITE 2: note = D-amino acid
  SITE 5: note = D-amino acid
  SITE 7: note = D-amino acid
GFFGRGRQ

SEQ ID NO: 699 - protein, 7 aa, synthetic construct
  SITE 1: note = Acetylated Residue
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
PKKKRKV

SEQ ID NO: 700 - protein, 8 aa, synthetic construct
FGFGRRRQ

SEQ ID NO: 701 - protein, 7 aa, synthetic construct
  SITE 1: note = Acetylated Residue
PKKKRKV

SEQ ID NO: 702 - protein, 8 aa, synthetic construct
FGFGRRRQ

SEQ ID NO: 703 - protein, 7 aa, synthetic construct
  SITE 1: note = Acetylated Residue
  SITE 2: note = Trifluoroacetyl lysine
  SITE 3: note = Trifluoroacetyl lysine
  SITE 4: note = Trifluoroacetyl lysine
  SITE 6: note = Trifluoroacetyl lysine
PKKKRKV

SEQ ID NO: 704 - protein, 8 aa, synthetic construct
FGFRRRRQ

SEQ ID NO: 705 - protein, 8 aa, synthetic construct
FGFGRGRQ

SEQ ID NO: 706 - protein, 8 aa, synthetic construct
  SITE 2: note = D-amino acid
  SITE 7: note = D-amino acid
  SITE 5: note = D-amino acid
GFFGRGRQ

SEQ ID NO: 707 - protein, 8 aa, synthetic construct
  SITE 2: note = D-amino acid
  VARIANT 5: note = The number of carbons in the side chain may vary
  VARIANT 7: note = The number of carbons in the side chain may vary
FFFGRGRQ

SEQ ID NO: 708 - protein, 8 aa, synthetic construct
  SITE 2: note = D-amino acid
  SITE 3: note = 3-(2-naphthyl)-alanine
  SITE 5: note = D-amino acid
  SITE 7: note = D-amino acid
FFXGRGRQ

SEQ ID NO: 709 - DNA, 23 bp, synthetic construct
  source 1..23: mol_type = other DNA
  misc_feature 1..23: note = Phosphorodiamidate morpholino backbone
aaacgccgccatttctcaacaga

SEQ ID NO: 710 - protein, 7 aa, synthetic construct
  SITE 1: note = Acetylated Residue
PKKKRKV

SEQ ID NO: 711 - protein, 10 aa, synthetic construct
FGFGRGRGRQ

SEQ ID NO: 712 - protein, 8 aa, synthetic construct
  SITE 6: note = D-amino acid
FGFGRRRQ

SEQ ID NO: 713 - protein, 6 aa, synthetic construct
KKKRKG